(19) United States
(12) Patent Application Publication
(10) Pub. No.: US 2025/0009811 A1
(43) Pub. Date: Jan. 9, 2025

(54) COMPOSITIONS AND METHODS FOR SUPPRESSING PATHOGENIC ORGANISMS

(71) Applicant: Vedanta Biosciences, Inc., Cambridge, MA (US)

(72) Inventors: Silvia Caballero, Cambridge, MA (US); Cintia Felix, Nashua, NH (US)

(73) Assignee: Vedanta Biosciences, Inc., Cambridge, MA (US)

(21) Appl. No.: 18/748,761

(22) Filed: Jun. 20, 2024

Related U.S. Application Data
(63) Continuation of application No. 16/771,075, filed on Jun. 9, 2020, now Pat. No. 12,053,495.
(60) Provisional application No. 62/769,461, filed on Nov. 19, 2018.
(60) Provisional application No. 62/703,917, filed on Jul. 27, 2018.
(60) Provisional application No. 62/643,554, filed on Mar. 15, 2018.
(60) Provisional application No. 62/626,908, filed on Feb. 6, 2018.
(60) Provisional application No. 62/616,394, filed on Jan. 11, 2018.
(60) Provisional application No. 62/596,988, filed on Dec. 11, 2017.

(51) Int. Cl. A61K 35/74; A61K 9/00; A61K 35/742; A61K 35/744; A61K 35/745; A61K 35/747; A61P 31/04

(52) U.S. Cl. CPC A61K 35/74; A61K 9/0031; A61K 9/0053; A61K 35/742; A61K 35/744; A61K 35/745; A61K 35/747; A61P 31/04

(57) ABSTRACT

Provided herein are compositions and methods for the suppression of multi-drug resistant organisms. Provided herein are compositions and methods for treating diseases or disorders associated with bacterial colonization or treating diseases or disorders associated with an immune response induced by bacteria. Also provided herein are compositions and methods for suppressing colonization of the intestine of subject with oral microbiome bacteria.

### RELATED APPLICATIONS

This application is a continuation of U.S. application Ser. No. 16/771,075, which is a national stage filing under 35 U.S.C § 371 of international PCT application, PCT/US2018/065031, filed Dec. 11, 2018, which claims the benefit under 35 U.S.C. § 119(e) to U.S. provisional application No. 62/596,988, filed Dec. 11, 2017; U.S. provisional application No. 62/616,394, filed Jan. 11, 2018; U.S. provisional application No. 62/626,908, filed Feb. 6, 2018; U.S. provisional application No. 62/643,554, filed Mar. 15, 2018; U.S. provisional application No. 62/703,917, filed Jul. 27, 2018; and U.S. provisional application No. 62/769,461, filed Nov. 19, 2018. The entire contents of each of these referenced applications are incorporated by reference herein.

### REFERENCE TO AN ELECTRONIC SEQUENCE LISTING

The contents of the electronic sequence listing (P074570014US07-SEQ-NTJ.xml; Size: 192,524 bytes; and Date of Creation: Jun. 13, 2024) is herein incorporated by reference in their entirety.

### FIELD OF INVENTION

Provided herein are compositions and methods for suppressing multi-resistant organisms. Provided herein are compositions and methods for treating diseases or disorders associated with bacterial colonization or treating diseases or disorders associated with an immune response induced by bacteria. Also provided herein are compositions and methods for suppressing oral bacterial colonization of the intestine of subject.

### BACKGROUND OF THE INVENTION

Multidrug resistant organisms (MDROs; “superbugs”), microorganisms that have developed resistance to one or more classes of antimicrobial agents, such as antibiotics, are emerging as serious global health threat. It is estimated that over 2 million people in the United States contract serious bacterial infections that are resistant to one or more antibiotics each year (CDC, Antibiotic Resistance Threats in the United States, 2013. Publication No. CS239559-B). Treatment options for subjects with MDROs are extremely limited; prevention of transmission is critical. The most important factor contributing to the generation and propagation of MDROs is the use and overuse/misuse of antibiotics and it is thought that the problem will increase in severity as further pathogenic organisms with antibiotic resistance arise (CDC, 2013; WHO 2017).

### SUMMARY OF THE INVENTION

Provided herein are compositions and methods for suppressing multi-drug resistant organisms in a subject.

According to one aspect, compositions are provided that include two or more purified bacterial strains of species selected from the group consisting of Alistipes putredinis, Bacteroides uniformis, Bacteroides vulgatus, Bifidobacterium longum, Bifidobacterium adolescentis, Blautia obeum, Blautia wexlerae, Blautia producta, Clostridium hathewayi, Clostridium bolteae, Collinsella aerofaciens, Coprococcus comes, Dorea longicatena, Eubacterium halli, Faecalibacterium prausnitzii, Parabacteroides merdae, Parabacteroides distasonis, Prevotella copri, Roseburia faecis, and Ruminococcus faecis.

According to another aspect, compositions are provided that include two or more purified bacterial strains of species selected from the group consisting of Alistipes putredinis, Bacteroides uniformis, Bacteroides vulgatus, Parabacteroides merdae, Bifidobacterium longum, Bifidobacterium adolescentis, Blautia obeum, Blautia wexlerae, Clostridium bolteae, Coprococcus comes, Dorea longicatena, Eubacterium halli, Faecalibacterium prausnitzii, Parabacteroides distasonis, Prevotella copri, Roseburia faecis, and Ruminococcus faecis.

According to another aspect, compositions are provided that include two or more purified bacterial strains of species selected from the group consisting of Alistipes putredinis, Bacteroides vulgatus, Bifidobacterium longum, Blautia obeum, Blautia wexlerae, Clostridium bolteae, Parabacteroides distasonis, Collinsella aerofaciens, Coprococcus comes, Dorea longicatena, Eubacterium halli, Faecalibacterium prausnitzii, Parabacteroides merdae, Prevotella copri, Roseburia faecis, and Ruminococcus faecis.

According to another aspect, compositions are provided that include two or more purified bacterial strains of species selected from the group consisting of Bacteroides uniformis, Bacteroides vulgatus, Parabacteroides merdae, Bifidobacterium longum, Bifidobacterium adolescentis, Blautia obeum, Blautia wexlerae, Clostridium bolteae, Dorea longicatena, Eubacterium halli, Faecalibacterium prausnitzii, Parabacteroides distasonis, Prevotella copri, and Ruminococcus faecis.

According to another aspect, compositions are provided that include two or more purified bacterial strains of species selected from the group consisting of Bacteroides vulgatus, Parabacteroides merdae, Bifidobacterium longum, Blautia obeum, Blautia producta, Clostridium hathewayi, Clostridium bolteae, Dorea longicatena, Eubacterium halli, Faecalibacterium prausnitzii, Parabacteroides distasonis, Prevotella copri, and Ruminococcus faecis.

According to another aspect, compositions are provided that include two or more purified bacterial strains of species selected from the group consisting of Bacteroides uniformis, Bacteroides vulgatus, Bifidobacterium adolescentis, Blautia producta, Clostridium hathewayi, Clostridium innocuum, Dorea longicatena, Eubacterium halli, Faecalibacterium prausnitzii, Parabacteroides merdae, Prevotella copri, and Ruminococcus faecis.

According to another aspect, compositions are provided that include two or more purified bacterial strains of species selected from the group consisting of Bacteroides vulgatus, Parabacteroides merdae, Bifidobacterium longum, Blautia obeum, Dorea longicatena, Eubacterium halli, Faecalibacterium prausnitzii, Parabacteroides distasonis, and Ruminococcus faecis.

According to another aspect, compositions are provided that include two or more purified bacterial strains of species selected from the group consisting of Bacteroides vulgatus, Bifidobacterium adolescentis, Blautia obeum, Blautia wexlerae, Clostridium bolteae, Parabacteroides distasonis, Collinsella aerofaciens, Eubacterium halli, Parabacteroides merdae, and Prevotella copri.

According to another aspect, compositions are provided that include two or more purified bacterial strains of species selected from the group consisting of Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, Bacteroides cellulosyliticus, Eubacterium fissicatena, Lachnospiraceae bacterium, Escherichia coli, Lactococcus lactus, Lactobacillus ruminis, Lactobacillus animalis, or Lactobacillus rhamnosus.

According to another aspect, compositions are provided that include two or more purified bacterial strains of species selected from the group consisting of Escherichia coli, Lactococcus lactus, Lactobacillus ruminis, Lactobacillus animalis, or Lactobacillus rhamnosus.

According to another aspect, compositions are provided that include two or more purified bacterial strains of species selected from the group consisting of Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, or Bacteroides cellulosyliticus.

According to another aspect, compositions are provided that include two or more purified bacterial strains of species selected from the group consisting of Eubacterium fissicatena, Lachnospiraceae bacterium, Escherichia coli, Lactococcus lactus, Lactobacillus ruminis, Lactobacillus animalis, or Lactobacillus rhamnosus.

According to another aspect, compositions are provided that include two or more purified bacterial strains, wherein the two or more purified bacterial strains comprise 16S rDNA sequences having at least 97% sequence identity with nucleic acid sequences selected from the group consisting of SEQ ID NOs: 1-22.

According to another aspect, compositions are provided that include two or more purified bacterial strains, wherein the two or more purified bacterial strains comprise 16S rDNA sequences having at least 97% sequence identity with nucleic acid sequences selected from the group consisting of SEQ ID NOs:1-8, 11, 12, 14-17, and 19-22.

According to another aspect, compositions are provided that include two or more purified bacterial strains, wherein the two or more purified bacterial strains comprise 16S rDNA sequences having at least 97% sequence identity with nucleic acid sequences selected from the group consisting of SEQ ID NOs: 1, 3-5, 7, 8, 11, 13-18, and 20-22.

According to another aspect, compositions are provided that include two or more purified bacterial strains, wherein the two or more purified bacterial strains comprise 16S rDNA sequences having at least 97% sequence identity with nucleic acid sequences selected from the group consisting of SEQ ID NOs: 2-8, 11, 12, 15-17, 19, 20, and 22.

According to another aspect, compositions are provided that include two or more purified bacterial strains, wherein the two or more purified bacterial strains comprise 16S rDNA sequences having at least 97% sequence identity with nucleic acid sequences selected from the group consisting of SEQ ID NOs: 3-5, 7, 9-11, 15-17, 19, 20, and 22.

According to another aspect, compositions are provided that include two or more purified bacterial strains, wherein the two or more purified bacterial strains comprise 16S rDNA sequences having at least 97% sequence identity with nucleic acid sequences selected from the group consisting of SEQ ID NOs: 2, 3, 6, 9, 10, 12, 15-18, 20, and 22.

According to another aspect, compositions are provided that include two or more purified bacterial strains, wherein the two or more purified bacterial strains comprise 16S rDNA sequences having at least 97% sequence identity with nucleic acid sequences selected from the group consisting of SEQ ID NOs: 3-5, 7, 12, 15-17, 19, and 22 According to another aspect, compositions are provided that include two or more purified bacterial strains, wherein the two or more purified bacterial strains comprise 16S rDNA sequences having at least 97% sequence identity with nucleic acid sequences selected from the group consisting of SEQ ID NOs: 3, 6-8, 11-13, 16, 18, and 20.

According to another aspect, compositions are provided that include two or more purified bacterial strains of species selected from the group consisting of Bifidobacterium longum, Bifidobacterium adolescentis, Blautia wexlerae, Bacteroides vulgatus, Bacteroides uniformis, Collinsella aerofaciens, Faecalibacterium prausnitzii, Blautia obeum, Parabacteroides merdae, Parabacteroides distasonis, Roseburia faecis, Coprococcus comes, Dorea longicatena, and Eubacterium hallii.

According to another aspect, compositions are provided that include one or more purified bacterial strains of species selected from the group consisting of Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, Bacteroides cellulosyliticus, Eubacterium fissicatena, and Lachnospiraceae bacterium.

According to another aspect, compositions are provided that include one or more purified bacterial strains of species selected from the group consisting of Flavonifractor plautii, Blautia producta, Clostridium ramosum, Barnesiella spp, and Clostridium symbiosum.

According to another aspect, compositions are provided that consist of one or more purified bacterial strains of species selected from the group consisting of Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, Bacteroides cellulosyliticus, Eubacterium fissicatena, and Lachnospiraceae bacterium.

According to another aspect, compositions are provided that consist of one or more purified bacterial strains of species selected from the group consisting of Flavonifractor plautii, Blautia producta, and Clostridium ramosum.

According to another aspect, compositions are provided that comprise two or more purified bacterial strains of species selected from the group consisting of Bacteroides caccae, Bacteroides intestinalis (Bacteroides cellulosyticus), Bacteroidesfaecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Clostridiales bacterium VE202-06 (Blautia producta, Blautia coccoides), Clostridium citroniae, Clostridium sp. C105KS014 (Clostridium clostridioforme), Clostridiales bacterium VE202-21 (Eubacterium contortum, Clostridium innocuum), Erysipelotrichaceae bacterium 6_1_45 (Clostridium innocuum), Paeniclostridium sordellii, Coprococcus comes, Dorea longicatena, Erysipelatoclostridium ramosum, Eubacterium rectale, Odoribacter sp. UNK.MGS-12, Bacteroides sp. 1_1_14 (Parabacteroides merdae), Bacteroides sp. UNK.MGS-14 (Parabacteroides merdae), Bacteroides xylanisolvens, Blautia obeum, Alistipes putredinis, Collinsella aerofaciens, Eubacterium hallii, Alistipes shahii, Anaerostipes caccae, hascolarctobacterium faecis, Agathobaculum, Bacteroides sp. 2_1_56FAA (Bacteroides. fragilis), Fusobacterium mortiferum, Paraclostridium bifermentans, and Escherichia sp. 3_2_53FAA.

According to another aspect, compositions are provided that comprise two or more purified bacterial strains, wherein the two or more purified bacterial strains comprise 16S rDNA sequences having at least 97% sequence identity with nucleic acid sequences selected from the group consisting of SEQ ID NOs: 42-77.

According to another aspect, compositions are provided that comprise two or more purified bacterial strains of species selected from the group consisting of Bacteroides caccae, Bacteroides intestinalis (Bacteroides cellulosyticus), Bacteroides faecis, Bacteroides ovatus, Bacteroides thetaiotaomicron, Bacteroides uniformis, Bacteroides vulgatus, Clostridiales bacterium VE202-06 (Blautia producta, Blautia coccoides), Clostridium citroniae, Clostridium sp. C105KS014 (Clostridium clostridioforme), Clostridiales bacterium VE202-21 (Eubacterium contortum, Clostridium innocuum), Erysipelotrichaceae bacterium 6_1_45 (Clostridium innocuum), Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, Fusobacterium mortiferum, and Escherichia sp. 3_2_53FAA

According to another aspect, compositions are provided that comprise two or more purified bacterial strains comprise 16S rDNA sequences having at least 97% sequence identity with nucleic acid sequences selected from the group consisting of 42-48, 52-56, 58-61, 65-67, 69, 70, 75, and 77.

According to another aspect, compositions are provided that include at least the 5, 10, 20, 23, or 36 most abundant bacterial strains present in a spore forming fraction of a fecal sample obtained from a subject. According to another aspect, compositions are provided that include at least the 5, 10, 20, 23, or 36 most abundant bacterial species present in a spore forming fraction of a fecal sample obtained from a subject.

According to another aspect, compositions are provided that include at least the 5, 10, 20, 23, or 36 most abundant bacterial strains present in a non-spore forming fraction of a fecal sample obtained from a subject. According to another aspect, compositions are provided that include at least the 5, 10, 20, 23, or 36 most abundant bacterial species present in a non-spore forming fraction of a fecal sample obtained from a subject.

According to another aspect, compositions are provided that include at least 5, 10, 20, 23, or 36 bacterial strains present in a spore forming fraction of a fecal sample obtained from a subject, wherein the bacterial species suppress the replication, survival, and/or colonization of one or more pathogenic organisms. According to another aspect, compositions are provided that include at least 5, 10, 20, 23, or 36 bacterial species present in a spore forming fraction of a fecal sample obtained from a subject, wherein the bacterial species suppress the replication, survival, and/or colonization of one or more pathogenic organisms.

According to another aspect, compositions are provided that include at least 5, 10, 20, 23, or 36 bacterial strains present in a non-spore forming fraction of a fecal sample obtained from a subject, wherein the bacterial species suppress the replication, survival, and/or colonization of one or more pathogenic organisms. According to another aspect, compositions are provided that include at least 5, 10, 20, 23, or 36 bacterial species present in a non-spore forming fraction of a fecal sample obtained from a subject, wherein the bacterial species suppress the replication, survival, and/or colonization of one or more pathogenic organisms.

In some embodiments, the foregoing compositions include bacterial strains that originate from more than one human donor.

In some embodiments, the foregoing compositions include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or at least 22 purified bacterial strains.

In some embodiments, the foregoing compositions suppress the replication, survival, and/or colonization of one or more pathogenic organisms. In some embodiments, the pathogenic organism is susceptible to antibiotics. In some embodiments, the pathogenic organism is resistant to antibiotics. In some embodiments, the pathogenic organism is a multi-drug resistant organism. In some embodiments, the multi-drug resistant organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus. In some embodiments, the pathogenic organism is an oral microbiome bacteria.

In some embodiments, the foregoing compositions suppress the replication, survival, and/or colonization of the intestine by one or more bacteria associated with induction of a Th1 immune response.

In some embodiments, the foregoing compositions suppress the replication, survival, and/or colonization of the intestine by one or more oral microbiome bacteria.

In some embodiments, in the foregoing compositions the bacterial strains are lyophilized. In some embodiments, in the foregoing compositions the bacterial strains are spray-dried.

According to another aspect, pharmaceutical compositions are provided that include any of the foregoing compositions and further include a pharmaceutically acceptable excipient. In some embodiments, the pharmaceutical composition is formulated for oral delivery. In some embodiments, the pharmaceutical composition is formulated for rectal delivery. In some embodiments, any of the foregoing pharmaceutical compositions is formulated for delivery to the intestine. In some embodiments, any of the foregoing pharmaceutical compositions is formulated for delivery to the colon.

According to another aspect, food products are provided that include any of the foregoing compositions and a nutrient.

According to another aspect, methods of suppressing a pathogenic organism in a subject are provided. The methods include administering to the subject a therapeutically effective amount of any of the foregoing compositions, pharmaceutical compositions or food products. In some embodiments, the pathogenic organism is susceptible to antibiotics. In some embodiments, the pathogenic organism is resistant to antibiotics. In some embodiments, the pathogenic organism is Clostridium difficile. In some embodiments, the pathogenic organism is a multi-drug resistant organism.

In some embodiments, of the foregoing methods of suppressing a pathogenic organism the pathogenic organism is an oral microbiome bacteria. In some embodiments, the oral microbiome bacteria is pathogenic when it is present in the intestine.

In some embodiments of the foregoing methods of suppressing a pathogenic organism, the pathogenic organism is Klebsiella pneumoniae. In some embodiments, the Klebsiella pneumoniae is multi-drug resistant. In some embodiments, the multi-drug resistant Klebsiella pneumoniae is carbapenem-resistant Klebsiella pneumoniae. In some embodiments, the Klebsiella pneumoniae induces a Th1 response. In some embodiments, the Klebsiella pneumoniae is strain BAA-2552, strain KP-1, strain 700721, strain 13882, strain 34E1, strain BAA-1705, strain 700603, or strain Kp-2H7. In some embodiments, the Klebsiella pneumoniae is strain Kp-2H7.

In some embodiments of the foregoing methods of suppressing a pathogenic organism, the subject is human. In some embodiments of the foregoing methods of suppressing a pathogenic organism, the composition is administered to the subject more than once. In some embodiments of the foregoing methods of suppressing a pathogenic organism, the composition is administered to the subject by oral administration. In some embodiments of the foregoing methods of suppressing a pathogenic organism, the composition is administered to the subject by rectal administration. In some embodiments of the foregoing methods of suppressing a pathogenic organism, the administering suppresses the replication, survival, and/or colonization of the pathogenic organism.

In some embodiments of the foregoing methods of suppressing a pathogenic organism, the pathogenic organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus.

In some embodiments of the foregoing methods of suppressing a pathogenic organism, the methods also include administering one or more additional compositions comprising bacteria.

According to another aspect, methods of treating a disease or disorder associated with bacterial colonization in a subject are provided. The methods include administering to the subject a therapeutically effective amount of any of the foregoing compositions, pharmaceutical compositions or food products. In some embodiments, the disease or disorder is associated with a Th1 immune response.

In some embodiments of the foregoing methods of treating a disease or disorder associated with bacterial colonization, the bacterial colonization induces a Th1 immune response in the subject.

In some embodiments of the foregoing methods of treating a disease or disorder associated with bacterial colonization, the disease or disorder is an autoimmune disease or an inflammatory bowel disorder. In some embodiments, the inflammatory bowel disorder is ulcerative colitis or Crohn's disease.

In some embodiments of the foregoing methods of treating a disease or disorder associated with bacterial colonization, the disease or disorder is non-alcoholic steatohepatitis (NASH), primary sclerosing cholangitis (PSC), non-alcoholic fatty liver disease (NAFLD), gastroesophageal reflux disease (GERD), or alcoholism.

In some embodiments of the foregoing methods of treating a disease or disorder associated with bacterial colonization, the subject has a disease or disorder associated with use of a proton pump inhibitor.

In some embodiments of the foregoing methods of treating a disease or disorder associated with bacterial colonization, the subject is human. In some embodiments of the foregoing methods of treating a disease or disorder associated with bacterial colonization, the composition is administered to the subject more than once. In some embodiments of the foregoing methods of treating a disease or disorder associated with bacterial colonization, the composition is administered to the subject by oral administration. In some embodiments of the foregoing methods of treating a disease or disorder associated with bacterial colonization, the composition is administered to the subject by rectal administration. In some embodiments of the foregoing methods of treating a disease or disorder associated with bacterial colonization, the administering suppresses the replication, survival, and/or colonization of the bacteria.

In some embodiments of the foregoing methods of treating a disease or disorder associated with bacterial colonization, the bacteria is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, Clindamycin resistant Group B Streptococcus, Clostridium difficile, multi-drug resistant Klebsiella pneumoniae, carbapenem-resistant Klebsiella pneumoniae, Klebsiella pneumoniae strain BAA-2552, Klebsiella pneumoniae strain KP-1, Klebsiella pneumoniae strain 700721, Klebsiella pneumoniae strain 13882, Klebsiella pneumoniae strain 34E1, Klebsiella pneumoniae strain BAA-1705, Klebsiella pneumoniae strain 700603, or Klebsiella pneumoniae strain Kp-2H7.

In some embodiments of the foregoing methods of treating a disease or disorder associated with bacterial colonization, the methods also include administering one or more additional compositions comprising bacteria.

According to another aspect, methods of treating a disease or disorder associated with an immune response induced by bacteria in a subject are provided. The methods include administering to the subject a therapeutically effective amount of any of the foregoing compositions, pharmaceutical compositions or food products.

In some embodiments of the foregoing methods of treating a disease or disorder associated with an immune response induced by bacteria, the subject is human.

In some embodiments of the foregoing methods of treating a disease or disorder associated with an immune response induced by bacteria, the composition is administered to the subject more than once. In some embodiments of the foregoing methods of treating a disease or disorder associated with an immune response induced by bacteria, the composition is administered to the subject by oral administration. In some embodiments of the foregoing methods of treating a disease or disorder associated with an immune response induced by bacteria, the composition is administered to the subject by rectal administration. In some embodiments of the foregoing methods of treating a disease or disorder associated with an immune response induced by bacteria, the administering suppresses the replication, survival, and/or colonization of the bacteria.

In some embodiments of the foregoing methods of treating a disease or disorder associated with an immune response induced by bacteria, the bacteria is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, Clindamycin resistant Group B Streptococcus, Clostridium difficile, multi-drug resistant Klebsiella pneumoniae, carbapenem-resistant Klebsiella pneumoniae, Klebsiella pneumoniae strain BAA-2552, Klebsiella pneumoniae strain KP-1, Klebsiella pneumoniae strain 700721, Klebsiella pneumoniae strain 13882, Klebsiella pneumoniae strain 34E1, Klebsiella pneumoniae strain BAA-1705, Klebsiella pneumoniae strain 700603, or Klebsiella pneumoniae strain Kp-2H7.

In some embodiments of the foregoing methods of treating a disease or disorder associated with an immune response induced by bacteria, the methods also include administering one or more additional compositions comprising bacteria.

According to another aspect, methods of suppressing colonization of the intestine of a subject with oral microbiome bacteria are provided. The methods include administering to the subject a therapeutically effective amount of any of the foregoing compositions, pharmaceutical compositions or food products.

In some embodiments of the foregoing methods of suppressing colonization of the intestine of a subject with oral microbiome bacteria, the subject is human.

In some embodiments of the foregoing methods of suppressing colonization of the intestine of a subject with oral microbiome bacteria, the composition is administered to the subject more than once.

In some embodiments of the foregoing methods of suppressing colonization of the intestine of a subject with oral microbiome bacteria, the composition is administered to the subject by oral administration.

In some embodiments of the foregoing methods of suppressing colonization of the intestine of a subject with oral microbiome bacteria, the composition is administered to the subject by rectal administration.

In some embodiments of the foregoing methods of suppressing colonization of the intestine of a subject with oral microbiome bacteria, the administering suppresses the replication, survival, and/or colonization of the bacteria.

In some embodiments of the foregoing methods of suppressing colonization of the intestine of a subject with oral microbiome bacteria, the oral microbiome bacteria is Carbapenem Resistant Enterobacteriaceae (CRE), Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, Clindamycin resistant Group B Streptococcus, multi-drug resistant Klebsiella pneumoniae, carbapenem-resistant Klebsiella pneumoniae, Klebsiella pneumoniae strain BAA-2552, Klebsiella pneumoniae strain KP-1, Klebsiella pneumoniae strain 700721, Klebsiella pneumoniae strain 13882, Klebsiella pneumoniae strain 34E1, Klebsiella pneumoniae strain BAA-1705, Klebsiella pneumoniae strain 700603, or Klebsiella pneumoniae strain Kp-2H7.

In some embodiments of the foregoing methods of suppressing colonization of the intestine of a subject with oral microbiome bacteria, the methods also include administering one or more additional compositions comprising bacteria.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIGS. 1A-1E show a mouse model of mono- and co-colonization of VRE and CRE. FIG. 1A presents the timeline of the mouse model of colonization. FIG. 1B shows CFU levels of CRE in CRE mono-colonized mice. FIG. 1C shows CFU levels of VRE in VRE mono-colonized mice. FIG. 1D shows CFU levels of CRE in CRE and VRE co-colonized mice. FIG. 1E shows CFU levels of VRE in CRE and VRE co-colonized mice.

FIGS. 2A and 2B show FMT samples from a donor reduce the CRE burden in colonized mice. FIG. 2A presents the experimental timeline. FIG. 2B shows K. pneumoniae CFU levels in mice at day 18 post fecal matter treatment (FMT).

FIGS. 3A and 3B show stool fractions from a donor reduce the CRE burden in colonized mice. FIG. 3A presents the experimental timeline. FIG. 3B shows K. pneumoniae CFU levels in mice at day 18 following the first fecal matter/stool fraction treatment. Stool fractions were enriched in non-spore forming (“NSP”) or spore-forming (“SP”) bacteria.

FIG. 4 is a table showing the bacterial strains present in each of live bacterial products (LBP) 1-7 tested.

FIGS. 5A-5D show the K. pneumoniae CFU levels in mice feces following administration of the indicated LBP or FMT. FIG. 5A shows K. pneumoniae CFU levels on day 0 (“D0”) prior to administration of the indicated LBP or FMT. FIG. 5B shows K. pneumoniae CFU levels on day 4 (“D4”) following administration of the indicated LBP or FMT. FIG. 5C shows K. pneumoniae CFU levels on day 7 (“D7”) following administration of the indicated LBP or FMT. FIG. 5D shows K. pneumoniae CFU levels on day 12 (“D12”) following administration of the indicated LBP or FMT.

FIG. 6 shows the K. pneumoniae levels in fecal samples of decolonized mice on day 26 post fecal matter treatment (“D26pFMT”), prior to antibiotic treatment (“pre abx DO”, corresponding to day 35 post fecal matter treatment (“D35pFMT”)), day 7 (“D7”) following antibiotic treatment, and days 11-15 (“D11-15”) following antibiotic treatment. Ampicillin (“Amp”) was administered from day 0 to day 7.

FIG. 7 is a table presenting bacterial strains identified in stool samples from donors and present in the live bacteria products shown in FIG. 4.

FIG. 8 shows examples of a soft agar overlay assay to identify strains having pathogen-antagonistic activity against CRE.

FIG. 9 is a table presenting results from a soft agar overlay assay demonstrated in FIG. 8. Bacterial strains having strong, medium, weak, or no detectable antagonistic activity towards CRE are shown.

FIG. 10 shows an example agar overlay screen to identify pathogen-antagonistic strains.

FIG. 11 show a schematic depicting a genome alignment between two strains of the Blautia producta species, a first strain having pathogen-antagonizing activity (Strain 2) and a second strain of the same bacterial species that does not have pathogen-antagonizing activity (Strain 10). The alignment identified a region of differential homology encoding bacteriocin-associated genes that were present in Strain 2 but absent in Strain 10.

FIGS. 12A and 12B show FMT samples from a donor reduce the VRE burden in colonized mice. FIG. 12A presents the experimental timeline. FIG. 12B shows VRE CFU levels in mice at the days 25-28 post fecal matter treatment.

FIGS. 13A and 13B show stool fractions from a donor reduce the VRE burden in colonized mice. FIG. 13A presents the experimental time line. FIG. 13B shows VRE CFU levels in mice at day 25 following the three doses of the fecal matter/stool fraction treatment. Stool fractions were enriched in non-spore forming (“NSP”) or spore-forming (“SP”) bacteria.

FIG. 14 shows VRE CFU levels in fecal samples of mice prior to antibiotic treatment (“pre abx D0”, corresponding to day 43 post fecal matter treatment (“D43PFMT”)), day 3 (“D3”) following antibiotic treatment, and day 7 (“D7”) following antibiotic treatment. Ampicillin was administered from day 0 to day 7. Mice that were not decolonized are labeled with identification numbers at each of the time points.

FIG. 15 shows VRE CFU levels in fecal samples from mice that previously cleared VRE and were re-challenged with VRE. VRE CFU levels are shown prior the second challenge with VRE (day 0, corresponding to day 50 post fecal matter treatment “d50 post FMT”) and three days after the second challenge (“d3”).

FIG. 16 shows examples of a soft agar overlay assay to identify strains with pathogen-antagonistic activity against VRE.

FIG. 17 is a table presenting results from a soft agar overlay assay depicted in FIG. 16. Bacterial strains having strong or no antagonistic activity towards VRE are shown.

FIG. 18 shows a work-flow diagram of a broth-based competition assay, as described in Example 5.

FIGS. 19 and 20 show the ability of the indicated bacterial strains to suppress CRE growth (L.o.D. is Limit of Detection).

FIG. 21 shows a work-flow diagram of a broth-based competition assay with optical detection, as described in Example 5.

FIG. 22 shows the ability of the indicated bacterial strains to suppress CRE growth (RFU is Relative Fluorescence Unit).

FIGS. 23A and 23B show FMT and stool fractions enriched in non-spore-forming (“NSP”) bacteria from a healthy donor (donor 4, also referred to as “D14”) reduce the CRE burden in colonized mice. FIG. 23A presents the experimental timeline. FIG. 23B shows K. pneumoniae CFU levels in mice at the indicated time points.

FIG. 24 shows CRE CFU levels in fecal samples from mice that previously cleared CRE and were re-challenged with CRE. CRE CFU levels are shown prior to the second challenged with CRE (day 0, corresponding to day 29 post stool fraction treatment/fecal matter treatment (“D29 post SFL/FMT”) and three days after the second challenge (“D3”).

FIGS. 25A-25C show the ability of the indicated bacterial strains to suppress growth of different CRE strains. FIG. 25A shows inhibition of K. pneumoniae strain ATCC BAA-2814 (KPC). FIG. 25B shows inhibition of K. pneumoniae strain ATCC BAA-1705 (KPC). FIG. 25C shows inhibition of K. pneumoniae strain ATCC BAA-2146 (NDM-1). L.o.D. is Limit of Detection.

FIGS. 26A-26C show the efficacy of FMT against CRE. FIG. 26A shows inhibition of CRE by, from left-to-right for each time point: control (PBS), single doses of FMT from donors D1 (also referred to as “donor 3”), D2 (also referred to as “donor 1”), D11 (also referred to as “donor 2”), and D14 (also referred to as “donor 4”). Results are shown at 0 and 16 days post-treatment. FIG. 26B shows inhibition of CRE by, from left-to-right for each time point: single dose of FMT from donor D14 and triple dose of FMT from donor D14. Results are shown at 0 and 16 days post-treatment. FIG. 26C shows inhibition of CRE by, from left-to-right: control (PBS), single dose of FMT from donor D11, triple dose of FMT from donor D11, single dose of FMT from donor D14, triple dose of FMT from donor D14, single dose of FMT from donor D2, and triple dose of FMT from donor D2. Results are shown at 10 days post-treatment. L.o.D. is Limit of Detection.

FIG. 27 shows that a non-spore forming (“NSF”) enriched stool fraction from a donor (donor 4, “D14”) reduces the CRE and VRE CFU levels in mice at day 25 following the three doses of the fecal matter/stool fraction treatment. CRE and VRE CFU levels for control (PBS) and FMT are also shown. L.o.D. is Limit of Detection.

FIG. 28 shows the number of screened strains and number of strains in the listed bacterial genera that are active against 5 Klebsiella strains.

FIG. 29 shows non-spore forming (“NSF”) enriched stool fractions or FMT reduce the CRE burden in mice that were treated with ampicillin for 7 days and then challenged with CRE. Three days following CRE challenge, mice were administered 3 consecutive doses of control (PBS), bacterial compositions from donors (donor 3 (“D1”), donor 2, donor 5, donor 1, or donor 4 (“D14”)) or human fecal material (FMT). CRE CFU levels were quantified at 0 and 17 days post administration (“post-treatment”). At each time point, the data from left-to-right are control (PBS), donor 3 (“D1”), donor 2, donor 5, donor 1, donor 4 (“D14”), and FMT. L.o.D. is Limit of Detection.

FIG. 30 shows the relative abundance and persistence of strains from the non-spore forming fraction from donor 4 “D14” in mice. Mice were treated with antibiotics, challenged with carbapenem-resistant Klebsiella pneumoniae, and then administered the non-spore forming fraction (“NSF”) from donor 4 “D14”. Fecal samples were collected from the mice treated with D4 NSF at the indicated treatment time points (pre-donor 4 “D14” NSF, day 3 post-NSF administration (“post treatment”), day 7 post-NSF administration, and day 10 post-NSF administration). The NSF inoculum (input) and fecal samples collected from the mice were whole-genome sequenced using an Illumina MiSeq sequencer, and the bacterial were taxonomically classified for organism relative abundances by One Codex. The relative abundances are shown at the genus level. Due to filtering of the data, the genera do not sum to 100%.

FIG. 31 shows that administration of the non-spore forming fraction from donor 4 (D14 NSF) or a composition of 36 bacterial strains that correspond to 36 strains found in donor 4 (D14 NSF-36) reduced CRE CFU levels. CRE CFU levels were quantified at day 0 and days 14 and 21 following administration of 3 doses of the treatment, shown from left to right for each time point: control (PBS), D14 NSF-36, and D14 NSF. L.o.D. is Limit of Detection.

FIG. 32 shows the in vitro activity of a subset of bacterial strains from the composition of 36 bacterial strains found in donor 4 (shown on the x-axis) against 3 different Klebsiella pneumoniae strains (from left to right for each candidate strain: Kp 2814, Kp OXA-48, and Kp NDM-1) (RFU≥5×10⁴). P. faeces and Agathobaculum sp. are representative inactive strains. Positive and negative controls for in vitro activity against Klebsiella pneumoniae are also shown. RFU is relative fluorescence units.

FIG. 33 shows C57BL/6J mice that were treated with antibiotics in the drinking water and challenged with 10⁵ CFU of CRE (K. pneumoniae ATCC BAA-2814) and administered 3 consecutive doses of one of the following three treatments beginning on day 3 post challenge: PBS; D14-23mix (23 strains found in donor 4 (“D14”)), or D14-36mix (36 strains found in donor 4 (“D14”)). Fecal pellets from all mice were collected various time points post treatment and plated on selective media for CRE quantification. Data shown represents D22 post treatment. L.o.D., limit of detection.

FIG. 34 shows that administration of a composition of 36 bacterial strains that correspond to 36 strains found in donor 4 (D14-36 mix) or the non-spore forming fraction of donor 4 (D14-NSF) reduced colonization with Klebsiella pneumoniae 2H7 (KP 2H7). KP 2H7 levels were quantified at days 0 and 21. For each time point, from left to right: control (PBS), D14-36 mix, and D14-NSF. L.o.D. is Limit of Detection.

### DETAILED DESCRIPTION OF THE INVENTION

Suppressing or preventing undesired bacteria in a subject or suppressing or preventing colonization of bacteria in a particular region of the body can be challenging. Bacterial colonization may induce immune responses (local or systemic) in the subject, which may lead to serious disease. In particular, for multi-drug resistant organisms elimination with many conventional therapeutics, such as antibiotics, may not be possible due to resistance or tolerance of the therapeutic. Additionally, it has been recently appreciated that intestinal colonization by bacteria of the oral microbiome may influence the immune environment of the intestine, such as induce Th1-dominated immune responses and lead to chronic inflammation and inflammatory conditions (see, e.g., Atarashi et al. Science (2017) 358 (359-365)). Normal bacterial colonization of different regions of the body, such as the oral cavity, may provide a reservoir of bacteria that can migrate and colonize other regions, such as the intestines.

Provided herein are compositions and methods for suppressing colonization by multi-drug resistant organisms. Also provided herein are compositions and methods for suppressing colonization of the intestine of subject with bacteria of the oral microbiome. Provided herein are compositions and methods for treating diseases or disorders associated with bacterial colonization or treating diseases or disorders associated with an immune response induced by bacteria.

In some embodiments, the one or more of the bacterial strains of the compositions provided herein colonize or recolonize the intestinal tract or parts of the intestinal tract (e.g., the colon or the cecum) of the subject. Such colonization or recolonization may also be referred to as grafting. In some embodiments, the one or more of the bacterial strains of the compositions recolonize the intestinal tract (e.g., the colon or the cecum) of the subject, for example after another organism or population of organisms has been partially or completely removed. In some embodiments, one or more of the bacterial strains of the compositions recolonize the intestinal track (e.g., the colon or the cecum) after one or more multi-drug resistant organism or other organism (e.g., bacteria that induce an immune response) has been removed. In some embodiments, the recolonization of the intestinal tract or parts thereof by the bacterial strains of the compositions described herein prevents or suppresses colonization by undesired organisms (e.g., multi-drug resistant organisms, oral microbiome bacteria, bacteria that induce immune responses, pathobionts).

In some embodiments, the one or more of the bacterial strains of the compositions can “outgrow” a pathogen or undesired bacteria, such as a multi-drug resistant organism, oral microbiome bacteria, bacteria that induce immune responses. Thus, in some embodiments, if a pathogen or undesired bacteria (e.g., a multi-drug resistant organism, oral microbiome bacteria, bacteria that induce immune responses) and one or more bacteria of compositions provided herein are both present in the intestinal tract (e.g., the colon or the cecum), the one or more bacteria of compositions provided herein grow faster (e.g., have a shorter doubling time) than the pathogen, thereby preventing the pathogen from accumulating in the intestinal tract (e.g., the colon or the cecum). In some embodiments, the faster growth results because the one or more bacteria of the compositions provided herein are better at grafting in the intestinal tract (e.g., the colon or the cecum). In some embodiments, the faster growth results because the one or more bacteria of the compositions provided herein are better at metabolizing nutrients present in the intestinal tract (e.g., the colon or the cecum). In some embodiments, the compositions of bacterial strains provided herein prevent or inhibit replication of the pathogen. In some embodiments, the compositions of bacterial strains provided herein induce death of (kill) the pathogen. In some embodiments, the bacterial strains of the compositions provided herein can treat pathogenic infections, because of the synergy between the bacterial strains.

In some embodiments, the bacterial compositions described herein prevent recolonization by a pathogen or undesired bacteria (e.g., a multi-drug resistant organism, oral microbiome bacteria, bacteria that induce immune responses, pathobionts). For example, in some embodiments, the pathogen or undesired bacteria has been reduced or eliminated from the subject, for example, using a first therapeutic agent, and the bacterial compositions described herein are administered to prevent recolonization of the subject. In some embodiments, the bacterial compositions described herein reduce or eliminate a pathogen or undesired bacteria from the subject and prevent recolonization of the subject.

In some embodiments, the combination of bacterial strains of the compositions provided herein is superior in the use of nutrients when compared to the pathogen or undesired bacteria, thereby suppressing the growth of the pathogen or undesired bacteria. In some embodiments, the combination of bacterial strains of the compositions provided herein is superior in grafting when compared to the pathogen or undesired bacteria, thereby suppressing the growth of the pathogen or undesired bacteria. In some embodiments, the combination of bacterial strains of the compositions provided herein is superior in the use of nutrients and in grafting when compared to the pathogen or undesired bacteria, thereby suppressing the growth of the pathogen or undesired bacteria. In some embodiments, the combination of bacterial strains of the compositions provided herein inhibits the growth, survival, and/or colonization of the pathogen or undesired bacteria.

In some embodiments, the combination of bacterial strains of the compositions provided herein has antagonizing or inhibitory activity towards the pathogen or undesired bacteria, thereby inhibiting the growth, survival, and/or colonization of the pathogen or undesired bacteria. In some embodiments, at least one bacterial strain of the compositions provided herein has antagonizing or inhibitory activity towards the pathogen or undesired bacteria, thereby inhibiting the growth, survival, and/or colonization of the pathogen or undesired bacteria.

In some embodiments, the synergistic effect is provided by the capacity of the combination to colonize specific niches in the intestinal tract (e.g., the colon or the cecum). In some embodiments, the synergistic effect is provided by the capacity of the combination to metabolize specific nutrients. In some embodiments, the synergistic effect is provided by the capacity of the combination to provide specific metabolites to the environment.

The bacterial strains used in the compositions provided herein generally are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated and grown up individually. The bacterial compositions that are grown up individually may subsequently be combined to provide the compositions of the disclosure. It should be appreciated that the origin of the bacterial strains of the compositions provided herein is not limited to the human microbiome from a healthy individual. In some embodiments, the bacterial strains originate from a human with a microbiome in dysbiosis. In some embodiments, the bacteria originate from a spore-forming fraction of the microbiome. In some embodiments, the bacteria originate from a non-spore-forming fraction of the microbiome. In some embodiments, the bacterial strains originate from non-human animals or the environment (e.g., soil or surface water). In some embodiments, the combinations of bacterial strains provided herein originate from multiple sources (e.g., human and non-human animals).

In some embodiments, the bacteria of the compositions provided herein are anaerobic bacteria. In some embodiments, the bacteria of the compositions provided herein are obligate anaerobic bacteria. In some embodiments, the bacteria of the compositions provided herein are clostridia. Clostridia may be classified into phylogenetic clusters with other closely related strains and species. (See e.g., Rajilic-Stojanovic, M., and de Vos, W. M. FEMS Microbiol Rev 38, (2014) 996-1047). In general, clostridia are classified as belonging to a specific cluster based on their 16S rRNA (or 16S rDNA) nucleic acid sequence. Methods for determining the identity of specific bacterial species based on their 16S rRNA (or 16S rDNA) nucleic acid sequence are well known in the art (See e.g., Jumpstart Consortium Human Microbiome Project Data Generation Working, G. PLoS One (2012) 7, e39315).

In some embodiments, the bacteria of the compositions provided herein are Bacteroides.

In one aspect, the disclosure provides composition comprising one or more bacterial strains comprising a 16S rDNA sequence with nucleic acid sequences selected from the group consisting of SEQ ID NOs: 1-22 or any of the other 16S sequences provided herein. In one aspect, the disclosure provides composition comprising two or more bacterial strains comprising a 16S rDNA sequence with nucleic acid sequences selected from the group consisting of SEQ ID NOs: 1-22 or any of the other 16S sequences provided herein. It should be appreciated that SEQ ID NOs: 1-22 or any of the other 16S sequences provided herein may include both full length and partial 16S rDNA sequences.

In one aspect, the disclosure provides compositions comprising two or more bacterial strains comprising 16S rDNA sequences selected from the group consisting of SEQ ID NOs: 1-22. In one aspect, the disclosure provides compositions comprising as active ingredients two or more bacterial strains comprising 16S rDNA sequences selected from the group consisting of SEQ ID NOs: 1-22. It should be appreciated that for all compositions provided herein, in some embodiments, the bacterial strain or bacterial strains are the active ingredient(s) of the composition.

It should be appreciated that for all compositions provided herein, in some embodiments, the bacterial strains are purified. Thus, for example the disclosure provides purified bacterial strains comprising 16S rDNA sequences with nucleic acid sequences selected from the group consisting of SEQ ID NOs: 1-22. In addition, for example, the disclosure provides compositions comprising purified bacterial strains comprising a 16S rDNA sequence with a nucleic acid sequence selected from the group consisting of SEQ ID NOs: 1-22. The bacterial strains disclosed herein originally may have been obtained and purified from the microbiota of one or more human individuals or obtained from sources other than the human microbiota, including soil and non-human microbiota. As provided herein, in some embodiments, bacteria isolated from the human microbiota, non-human microbiota, soil, or any alternative source are purified prior to use in the compositions and methods provided herein.

In one aspect, the disclosure provides compositions comprising one or more purified bacterial strains wherein the one or more purified bacterial strains comprise 16S rDNA sequences having at least 97% sequence identity with nucleic acid sequences selected from the group consisting of SEQ ID NOs: 1-22. In one aspect, the disclosure provides compositions comprising two or more purified bacterial strains wherein the two or more purified bacterial strains comprise 16S rDNA sequences having at least 97% sequence identity with nucleic acid sequences selected from the group consisting of SEQ ID NOs: 1-22. As discussed above, in some embodiments, the bacterial strains are the active ingredient of the composition. Thus, in some embodiments, the disclosure provides compositions comprising as an active ingredient two or more purified bacterial strains wherein the two or more purified bacterial strains comprise 16S rDNA sequences having at least 97% sequence identity with nucleic acid sequences selected from the group consisting of SEQ ID NOs: 1-22.

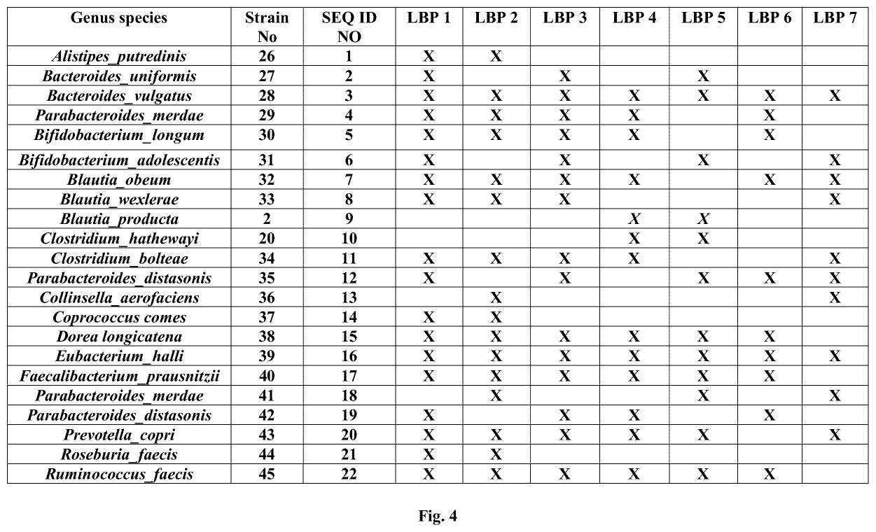

In one aspect, the disclosure provides bacterial strains and combinations of bacterial strains that have a high percent of sequence identity or have a high percent of homology with bacterial strains comprising 16S rDNA sequences selected from the group consisting of SEQ ID NOs: 1-22. As discussed previously, in some embodiments, the bacterial strains are purified. The bacterial strains disclosed herein that have a 16S rDNA sequence with a nucleic acid sequence selected from the group consisting of SEQ ID NOs: 1-22 have a high percent of sequence identity or homology (e.g., greater than 90%) with 16S rDNA sequences of bacterial strains that have been described in various databases (See e.g., the National Center for Biotechnology Information). FIG. 4 and Tables 1-4 provide the closest known species by sequence identity when the 16S rDNA sequences comprising SEQ ID NOs: 1-22 are compared to 16S rDNA sequences of bacterial species available in public databases. By way of example, the bacterial strain comprising a 16S rDNA sequence with SEQ ID NO: 1 (also referred to herein as “Strain 26”) disclosed herein has the highest sequence identity with a bacterial strain of the species Alistipes putredinis. While the bacterial strain with 16S rDNA sequence of SEQ ID NO: 1 has sequence identity with other published bacterial strains as well, the highest sequence identity is with a bacterial strain of the species Alistipes putredinis. It should be appreciated that multiple bacterial strains disclosed herein may have the highest sequence identity with the same species (e.g., both SEQ ID NO: 4 and SEQ ID NO: 18 have the highest sequence identity with a 16S rDNA sequence of a strain of the species Parabacteroides merdae; and both SEQ ID NO: 12 and SEQ ID NO: 19 have the highest sequence identity with a 16S rDNA sequence of a strain of the species Parabacteroides distasonis).

It should further be appreciated that the bacterial strains disclosed herein that have a 16S rDNA sequence with a nucleic acid sequence selected from the group consisting of SEQ ID NO: 1-22 may have a high level of sequence identity or homology to other strains based on their whole genome sequence or subset of their whole genome sequence.

TABLE A

| | | 16 S rDNA |
|---|---|---|
| | | SEQ |
| Genus species | Strain No | ID NO |
| Alistipes_putredinis | 26 | 1 |
| Bacteroides_uniformis | 27 | 2 |
| Bacteroides_vulgatus | 28 | 3 |
| Parabacteroides_merdae | 29 | 4 |
| Bifidobacterium_longum | 30 | 5 |
| Bifidobacterium_adolescentis | 31 | 6 |
| Blautia_obeum | 32 | 7 |
| Blautia_wexlerae | 33 | 8 |
| Blautia_producta | 2 | 9 |
| Clostridium_hathewayi | 20 | 10 |
| Clostridium_bolteae | 34 | 11 |
| Parabacteroides_distasonis | 35 | 12 |
| Collinsella_aerofaciens | 36 | 13 |
| Coprococcus comes | 37 | 14 |
| Dorea longicatena | 38 | 15 |
| Eubacterium_halli | 39 | 16 |
| Faecalibacterium_prausnitzii | 40 | 17 |
| Parabacteroides_merdae | 41 | 18 |
| Parabacteroides_distasonis | 42 | 19 |
| Prevotella_copri | 43 | 20 |
| Roseburia_faecis | 44 | 21 |
| Ruminococcus_faecis | 45 | 22 |

In one aspect, the disclosure provides compositions comprising one or more bacterial species selected from the group consisting of Alistipes putredinis, Bacteroides uniformis, Bacteroides vulgatus, Barnesielia intestinihominis, Bifidobacterium longum, Bifidobacterium adolescentis, Blautia obeum, Blautia wexlerae, Blautia producta, Clostridium hathewayi, Clostridium bolteae, Clostridium innocuum, Collinsella aerofaciens, Coprococcus comes, Dorea longicatena, Eubacterium halli, Faecalibacterium prausnitzii, Parabacteroides merdae, Parabacteroides distasonis, Prevotella copri, Roseburia faecis, and Ruminococcus.

In one aspect, the disclosure provides composition comprising one or more bacterial strains selected from the group consisting of strains 26-45 (See, FIG. 4 and Table A). In one aspect, the disclosure provides compositions comprising two or more bacterial species selected from the group consisting of Alistipes putredinis, Bacteroides uniformis, Bacteroides vulgatus, Barnesiella intestinihominis, Bifidobacterium longum, Bifidobacterium adolescentis, Blautia obeum, Blautia wexlerae, Blautia producta, Clostridium hathewayi, Clostridium bolteae, Clostridium innocuum, Collinsella aerofaciens, Coprococcus comes, Dorea longicatena, Eubacterium halli, Faecalibacterium prausnitzii, Parabacteroides merdae, Parabacteroides distasonis, Prevotella copri, Roseburia faecis, and Ruminococcus faecis. Clostridium hathewayi also can be referred to herein as Hungatella effluvia.

It should be appreciated that the compositions may contain multiple strains of a particular bacterial species. For example, in some embodiments, the composition may comprise two strains of Parabacteroides distasonis and/or two strains of Parabacteroides merdae.

The disclosure also encompasses compositions comprising bacterial strains having close sequence identity or homology to and/or fall within the species of Alistipes putredinis, Bacteroides uniformis, Bacteroides vulgatus, Barnesiella intestinihominis, Bifidobacterium longum, Bifidobacterium adolescentis, Blautia obeum, Blautia wexlerae, Blautia producta, Clostridium hathewayi, Clostridium bolteae, Clostridium innocuum, Collinsella aerofaciens, Coprococcus comes, Dorea longicatena, Eubacterium halli, Faecalibacterium prausnitzii, Parabacteroides merdae, Parabacteroides distasonis, Prevotella copri, Roseburia faecis, and Ruminococcus faecis. In some embodiments, the compositions include two or more bacterial strains comprising 16S rDNA sequences having at least 97% sequence identity or homology with the nucleic acid sequences selected from the group consisting of SEQ ID NOs: 1-22.

In some embodiments, the compositions disclosed herein comprise two or more bacterial strains. In some embodiments, the compositions described herein comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 21, at least 22, at least 23, at least 24, at least 25, at least 26, at least 27, at least 28, at least 29, at least 30, at least 31, at least 32, at least 33, at least 34, at least 35, at least 36 or more bacterial strains (e.g., purified bacterial strains).

In some embodiments, the composition suppresses the replication, survival, and/or colonization of one or more pathogenic organism. In some instances, the pathogenic organism is susceptible to antibiotics, while in other instances, the pathogenic organism is resistant to antibiotics. In some embodiments, the pathogenic organism is a multi-drug resistant organism, which are described elsewhere herein. In some embodiments, the pathogenic organism is an oral microbiome bacteria. It should be noted that oral microbiome bacteria are not necessarily pathogenic, but may become so when located elsewhere, such as in the gastrointestinal tract. The amount of suppression of replication, survival, and/or colonization of the one or more pathogenic organism can be measured or identified using standard assays known in the art, some of which are further described and exemplified herein.

In some embodiments, the pathogenic organism is Clostridium difficile.

In some embodiments, the pathogenic organism is Klebsiella pneumoniae. In some embodiments, the Klebsiella pneumoniae is multi-drug resistant. In some embodiments, the multi-drug resistant Klebsiella pneumoniae is carbapenem-resistant Klebsiella pneumoniae. In some embodiments, the Klebsiella organism induces a Th1 response. In some embodiments, the Klebsiella pneumoniae is multi-drug resistant and induces a Th1 response. In some embodiments, the Klebsiella pneumoniae is one or more of those described in Atarashi et al. Science 358, 359-365 (2017), such as strain BAA-2552, strain KP-1, strain 700721, strain 13882, strain 34E1, strain BAA-1705, strain 700603, and/or strain Kp-2H7. In one particular embodiment, the Klebsiella pneumoniae is strain Kp-2H7.

In some embodiments, the pathogenic organism is a pathobiont, i.e., a potentially pathologenic organism which, under normal circumstances, lives as a symbiont.

It should be appreciated that the terms “bacteria” and “bacterial strains” as used herein are interchangeable. The compositions described herein containing multiple purified bacterial strains may also be referred to as “live bacterial products.”

In one aspect, the disclosure provides live bacterial product 1 (LBP 1) (see, e.g., FIG. 4, Table 1). As shown in FIG. 4 and Table 1, live bacterial product 1 contains bacterial strains that comprise 16S rDNA sequences with nucleic acid sequences SEQ ID NOs: 1-8, 11, 12, 14-17, and 19-22. In some embodiments, the compositions comprise at least two (e.g., 2, 3, 4, 5, or more) bacterial strains that comprise 16S rDNA sequences with nucleic acid sequences SEQ ID NO: 1-8, 11, 12, 14-17, and 19-22. In some embodiments, the composition comprises 18 purified bacterial strains, wherein the bacterial strains comprise 16S rDNA sequences with nucleic acid sequences SEQ ID NOs: 1-8, 11, 12, 14-17, and 19-22. In some embodiments, the composition consists of 18 purified bacterial strains, wherein the bacterial strains comprise 16S rDNA sequences with nucleic acid sequences SEQ ID NOs: 1-8, 11, 12, 14-17, and 19-22. In some embodiments, the composition essentially consists of 18 purified bacterial strains, wherein the bacterial strains comprise 16S rDNA sequences with nucleic acid sequences SEQ ID NOs: 1-8, 11, 12, 14-17, and 19-22. As used herein, “essentially consists of” (and like phrases) refers to a composition that includes no additional bacterial strains.

In some embodiments, the disclosure provides compositions with bacterial strains that comprise 16S rDNA sequences having at least 97% sequence identity with nucleic acid sequences selected from the group consisting of SEQ ID NOs: 1-8, 11, 12, 14-17, and 19-22. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 1-8, 11, 12, 14-17, and 19-22. In some embodiments, the compositions comprise 18 purified bacterial strains, wherein the bacterial strains comprise 16S rDNA sequences having at least 97% sequence identity with nucleic acid sequences SEQ ID NOs: 1-8, 11, 12, 14-17, and 19-22. In some embodiments, the compositions consist of 18 purified bacterial strains, wherein the bacterial strains comprise 16S rDNA sequences having at least 97% sequence identity with nucleic acid sequences SEQ ID NOs: 1-8, 11, 12, 14-17, and 19-22. In some embodiments, the compositions essentially consist of 18 purified bacterial strains, wherein the bacterial strains comprise 16S rDNA sequences having at least 97% sequence identity with nucleic acid sequences SEQ ID NOs: 1-8, 11, 12, 14-17, and 19-22.

The bacterial strains in live bacterial product 1 are related to the following species: Alistipes putredinis, Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Coprococcus comes, Dorea longicatena, Eubacterium halli, Faecalibacterium prausnitzii, Parabacteroides distasonis, Prevotella copri, Roseburia faecis, and Ruminococcus faecis (see, e.g., Table 1). It should be appreciated that multiple bacterial strains of the compositions described herein can have the same related bacterial species. For example, the bacterial strains having 16S rDNA sequences with nucleic acid sequences SEQ ID NO: 12 and SEQ ID NO: 19 both have Parabacteroides distasonis as the related species. In some embodiments, the compositions comprise two or more (e.g., 2, 3, 4, 5, or more) bacterial species selected from the group consisting of Alistipes putredinis, Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Coprococcus comes, Dorea longicatena, Eubacterium halli, Faecalibacterium prausnitzii, Parabacteroides distasonis, Prevotella copri, Roseburia faecis, and Ruminococcus faecis. In some embodiments, the compositions comprise 18 bacterial species: Alistipes putredinis, Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Coprococcus comes, Dorea longicatena, Eubacterium halli, Faecalibacterium prausnitzii, Parabacteroides distasonis, Prevotella copri, Roseburia faecis, and Ruminococcus faecis. In some embodiments, the compositions consist of 18 bacterial species: Alistipes putredinis, Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Coprococcus comes, Dorea longicatena, Eubacterium halli, Faecalibacterium prausnitzii, Parabacteroides distasonis, Prevotella copri, Roseburia faecis, and Ruminococcus faecis. In some embodiments, the compositions essentially consist of 18 bacterial species: Alistipes putredinis, Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Coprococcus comes, Dorea longicatena, Eubacterium halli, Faecalibacterium prausnitzii, Parabacteroides distasonis, Prevotella copri, Roseburia faecis, and Ruminococcus faecis.

In one aspect, the disclosure provides composition comprising one or more bacterial strains selected from the group consisting of strains 26-33, 34, 35, 37-40 and 42-45 (See Table 1).

TABLE 1
Live bacterial product 1

| | | 16S rDNA |
|---|---|---|
| | | SEQ |
| Genus species | Strain No. | ID NO |
| Alistipes_putredinis | Strain 26 | 1 |
| Bacteroides_uniformis | Strain 27 | 2 |
| Bacteroides_vulgatus | Strain 28 | 3 |
| Parabacteroides merdae | Strain 29 | 4 |
| Bifidobacterium_longum | Strain 30 | 5 |
| Bifidobacterium_adolescentis | Strain 31 | 6 |
| Blautia_obeum | Strain 32 | 7 |
| Blautia_wexlerae | Strain 33 | 8 |
| Clostridium_bolteae | Strain 34 | 11 |
| Parabacteroides distasonis | Strain 35 | 12 |
| Coprococcus comes | Strain 37 | 14 |
| Dorea longicatena | Strain 38 | 15 |
| Eubacterium_halli | Strain 39 | 16 |
| Faecalibacterium_prausnitzii | Strain 40 | 17 |
| Parabacteroides_distasonis | Strain 42 | 19 |
| Prevotella_copri | Strain 43 | 20 |
| Roseburia_faecis | Strain 44 | 21 |
| Ruminococcus_faecis | Strain 45 | 22 |

In one aspect, the disclosure provides live bacterial product 2 (LBP 2) (see, e.g., FIG. 4, Table 2). As shown in FIG. 4 and Table 2, live bacterial product 2 contains bacterial strains that comprise 16S rDNA sequences with nucleic acid sequences SEQ ID NOs: 1, 3-5, 7, 8, 11, 13-18, and 20-22. In some embodiments, the compositions comprise at least two (e.g., 2, 3, 4, 5, or more) bacterial strains that comprise 16S rDNA sequences with nucleic acid sequences SEQ ID NOs: 1, 3-5, 7, 8, 11, 13-18, and 20-22. In some embodiments, the composition comprises 16 purified bacterial strains, wherein the bacterial strains comprise 16S rDNA sequences with nucleic acid sequences SEQ ID NOs: 1, 3-5, 7, 8, 11, 13-18, and 20-22. In some embodiments, the composition consists of 16 purified bacterial strains, wherein the bacterial strains comprise 16S rDNA sequences with nucleic acid sequences SEQ ID NOs: 1, 3-5, 7, 8, 11, 13-18, and 20-22. In some embodiments, the composition essentially consists of 16 purified bacterial strains, wherein the bacterial strains comprise 16S rDNA sequences with nucleic acid sequences SEQ ID NOs: 1, 3-5, 7, 8, 11, 13-18, and 20-22. As used herein, “essentially consists of” (and like phrases) refers to a composition that includes no additional bacterial strains.

In some embodiments, the disclosure provides compositions with bacterial strains that comprise 16S rDNA sequences having at least 97% sequence identity with nucleic acid sequences selected from the group consisting of SEQ ID NOs: 1, 3-5, 7, 8, 11, 13-18, and 20-22. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 1, 3-5, 7, 8, 11, 13-18, and 20-22. In some embodiments, the compositions comprise 16 purified bacterial strains, wherein the bacterial strains comprise 16S rDNA sequences having at least 97% sequence identity with nucleic acid sequences SEQ ID NOs: 1, 3-5, 7, 8, 11, 13-18, and 20-22. In some embodiments, the compositions consist of 16 purified bacterial strains, wherein the bacterial strains comprise 16S rDNA sequences having at least 97% sequence identity with nucleic acid sequences SEQ ID NOs: 1, 3-5, 7, 8, 11, 13-18, and 20-22. In some embodiments, the compositions essentially consist of 16 purified bacterial strains, wherein the bacterial strains comprise 16S rDNA sequences having at least 97% sequence identity with nucleic acid sequences SEQ ID NOs: 1, 3-5, 7, 8, 11, 13-18, and 20-22.

The bacterial strains in live bacterial product 2 are related to the following species: Alistipes putredinis, Bacteroides vulgatus, Barnesiella intestinihominis, Bifidobacterium longum, Blautia obeum, Blautia wexlerae, Clostridium bolteae, Parabacteroides distasonis, Collinsella aerofaciens, Coprococcus comes, Dorea longicatena, Eubacterium halli, Faecalibacterium prausnitzii, Parabacteroides merdae, Prevotella copri, Roseburia faecis, and Ruminococcus faecis (see, e.g., Table 2). It should be appreciated that multiple bacterial strains of the compositions described herein can have the same related bacterial species. For example, the bacterial strains having 16S rDNA sequences with nucleic acid sequences SEQ ID NO: 4 and SEQ ID NO: 18 both have Parabacteroides merdae as the related species. In some embodiments, the compositions comprise two or more (e.g., 2, 3, 4, 5, or more) bacterial species selected from the group consisting of Alistipes putredinis, Bacteroides vulgatus, Barnesiella intestinihominis, Bifidobacterium longum, Blautia obeum, Blautia wexlerae, Clostridium bolteae, Parabacteroides distasonis, Collinsella aerofaciens, Coprococcus comes, Dorea longicatena, Eubacterium halli, Faecalibacterium prausnitzii, Parabacteroides merdae, Prevotella copri, Roseburia faecis, and Ruminococcus faecis. In some embodiments, the compositions comprise 16 bacterial species: Alistipes putredinis, Bacteroides vulgatus, Barnesiella intestinihominis, Bifidobacterium longum, Blautia obeum, Blautia wexlerae, Clostridium bolteae, Parabacteroides distasonis, Collinsella aerofaciens, Coprococcus comes, Dorea longicatena, Eubacterium halli, Faecalibacterium prausnitzii, Parabacteroides merdae, Prevotella copri, Roseburia faecis, and Ruminococcus faecis. In some embodiments, the compositions consist of 16 bacterial species: Alistipes putredinis, Bacteroides vulgatus, Barnesiella intestinihominis, Bifidobacterium longum, Blautia obeum, Blautia wexlerae, Clostridium bolteae, Parabacteroides distasonis, Collinsella aerofaciens, Coprococcus comes, Dorea longicatena, Eubacterium halli, Faecalibacterium prausnitzii, Parabacteroides merdae, Prevotella copri, Roseburia faecis, and Ruminococcus faecis. In some embodiments, the compositions essentially consist 16 bacterial species: Alistipes putredinis, Bacteroides vulgatus, Barnesiella intestinihominis, Bifidobacterium longum, Blautia obeum, Blautia wexlerae, Clostridium bolteae, Parabacteroides distasonis, Collinsella aerofaciens, Coprococcus comes, Dorea longicatena, Eubacterium halli, Faecalibacterium prausnitzii, Parabacteroides merdae, Prevotella copri, Roseburia faecis, and Ruminococcus faecis.

In one aspect, the disclosure provides compositions comprising one or more bacterial strains selected from the group consisting of strains 26, 28-30, 32, 33, 34, 36-41 and 43-45 (See Table 2).

TABLE 2
Live bacterial product 2

| | | 16S rDNA |
|---|---|---|
| | | SEQ |
| Genus species | Strain No. | ID NO: |
| Alistipes_putredinis | Strain 26 | 1 |
| Bacteroides_vulgatus | Strain 28 | 3 |
| Parabacteroides merdae | Strain 29 | 4 |
| Bifidobacterium_longum | Strain 30 | 5 |
| Blautia_obeum | Strain 32 | 7 |
| Blautia_wexlerae | Strain 33 | 8 |
| Clostridium_bolteae | Strain 34 | 11 |
| Collinsella_aerofaciens | Strain 36 | 13 |
| Coprococcus comes | Strain 37 | 14 |
| Dorea longicatena | Strain 38 | 15 |
| Eubacterium_halli | Strain 39 | 16 |
| Faecalibacterium_prausnitzii | Strain 40 | 17 |
| Parabacteroides_merdae | Strain 41 | 18 |
| Prevotella_copri | Strain 43 | 20 |
| Roseburia_faecis | Strain 44 | 21 |
| Ruminococcus_faecis | Strain 45 | 22 |

In one aspect, the disclosure provides live bacterial product 3 (LBP 3) (see, e.g., FIG. 4, Table 3). As shown in FIG. 4 and Table 3, live bacterial product 3 contains bacterial strains that comprise 16S rDNA sequences with nucleic acid sequences SEQ ID NOs: 2-8, 11, 12, 15-17, 19, 20, and 22. In some embodiments, the compositions comprise at least two (e.g., 2, 3, 4, 5, or more) bacterial strains that comprise 16S rDNA sequences with nucleic acid sequences SEQ ID NOs: 2-8, 11, 12, 15-17, 19, 20, and 22. In some embodiments, the composition comprises 15 purified bacterial strains, wherein the bacterial strains comprise 16S rDNA sequences with nucleic acid sequences SEQ ID NOs: 2-8, 11, 12, 15-17, 19, 20, and 22. In some embodiments, the composition consists of 15 purified bacterial strains, wherein the bacterial strains comprise 16S rDNA sequences with nucleic acid sequences SEQ ID NOs: 2-8, 11, 12, 15-17, 19, 20, and 22. In some embodiments, the composition essentially consists of 15 purified bacterial strains, wherein the bacterial strains comprise 16S rDNA sequences with nucleic acid sequences SEQ ID NOs: 2-8, 11, 12, 15-17, 19, 20, and 22.

In some embodiments, the disclosure provides compositions with bacterial strains that comprise 16S rDNA sequences having at least 97% sequence identity with nucleic acid sequences selected from the group consisting of SEQ ID NOs: 2-8, 11, 12, 15-17, 19, 20, and 22. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 2-8, 11, 12, 15-17, 19, 20, and 22. In some embodiments, the compositions comprise 15 purified bacterial strains, wherein the bacterial strains comprise 16S rDNA sequences having at least 97% sequence identity with nucleic acid sequences SEQ ID NOs: 2-8, 11, 12, 15-17, 19, 20, and 22. In some embodiments, the compositions consist of 15 purified bacterial strains, wherein the bacterial strains comprise 16S rDNA sequences having at least 97% sequence identity with nucleic acid sequences SEQ ID NOs: 2-8, 11, 12, 15-17, 19, 20, and 22.

In some embodiments, the compositions essentially consist of 15 purified bacterial strains, wherein the bacterial strains comprise 16S rDNA sequences having at least 97% sequence identity with nucleic acid sequences SEQ ID NOs: 2-8, 11, 12, 15-17, 19, 20, and 22.

The bacterial strains in live bacterial product 3 are related to the following species: Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Dorea longicatena, Eubacterium halli, Faecalibacterium prausnitzii, Parabacteroides distasonis, Prevotella copri, and Ruminococcus faecis (see, e.g., Table 3). It should be appreciated that multiple bacterial strains of the compositions described herein can have the same related bacterial species. For example, the bacterial strains having 16S rDNA sequences with nucleic acid sequences SEQ ID NO: 12 and SEQ ID NO: 119 both have Parabacteroides distasonis as the related species. In some embodiments, the compositions comprise two or more (e.g., 2, 3, 4, 5, or more) bacterial species selected from the group consisting of Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Dorea longicatena, Eubacterium halli, Faecalibacterium prausnitzii, Parabacteroides distasonis, Prevotella copri, and Ruminococcus faecis. In some embodiments, the compositions comprise 15 bacterial species: Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Dorea longicatena, Eubacterium halli, Faecalibacterium prausnitzii, Parabacteroides distasonis, Prevotella copri, and Ruminococcus faecis. In some embodiments, the compositions consist of 15 bacterial species: Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Dorea longicatena, Eubacterium halli, Faecalibacterium prausnitzii, Parabacteroides distasonis, Prevotella copri, and Ruminococcus faecis. In some embodiments, the compositions essentially consist 15 bacterial species: Bacteroides uniformis, Bacteroides vulgatus, Barnesiella intestinihominis, Parabacteroides merdae, Bifidobacterium longum, Bifidobacterium adolescentis, Blautia obeum, Blautia wexlerae, Clostridium bolteae, Clostridium innocuum, Dorea longicatena, Eubacterium halli, Faecalibacterium prausnitzii, Parabacteroides distasonis, Prevotella copri, and Ruminococcus faecis.

In one aspect, the disclosure provides composition comprising one or more bacterial strains selected from the group consisting of strains 27-33, 34, 35, 38-40, 42, 43, and 45 (See Table 3).

TABLE 3
Live bacterial product 3

| | | 16S rDNA |
|---|---|---|
| | | SEQ |
| Genus species | Strain No. | ID NO |
| Bacteroides_uniformis | Strain 27 | 2 |
| Bacteroides_vulgatus | Strain 28 | 3 |
| Parabacteroides merdae | Strain 29 | 4 |
| Bifidobacterium_longum | Strain 30 | 5 |
| Bifidobacterium_adolescentis | Strain 31 | 6 |
| Blautia_obeum | Strain 32 | 7 |
| Blautia_wexlerae | Strain 33 | 8 |
| Clostridium_bolteae | Strain 34 | 11 |
| Parabacteroides distasonis | Strain 35 | 12 |
| Dorea longicatena | Strain 38 | 15 |
| Eubacterium_halli | Strain 39 | 16 |
| Faecalibacterium_prausnitzii | Strain 40 | 17 |
| Parabacteroides_distasonis | Strain 42 | 19 |
| Prevotella_copri | Strain 43 | 20 |
| Ruminococcus_faecis | Strain 45 | 22 |

In one aspect, the disclosure provides live bacterial product 4 (LBP 4) (see, e.g., FIG. 4, Table 4). As shown in FIG. 4 and Table 4, live bacterial product 4 contains bacterial strains that comprise 16S rDNA sequences with nucleic acid sequences SEQ ID NOs: 3-5, 7, 9-11, 15-17, 19, 20, and 22. In some embodiments, the compositions comprise at least two (e.g., 2, 3, 4, 5, or more) bacterial strains that comprise 16S rDNA sequences with nucleic acid sequences SEQ ID NOs: 3-5, 7, 9-11, 15-17, 19, 20, and 22. In some embodiments, the composition comprises 13 purified bacterial strains, wherein the bacterial strains comprise 16S rDNA sequences with nucleic acid sequences SEQ ID NOs: 3-5, 7, 9-11, 15-17, 19, 20, and 22. In some embodiments, the composition consists of 13 purified bacterial strains, wherein the bacterial strains comprise 16S rDNA sequences with nucleic acid sequences SEQ ID NOs: 3-5, 7, 9-11, 15-17, 19, 20, and 22. In some embodiments, the composition essentially consists of 13 purified bacterial strains, wherein the bacterial strains comprise 16S rDNA sequences with nucleic acid sequences SEQ ID NOs: 3-5, 7, 9-11, 15-17, 19, 20, and 22.

In some embodiments, the disclosure provides compositions with bacterial strains that comprise 16S rDNA sequences having at least 97% sequence identity with nucleic acid sequences selected from the group consisting of SEQ ID NOs: 3-5, 7, 9-11, 15-17, 19, 20, and 22. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 3-5, 7, 9-11, 15-17, 19, 20, and 22. In some embodiments, the compositions comprise 13 purified bacterial strains, wherein the bacterial strains comprise 16S rDNA sequences having at least 97% sequence identity with nucleic acid sequences SEQ ID NOs: 3-5, 7, 9-11, 15-17, 19, 20, and 22. In some embodiments, the compositions consist of 13 purified bacterial strains, wherein the bacterial strains comprise 16S rDNA sequences having at least 97% sequence identity with nucleic acid sequences SEQ ID NOs: 3-5, 7, 9-11, 15-17, 19, 20, and 22. In some embodiments, the compositions essentially consist of 13 purified bacterial strains, wherein the bacterial strains comprise 16S rDNA sequences having at least 97% sequence identity with nucleic acid sequences SEQ ID NOs: 3-5, 7, 9-11, 15-17, 19, 20, and 22.

The bacterial strains in live bacterial product 4 are related to the following species: Bacteroides vulgatus, Barnesiella intestinihominis, Parabacteroides merdae, Bifidobacterium longum, Blautia obeum, Blautia producta, Clostridium hathewayi, Clostridium bolteae, Dorea longicatena, Eubacterium halli, Faecalibacterium prausnitzii, Parabacteroides distasonis, Prevotella copri, and Ruminococcus faecis (see, e.g., Table 4). In some embodiments, the compositions comprise two or more (e.g., 2, 3, 4, 5, or more) bacterial species selected from the group consisting of Bacteroides vulgatus, Barnesiella intestinihominis, Parabacteroides merdae, Bifidobacterium longum, Blautia obeum, Blautia producta, Clostridium hathewayi, Clostridium bolteae, Dorea longicatena, Eubacterium halli, Faecalibacterium prausnitzii, Parabacteroides distasonis, Prevotella copri, and Ruminococcus faecis. In some embodiments, the compositions comprise 13 bacterial species: Bacteroides vulgatus, Barnesiella intestinihominis, Parabacteroides merdae, Bifidobacterium longum, Blautia obeum, Blautia producta, Clostridium hathewayi, Clostridium bolteae, Dorea longicatena, Eubacterium halli, Faecalibacterium prausnitzii, Parabacteroides distasonis, Prevotella copri, and Ruminococcus faecis. In some embodiments, the compositions consist of 13 bacterial species: Bacteroides vulgatus, Barnesiella intestinihominis, Parabacteroides merdae, Bifidobacterium longum, Blautia obeum, Blautia producta, Clostridium hathewayi, Clostridium bolteae, Dorea longicatena, Eubacterium halli, Faecalibacterium prausnitzii, Parabacteroides distasonis, Prevotella copri, and Ruminococcus faecis. In some embodiments, the compositions essentially consist 13 bacterial species: Bacteroides vulgatus, Barnesiella intestinihominis, Parabacteroides merdae, Bifidobacterium longum, Blautia obeum, Blautia producta, Clostridium hathewayi, Clostridium bolteae, Dorea longicatena, Eubacterium halli, Faecalibacterium prausnitzii, Parabacteroides distasonis, Prevotella copri, and Ruminococcus faecis.

In one aspect, the disclosure provides composition comprising one or more bacterial strains selected from the group consisting of strains 28-30, 32, 2, 20, 34, 38-40, 42, 43, and 45 (See Table 4).

TABLE 4
Live bacterial product 4

| | | 16S rDNA |
|---|---|---|
| | | SEQ |
| Genus species | Strain No. | ID NO |
| Bacteroides_vulgatus | Strain 28 | 3 |
| Parabacteroides merdae | Strain 29 | 4 |
| Bifidobacterium_longum | Strain 30 | 5 |
| Blautia_obeum | Strain 32 | 7 |
| Blautia_producta | Strain 2 | 9 |
| Clostridium_hathewayi | Strain 20 | 10 |
| Clostridium_bolteae | Strain 34 | 11 |
| Dorea longicatena | Strain 38 | 15 |
| Eubacterium_halli | Strain 39 | 16 |
| Faecalibacterium_prausnitzii | Strain 40 | 17 |
| Parabacteroides_distasonis | Strain 42 | 19 |
| Prevotella_copri | Strain 43 | 20 |
| Ruminococcus_faecis | Strain 45 | 22 |

In one aspect, the disclosure provides live bacterial product 5 (LBP 5) (see, e.g., FIG. 4, Table 5). As shown in FIG. 4 and Table 5, live bacterial product 5 contains bacterial strains that comprise 16S rDNA sequences with nucleic acid sequences SEQ ID NOs: 2, 3, 6, 9, 10, 12, 15-18, 20, and 22. In some embodiments, the compositions comprise at least two (e.g., 2, 3, 4, 5, or more) bacterial strains that comprise 16S rDNA sequences with nucleic acid sequences SEQ ID NOs: 2, 3, 6, 9, 10, 12, 15-18, 20, and 22. In some embodiments, the composition comprises 12 purified bacterial strains, wherein the bacterial strains comprise 16S rDNA sequences with nucleic acid sequences SEQ ID NOs: 2, 3, 6, 9, 10, 12, 15-18, 20, and 22. In some embodiments, the composition consists of 12 purified bacterial strains, wherein the bacterial strains comprise 16S rDNA sequences with nucleic acid sequences SEQ ID NOs: 2, 3, 6, 9, 10, 12, 15-18, 20, and 22. In some embodiments, the composition essentially consists of 12 purified bacterial strains, wherein the bacterial strains comprise 16S rDNA sequences with nucleic acid sequences SEQ ID NOs: 2, 3, 6, 9, 10, 12, 15-18, 20, and 22.

In some embodiments, the disclosure provides compositions with bacterial strains that comprise 16S rDNA sequences having at least 97% sequence identity with nucleic acid sequences selected from the group consisting of SEQ ID NOs: 2, 3, 6, 9, 10, 12, 15-18, 20, and 22. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 2, 3, 6, 9, 10, 12, 15-18, 20, and 22. In some embodiments, the compositions comprise 12 purified bacterial strains, wherein the bacterial strains comprise 16S rDNA sequences having at least 97% sequence identity with nucleic acid sequences SEQ ID NOs: 2, 3, 6, 9, 10, 12, 15-18, 20, and 22. In some embodiments, the compositions consist of 12 purified bacterial strains, wherein the bacterial strains comprise 16S rDNA sequences having at least 97% sequence identity with nucleic acid sequences SEQ ID NOs: 2, 3, 6, 9, 10, 12, 15-18, 20, and 22. In some embodiments, the compositions essentially consist of 12 purified bacterial strains, wherein the bacterial strains comprise 16S rDNA sequences having at least 97% sequence identity with nucleic acid sequences SEQ ID NOs: 2, 3, 6, 9, 10, 12, 15-18, 20, and 22.

The bacterial strains in live bacterial product 5 are related to the following species: Bacteroides uniformis, Bacteroides vulgatus, Bifidobacterium adolescentis, Blautia producta, Clostridium hathewayi, Clostridium innocuum, Dorea longicatena, Eubacterium halli, Faecalibacterium prausnitzii, Parabacteroides merdae, Prevotella copri, and Ruminococcus faecis (see, e.g., Table 5). In some embodiments, the compositions comprise two or more (e.g., 2, 3, 4, 5, or more) bacterial species selected from the group consisting of Bacteroides uniformis, Bacteroides vulgatus, Bifidobacterium adolescentis, Blautia producta, Clostridium hathewayi, Clostridium innocuum, Dorea longicatena, Eubacterium halli, Faecalibacterium prausnitzii, Parabacteroides merdae, Prevotella copri, and Ruminococcus faecis. In some embodiments, the compositions comprise 12 bacterial species: Bacteroides uniformis, Bacteroides vulgatus, Bifidobacterium adolescentis, Blautia producta, Clostridium hathewayi, Clostridium innocuum, Dorea longicatena, Eubacterium halli, Faecalibacterium prausnitzii, Parabacteroides merdae, Prevotella copri, and Ruminococcus faecis. In some embodiments, the compositions consist of 12 bacterial species: Bacteroides uniformis, Bacteroides vulgatus, Bifidobacterium adolescentis, Blautia producta, Clostridium hathewayi, Clostridium innocuum, Dorea longicatena, Eubacterium halli, Faecalibacterium prausnitzii, Parabacteroides merdae, Prevotella copri, and Ruminococcus faecis. In some embodiments, the compositions essentially consist of 12 bacterial species: Bacteroides uniformis, Bacteroides vulgatus, Bifidobacterium adolescentis, Blautia producta, Clostridium hathewayi, Clostridium innocuum, Dorea longicatena, Eubacterium halli, Faecalibacterium prausnitzii, Parabacteroides merdae, Prevotella copri, and Ruminococcus faecis.

In one aspect, the disclosure provides compositions comprising one or more bacterial strains selected from the group consisting of strains 27, 28, 31, 2, 20, 35, 38-41, 43, and 45 (See, Table 5).

TABLE 5
Live bacterial product 5

| | | 16S rDNA |
|---|---|---|
| | | SEQ |
| Genus species | Strain No | ID NO |
| Bacteroides_uniformis | Strain 27 | 2 |
| Bacteroides_vulgatus | Strain 28 | 3 |
| Bifidobacterium_adolescentis | Strain 31 | 6 |
| Blautia_producta | Strain 2 | 9 |
| Clostridium_hathewayi | Strain 20 | 10 |
| Parabacteroides distasonis | Strain 35 | 12 |
| Dorea longicatena | Strain 38 | 15 |
| Eubacterium_halli | Strain 39 | 16 |
| Faecalibacterium_prausnitzii | Strain 40 | 17 |
| Parabacteroides_merdae | Strain 41 | 18 |
| Prevotella_copri | Strain 43 | 20 |
| Ruminococcus_faecis | Strain 45 | 22 |

In one aspect, the disclosure provides live bacterial product 6 (LBP 6) (see, e.g., FIG. 4, Table 6). As shown in FIG. 4 and Table 6, live bacterial product 6 contains bacterial strains that comprise 16S rDNA sequences with nucleic acid sequences SEQ ID NOs: 3-5, 7, 12, 15-17, 19, and 22. In some embodiments, the compositions comprise at least two (e.g., 2, 3, 4, 5, or more) bacterial strains that comprise 16S rDNA sequences with nucleic acid sequences SEQ ID NOs: 3-5, 7, 12, 15-17, 19, and 22. In some embodiments, the composition comprises 10 purified bacterial strains, wherein the bacterial strains comprise 16S rDNA sequences with nucleic acid sequences SEQ ID NOs: 3-5, 7, 12, 15-17, 19, and 22. In some embodiments, the composition consists of 10 purified bacterial strains, wherein the bacterial strains comprise 16S rDNA sequences with nucleic acid sequences SEQ ID NOs: 3-5, 7, 12, 15-17, 19, and 22. In some embodiments, the composition essentially consists of 10 purified bacterial strains, wherein the bacterial strains comprise 16S rDNA sequences with nucleic acid sequences SEQ ID NOs: 3-5, 7, 12, 15-17, 19, and 22.

In some embodiments, the disclosure provides compositions with bacterial strains that comprise 16S rDNA sequences having at least 97% sequence identity with nucleic acid sequences selected from the group consisting of SEQ ID NOs: 3-5, 7, 12, 15-17, 19, and 22. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 3-5, 7, 12, 15-17, 19, and 22. In some embodiments, the compositions comprise 10 purified bacterial strains, wherein the bacterial strains comprise 16S rDNA sequences having at least 97% sequence identity with nucleic acid sequences SEQ ID NOs: 3-5, 7, 12, 15-17, 19, and 22. In some embodiments, the compositions consist of 10 purified bacterial strains, wherein the bacterial strains comprise 16S rDNA sequences having at least 97% sequence identity with nucleic acid sequences SEQ ID NOs: 3-5, 7, 12, 15-17, 19, and 22. In some embodiments, the compositions essentially consist of 10 purified bacterial strains, wherein the bacterial strains comprise 16S rDNA sequences having at least 97% sequence identity with nucleic acid sequences SEQ ID NOs: 3-5, 7, 12, 15-17, 19, and 22.

The bacterial strains in live bacterial product 6 are related to the following species: Bacteroides vulgatus, Barnesiella intestinihominis, Parabacteroides merdae, Bifidobacterium longum, Blautia obeum, Clostridium innocuum, Dorea longicatena, Eubacterium halli, Faecalibacterium prausnitzii, Parabacteroides distasonis, and Ruminococcus faecis (see, e.g., Table 6). It should be appreciated that multiple bacterial strains of the compositions described herein can have the same related bacterial species. For example, the bacterial strains having 16S rDNA sequences with nucleic acid sequences SEQ ID NO: 12 and SEQ ID NO: 119 both have Parabacteroides distasonis as the related species. In some embodiments, the compositions comprise two or more (e.g., 2, 3, 4, 5, or more) bacterial species selected from the group consisting of Bacteroides vulgatus, Barnesiella intestinihominis, Parabacteroides merdae, Bifidobacterium longum, Blautia obeum, Clostridium innocuum, Dorea longicatena, Eubacterium halli, Faecalibacterium prausnitzii, Parabacteroides distasonis, and Ruminococcus faecis. In some embodiments, the compositions comprise 10 bacterial species: Bacteroides vulgatus, Barnesiella intestinihominis, Parabacteroides merdae, Bifidobacterium longum, Blautia obeum, Clostridium innocuum, Dorea longicatena, Eubacterium halli, Faecalibacterium prausnitzii, Parabacteroides distasonis, and Ruminococcus faecis. In some embodiments, the compositions consist of 10 bacterial species: Bacteroides vulgatus, Barnesiella intestinihominis, Parabacteroides merdae, Bifidobacterium longum, Blautia obeum, Clostridium innocuum, Dorea longicatena, Eubacterium halli, Faecalibacterium prausnitzii, Parabacteroides distasonis, and Ruminococcus faecis. In some embodiments, the compositions essentially consist 10 bacterial species: Bacteroides vulgatus, Barnesiella intestinihominis, Parabacteroides merdae, Bifidobacterium longum, Blautia obeum, Clostridium innocuum, Dorea longicatena, Eubacterium halli, Faecalibacterium prausnitzii, Parabacteroides distasonis, and Ruminococcus faecis.

In one aspect, the disclosure provides compositions comprising one or more bacterial strains selected from the group consisting of strains 28-30, 32, 35, 38-40, 42 and 45 (See Table 6).

TABLE 6
Live bacterial product 6

| | | 16S rDNA |
|---|---|---|
| | | SEQ |
| Genus species | Strain No. | ID NO |
| Bacteroides_vulgatus | Strain 28 | 3 |
| Parabacteroides merdae | Strain 29 | 4 |
| Bifidobacterium_longum | Strain 30 | 5 |
| Blautia_obeum | Strain 32 | 7 |
| Parabacteroides distasonis | Strain 35 | 12 |
| Dorea longicatena | Strain 38 | 15 |
| Eubacterium_halli | Strain 39 | 16 |
| Faecalibacterium_prausnitzii | Strain 40 | 17 |
| Parabacteroides_distasonis | Strain 42 | 19 |
| Ruminococcus_faecis | Strain 45 | 22 |

In one aspect, the disclosure provides live bacterial product 7 (LBP 7) (see, e.g., FIG. 4, Table 7). As shown in FIG. 4 and Table 7, live bacterial product 7 contains bacterial strains that comprise 16S rDNA sequences with nucleic acid sequences SEQ ID NOs: 3, 6-8, 11-13, 16, 18, and 20. In some embodiments, the compositions comprise at least two (e.g., 2, 3, 4, 5, or more) bacterial strains that comprise 16S rDNA sequences with nucleic acid sequences SEQ ID NOs: 3, 6-8, 11-13, 16, 18, and 20. In some embodiments, the composition comprises 10 purified bacterial strains, wherein the bacterial strains comprise 16S rDNA sequences with nucleic acid sequences SEQ ID NOs: 3, 6-8, 11-13, 16, 18, and 20. In some embodiments, the composition consists of 10 purified bacterial strains, wherein the bacterial strains comprise 16S rDNA sequences with nucleic acid sequences SEQ ID NOs: 3, 6-8, 11-13, 16, 18, and 20. In some embodiments, the composition essentially consists of 10 purified bacterial strains, wherein the bacterial strains comprise 16S rDNA sequences with nucleic acid sequences SEQ ID NOs: 3, 6-8, 11-13, 16, 18, and 20.

In some embodiments, the disclosure provides compositions with bacterial strains that comprise 16S rDNA sequences having at least 97% sequence identity with nucleic acid sequences selected from the group consisting of SEQ ID NOs: 3, 6-8, 11-13, 16, 18, and 20. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 3, 6-8, 11-13, 16, 18, and 20. In some embodiments, the compositions comprise 10 purified bacterial strains, wherein the bacterial strains comprise 16S rDNA sequences having at least 97% sequence identity with nucleic acid sequences SEQ ID NOs: 3, 6-8, 11-13, 16, 18, and 20. In some embodiments, the compositions consist of 10 purified bacterial strains, wherein the bacterial strains comprise 16S rDNA sequences having at least 97% sequence identity with nucleic acid sequences SEQ ID NOs: 3, 6-8, 11-13, 16, 18, and 20. In some embodiments, the compositions essentially consist of 10 purified bacterial strains, wherein the bacterial strains comprise 16S rDNA sequences having at least 97% sequence identity with nucleic acid sequences SEQ ID NOs: 3, 6-8, 11-13, 16, 18, and 20.

The bacterial strains in live bacterial product 7 are related to the following species: Bacteroides vulgatus, Bifidobacterium adolescentis, Blautia obeum, Blautia wexlerae, Clostridium bolteae, Clostridium innocuum, Parabacteroides distasonis, Collinsella aerofaciens, Eubacterium halli, Parabacteroides merdae, and Prevotella copri (see, e.g., Table 7). In some embodiments, the compositions comprise two or more (e.g., 2, 3, 4, 5, or more) bacterial species selected from the group consisting of Bacteroides vulgatus, Bifidobacterium adolescentis, Blautia obeum, Blautia wexlerae, Clostridium bolteae, Clostridium innocuum, Parabacteroides distasonis, Collinsella aerofaciens, Eubacterium halli, Parabacteroides merdae, and Prevotella copri. In some embodiments, the compositions comprise 10 bacterial species: Bacteroides vulgatus, Bifidobacterium adolescentis, Blautia obeum, Blautia wexlerae, Clostridium bolteae, Clostridium innocuum, Parabacteroides distasonis, Collinsella aerofaciens, Eubacterium halli, Parabacteroides merdae, and Prevotella copri. In some embodiments, the compositions consist of 10 bacterial species: Bacteroides vulgatus, Bifidobacterium adolescentis, Blautia obeum, Blautia wexlerae, Clostridium bolteae, Clostridium innocuum, Parabacteroides distasonis, Collinsella aerofaciens, Eubacterium halli, Parabacteroides merdae, and Prevotella copri. In some embodiments, the compositions essentially consist 10 bacterial species: Bacteroides vulgatus, Bifidobacterium adolescentis, Blautia obeum, Blautia wexlerae, Clostridium bolteae, Clostridium innocuum, Parabacteroides distasonis, Collinsella aerofaciens, Eubacterium halli, Parabacteroides merdae, and Prevotella copri.

In one aspect, the disclosure provides compositions comprising one or more bacterial strains selected from the group consisting of strains 28, 31-33, 34-36, 39, 41 and 43 (See Table 7).

TABLE 7
Live bacterial product 7

| | | 16S rDNA |
|---|---|---|
| | | SEQ |
| Genus species | Strain No. | ID NO |
| Bacteroides_vulgatus | Strain 28 | 3 |
| Bifidobacterium_adolescentis | Strain 31 | 6 |
| Blautia_obeum | Strain 32 | 7 |
| Blautia_wexlerae | Strain 33 | 8 |
| Clostridium_bolteae | Strain 34 | 11 |
| Parabacteroides distasonis | Strain 35 | 12 |
| Collinsella_aerofaciens | Strain 36 | 13 |
| Eubacterium_halli | Strain 39 | 16 |
| Parabacteroides_merdae | Strain 41 | 18 |
| Prevotella_copri | Strain 43 | 20 |

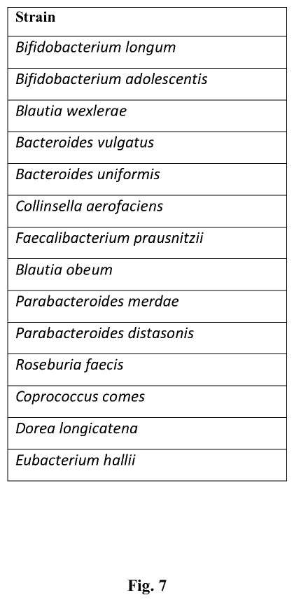

In one aspect, the composition comprises bacterial strains of species identified in stool samples from donors and also present in the live bacteria products (e.g., LBP 1-7) provided herein. In one aspect, the composition comprises one or more bacterial strains, wherein the bacterial strains are related to the following species: Bifidobacterium longum, Bifidobacterium adolescentis, Blautia wexlerae, Bacteroides vulgatus, Bacteroides uniformis, Collinsella aerofaciens, Faecalibacterium prausnitzii, Blautia obeum, Parabacteroides merdae, Parabacteroides distasonis, Roseburia faecis, Coprococcus comes, Dorea longicatena, and Eubacterium hallii (see, e.g., FIG. 7). In some embodiments, the composition comprises at least two bacterial strains, wherein the bacterial strains are related to the following species: Bifidobacterium longum, Bifidobacterium adolescentis, Blautia wexlerae, Bacteroides vulgatus, Bacteroides uniformis, Collinsella aerofaciens, Faecalibacterium prausnitzii, Blautia obeum, Parabacteroides merdae, Parabacteroides distasonis, Roseburia faecis, Coprococcus comes, Dorea longicatena, and Eubacterium hallii (see, e.g., FIG. 7). In some embodiments, the compositions comprise two or more (e.g., 2, 3, 4, 5, or more) bacterial species selected from the group consisting of Bifidobacterium longum, Bifidobacterium adolescentis, Blautia wexlerae, Bacteroides vulgatus, Bacteroides uniformis, Collinsella aerofaciens, Faecalibacterium prausnitzii, Blautia obeum, Parabacteroides merdae, Parabacteroides distasonis, Roseburia faecis, Coprococcus comes, Dorea longicatena, and Eubacterium hallii. In some embodiments, the compositions consist of two or more (e.g., 2, 3, 4, 5, or more) bacterial species selected from the group consisting of Bifidobacterium longum, Bifidobacterium adolescentis, Blautia wexlerae, Bacteroides vulgatus, Bacteroides uniformis, Collinsella aerofaciens, Faecalibacterium prausnitzii, Blautia obeum, Parabacteroides merdae, Parabacteroides distasonis, Roseburia faecis, Coprococcus comes, Dorea longicatena, and Eubacterium hallii. In some embodiments, the compositions essentially consist of two or more (e.g., 2, 3, 4, 5, or more) bacterial species selected from the group consisting of Bifidobacterium longum, Bifidobacterium adolescentis, Blautia wexlerae, Bacteroides vulgatus, Bacteroides uniformis, Collinsella aerofaciens, Faecalibacterium prausnitzii, Blautia obeum, Parabacteroides merdae, Parabacteroides distasonis, Roseburia faecis, Coprococcus comes, Dorea longicatena, and Eubacterium hallii.

In some embodiments, bacterial strains that are considered pathogen-antagonistic bacterial strains may be selected and included in any of the bacterial compositions described herein. Pathogen-antagonistic strains may be identified by any method known in the art. For example, in some embodiments, bacterial strains are evaluated for pathogen-antagonizing activity using assays, such as a soft agar overlay assay, as described in the Examples. Briefly, a soft agar overlay assay involves growing bacterial isolates (test strains) and spotting them onto agar plates (for example, tryptone soy agar, TSA). A second layer of soft agar (e.g., 0.7% w/v agar) is seeded with an inoculum of the target strain. The plate is incubated, and a zone of inhibition of bacterial growth is indicative of suppression (antagonizing activity) of the target strain. Inhibitory cultures may be subjected to 16S rRNA gene sequencing for strain identification. Alternatively, or in addition, growth competition assays may be used to evaluated pathogen-antagonistic activity of bacterial strains. Briefly, growth competition assays (or fitness assays or competition assays) involve co-culturing two or more candidate bacterial strains. The co-cultured strains are allowed to compete for limited resources and then plating diluted samples on selective growth media to determine relative fitness (see, e.g., Lenski et al., Proc. Natl. Sci. USA 91:6808-6814 (1994)).

In some embodiments, the compositions described herein comprise one or more (e.g., 1, 2, 3, 4, 5, or more) purified bacterial strain that has been identified as a pathogen-antagonizing strain. In some embodiments, the compositions described herein comprise one or more (e.g., 1, 2, 3, 4, 5, or more) purified bacterial strain that has been identified as have antagonistic or inhibitory activity towards Carbapenem Resistant Enterobacteriaceae (CRE). In some embodiments, the compositions described herein comprise one or more (e.g., 1, 2, 3, 4, 5, or more) purified bacterial strain that has been identified as have antagonistic or inhibitory activity towards Vancomycin Resistant Enterococci (VRE). In some embodiments, the compositions described herein comprise one or more (e.g., 1, 2, 3, 4, 5 or more) purified bacterial strain selected from the group consisting of the following species Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, Bacteroides cellulosyliticus, Eubacterium fissicatena, and Lachnospiraceae bacterium. In some embodiments, the compositions described herein consists of one or more (e.g., 1, 2, 3, 4, 5 or more) purified bacterial strain selected from the group consisting of the following species Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, Bacteroides cellulosyliticus, Eubacterium fissicatena, and Lachnospiraceae bacterium. In some embodiments, the “Barnesiella spp” is referred to as a “Parabacteroides spp”.

In some embodiments, the compositions described herein comprise one or more (e.g., 1, 2, 3, 4, 5, or more) purified bacterial strain that has been identified as a pathogen-antagonizing strain. In some embodiments, the compositions described herein comprise one or more (e.g., 1, 2, 3, 4, 5, or more) purified bacterial strain listed in Tables B, C, D and E (See also FIGS. 9, 17, 19, 20, and 22.

TABLE B
Strains antagonistic to CRE as identified by soft agar assay

| | | 16 S rDNA |
|---|---|---|
| | | SEQ |
| Strain | Classification | ID NO |
| Strain 1 | Flavonifractor plautii | 23 |
| Strain 2 | Blautia producta-1 | 9 |
| Strain 3 | Blautia producta-2 | 24 |
| Strain 4 | Blautia producta-3 | 25 |
| Strain 5 | Clostridium ramosum | 26 |
| Strain 6 | Flavonifractor plautii | 27 |
| Strain 7 | Barnesiella | 28 |
| Strain 8 | Clostridium symbiosum | 29 |

TABLE C
Strains antagonistic to VRE as identified by soft agar assay

| | | 16S rDNA |
|---|---|---|
| | | SEQ |
| Strain | Classification | ID NO |
| Strain 23 | Eubacterium fissicatena | 30 |
| Strain 25 | Lachnospiraceae bacterium | 31 |

TABLE D
Strains antagonistic to CRE as identified
by broth competition assay

| | | 16S rDNA |
|---|---|---|
| | | SEQ |
| Strain ID | Closest relative | ID NO |
| Strain 1 | Flavonifractor plautii | 23 |
| Strain 18 | Dorea longicatena | 32 |
| Strain 10 | Blautia producta | 33 |
| Strain 2 | Blautia producta | 9 |
| Strain 46 | Escherichia coli | 34 |
| Strain 47 | Lactococcus lactis | 35 |
| Strain 48 | Lactobacillus ruminis | 36 |

TABLE E
Strains antagonistic to CRE as identified
by broth competition assay

| | | 16 S rDNA |
|---|---|---|
| | | SEQ |
| Strain ID | Closest relative | ID NO |
| Strain 47 | Lactococcus lactis | 35 |
| Strain 18 | Dorea longicatena | 32 |
| Strain 48 | Lactobacillus ruminis | 36 |
| Strain 49 | Lactobacillus ruminis | 37 |
| Strain 50 | Lactobacillus animalis | 38 |
| Strain 51 | Lactobacillus rhamnosus | 39 |
| Strain 52 | Lactobacillus rhamnosus | 40 |
| Strain 53 | Lactobacillus rhamnosus | 41 |

In some embodiments, the compositions described herein comprise one or more (e.g., 1, 2, 3, 4, 5, or more) purified bacterial strain that has been identified as a pathogen-antagonizing strain. In some embodiments, the compositions described herein comprise one or more (e.g., 1, 2, 3, 4, 5, or more) purified bacterial strain that has been identified as have antagonistic or inhibitory activity towards Carbapenem Resistant Enterobacteriaceae (CRE). In some embodiments, the compositions described herein comprise one or more (e.g., 1, 2, 3, 4, 5, or more) purified bacterial strain that has been identified as have antagonistic or inhibitory activity towards Vancomycin Resistant Enterococci (VRE). In some embodiments, the compositions described herein comprise one or more (e.g., 1, 2, 3, 4, 5 or more) purified bacterial strain selected from the group consisting of the following species Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, Bacteroides cellulosyliticus, Eubacterium fissicatena, Lachnospiraceae bacterium, Escherichia coli, Lactococcus lactus, Lactobacillus ruminis, Lactobacillus animalis, or Lactobacillus rhamnosus. In some embodiments, the compositions described herein consists of one or more (e.g., 1, 2, 3, 4, 5 or more) purified bacterial strain selected from the group consisting of the following species Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, Bacteroides cellulosyliticus, Eubacterium fissicatena, Lachnospiraceae bacterium, Escherichia coli, Lactococcus lactus, Lactobacillus ruminis, Lactobacillus animalis, or Lactobacillus rhamnosus.

In one aspect, the disclosure provides composition comprising one or more bacterial strains selected from the group consisting of strains 1-8, 23, 25, 18, 10, and 46-53 (See Tables B-E).

In some embodiments, the compositions described herein comprise one or more (e.g., 1, 2, 3, 4, 5, or more) purified bacterial strain that has been identified as a pathogen-antagonizing strain. In some embodiments, the compositions described herein comprise one or more (e.g., 1, 2, 3, 4, 5, or more) purified bacterial strain that has been identified as have antagonistic or inhibitory activity towards Carbapenem Resistant Enterobacteriaceae (CRE). In some embodiments, the compositions described herein comprise one or more (e.g., 1, 2, 3, 4, 5, or more) purified bacterial strain that has been identified as have antagonistic or inhibitory activity towards Vancomycin Resistant Enterococci (VRE). In some embodiments, the compositions described herein comprise one or more (e.g., 1, 2, 3, 4, 5 or more) purified bacterial strain selected from the group consisting of the following species Escherichia coli, Lactococcus lactus, Lactobacillus ruminis, Lactobacillus, animalis, or Lactobacillus rhamnosus. In some embodiments, the compositions described herein consists of one or more (e.g., 1, 2, 3, 4, 5 or more) purified bacterial strain selected from the group consisting of the following species Escherichia coli, Lactococcus lactus, Lactobacillus ruminis, Lactobacillus animalis, or Lactobacillus rhamnosus.

In one aspect, the disclosure provides composition comprising one or more bacterial strains selected from the group consisting of strains 46-53 (See Tables D-E).

In some embodiments, the compositions described herein comprise one or more (e.g., 1, 2, 3, 4, 5, or more) purified bacterial strain that has been identified as a pathogen-antagonizing strain. In some embodiments, the compositions described herein comprise one or more (e.g., 1, 2, 3, 4, 5, or more) purified bacterial strain that has been identified as have antagonistic or inhibitory activity towards Vancomycin Resistant Enterococci (VRE). In some embodiments, the compositions that have antagonistic or inhibitory activity towards Vancomycin Resistant Enterococci (VRE) described herein comprise one or two purified bacterial strains selected from the group consisting of the following species: Eubacterium fissicatena and Lachnospiraceae bacterium. In some embodiments, the compositions that have antagonistic or inhibitory activity towards Vancomycin Resistant Enterococci (VRE) described herein consist of one or two purified bacterial strains selected from the group consisting of the following species: Eubacterium fissicatena and Lachnospiraceae bacterium.

In one aspect, the disclosure provides composition comprising one or more bacterial 20 strains selected from the group consisting of strains 23 and 25 (See Table C).

In some embodiments, the compositions described herein comprise one or more (e.g., 1, 2, 3, 4, 5, or more) purified bacterial strain that has been identified as a pathogen-antagonizing strain. In some embodiments, the compositions described herein comprise one or more (e.g., 1, 2, 3, 4, 5, or more) purified bacterial strain that has been identified as have antagonistic or inhibitory activity towards Carbapenem Resistant Enterobacteriaceae (CRE). In some embodiments, the compositions described herein that have antagonistic or inhibitory activity towards Carbapenem Resistant Enterobacteriaceae (CRE) comprise one or more (e.g., 1, 2, 3, 4, 5 or more) purified bacterial strain selected from the group consisting of the following species Flavonifractor plautii, Blautia producta, Clostridium ramosum, Barnesiella spp, Clostridium symbiosum, Anaerotruncus colihominis, Clostridium innocuum, Clostridium indolis, Bacteroides ovatus, Bacteroides cellulosyliticus, Escherichia coli, Lactococcus lactus, Lactobacillus ruminis, Lactobacillus animalis, and Lactobacillus rhamnosus. In some embodiments, the compositions described herein comprise one or more (e.g., 1, 2, 3, 4, 5, or more) purified bacterial strain that has been identified as having antagonistic or inhibitory activity towards Carbapenem Resistant Enterobacteriaceae (CRE). In some embodiments, the compositions described herein that have antagonistic or inhibitory activity towards Carbapenem Resistant Enterobacteriaceae (CRE) consist of one or more (e.g., 1, 2, 3, 4, 5 or more) purified bacterial strain selected from the group consisting of the following species: Escherichia coli, Lactococcus lactus, Lactobacillus ruminis, Lactobacillus animalis, and Lactobacillus rhamnosus.

In one aspect, the disclosure provides composition comprising one or more bacterial strains selected from the group consisting of strains 1, 2, 18, 10, and 46-53 (See Tables B and D-E). In one aspect, the disclosure provides compositions comprising bacterial strains that comprise 16S rDNA sequences with nucleic acid sequences SEQ ID NOs: 1-41. In some embodiments, the compositions comprise at least two (e.g., 2, 3, 4, 5, or more) bacterial strains that comprise 16S rDNA sequences with nucleic acid sequences SEQ ID NOs: 1-41. In some embodiments, the composition comprises 16 purified bacterial strains, wherein the bacterial strains comprise 16S rDNA sequences with nucleic acid sequences SEQ ID NOs: 1-41. In some embodiments, the composition consists of 16 purified bacterial strains, wherein the bacterial strains comprise 16S rDNA sequences with nucleic acid sequences SEQ ID NOs: 1-41. In some embodiments, the composition essentially consists of 16 purified bacterial strains, wherein the bacterial strains comprise 16S rDNA sequences with nucleic acid sequences SEQ ID NOs: 1-41. As used herein, “essentially consists of” (and like phrases) refers to a composition that includes no additional bacterial strains.

In some embodiments, the disclosure provides compositions with bacterial strains that comprise 16S rDNA sequences having at least 97% sequence identity with nucleic acid sequences selected from the group consisting of SEQ ID NOs: 1-41. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 1-41. In some embodiments, the compositions comprise 16 purified bacterial strains, wherein the bacterial strains comprise 16S rDNA sequences having at least 97% sequence identity with nucleic acid sequences SEQ ID NOs: 1-41. In some embodiments, the compositions consist of 16 purified bacterial strains, wherein the bacterial strains comprise 16S rDNA sequences having at least 97% sequence identity with nucleic acid sequences SEQ ID NOs: 1-41. In some embodiments, the compositions essentially consist of 16 purified bacterial strains, wherein the bacterial strains comprise 16S rDNA sequences having at least 97% sequence identity with nucleic acid sequences SEQ ID NOs: 1-41.

In one aspect, the disclosure provides compositions comprising bacterial strains that comprise 16S rDNA sequences with nucleic acid sequences SEQ ID NOs: 9, 23, and 24-41. In some embodiments, the compositions comprise at least two (e.g., 2, 3, 4, 5, or more) bacterial strains that comprise 16S rDNA sequences with nucleic acid sequences SEQ ID NOs: 9, 23, and 24-41. In some embodiments, the composition comprises 16 purified bacterial strains, wherein the bacterial strains comprise 16S rDNA sequences with nucleic acid sequences SEQ ID NOs: 9, 23, and 24-41. In some embodiments, the composition consists of 16 purified bacterial strains, wherein the bacterial strains comprise 16S rDNA sequences with nucleic acid sequences SEQ ID NOs: 9, 23, and 24-41. In some embodiments, the composition essentially consists of 16 purified bacterial strains, wherein the bacterial strains comprise 16S rDNA sequences with nucleic acid sequences SEQ ID NOs: 9, 23, and 24-41. As used herein, “essentially consists of” (and like phrases) refers to a composition that includes no additional bacterial strains.

In some embodiments, the disclosure provides compositions with bacterial strains that comprise 16S rDNA sequences having at least 97% sequence identity with nucleic acid sequences selected from the group consisting of SEQ ID NOs: 9, 23, and 24-41. In some embodiments, the disclosure provides compositions comprising two or more (e.g., 2, 3, 4, 5, or more) purified bacterial strains that comprise 16S rDNA sequences having at least 97% sequence identity with nucleic acid sequences selected from the group consisting of SEQ ID NOs: 9, 23, and 24-41. In some embodiments, the compositions comprise 16 purified bacterial strains, wherein the bacterial strains comprise 16S rDNA sequences having at least 97% sequence identity with nucleic acid sequences SEQ ID NOs: 9, 23, and 24-41. In some embodiments, the compositions consist of 16 purified bacterial strains, wherein the bacterial strains comprise 16S rDNA sequences having at least 97% sequence identity with nucleic acid sequences SEQ ID NOs: 9, 23, and 24-41. In some embodiments, the compositions essentially consist of 16 purified bacterial strains, wherein the bacterial strains comprise 16S rDNA sequences having at least 97% sequence identity with nucleic acid sequences SEQ ID NOs: 9, 23, and 24-41.

In some embodiments, the compositions comprise two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more) bacterial species selected from the group consisting of Bacteroides caccae, Bacteroides intestinalis (Bacteroides cellulosyticus), Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Clostridiales bacterium VE202-06 (Blautia coccoides, Blautia producta), Clostridium citroniae, Clostridium sp. C105KSO14 (Clostridium clostridioforme), Clostridiales bacterium VE202-21 (Eubacterium contortum, Clostridium innocuum), Erysipelotrichaceae bacterium 6_1_45 (Clostridium innocuum), Paeniclostridium sordellii, Coprococcus comes, Dorea longicatena, Erysipelatoclostridium ramosum, Eubacterium rectale, Odoribacter sp. UNK.MGS-12, Bacteroides sp. 1_1_14 (Parabacteroides merdae), Bacteroides sp. UNK.MGS-14 (Parabacteroides merdae), Bacteroides xylanisolvens, Blautia obeum, Alistipes putredinis, Collinsella aerofaciens, Eubacterium hallii, Alistipes shahii, Anaerostipes caccae, hascolarctobacterium faecis, Agathobaculum, Bacteroides sp. 2_1_56FAA (Bacteroides. fragilis), Fusobacterium mortiferum, Paraclostridium bifermentans, and Escherichia sp. 3_2_53FAA.

In some embodiments, the compositions comprise 36 bacterial species: Bacteroides caccae, Bacteroides intestinalis (Bacteroides cellulosyticus), Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Clostridiales bacterium VE202-06 (Blautia coccoides, Blautia producta), Clostridium citroniae, Clostridium sp. C105KSO14 (Clostridium clostridioforme), Clostridiales bacterium VE202-21 (Eubacterium contortum, Clostridium innocuum), Erysipelotrichaceae bacterium 6_1_45 (Clostridium innocuum), Paeniclostridium sordellii, Coprococcus comes, Dorea longicatena, Erysipelatoclostridium ramosum, Eubacterium rectale, Odoribacter sp. UNK.MGS-12, Bacteroides sp. 1_1_14 (Parabacteroides merdae), Bacteroides sp. UNK.MGS-14 (Parabacteroides merdae), Bacteroides xylanisolvens, Blautia obeum, Alistipes putredinis, Collinsella aerofaciens, Eubacterium hallii, Alistipes shahii, Anaerostipes caccae, hascolarctobacterium faecis, Agathobaculum, Bacteroides sp. 2_1_56FAA (Bacteroides. fragilis), Fusobacterium mortiferum, Paraclostridium bifermentans, and Escherichia sp. 3_2_53FAA. In some embodiments, the compositions consist of 36 bacterial species: Bacteroides caccae, Bacteroides intestinalis (Bacteroides cellulosyticus), Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Clostridiales bacterium VE202-06 (Blautia coccoides, Blautia producta, Clostridium citroniae, Clostridium sp. C105KSO]4 (Clostridium clostridioforme), Clostridiales bacterium VE202-21 (Eubacterium contortum, Clostridium innocuum), Erysipelotrichaceae bacterium 6_1_45 (Clostridium innocuum), Paeniclostridium sordellii, Coprococcus comes, Dorea longicatena, Erysipelatoclostridium ramosum, Eubacterium rectale, Odoribacter sp. UNK.MGS-12, Bacteroides sp. 1_1_14 (Parabacteroides merdae), Bacteroides sp. UNKMGS-14 (Parabacteroides merdae), Bacteroides xylanisolvens, Blautia obeum, Alistipes putredinis, Collinsella aerofaciens, Eubacterium hallii, Alistipes shahii, Anaerostipes caccae, Phascolarctobacterium faecis, Agathobaculum, Bacteroides sp. 2_1_56FAA (Bacteroides. fragilis), Fusobacterium mortiferum, Paraclostridium bifermentans, and Escherichia sp. 3_2_53FAA. In some embodiments, the compositions essentially consist of 36 bacterial species: Bacteroides caccae, Bacteroides intestinalis (Bacteroides cellulosyticus), Bacteroides faecis, Bacteroides ovatus, Bacteroides thetaiotaomicron, Bacteroides uniformis, Bacteroides vulgatus, Bifidobacterium adolescentis, Bifidobacterium longum, Bifidobacterium pseudocatenulatum, Clostridiales bacterium VE202-06 (Blautia coccoides, Blautia producta), Clostridium citroniae, Clostridium sp. C105KSO14 (Clostridium clostridioforme), Clostridiales bacterium VE202-21 (Eubacterium contortum, Clostridium innocuum), Erysipelotrichaceae bacterium 6_1_45 (Clostridium innocuum), Paeniclostridium sordellii, Coprococcus comes, Dorea longicatena, Erysipelatoclostridium ramosum, Eubacterium rectale, Odoribacter sp. UNKMGS-12, Bacteroides sp. 1_1_14 (Parabacteroides merdae), Bacteroides sp. UNKMGS-14 (Parabacteroides merdae), Bacteroides xylanisolvens, Blautia obeum, Alistipes putredinis, Collinsella aerofaciens, Eubacterium hallii, Alistipes shahii, Anaerostipes caccae, Phascolarctobacterium faecis, Agathobaculum, Bacteroides sp. 2_1_56FAA (Bacteroides. fragilis), Fusobacterium mortiferum, Paraclostridium bifermentans, and Escherichia sp. 3_2_53FAA.

In one aspect, the disclosure provides compositions comprising bacterial strains that comprise 16S rDNA sequences with nucleic acid sequences selected from the group consisting of SEQ ID NOs: 42-77. In some embodiments, the compositions comprise at least two (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more) bacterial strains that comprise 16S rDNA sequences with nucleic acid sequences selected from the group consisting of SEQ ID NOs: 42-77. In some embodiments, the composition comprises 36 purified bacterial strains, wherein the bacterial strains comprise 16S rDNA sequences with nucleic acid sequences SEQ ID NOs: 42-77. In some embodiments, the composition consists of 36 purified bacterial strains, wherein the bacterial strains comprise 16S rDNA sequences with nucleic acid sequences SEQ ID NOs: 42-77. In some embodiments, the composition essentially consists of 36 purified bacterial strains, wherein the bacterial strains comprise 16S rDNA sequences with nucleic acid sequences SEQ ID NOs: 42-77. As used herein, “essentially consists of” (and like phrases) refers to a composition that includes no additional bacterial strains.

In some embodiments, the disclosure provides compositions with bacterial strains that comprise 16S rDNA sequences having at least 97% sequence identity with nucleic acid sequences selected from the group consisting of SEQ ID NOs: 42-77. In some embodiments, the disclosure provides compositions comprising two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more) purified bacterial strains that comprise 16S rDNA sequences having at least 97% sequence identity with nucleic acid sequences selected from the group consisting of SEQ ID NOs: 42-77. In some embodiments, the compositions comprise 36 purified bacterial strains, wherein the bacterial strains comprise 16S rDNA sequences having at least 97% sequence identity with nucleic acid sequences SEQ ID NOs: 42-77. In some embodiments, the compositions consist of 36 purified bacterial strains, wherein the bacterial strains comprise 16S rDNA sequences having at least 97% sequence identity with nucleic acid sequences SEQ ID NOs: 42-77. In some embodiments, the compositions essentially consist of 36 purified bacterial strains, wherein the bacterial strains comprise 16S rDNA sequences having at least 97% sequence identity with nucleic acid sequences SEQ ID NOs: 42-77. As used herein, “essentially consists of” (and like phrases) refers to a composition that includes no additional bacterial strains.

In some embodiments, the compositions comprise 23 bacterial species: Bacteroides caccae, Bacteroides intestinalis (Bacteroides cellulosyticus), Bacteroides faecis, Bacteroides ovatus, Bacteroides thetaiotaomicron, Bacteroides uniformis, Bacteroides vulgatus, Clostridiales bacterium VE202-06 (Blautia coccoides, Blautia producta), Clostridium citroniae, Clostridium sp. C105KSO14 (Clostridium clostridioforme), Clostridiales bacterium VE202-21 (Eubacterium contortum, Clostridium innocuum), Erysipelotrichaceae bacterium 6_1_45 (Clostridium innocuum), Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, Fusobacterium mortiferum, and Escherichia sp. 3_2_53FAA. In some embodiments, the compositions consist of 23 bacterial species: Bacteroides caccae, Bacteroides intestinalis (Bacteroides cellulosyticus), Bacteroides faecis, Bacteroides ovatus, Bacteroides thetaiotaomicron, Bacteroides uniformis, Bacteroides vulgatus, Clostridiales bacterium VE202-06 (Blautia coccoides, Blautia producta), Clostridium citroniae, Clostridium sp. C105KSO14 (Clostridium clostridioforme), Clostridiales bacterium VE202-21 (Eubacterium contortum, Clostridium innocuum), Erysipelotrichaceae bacterium 6_1_45 (Clostridium innocuum), Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, Fusobacterium mortiferum, and Escherichia sp. 3_2_53FAA. In some embodiments, the compositions essentially consist 23 bacterial species: Bacteroides caccae, Bacteroides intestinalis (Bacteroides cellulosyticus), Bacteroides faecis, Bacteroides ovatus, Bacteroides thetaiotaomicron, Bacteroides uniformis, Bacteroides vulgatus, Clostridiales bacterium VE202-06 (Blautia coccoides, Blautia producta), Clostridium citroniae, Clostridium sp. C105KSO14 (Clostridium clostridioforme), Clostridiales bacterium VE202-21 (Eubacterium contortum, Clostridium innocuum), Erysipelotrichaceae bacterium 6_1_45 (Clostridium innocuum), Coprococcus comes, Dorea longicatena, Erysipelatoclostridium ramosum, Eubacterium rectale, Bacteroides xylanisolvens, Blautia obeum, Alistipes putredinis, Eubacterium hallii, Alistipes shahii, Fusobacterium mortiferum, and Escherichia sp. 3_2_53FAA.

In one aspect, the disclosure provides compositions comprising bacterial strains that comprise 16S rDNA sequences with nucleic acid sequences selected from the group consisting of SEQ ID NOs: 42-48, 52-56, 58-61, 65-67, 69, 70, 75, and 77. In some embodiments, the compositions comprise at least two (e.g., 2, 3, 4, 5, 6, 7, 8, 9, 10, 11, 12, 13, 14, 15, 16, 17, 18, 19, 20, 21, 22, 23 or more) bacterial strains that comprise 16S rDNA sequences with nucleic acid sequences selected from the group consisting of SEQ ID NOs: 42-48, 52-56, 58-61, 65-67, 69, 70, 75, and 77. In some embodiments, the composition comprises 23 purified bacterial strains, wherein the bacterial strains comprise 16S rDNA sequences with nucleic acid sequences SEQ ID NOs: 42-48, 52-56, 58-61, 65-67, 69, 70, 75, and 77. In some embodiments, the composition consists of 23 purified bacterial strains, wherein the bacterial strains comprise 16S rDNA sequences with nucleic acid sequences SEQ ID NOs: 42-48, 52-56, 58-61, 65-67, 69, 70, 75, and 77. In some embodiments, the composition essentially consists of 23 purified bacterial strains, wherein the bacterial strains comprise 16S rDNA sequences with nucleic acid sequences SEQ ID NOs: 42-48, 52-56, 58-61, 65-67, 69, 70, 75, and 77. As used herein, “essentially consists of” (and like phrases) refers to a composition that includes no additional bacterial strains.

In some embodiments, the disclosure provides compositions with bacterial strains that comprise 16S rDNA sequences having at least 97% sequence identity with nucleic acid sequences selected from the group consisting of SEQ ID NOs: 42-48, 52-56, 58-61, 65-67, 69, 70, 75, and 77. In some embodiments, the disclosure provides compositions comprising two or more (e.g., 2, 3, 4, 5, 6, 7, 8, 9, 10, 11, 12, 13, 14, 15, 16, 17, 18, 19, 20, 21, 22, 23 or more) purified bacterial strains that comprise 16S rDNA sequences having at least 97% sequence identity with nucleic acid sequences selected from the group consisting of SEQ ID NOs: 42-48, 52-56, 58-61, 65-67, 69, 70, 75, and 77. In some embodiments, the compositions comprise 23 purified bacterial strains, wherein the bacterial strains comprise 16S rDNA sequences having at least 97% sequence identity with nucleic acid sequences SEQ ID NOs: 42-48, 52-56, 58-61, 65-67, 69, 70, 75, and 77. In some embodiments, the compositions consist of 23 purified bacterial strains, wherein the bacterial strains comprise 16S rDNA sequences having at least 97% sequence identity with nucleic acid sequences SEQ ID NOs: 42-48, 52-56, 58-61, 65-67, 69, 70, 75, and 77. In some embodiments, the compositions essentially consist of 23 purified bacterial strains, wherein the bacterial strains comprise 16S rDNA sequences having at least 97% sequence identity with nucleic acid sequences SEQ ID NOs: 42-48, 52-56, 58-61, 65-67, 69, 70, 75, and 77.

In some embodiments, the composition comprises at least the 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more most abundant bacterial strains present in a spore forming fraction of a fecal sample obtained from a subject. In some embodiments, the composition comprises at least the 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more most abundant bacterial species present in a spore forming fraction of a fecal sample obtained from a subject.

In some embodiments, the composition comprises at least the 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more most abundant bacterial strains present in a non-spore forming fraction of a fecal sample obtained from a subject. In some embodiments, the composition comprises at least the 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more most abundant bacterial species present in a non-spore forming fraction of a fecal sample obtained from a subject.

In some embodiments, the composition comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more bacterial strains present in a spore forming fraction of a fecal sample obtained from a subject, wherein the bacterial species suppress the replication, survival, and/or colonization of one or more pathogenic organisms. In some embodiments, the composition comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more bacterial species present in a spore forming fraction of a fecal sample obtained from a subject, wherein the bacterial species suppress the replication, survival, and/or colonization of one or more pathogenic organisms.

In some embodiments, the composition comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more bacterial strains present in a non-spore forming fraction of a fecal sample obtained from a subject, wherein the bacterial species suppress the replication, survival, and/or colonization of one or more pathogenic organisms. In some embodiments, the composition comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more bacterial species present in a non-spore forming fraction of a fecal sample obtained from a subject, wherein the bacterial species suppress the replication, survival, and/or colonization of one or more pathogenic organisms.

As will be appreciated by one of skill in the art, any one or more bacterial strain identified as having pathogen-antagonizing activity may be included in any of the compositions described herein.

In some embodiments, bacterial strains found to be present in a sample obtained from a donor may be identified and selected to be included in any of the bacterial compositions described herein. In some embodiments, a fecal sample or stool fraction thereof is analyzed to determine the bacterial composition of the sample or fraction thereof and identify the most abundant bacterial strains. For example, as described in Example 2, nucleic acids obtained from a fecal sample or stool fraction (e.g., a spore forming fraction or a non-spore forming fraction) may be sequenced to identify the bacterial strains present in the fecal sample or stool fraction. The relative abundance of the bacterial strains may be determined using nucleic acid sequencing data, for example by determining the number of reads associated with a bacterial strain relative to the number of reads of a control or the total number of reads in the sequencing reaction. The bacterial strains in the sample may be ranked based on the relative abundance in the sample. Tables 8-11 show the most abundant bacterial species identified in spore forming fractions (Tables 8 and 10) and non-spore forming fractions (Tables 9 and 11) obtained from two example donors. Any one or more of the bacterial strains identified in a fecal sample or stool fraction from a donor may be selected and included in a bacterial composition as described herein.

In one aspect, the compositions comprise a fraction of a fecal sample. In some embodiments, the compositions comprise a non-spore forming fraction of a fecal sample. In some embodiments, the compositions comprise a spore forming fraction of a fecal sample.

In some embodiments, the compositions comprise the most abundant bacterial species present in a fecal sample or stool sample from a donor. In some embodiments, the compositions comprise the 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more of the most abundant bacterial species present in a fecal sample or stool sample obtained from a donor or a fecal sample or stool sample obtained from more than one donor. In some embodiments, the compositions comprise the 5 most abundant bacterial species present in a fecal sample or stool sample obtained from a donor or a fecal sample or stool sample obtained from more than one donor. In some embodiments, the compositions comprise the 10 most abundant bacterial species present in a fecal sample or stool sample obtained from a donor or a fecal sample or stool sample obtained from more than one donor. In some embodiments, the compositions comprise the 15 most abundant bacterial species present in a fecal sample or stool sample obtained from a donor or a fecal sample or stool sample obtained from more than one donor. In some embodiments, the compositions comprise the 20 most abundant bacterial species present in a fecal sample or stool sample obtained from a donor or a fecal sample or stool sample obtained from more than one donor. In some embodiments, the compositions comprise the 23 most abundant bacterial species present in a fecal sample or stool sample obtained from a donor or a fecal sample or stool sample obtained from more than one donor. In some embodiments, the compositions comprise the 36 most abundant bacterial species present in a fecal sample or stool sample obtained from a donor or a fecal sample or stool sample obtained from more than one donor.

In some embodiments, the compositions comprise the 5 most abundant bacterial species presented in any of Tables 8-12. In some embodiments, the compositions comprise the 10 most abundant bacterial species presented in any of Tables 8-12. In some embodiments, the compositions comprise the 15 most abundant bacterial species presented in any of Tables 8-12. In some embodiments, the compositions comprise the 20 most abundant bacterial species presented in any of Tables 8-12. In some embodiments, the compositions comprise the 23 most abundant bacterial species presented in any of Tables 8-12. In some embodiments, the compositions comprise the 36 most abundant bacterial species presented in any of Tables 8-12.

In some embodiments, the compositions comprise one or more bacterial strains identified in the non-spore and/or spore forming fractions (Tables 8-12) and are found in any of the live bacterial products presented herein (Tables 1-7). In some embodiments, the compositions comprise one or more bacterial strains from species selected from the group consisting of Bifidobacterium longum, Bifidobacterium adolescentis, Blautia wexlerae, Bacteroides vulgatus, Bacteroides umiformis, Collinsella aerofaciens, Faecalibacterium prausnitzii, Blautia obeum, Parabacteroides merdae, Parabacteroides distasonis, Roseburia faecis, Coprococcus comes, Dorea longicatena, and Eubacterium hallii (see also FIG. 7).

Aspects of the disclosure relate to bacterial strains with 16S rDNA sequences that have sequence identity to a nucleic acid sequence of any one of the sequences of the bacterial strains or species described herein. The terms “identical,” or percent “identity,” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over the entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

In some embodiments, the bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity relative to any of the strains or bacterial species described herein over a specified region or over the entire sequence. It would be appreciated by one of skill in the art that the term “sequence identity” or “percent sequence identity,” in the context of two or more nucleic acid sequences or amino acid sequences, refers to a measure of similarity between two or more sequences or portion(s) thereof.

In some embodiments, the composition includes two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more) bacterial strains, wherein the two or more bacterial strains contain 16S rDNA sequences having at least 97% sequence identity with nucleic acid sequences selected from SEQ ID NOs: 1-22.

In some embodiments, the composition includes two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more) bacterial strains, wherein the two or more bacterial strains contain 16S rDNA sequences having at least 97% sequence identity with nucleic acid sequences selected from SEQ ID NOs: 42-77.

In some embodiments, the composition includes two or more (e.g., 2, 3, 4, 5, 6, 7, 8, 9, 10, 11, 12, 13, 14, 15, 16, 17, 18, 19, 20, 21, 22, 23, or more) bacterial strains, wherein the two or more bacterial strains contain 16S rDNA sequences having at least 97% sequence identity with nucleic acid sequences selected from SEQ ID NOs: 42-48, 52-56, 58-61, 65-67, 69, 70, 75, 77.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman. Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group. Madison. WI), or by manual alignment and visual inspection (see. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

In one aspect, the 16S rDNA sequences of purified bacterial strains were compared to 16S rDNA sequences of known bacterial species/strains in a bacterial genome database to identify the closest known related bacterial species to the bacterial strains disclosed herein. It should be appreciated that multiple bacterial strains of the compositions disclosed herein may have the same closest related bacterial species.

In some embodiments, the compositions described herein comprise spore forming and non-spore forming bacterial strains. In some embodiments, the compositions described herein comprise spore forming bacterial strains. In some embodiments, the compositions described herein comprise only spore forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore form may germinate when they are introduced in the intestine.

In some embodiments, at least one (e.g., 1, 2, 3, 4, 5, or more) of the bacterial strains in the composition is a spore former. In some embodiments, at least one (e.g., 1, 2, 3, 4, 5, or more) of the bacterial strains in the composition is in spore form. In some embodiments, at least one (e.g., 1, 2, 3, 4, 5, or more) of the bacterial strains in the composition is a non-spore former. In some embodiments, at least one (e.g., 1, 2, 3, 4, 5, or more) of the bacterial strains in the composition is in vegetative form. As discussed above, spore forming bacteria can also be in vegetative form. In some embodiments, at least one (e.g., 1, 2, 3, 4, 5, or more) of the bacterial strains in the composition is in spore form and at least one (e.g., 1, 2, 3, 4, 5, or more) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form.

In some embodiments, the compositions comprise bacterial strains that are spore forming bacterial strains. In some embodiments, the compositions comprise bacterial strains that are non-spore forming bacterial strains. In some embodiments, the compositions comprise bacterial strains that are spore forming bacterial strains and bacterial strains that are non-spore forming bacterial strains. In some embodiments, the compositions comprise a mixture of bacterial strains wherein at least 10% of the bacterial strains are spore forming bacterial strains, at least 20% of the bacterial strains are spore forming bacterial strains, at least 30% of the bacterial strains are spore forming bacterial strains, at least 40% of the bacterial strains are spore forming bacterial strains, at least 50% of the bacterial strains are spore forming bacterial strains, at least 60% of the bacterial strains are spore forming bacterial strains, at least 70% of the bacterial strains are spore forming bacterial strains, at least 80% of the bacterial strains are spore forming bacterial strains, at least 90% of the bacterial strains are spore forming bacterial strains bacteria or up to 100% spore forming bacterial strains. Whether a bacterial strain is a spore forming strain can be determined for instance by evaluating the genome of the bacterial strain for the presence of sporulation genes. However, it should be appreciated that not all bacteria that are predicted to encode spore forming genes can be made to sporulate. In addition, whether a bacterial strain is a spore forming strain can be determined by exposing the bacterial strain to stress conditions, e.g., heat or exposure to chemicals (e.g., ethanol or chloroform), that are known to induce sporulation.

It should be appreciated that spore forming bacteria can be in spore form or in vegetative form. In some embodiments of the compositions provided herein, the spore forming bacteria are in spore form. In some embodiments, the spore forming bacteria are in vegetative form. In some embodiments, the spore forming bacteria are both present in spore form and in vegetative form. In some embodiments, compositions comprise spore forming bacteria and at least 10% of the spore forming bacteria are in spore format, at least 20% of the spore forming bacteria are in spore format, at least 30% of the spore forming bacteria are in spore format, at least 40% of the spore forming bacteria are in spore format, at least 50% of the spore forming bacteria are in spore format, at least 60% of the spore forming bacteria are in spore format, at least 70% of the spore forming bacteria are in spore format, at least 80% of the spore forming bacteria are in spore format, at least 90% of the spore forming bacteria are in spore format, or up to 100% of the spore forming bacteria are in spore format.

It is envisioned that the bacterial strains of the compositions provided herein are alive and will be alive when they reach the target area (e.g., the intestines). Bacterial spores are considered to be alive in this regards. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

Methods of inducing sporulation of spore-forming bacterial strains are well known in the art (See e.g., Paredes-Sabja et al., Trends Microbiol. (2011) 19(2):85-94). Generally, bacterial strains that are spore-formers can be made to go into spore form by stressing the bacterial strains. Non-limiting examples of stresses that can induce sporulation are an increase in temperature, change in the nutrients available and/or exposure to chemicals (e.g., ethanol or chloroform). It should be noted that bacteria that are non-spore formers, for instance because they are missing sporulation genes, cannot be made to sporulate by stress. To prepare compositions in which all the bacterial strains are in the spore form, the composition or bacterial cultures used to prepare the composition may be subjected to treatment to kill any bacteria not in spore form (e.g., in vegetative form), for example by exposing the composition to heat and are chemically breaking down the non-spore bacteria. The bacteria in spore format can subsequently be separated from the non-spore bacteria for instance by filtration.

The amount of spores can be quantified using techniques know in the art. These techniques include phase contrast microscopy for enumerating spores using a hemocytometer. In addition, the viability of spores can be determined by plating the spores and growing the spores. For instance, spores can be plated in appropriate media and incubated in the anaerobic chamber for a period of time (e.g., 48-96 hrs.). Viability can subsequently be determined by quantifying the colony forming units which correspond to spores that germinated. For instance, spores can be plated on TCCFA plates (Taurocholate, cycloserine, cefoxintin, fructose agar plates), in which taurocholate helps the spores to germinate. In addition, spores can be quantified using the dipicolinic assay (DPA assay). DPA is an agent that allows for spore selection and is a clear indicator of endospores. When complexed with terbium, bright green luminescence is observed.

In some embodiments, the compositions comprise bacterial strains that are non-spore forming bacterial strains. In some embodiments, the compositions comprise bacterial strains that are spore forming bacterial strains and bacterial strains that are non-spore forming bacterial strains. In some embodiments, the compositions comprise a mixture of bacterial strains wherein at least 10% of the bacterial strains are non-spore forming bacterial strains, at least 20% of the bacterial strains are non-spore forming bacterial strains, at least 30% of the bacterial strains are non-spore forming bacterial strains, at least 40% of the bacterial strains are non-spore forming bacterial strains, at least 50% of the bacterial strains are non-spore forming bacterial strains, at least 60% of the bacterial strains are non-spore forming bacterial strains, at least 70% of the bacterial strains are non-spore forming bacterial strains, at least 80% of the bacterial strains are non-spore forming bacterial strains, at least 90% of the bacterial strains are non-spore forming bacterial strains bacteria, or up to 100% non-spore forming bacterial strains.

In any of the compositions provided herein, the bacterial strains may be purified. In any of the compositions provided herein, the bacterial strains may be isolated. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. However, bacterial strains can also be isolated from individuals that are considered not to be healthy. In some embodiments, the compositions include strains originating from multiple individuals.

As used herein, the term “isolated” refers to a bacteria or bacterial strain that has been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected.

As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

In some embodiments, the bacterial strains of the compositions provided herein are obligate anaerobes. In some embodiments, the bacterial strains of the compositions provided herein are facultative anaerobes.

Aspects of the present disclosure are related to methods for suppressing multi-drug resistant organisms in a subject. Aspects of the present disclosure are related to methods of suppressing or preventing colonization of the intestine with oral microbiome bacteria. Aspects of the present disclosure are related to methods for treating a disease or disorder associated with bacterial colonization in a subject. Aspects of the present disclosure are related to method for treating a disease or disorder associate with an immune response induced by bacteria in a subject. The methods described herein involve administering to a subject a therapeutically effective amount of any of the compositions described herein. As used herein, a “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human.

In some embodiments, the methods described herein are for suppressing undesired bacteria. As used herein, the term “suppressing” refers to any form of inhibiting an undesired bacteria. In some embodiments, the methods described herein reduce/inhibit or prevent the colonization, replication, proliferation, and/or survival of the undesired bacteria (e.g., multi-drug resistant organisms, oral microbiome bacteria). In some embodiments, the methods described herein directly or indirectly induce death of the undesired bacteria (e.g., multi-drug resistant organisms, oral microbiome bacteria).

In some embodiments, administration of the compositions described herein reduces/inhibits or prevents the colonization, re-colonization, replication, proliferation, and/or survival of multi-drug resistant organisms. In some embodiments, administration of the compositions described herein allows for colonization of the gastrointestinal tract of the subject by the bacterial strain(s) of the compositions thereby preventing colonization by multi-drug resistant organisms.

In some embodiments, the subject is a carrier of a multi-drug resistant organism and is suffering from the effects of the infection. In some embodiments the subject is an asymptomatic carrier of a multi-drug resistant organism. In some embodiments, the subject has experienced recurrent or chronic colonization with a multi-drug resistant organism. In some embodiments, the subject is suffering from a first occurrence of a particular multi-drug resistant organism. In some embodiments, the subject is at risk of colonization with a multi-drug resistant organism, such as prior antibiotic use. In some embodiments, the subject has a risk factor associated with colonization with a multidrug resistant organism. In some embodiments, the subject has had a previous infection or colonization with a multi-drug resistant organism. In some embodiments, the subject has been treated with antibiotics which resulted in the recurrence of the multi-drug resistant organism.

In some embodiments, the subject is to undergo a procedure that puts the subject at a higher risk of colonization and the compositions are administered prophylactically. In some embodiments, the subject has a disease or disorder associated with use of a proton pump inhibitor, which may increase the likelihood of an oral bacterium migrating to the intestine. In some embodiments, the compositions provided herein are administered to a subject to lower the risk of becoming colonized with a multidrug resistant organism. In some embodiments, the bacterial compositions provided herein administered to a subject that is receiving a proton pump inhibitor.

Individuals may be at risk of acquiring a multi-drug resistant organism if they have recently received antimicrobials, are in an immunosuppressed state (e.g., on chemotherapy, have a malignancy, undergoing or received a transplant), have a chronic disease or inflammatory condition (such as diabetes, renal disease, etc.), are older, are undergoing hemodialysis, surgery or other invasive procedures, have indwelling device(s), and/or are living in a long-term care facility or are hospitalized. In some embodiments, the subject is colonized with a multi-drug resistant organism. Skin and mucosal colonization are common (Cassone et al., Curr Geriatr Rep. 2015; 4(1): 87-89), but multi-drug resistant organisms may also colonize the gastrointestinal (GI) tract and oral cavity, causing inflammation (Atarashi et al., Science (2017)). Colonization can lead to significant infections, such as in the skin, lungs, urinary tract, or bloodstream, which may result in serious complications, including death (CDC, 2013). In some instances, multi-drug resistant organisms may be ingested, leading to consequences throughout the digestive system. In some embodiments, a multi-drug resistant organism may colonize the oral cavity.

In some embodiments, the multi-drug resistant organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus. In some embodiments, the multi-drug resistant organism is Vancomycin Resistant Enterococci (VRE). In some embodiments, the multi-drug resistant organism is Carbapenem Resistant Enterobacteriaceae (CRE).

In some embodiments, administration of the compositions described herein reduces/inhibits or prevents intestinal colonization with oral microbiome bacteria. In some embodiments, administration of the compositions described herein reduces/inhibits or prevents the colonization, replication, proliferation, and/or survival of oral microbiome bacteria in the intestinal tract of the subject. In some embodiments, administration of the compositions described herein allows for colonization of the gastrointestinal tract of the subject by the bacterial strain(s) of the compositions thereby preventing colonization by oral microbiome bacteria.

In some embodiments, the subject is a carrier of an oral bacterium and is suffering from the effects of the infection. In some embodiments the subject is an asymptomatic carrier of an oral bacterium. In some embodiments, the subject has experienced recurrent or chronic colonization with an oral bacterium. In some embodiments, the subject is at risk of colonization with an oral bacterium. In some embodiments, the subject has a risk factor associated with colonization with an oral bacterium. In some embodiments, the subject is taking a proton pump inhibitor. In some embodiments, the subject has had a previous infection or colonization with an oral bacterium.

In some embodiments, the subject is to undergo a procedure that puts the subject at a higher risk of colonization and the compositions are administered prophylactically. In some embodiments, the compositions provided herein are administered to a subject to lower the risk of becoming colonized with an oral bacterium.

Over 700 bacterial species or phylotypes have been found in the oral cavity; however, over 50% have not yet been cultivated. A number of phyla have been identified in the oral microbiome Actinobacteria, Arachnia, Bacteroidetes, Bifidobacterium, Chlamydiae, Chloroflexi, Eubacterium, Euryarchaeota, Fusobacterium, Firmicutes, Fusobacteria, Lactobacillus, Leptotrichia, Peptococcus, Peptostreptococcus, Propionibacterium, Proteobacteria, Selenomonas, Spirochaetes, SRI, Synergistetes, Tenericutes, Treponema, TM7, and Veillonella (Dewhirst et al., J of Bacteriology, 2010, 192(19): 5002-5010). Examples of oral microbiome bacteria include, without limitation, Streptococcus sanguis, Streptococcus salivarius, Streptococcus mitis, Streptococcus mutans, Treponema denticola, Eikenella corrodens, Streptococcus gordonii, Streptococcus oralis, Acinomyces maeslundii, and Bacteroides melaningenicus. A list of example species found in the human oral cavity can be found on the Human Oral Microbiome Database (homd.org). In some embodiments, the oral microbiome bacteria may be pathogenic. In some embodiments, the oral microbiome bacteria may be pathogenic if the bacteria gain access to another site of the body. In some embodiments, the oral microbiome bacteria are not pathogenic.

In some embodiments, administration of the compositions described herein reduces/inhibits or prevents intestinal colonization with oral microbiome bacteria. In some embodiments, the oral microbiome bacteria is Fusobacterium nucleatum (See e.g., Yoneda et al. J Gastrointest Dig Syst (2016) 6:2). In some embodiments, the oral microbiome bacteria is Campylobacter concisus (See e.g., Yoneda et al. J Gastrointest Dig Syst (2016) 6:2). In some embodiments, the oral microbiome bacteria is Streptococcus mutans (See e.g., Yoneda et al. J Gastrointest Dig Syst (2016) 6:2). Additional oral microbiome bacteria are described in Table SlA of Atarashi et al. (Atarashi et al., Science 358, 359-365 (2017)), such as Rothia mucilaginosa, Neisseria subflava, Granulicatella para-adiacens, Streptococcus salivarius, Streptococcus mitis, Fusobacterium sp. 1_1_41FAA, Streptococcus oralis, Streptococcus salivarius, Neisseria subflava, Prevotella scopos, Veillonella parvula, Streptococcus sp. M143, Haemophilus parainfluenzae, Prevotella sp. CD3_34, Neisseria macacae, Prevotella histicola, Prevotella pallens, Streptococcus infantis, Streptococcus parasanguinis, Porphyromonas CW034, Streptococcus sp. oral strain T1-E5, Gemella sp. 933-88, Veillonella parvula, and Prevotella sp. C561.

In some embodiments, intestinal colonization with an oral microbiome bacterium induces a Th1 immune response in the subject. Examples of oral microbiome bacteria that may induce Th1 immune responses have been isolated, showing significant similarity (>96.3%) to the following species: Mogibacterium sp. CM96, Peptostreptococcus stomatis, Bifidobacterium sp. Group 111-3, Slackia exigua, Veillonella denticariosi, Atopobium parvulum, Veillonella sp. 2011_oral_VSA_A3, Campylobacter concisus, Actinomyces odontolyticus, Solobacterium moorei, Enterococcus faecium, Bifidobacterium dentium, Veillonella parvela, Fusobacterium sp. 3_1_33, Klebsiella aeromobilis, and Klebsiella pneumoniae (see, e.g., Atarashi et al., Science (2017) 358: 359-365, Schirmer et al., Cell Host and Microbe (2018) 24: 600-610).

In some embodiments, the bacterium that induces a Th1 immune response (e.g., IBD) in a subject is a pathobiont. “Pathobiont” refers to a potentially pathological (disease-causing) organism which, under normal circumstances, lives as a symbiont. Examples of pathibionts are a bacterium that is associated with chronic inflammatory conditions (e.g., IBD). Non-limiting examples of pathobionts include Shigella spp., Campylobacter spp., Cryptosporidium spp., Salmonella spp., Escherichia coli strains (e.g., Enteropathogenic E. coli, Enteroaggregative E. coli, Enterotoxigenic E. coli), Veillonella dispar, Aggregatibacter segnis, Campylobacter, Lachnospiraceae, Veillonella parvula, Haemophilus parainfluenzae, Megasphaera, Escherichia coli, Enterobacteriaceae spp., Enterococcus spp., Fusobacterium spp., Gemella spp., Veillonella spp., Pasteurella spp., Neisseria spp., Haemophilus spp., Campylobacter spp., and Bifidobacterium spp.

In some embodiments, the methods may involve determining whether an oral bacterium is present in the subject. In some embodiments, the methods may involve determining whether an oral bacterium colonizes the oral cavity of the subject. In some embodiments, a subject may be at risk of intestinal colonization if the oral bacterium is present in the oral cavity of the subject. In some embodiments, the methods involve administering the combinations described herein to the subject, if an oral bacterium is detected in the oral cavity of the subject.

In some embodiments, the methods may involve determining whether an oral bacterium is present in the intestine of the subject. In some embodiments, the methods involve administering the combinations described herein to the subject, if an oral bacterium is detected in the intestine of the subject.

In some embodiments, the methods are for treating a disease or disorder associated with bacterial colonization in a subject. In some embodiments, the methods are for treating a disease or disorder associated with an immune response induced by bacteria in a subject. In some embodiments, the methods are for treating a disease or disorder associated with an undesired immune response induced by bacteria in a subject.

In some embodiments, the methods may involve determining whether the subject is colonized with a bacteria. In some embodiments, the methods may involve determining whether the subject has or is experiencing an immune response induced by bacterial colonization. In some embodiments, a subject may be at risk of an immune response induced by bacterial colonization if the subject is colonized by the bacteria. In some embodiments, the methods involve administering the combinations described herein to the subject, if the subject is determined to be colonized by the bacteria. In some embodiments, the methods involve administering the combinations described herein to the subject, if the subject is determined to be experiencing or have experienced an immune response induced by bacterial colonization.

In some embodiments, the immune response induced by bacterial colonization is an Th1 immune response. As will be evident to one of skill in the art, Th1 immune responses are mediated the Th1 population of CD4+ cells. Th1 cells produce IFN-γ and other pro-inflammatory factors. The differentiation of CD4+ cells to Th1 cells is promoted by the presence of IL-2 and/or IL-12 and activation of the transcription factors STAT4 and T-bet. In some embodiments, the immune responses induced by bacterial colonization are Th1 pro-inflammatory responses. Any direct or indirect measure of Th1 immune response, such as the amount of IFN-γ or the number of Th1 cells, may be used to assess the level or extent of the immune response in a sample from a subject.

In some embodiments, the compositions provided herein are administered to a subject if the subject has an autoimmune disease. Examples of autoimmune diseases include, without limitation, inflammatory bowel disease (IBD), ulcerative colitis, Crohn's disease, sprue, autoimmune arthritis, rheumatoid arthritis, graft versus host disease, Type I diabetes, multiple sclerosis, osteoarthritis, juvenile chronic arthritis, Lyme arthritis, psoriatic arthritis, reactive arthritis, spondyloarthropathy, systemic lupus erythematosus, insulin dependent diabetes mellitus, thyroiditis, asthma, psoriasis, dermatitis scleroderma, atopic dermatitis, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlejn purpurea, microscopic vasculitis of the kidneys, chronic active hepatitis, uveitis, cachexia, acute transverse myelitis, Huntington's chorea, Parkinson's disease, Alzheimer's disease, stroke, primary biliary cirrhosis, hemolytic anemia, polyglandular deficiency type I syndrome and polyglandular deficiency type II syndrome, Schmidt's syndrome, adult (acute) respiratory distress syndrome, alopecia, alopecia areata, seronegative arthopathy, arthropathy, Reiter's disease, psoriatic arthropathy, chlamydia, yersinia and salmonella associated arthropathy, spondyloarhopathy, atheromatous disease/arteriosclerosis, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, common varied immunodeficiency (common variable hypogammaglobulinaemia), dilated cardiomyopathy, fibrotic lung disease, cryptogenic fibrosing alveolitis, postinflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, discoid lupus, erythematosus, male infertility idiopathic or NOS, sperm autoimmunity, insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, eosinophilic esophagitis, hypereosinophilic syndrome, eosinophilic gastroenteritis cutaneous lupus erythematosus, eosinophilic esophagitis, hypereosinophilic syndrome, and eosinophilic gastroenteritis, and diarrhea. In some embodiments, the autoimmune disease is inflammatory bowel disease (JBD). In some embodiments, the autoimmune disease is ulcerative colitis. In some embodiments, the autoimmune disease is Crohn's disease.

In some embodiments, the compositions provided herein are administered to a subject if the subject has non-alcoholic steatohepatitis (NASH), primary sclerosing cholangitis (PSC), non-alcoholic fatty liver disease (NAFLD), gastroesophageal reflux disease (GERD), or alcoholism.

In some embodiments, the compositions provided herein are administered to a subject if an immune response associated with bacterial colonization has been detected in the subject. In some embodiments, the methods involve determining whether the subject has an immune response induced by or associated with colonization with an undesired organism.

In some embodiments, the compositions provided herein are administered to a subject if the subject has a dysbiosis (e.g., has as microbiome associated with a disease state). In some embodiments, treatment with the compositions provided herein results in the change in the microbiome of the subject. In some embodiments, treatment with the compositions provided herein removes the dysbiosis in the subject resulting in a healthy microbiome. In some embodiments, treatment with the compositions provided herein removes the dysbiosis in the subject resulting in microbiome refractory or less susceptible to infection by a pathogen.

Any of the compositions described herein may be administered to a subject in a therapeutically effective amount or a dose of a therapeutically effective amount to treat or prevent a disease or disorder, for example associated with colonization with bacteria or an immune response associated with colonization with bacteria. The terms “treat” or “treatment” refer to reducing or alleviating one or more of the symptoms associated with colonization with bacteria or an immune response associated with colonization with bacteria. In some embodiments, any of the compositions described herein may be administered to a subject to prevent a disease or disorder. In some embodiments, any of the compositions described herein may be administered to a subject to prevent a Th1 related disease or disorder. In some embodiments, any of the compositions described herein may be administered to a subject to prevent IBD. The terms “prevent” or “prevention” encompass prophylactic administration and may reduce the incidence or likelihood of colonization with bacteria or an immune response associated with colonization with bacteria. For instance, in some embodiments, administration of the compositions provided herein result in a healthy microbiome that is refractory to pathogenic infection, thereby preventing the pathogenic infection or re-colonization with the pathogenic organism.

As used herein, a “therapeutically effective amount” may be used interchangeably with the term “effective amount.” A therapeutically effective amount or an effective amount of composition, such as a pharmaceutical composition, is any amount that results in a desired response or outcome in a subject, such as those described herein, including but not limited to reducing or preventing colonization with bacteria or an immune response associated with colonization with bacteria.

It should be appreciated that the term effective amount may be expressed in number of bacteria or bacterial spores to be administered. It should further be appreciated that the bacteria can multiply once administered. Thus, administration of even a relatively small amount of bacteria may have therapeutic effects.

In some embodiments, the therapeutically effective amount of any of the compositions described herein is an amount sufficient to enhance survival of the subject, reduce or prevent bacterial colonization of the subject, and/or reduce or inhibit toxin production by the pathogenic infection. In some embodiments, colonization may be assessed by detecting and/or quantifying the bacteria in a sample from the subject, such as a fecal sample. In some embodiments, the therapeutically effective amount is an amount sufficient to reduce the colonization bacteria (e.g., multi-drug resistant organism, oral microbiome bacteria) in a fecal sample from the subject by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more, as compared to the bacterial burden in a subject that has not received any of the compositions described herein, or as compared to a fecal sample from the same subject that was collected prior to administration of any of the compositions.

In some embodiments, the compositions provided herein reduce an immune response associated with bacterial colonization or induced by bacterial colonization. In some embodiments, the therapeutically effective amount is an amount sufficient to reduce an immune response associated with bacterial colonization or induced by bacterial colonization by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the immune response associated with bacterial colonization or induced by bacterial colonization prior to administration of any of the compositions.

In some embodiments, the therapeutically effective amount is an amount sufficient to recolonize or repopulate the gastrointestinal tract of the subject with non-pathogenic bacteria. In some embodiments, the therapeutically effective amount is an amount sufficient to graft one or more of the bacterial strains of the composition in the gastrointestinal tract of the subject. In some embodiments, a fecal sample is obtained from the subject to assess the bacterial burden of undesired bacteria (e.g., multi-drug resistant organisms, oral microbiome bacteria) and/or evaluate the efficacy of administration of the bacterial compositions described herein. In some embodiments, the microbiota of the subject (e.g., the identity and abundance of strains and/or species of the microbiota) may be assessed to determine a disease state of the subject and/or assess progress of the treatment. In some embodiments, the microbiota of the subject having a pathogenic infection is compared to the microbiota of a healthy subject, such as a subject that is not experiencing or has not experienced the pathogenic infection. In some embodiments, the microbiota of the subject having a pathogenic infection is compared to the microbiota of the same subject from a fecal sample obtained from the subject prior to the pathogenic infection.

In some embodiments, administration of the compositions provided herein results in a healthy microbiome that reduces or prevents colonization of the subject by any undesired organism. In some embodiments, administration of the compositions provided herein results in a healthy microbiome that reduces or prevents intestinal colonization of the subject by any undesired organism (e.g., multi-drug resistant organisms, oral microbiome bacteria). In some embodiments, administration of the compositions provided herein results in a healthy microbiome that reduces an immune response associated with bacterial colonization, such as colonization with undesired bacteria. In some embodiments, administration of the compositions provided herein results in a healthy microbiome that reduces a Th1 immune response in the subject.

Any of the compositions described herein may be administered in combination with one or more additional compositions that can suppress a Th1 response and/or induces the accumulation and/or proliferation of regulatory T cells, and/or Th17 cells. In some embodiments, any of the compositions described herein may be administered in combination with a composition that induces the proliferation and/or accumulation of regulatory T cells.

In some embodiments, any of the compositions described herein may be administered in combination with VE-202, a T-reg inducing composition of 17 bacterial strains, described for instance in Atarashi et al., Nature (2013) 500: 232-236. The 17 bacterial strains of VE-202 are represented by the following species: Clostridium saccharogumia, Flavonifractor plautii, Clostridium hathewayi, Blautia coccoides, Clostridium bolteae ATCC BAA-613, cf Clostridium sp. MLG055, Clostridium indolis, Anaerotruncus colihominis, Ruminococcus sp. ID8, Clostridium asparagiforme DSM 15981, Clostridium symbiosum, Clostridium ramosum, Eubacterium contortum, Lachnospiraceae bacterium 5_1_57FAA, Lachnospiraceae bacterium 3_1_57FAA_CT1, Clostridiales bacterium 1_7_47FAA, and Lachnospiraceae bacterium A4. It should be appreciated that subsets of the VE-202 bacteria can also induce Treg cells. Examples of subsets of VE202 that induce Treg cells are found for instance in Atarashi et al., Nature (2013) 500: 232-236 and corresponding Supplemental Information. In some embodiments, any of the compositions described herein may be administered in combination with any of the bacterial compositions as described in PCT Publication WO 2016/209806.

As used herein, the phrase “induces proliferation and/or accumulation of regulatory T cells” refers to an effect of inducing the differentiation of immature T cells into regulatory T cells, which differentiation leads to the proliferation and/or the accumulation of regulatory T cells. Further, the meaning of “induces proliferation and/or accumulation of regulatory T cells” includes in vivo effects, in vitro effects, and/or ex vivo effects. In some embodiments, the proliferation and/or accumulation of regulatory T cells may be assessed by detecting and/or quantifying the number of cells that express markers of regulatory T cells (e.g., Foxp3 and CD4), for example by flow cytometry. In some embodiments, the proliferation and/or accumulation of regulatory T cells may be assessed by determining the activity of the regulatory T cells, such as the production of cytokines (e.g., IL-10).

In some embodiments, any of the compositions described herein may be administered in combination with a composition that induces the accumulation and/or proliferation of Th17 cells, see e.g., compositions disclosed in PCT Publication WO 2015/156419.

In some embodiments, suppressing live bacterial product also repopulates the microbiota of the subject.

Any of the compositions described herein, including the pharmaceutical compositions and food products comprising the compositions, may contain bacterial strains in any form, for example in an aqueous form, such as a solution or a suspension, embedded in a semi-solid form, in a powdered form or freeze dried form. In some embodiments, the composition or the bacterial strains of the composition are lyophilized. In some embodiments, a subset of the bacterial strains in a composition is lyophilized. Methods of lyophilizing compositions, specifically compositions comprising bacteria, are well known in the art. See, e.g., U.S. Pat. Nos. 3,261,761; 4,205,132; PCT Publications WO 2014/029578 and WO 2012/098358, herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may be subsequently be combined with a pharmaceutical excipient. In some embodiments, the bacterial strain is a lyophilized cake. In some embodiments, the compositions comprising the one or more bacterial strains are a lyophilized cake.

In some embodiments, one or more of the bacterial strains of the compositions, including pharmaceutical compositions and food products, has been spray-dried. In some embodiments, a subset of the bacterial strains is spray-dried. The process of spray-drying refers to production of dry powder from a liquid comprising bacterial compositions (See, e.g., Ledet, et al., Spray Draying of Pharmaceuticals in “Lyophilized Biologics and Vaccines” pages 273-294, Springer). In general, the process involves rapidly drying the bacterial compositions with a hot gas. A bacterial strain may be combined with a pharmaceutical excipient prior to combining it with the other bacterial strains or multiple spray-dried bacterial strains may be combined while in spray-dried form and the mixture of bacterial strains, once combined, may be subsequently combined with a pharmaceutical excipient.

The bacterial strains of the composition can be manufactured using fermentation techniques well known in the art. In some embodiments, the active ingredients are manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the composition of bacterial strains may be formulated for administration as a pharmaceutical composition. The term “pharmaceutical composition” as used herein means a product that results from the mixing or combining of at least one active ingredient, such as any two or more purified bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions disclosed herein can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like. In some embodiments, the pharmaceutical compositions can be used by injection, such as by intravenous, intramuscular, subcutaneous, or intradermal administration.

In some embodiments, the compositions comprising bacterial strains are formulated for delivery to the intestines (e.g., the small intestine and/or the colon). In some embodiments, the compositions comprising bacterial strains are formulated with an enteric coating that increases the survival of the bacteria through the harsh environment in the stomach. The enteric coating is one which resists the action of gastric juices in the stomach so that the bacteria of the composition therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, AAPS PharmSciTech (2016) 17(1): 56-67).

The compositions comprising bacterial strains may also be formulated for rectal delivery to the intestine (e.g., the colon). Thus, in some embodiments, compositions comprising bacterial strains may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. (See e.g., Villena et al., Int J Pharm 2015, 487 (1-2): 314-9).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions comprising bacterial strains are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration, e.g. as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions disclosed herein can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect is achieved. In general, effective doses of the compositions, for the prophylactic or therapeutic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the composition is administered orally the subject once, twice, 3 times, 4 times, 5 times, 6 times, 7 times, 8 times, 9 times, or at least 10 times. In some embodiments, any of the compositions described herein are administered the subject in multiple doses at a regular interval, such as every 2 weeks, every month, every 2 months, every 3 months, every 4 months, every 5 months, every 6 months, or more.

The compositions, including the pharmaceutical compositions disclosed herein, include compositions that contain selected bacterial strains. The amount of bacteria, including the amount of bacteria of each of the bacterial strains, in the compositions, including pharmaceutical compositions, may be expressed in weight, number of bacteria and/or CFUs (colony forming units). In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacterial strains per dosage amount. In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that bacteria of each of the bacterial strains may be present in different amounts. Thus, for instance, as a non-limiting example, composition may include 10³ of bacteria A, 10⁴ of bacteria B and 10⁶ of bacteria C. In some embodiments, compositions, including pharmaceutical composition, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacterial strains per dosage amount. In some embodiments, compositions, including pharmaceutical compositions, comprise about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacterial strains combined per dosage amount. As discussed above, bacteria of each of the bacterial strains may be present in different amounts. In some embodiments, the compositions, including pharmaceutical compositions, contain about 10⁻⁷, about 10⁻⁶, about 10⁻⁵, about 10⁻⁴, about 10⁻³, about 10⁻², about 10⁻¹ or more grams of bacteria of each of the bacterial strains in the composition per dosage amount. In some embodiments, the compositions, including pharmaceutical compositions, contain about 10⁻⁷, about 10⁻⁶, about 10⁻⁵, about 10⁻⁴, about 10⁻³, about 10⁻², about 10⁻¹ or more grams of bacteria in total for all of the bacterial strains combined per dosage amount. In some embodiments, the dosage amount is one administration device (e.g., one table, pill or capsule). In some embodiment, the dosage amount is the amount that is administered in a particular period (e.g., one day or one week).

In some embodiments, the compositions, including pharmaceutical compositions, contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each of the bacterial strains per dosage amount. In some embodiments, the compositions, including pharmaceutical compositions, contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per dosage amount.

In some embodiments, the compositions, including pharmaceutical compositions, contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² CFUs of each of the bacterial strains per dosage amount. In some embodiments, the compositions, including pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total CFUs per dosage amount. §

In some embodiments, the compositions, including pharmaceutical compositions, contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of bacteria of each of the bacterial strains in the composition per dosage amount. In some embodiments, the compositions, including pharmaceutical compositions, disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10-3, between 10⁻⁷ and 10⁻⁴, between 10⁻⁶ and 10⁻⁴, between 10⁻⁵ and 10⁻⁴, between 10⁻⁷ and 10⁻⁵′ between 10⁻⁶ and 10⁻⁵, or between 10⁻⁷ and 10⁻⁶ grams of all of the bacteria combined (total) per dosage amount.

Aspects of the present disclosure also provide food products comprising any of the compositions described herein and a nutrient. Also with the scope of the present disclosure are food products comprising any of the bacterial strains described herein and a nutrient. Food products are, in general, intended for the consumption of a human or an animal. Any of the bacterial strains described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing bacterial strains described herein may be produced using methods known in the art and may contain the same amount of bacteria (e.g., by weight, amount or CFU) as the pharmaceutical compositions provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

The nucleic acid sequences of the 16S rDNA, or portion thereof, for the bacterial strains described herein are provided below:

>SEQ ID NO 22: Ruminococcus_faecis; Strain 45
GCGNTCGGTCACCTTCGGCAGCTCCCTCCTTACGGTTGGGTCACTGACTTCGGGCGTTACTGACTCCCATGGTGTG
ACGGGCGGTGTGTACAAGACCCGGGAACGTATTCACCGCGACATTCTGATTCGCGATTACTAGCGATTCCAGCTTC
ATGTAGTCGAGTTGCAGACTACAATCCGAACTGAGACGTTATTTTTGGGATTTGCTCGACCTCGCGGTTCTGCCTC
CCTTTGTTTACGCCATTGTAGCACGTGTGTAGCCCTGCTCATAAGGGGCATGATGATTTGACGTCATCCCCACCTT
CCTCCAGGTTATCCCTGGCAGTCTCTCTAGAGTGCCCGGCCAAACCGCTGGCTACTAAAGATAGGGGTTGCGCTCG
TTGCGGGACTTAACCCAACATCTCACGACACGAGCTGACGACAACCATGCACCACCTGTCATCCCTGTCCCGAAGG
AAAGGCAACATTACTTGCCGGTCAGGGAGATGTCAAGAGCAGGTAAGGTTCTTCGCGTTGCTTCGAATTAAACCAC
ATGCTCCACCGCTTGTGCGGGTCCCCGTCAATTCCTTTGAGTTTCATTCTTGCGAACGTACTCCCCAGGTGGACTA
CTTATTGCGTTTGCTGCGGCACCGAACAGCTTTGCTGCCCGACACCTAGTAGTCATCGTTTACGGCGTGGACTACC
AGGGTATCTAATCCTGTTTGCTCCCCACGCTTTCGAGCCTCAACGTCAGTTACCGTCCAGTAAGCCGCCTTCGCCA
CTGGTGTTCCTCCTAATATCTACGCATTTCACCGCTACACTAGGAATTCCGCTTACCTCTCCGGTACTCTAGATTG
ACAGTTTCCAATGCAGTCCCGGGGTTGAGCCCCGGGTTTTCACATCAGACTTGCCACTCCGTCTACGCTCCCTTTA
CACCCAGTAAATCCGGATAACGCTTGCACCATACGTATTACCGCGGCTGCTGGCACGTATTTAGCCTGTGCTTCTA
GTCAGGTACCGTCATTTTCTTCCCTGCTGATAGAGCTTTACATACCGAATACTTCATCCCCTCCCGCG
>SEQ ID NO 6: Bifidobacterium_adolescentis; Strain 31
GAATTCGAGTCTCACCTTAGACGGCTCCCCCCAAAAGGTTGGGCCACCGGCTTCGGGTGCTACCCACTTTCATGAC
TTGACGGGCGGTGTGTACAAGGCCCGGGAACGCATTCACCGCGGCGTTGCTGATCCGCGATTACTAGCGACTCCGC
CTTCATGGAGTCGGGTTGCAGACTCCAATCCGAACTGAGACCGGTTTTAAGGGATCCGCTCCACCTCGCGGTGTCG
CATCCCGTTGTACCGGCCATTGTAGCATGCGTGAAGCCCTGGACGTAAGGGGCATGATGATCTGACGTCATCCCCA
CCTTCCTCCGAGTTGACCCCGGCGGTCCCCCGTGAGTTCCCACCACGACGTGCTGGCAACACAGGGCGAGGGTTGC
GCTCGTTGCGGGACTTAACCCAACATCTCACGACACGAGCTGACGACGACCATGCACCACCTGTGAACCCGCCCCG
AAGGGAGGCCCCATCTCTGGGGCTGTCGGGAACATGTCAAGCCCAGGTAAGGTTCTTCGCGTTGCATCGAATTAAT
CCGCATGCTCCGCCGCTTGTGCGGGCCCCCGTCAATTTCTTTGAGTTTTAGCCTTGCGGCCGTACTCCCCAGGCGG
GATGCTTAACGCGTTGGCTCCGACACGGAGACCGTGGAATGGTCCCCACATCCAGCATCCACCGTTTACGGCGTGG
ACTACCAGGGTATCTAATCCTGTTCGCTCCCCACGCTTTCGCTCCTCAGCGTCAGTGACGGCCCAGAGACCTGCCT
TCGCCATTGGTGTTCTTCCCGATATCTACACATTCCACCGTTACACCGGGAATTCCAGTCTCCCCTACCGCACTCA
AGCCCGCCCGTACCCGGCGCGGATCCACCGTTAAGCGATGGACTTTCACACCGGACGCGACGAACCGCCTACGAGC
CCTTTACGCCCAATAATTCCGGATACGCTTGCACCCTACGTATTACCGCGGCTGCTGGCACGTAGTTAGCCGGTGC
TTATTCGAAAGGTACACTCACTCCGGAGGGCTTGCTTCCAGTCAAAA
>SEQ ID NO 5: Bifidobacterium_longum; Strain 30
GGGACGGATCTCCCTTAGACGGCTCCATCCCACAAGGGGTTAGGCCACCGGCTTCGGGTGCTGCCCACTTTCATGA
CTTGACGGGCGGTGTGTACAAGGCCCGGGAACGCATTCACCGCGACGTTGCTGATTCGCGATTACTAGCGACTCCG
CCTTCACGCAGTCGAGTTGCAGACTGCGATCCGAACTGAGACCGGTTTTCAGGGATCCGCTCCGCGTCGCCGCGTC
GCATCCCGTTGTACCGGCCATTGTAGCATGCGTGAAGCCCTGGACGTAAGGGGCATGATGATCTGACGTCATCCCC
ACCTTCCTCCGAGTTAACCCCGGCGGTCCCCCGTGAGTTCCCGGCATAATCCGCTGGCAACACGGGGCGAGGGTTG
CGCTCGTTGCGGGACTTAACCCAACATCTCACGACACGAGCTGACGACGACCATGCACCACCTGTGAACCCGCCCC
GAAGGGAAGCCGTATCTCTACGACCGTCGGGAACATGTCAAGCCCAGGTAAGGTTCTTCGCGTTGCATCGAATTAA
TCCGCATGCTCCGCCGCATGTGCGGGCCCCCGTCAATTTCTTTGAGTTTTAGCCTTGCGGCCGTACTCCCCAGGCG
GGATGCTTAACGCGTTAGCTCCGACACGGAACCCGTGGAACGGGCCCCACATCCAGCATCCACCGTTTACGGCGTG
GACTACCAGGGTATCTAATCCTGTTCGCTCCCCACGCTTTCGCTCCTCAGCGTCAGTAACGGCCCAGAGACCTGCC
TTCGCCATTGGTGTTCTTCCCGATATCTACACATTCCACCGTTACACCGGGAATTCCAGTCTCCCCTACCGCACTC
AACCCGCCGTACCGGCGCGGATCCCCGGTAAGCGATGGACTTTCACACCGGACGCGAGG
>SEQ ID NO 8: Blautia_wexlerae; Strain 33
GNGANTGGCGGCGTGCTTACCATGCAGTCGAACGGGAAATACTTCATTGAAACTTCGGTGGATTTAATTTATTTCT
AGTGGCGGACGGGTGAGTAACGCGTGGGTAACCTGCCTTATACAGGGGGATAACAGTCAGAAATGGCTGCTAATAC
CGCATAAGCGCACAGAGCTGCATGGCTCAGTGTGAAAAACTCCGGTGGTATAAGATGGACCCGCGTTGGATTAGCT
TGTTGGTGGGGTAACGGCCCACCAAGGCGACGATCCATAGCCGGCCTGAGAGGGTGAACGGCCACATTGGGACTGA
GACACGGCCCAGACTCCTACGGGAGGCAGCAGTGGGGAATATTGCACAATGGGGGAAACCCTGATGCAGCGACGCC
GCGTGAAGGAAGAAGTATCTCGGTATGTAAACTTCTATCAGCAGGGAAGATAGTGACGGTACCTGACTAAGAAGCC
CCGGCTAACTACGTGCCAGCAGCCGCGGTAATACGTAGGGGGCAAGCGTTATCCGGATTTACTGGGTGTAAAGGGA
GCGTAGACGGTGTGGCAAGTCTGATGTGAAAGGCATGGGCTCAACCTGTGGACTGCATTGGAAACTGTCATACTTG
AGTGCCGGAGGGGTAAGCGGAATTCCTAGTGTAGCGGTGAAATGCGTAGATATTAGGAGGAACACCAGTGGCGAAG
GCGGCTTACTGGACGGTAACTGACGTTGAGGCTCGAAAGCGTGGGGAGCAAACAGGATTAGATACCCTGGTAGTCC
ACGCCGTAAACGATGAATACTAGGTGTCGGGGGAGCATAGCTCTTCGGTGCCGTCGCAAACGCAGTAAGTATTCCA
CCTGGGGAGTACGTTCGCAAGAATGAAACTCAAAGGAATTGACGGGGACCCGCACAAGCGGTGGAGCATGTGGTTT
AATTCGAAGCAACGCGAAGAACCTTACCAAGTCTTGACATCCGCCTGACCGGATCCTTAATCGGGATCTTTCCTTC
GGGACAGGCGAGACAGGGGTGCATGGTTGTCGTCAGCTCGTGTCGTGAGATGTTGGGTAAGTCCGCACGAGCGCAA
CCCTATCCTCAGTAGCAGCATTTAAGTGGGCACTCTGGGGGAGACTGCC
>SEQ ID NO 2: Bacteroides uniformis; Strain 27
GAGCGCTAGGCTTACACATGCAAGTCGAGGGGCAGCATGAACTTAGCTTGCTAAGTTTGATGGCGACCGGCGCACG
GGTGAGTAACACGTATCCAACCTGCCGATGACTCGGGGATAGCCTTTCGAAAGAAAGATTAATACCCGATGGCATA
GTTCTTCCGCATGGTAGAACTATTAAAGAATTTCGGTCATCGATGGGGATGCGTTCCATTAGGTTGTTGGCGGGGT
AACGGCCCACCAAGCCTTCGATGGATAGGGGTTCTGAGAGGAAGGTCCCCCACATTGGAACTGAGACACGGTCCAA
ACTCCTACGGGAGGCAGCAGTGAGGAATATTGGTCAATGGACGAGAGTCTGAACCAGCCAAGTAGCGTGAAGGATG
ACTGCCCTATGGGTTGTAAACTTCTTTTATACGGGAATAAAGTGAGGCACGCGTGCCTTTTTGTATGTACCGTATG
AATAAGGATCGGCTAACTCCGTGCCAGCAGCCGCGGTAATACGGAGGATCCGAGCGTTATCCGGATTTATTGGGTT
TAAAGGGAGCGTAGGCGGACGCTTAAGTCAGTTGTGAAAGTTTGCGGCTCAACCGTAAAATTGCAGTTGATACTGG
GTGTCTTGAGTACAGTAGAGGCAGGCGGAATTCGTGGTGTAGCGGTGAAATGCTTAGATATCACGAAGAACTCCGA
TTGCGAAGGCAGCTTGCTGGACTGTAACTGACGCTGATGCTCGAAAGTGTGGGTATCAAACAGGATTAGATACCCT
GGTAGTCCACACAGTAAACGATGAATACTCGCTGTTTGCGATATACAGTAAGCGGCCAAGCGAAAGCGTTAAGTAT
TCCACCTGGGGAGTACGCCGGCAACGGTGAAACTCAAAGGAATTGACGGGGGCCCGCACAAGCGGGAGGAACATGT
GGTTTAATTCGATGATACGCGAGGAACCTTACCCGGGCTTGAATTGCAACTGAATGATGTGGAGACATGTCAGCCG
CAAGCAGTTGTGAAGGTGCTGCATGGTTGTCGTCAGCTCGTGCCGTGAGGTGTCGGCTTAAGTGCCATAACGAGCG
CAACCCTATCGTAGTACCAT
>SEQ ID NO 14: Coprococcus comes; Strain 37
TGGCTGCGGCGTGCTTACCATGCAAGTCGAACGAAGCACTTATCTTTGATTCTTCGGATGAAGAGGTTTGTGACTG
AGTGGCGGACGGGTGAGTAACGCGTGGGTAACCTGCCTCATACAGGGGGATAACAGTTAGAAATGACTGCTAATAC
CGCATAAGACCACGGAGCCGCATGGCTCAGTGGGAAAAACTCCGGTGGTATGAGATGGACCCGCGTCTGATTAGGT
AGTTGGTGGGGTAACGGCCTACCAAGCCAACGATCAGTAGCCGACCTGAGAGGGTGACCGGCCACATTGGGACTGA
GACACGGCCCAAACTCCTACGGGAGGCAGCAGTGGGGAATATTGCACAATGGGGGAAACCCTGATGCAGCGACGCC
GCGTGAGCGAAGAAGTATTTCGGTATGTAAAGCTCTATCAGCAGGGAAGAAAATGACGGTACCTGACTAAGAAGCA
CCGGCTAAATACGTGCCAGCAGCCGCGGTAATACGTATGGTGCAAGCGTTATCCGGATTTACTGGGTGTAAAGGGA
GCGTAGACGGCTGTGTAAGTCTGAAGTGAAAGCCCGGGGCTCAACCCCGGGACTGCTTTGGAAACTATGCAGCTAG
AGTGTCGGAGAGGTAAGTGGAATTCCCAGTGTAGCGGTGAAATGCGTAGATATTGGGAGGAACACCAGTGGCGAAG
GCGGCTTACTGGACGATGACTGACGTTGAGGCTCGAAAGCGTGGGGAGCAAACAGGGATTAGATACCCTGGTAGTC
CACGCCGTAAACGATGACTACTAAGTGTCGGGGAGCAAAAGCTCTTCGGTGCCGCAGCAAACGCAATAAGTAGTCC
ACCTGGGGGAGTACGTCGCAAGAATGAAACTCAAAGGAATTGACCGGGGACCCGCACAACGGTGGAGCATGTGGTT
TAATTC
>SEQ ID NO 18: Parabacteroides merdae; Strain 41
TAGATTTACCTAGGCCGATCCTTGCGGTTACGGACTTCAGGTACCCCCGGCTCCCATGGCTTGACGGGCGGTGTGT
ACAAGGCCCGGGAACGTATTCACCGCGCCATGGCTGATGCGCGATTACTAGCGAATCCAGCTTCACGGAGTCGAGT
TGCAGACTCCGATCCGAACTGAGACATGGTTTGGAGATTAGCATCCTGTCACCAGGTAGCTGCCCTTTGTCCATGC
CATTGTAACACGTGTGTCGCCCCGGATGTAAGGGCCGTGCTGATTTGACGTCATCCCCACCTTCCTCACAGCTTAC
GCTGGCAGTCTCACCAGAGTCCTCAGCTTCACCTGTTAGTAACTAGTGATAAGGGTTGCGCTCGTTATGGCACTTA
AGCCGACACCTCACGGCACGAGCTGACGACAACCATGCAGCACCTCGTAATCTGCTATTGCTAGAAAGAGTGTTTC
CACTCCGGTCAGACTACGTTCAAACCCGGGTAAGGTTCCTCGCGTATCATCGAATTAAACCACATGTTCCTCCGCT
TGTGCGGGCCCCCGTCAATTCCTTTGAGTTTCACCGTTGCCGGCGTACTCCCCAGGTGGATTACTTAACGCTTTCG
CTGTAGAGCTTACATTGTATCGCAAACTCCTAGTAATCATCGTTTACTGCGTGGACTACCAGGGTATCTAATCCTG
TTTGATCCCCACGCTTTCGTGCTTCAGTGTCAGTTATGGTTTAGTAAGCTGCCTTCGCAATCGGAGTTCTGCGTGA
TATCTATGCATTTCACCGCTACACCACGCATTCCGCCTACCTCAAACACACTCAAGTAACCCAGTTTCAACGGCAA
TTTTATGGTTGAGCCACAAACTTTCACCGCTGACTTAAATCACCACCTACGCACCCTTTAAACCCAATAAATCCGG
ATAACGCTCGCATCCTCCGTATTACCGCGGCTGCTGGCACGGAGTTAGCCGATGCTTATTCATAGGGTACATACAA
AAAGACACGTCCTCCACTTTATTCCCCTTTA
>SEQ ID NO 3: Bacteroides vulgatus; Strain 28
ACAGGTTTTTTCCCTAAGGGCGCTCCTCGCGGTTACGCACTTCAGGTACCCCCGGCTCCCATGGCTTGACGGGCGG
TGTGTACAAGGCCCGGGAACGTATTCACCGCGCCGTGGCTGATGCGCGATTACTAGCGAATCCAGCTTCGTGGAGT
CGGGTTGCAGACTCCAGTCCGAACTGAGAGAGGTTTTTGGGATTGGCATCCACTCGCGTGGTAGCGGCCCTCTGTA
CCCCCCATTGTAACACGTGTGTAGCCCCGGACGTAAGGGCCGTGCTGATTTGACGTCATCCCCACCTTCCTCACAT
CTTACGATGGCAGTCTTGTCAGAGTCCTCAGCGGAACCTGTTAGTAACTGACAACAAGGGTTGCGCTCGTTATGGC
ACTTAAGCCGACACCTCACGGCACGAGCTGACGACAACCATGCAGCACCTTCACAGATGCCTTGCGGCTTACGGCT
TTCACCGTAATTCATCTGCAATTTAAGCCCGGGTAAGGTTCCTCGCGTATCATCGAATTAAACCACATGTTCCTCC
GCTTGTGCGGGCCCCCGTCAATTCCTTTGAGTTTCACCGTTGCCGGCGTACTCCCCAGGTGGAATACTTAACGCTT
TCGCTTGGCCGCTTGCTGTAATGCACAAACAGCGAGTATTCATCGTTTACCGTGTGGACTACCAGGGTATCTAAAT
CCTGTTTGATACCCACACTTTCGAGCCTCAATGTCAGTTGCAGCTTAGCAGGCTGCCTTTATTATCGGAGTTCTTC
GTGATATCT
>SEQ ID NO 15: Dorea longicatena; Strain 38
CGGATCGGTCACCTTCGGCAGCTCCCTCCTTACGGTTGGGTCACTGACTTCGGGCGTTACTGACTCCCATGGTGTG
ACGGGCGGTGTGTACAAGACCCGGGAACGTATTCACCGCAGCATTCTGATCTGCGATTACTAGCGATTCCAGCTTC
ATGTAGTCGAGTTGCAGACTACAATCCGAACTGAGACGTTATTTTTGAGATTTGCTTACCCTCGCGAGTTCGCTTC
TCTTTGTTTACGCCATTGTAGCACGTGTGTAGCCCTGGTCATAAGGGGCATGATGATTTGACGTCATCCCCACCTT
CCTCCAGGTTATCCCTGGCAGTCTCTCCAGAGTGCCCAGCTTAACCTGCTGGCTACTGAAGATAGGGGTTGCGCTC
GTTGCGGGACTTAACCCAACATCTCACGACACGAGCTGACGACAACCATGCACCACCTGTCACCGATGTTCCGAAG
AAAAGCTTCCATTACGAAGCGGTCATCGGGATGTCAAGATCAGGTAAGGTTCTTCGCGTTGCTTCGAATTAAACCA
CATGCTCCACCGCTTGTGCGGGTCCCCGTCAATTCCTTTGAGTTTCATTCTTGCGAACGTACTCCCCAGGTGGACT
GCTTATTGCGTTAGCTGCGGCACCGAATGGCTTTGCCACCCGACACCTAGCAGTCATCGTTTACGGCGTGGACTAC
CAGGGTATCTAATCCTGTTTGCTCCCCACGCTTTCGAGCCTCAACGTCAGTCATCGTCCAGCAAGCCGCCTTCGCC
ACTGGTGTTCCTCCTAATATCTACGCATTTCACCGCTACACTAGGAATTCCACTTGCCTCTCCGACACTCTAGCTC
AGCAGTTCCAAATGCAGTCCCGGGGTTGAGCCCCGGGCTTTCACATCTGGCTTGCCGTGCCGTCTACGCTCCCTTT
ACACCCAGTAAATCCCGGATAACGCTTGCCCCCTACGTATTACCGGCGGCTGCTGGCACGTAGTTAGCCCGGGGCT
TCTTAGTCAAGGTACCGTCAT
>SEQ ID NO 13: Collinsella aerofaciens; Strain 36
CCGTCAACCTTCGGCGCCTCCCCCCTCGCGGTTGGGCCGGCGACTTCGGGTGCAGACGACTCGGGTGGTGTGACGG
GCGGTGTGTACAAGGCCCGGGAACGCATTCACCGCGGCATGCTGATCCGCGATTACTAGCAACTCCGACTTCATGG
GGGCGGGTTGCAGCCCCCAATCCGAACTGGGGCCGGCTTTCCGGGATCCGCTCCCCCTCGCGGGGTGGCATCCCTC
TGTACCGGCCATTGTAGCACGTGTGCAGCCCAGGGCATAAGGGGCATGATGACTTGACGTCGTCCCCGCCCTCCTC
CGCCTTGACGGCGGCGGTCCCGCGTGGGTTCCCGGCATCACCCGATGGCAACACGCGGCGGGGGTTGCGCTCGTTG
CGGGACTTAACCCAACATCTCACGACACGAGCTGACGACAGCCATGCACCACCTGTATGGGCTCCTCTCGGCCACG
GGGTCTCCCCCGCTTCACCCATATGTCAAGCCCTGGTAAGGTTCTTCGCGTTGCTTCGAATTAAGCCACATGCTCC
GCTGCTTGTGCGGGCCCCCGTCAATTCCTTTGAGTTTTAGCCTTGCGGCCGTACTCCCCAGGCGGGACGCTTAATG
CGTTGGCTGCGGCACGGGGGGATCGTCCCCCCACACCTAGCGTCCATCGTTTACGGCTGGGACTACCAGGGTATCT
AATCCTGTTCGCTCCCCCAGCTTTCGCGCCTCAGCGTCGGTCTCGGCCCAGAGGGCCGCCTTCGCCACCGGTGTTC
CACCCGATATCTGCGCATTCCACCGCTACACCGGGTGTTCCACCCTCCCCTACCGGACCCAAGCCGCGGAGGTTCC
GGGGGCTTCGGGGGGTTGAGCCCCCCGCTTCGACCCCGGCCTGCCGGGCCGCCTACGCGCGCTTTACGCCCAATGA
ATCCGGATAACGCTCGCCCCCTACGTATTACGCGGCTGCTGGCACGTAGTTAGCCGGGGCTTCTTCTGCAGGTACA
GTCTTGACTCTTCCCTGCTGAAAGCGGTTTACGA
>SEQ ID NO 19: Parabacteroides distasonis; Strain 42
TCAGCATGACCTAGGCCGATCCTCGCGGTTACGGACTTCAGGTACCCCCGGCTCCCATGGCTTGACGGGCGGTGTG
TACAAGGCCCGGGAACGTATTCACCGCGCCATGGCTGATGCGCGATTACTAGCGAATCCAGCTTCACGGAGTCGGG
TTGCAGACTCCGATCCGAACTGAGACGTGGTTTGGGGATTCGCTCCCTGTCGCCAGGTGGCTTCCCTTTGTCCACG
CCATTGTAACACGTGTGTCGCCCCGGATGTAAGGGCCGTGCTGATTTGACGTCATCCCCGCCTTCCTCGCAGCTTA
CGCTGGCAGTCCCACCAGAGTCCTCAGCATCACCTGTTAGTAACTAGTGGCAAGGGTTGCGCTCGTTATGGCACTT
AAGCCGACACCTCACGGCACGAGCTGACGACAACCATGCAGCACCTCGCAAACGGCTATTGCTAGAAGAGGTGTTT
CCACCTCGGTCCGAATGCGTTCAAACCCGGGTAAGGTTCCTCGCGTATCATCGAATTAAACCACATGTTCCTCCGC
TTGTGCGGGCCCCCGTCAATTCCTTTGAGTTTCACCGTTGCCGGCGTACTCCCCAGGTGGATCACTTAACGCTTTC
GCTGTGCCGCTTACACTGTATCGCAAACAGCTAGTGATCATCGTTTACTGCGTGGACTACCAGGGTATCTAATCCT
GTTTGATCCCCACGCTTTCGTGCATCAGCGTCAGTCATGGCTTGGCAGGCTGCCTTCGCAATCGGGGTTCTGCGTG
ATATCTAAGCATTTCACCGCTACACCACGCATTCCGCCTGCCTCAAACATACTCAAGCCTCCCAGTTTCAACGGCA
ATTCTATGGTTGAGCCACAGACTTTCACCGCTGACTTAAAAGGCCGCCTACGCACCCTTTAAACCCAATAAATCCG
GATAACGCTCGGATCCTCCGTATTACCGCGGCTGCTGGCACGGAGTTAGCCGATCCTTATTCATAAGGTACATACA
AAACAGGAAACGTCCACAACTTTATTCCCTTATAAAGAGGTTTACGAT
>SEQ ID NO 20: Prevotella copri; Strain 43
CTTAGCTTTCGCCTAGGCCGCTCCTTACGGTCACGGACTTTAGGCGCCCCCGGCTTTCATGGCTTGACGGGCGGTG
TGTACAAGGCCCGGGAACGTATTCACCGCGCCATGGCTGATGCGCGATTACTAGCGAATCCAGCTTCGTGGGGTCG
GGTTGCAGACCCCAGTCCGAACTGAGACAGGCTTTAAGGATTTGATCCTTTTTGCAAGGGACCGTCTCTCTGTACC
TGCCATTGTAACACGTGTGTAGCCCCGGACGTAAGGGCCGTGCTGATTTGACGTCATCCCCACCTTCCTCACACCT
TACGGTGGCAGTGTCCCCAGAGTGCCCAGCTTAACCTGATGGCAACTAAGGAGAGGGGTTGCGCTCGTTATGGCAC
TTAAGCCGACACCTCACGGCACGAGCTGACGACAACCATGCAGCACCTTCACAGAGGCCCCGAAGGGCGTCATTGT
CTCCAAATCCTTCCTCTGCAATTCAAGCCCGGGTAAGGTTCCTCGCGTATCATCGAATTAAACCACATGTTCCTCC
GCTTGTGCGGGCCCCCGTCAATTCCTTTGAGTTTCACCGTTGCCGGCGTACTCCCCAGGTGGGATGCTTAATGCTT
TCGCTTGGCCGCTGACCTATTCAGACCAACAGCGGGCATCCATCGTTTACCGTGCGGACTACCAGGGTATCTAATC
CTGTTCGATACCCGCACTTTCGAGCTTCAGCGTCAGTTGCGCTCCAGTGAGCTGCCTTCGCAATCGGAGTTCTTCG
TGATATCTAAGCATTTCACCGCTACACCACGAATTCCGCCCACTTTGTGCGTACTCAAGGAAACCAGTTCGCGCTG
CAGTGCAACGTTGAGCGTCTAATTTCACAACACGCTTAATCTCCGGCTACGCTCCCTTTAACCAAAAAAACCAGAT
AACGCCGGACCTCCGTATTACCGCGGCTGCTGGCCGGAATTAGCCGGCCCTATCATAAGGTACATGCAAAAAGCTA
CCAAACTCACTTTTTCCCTTTACAAGAGTTACAACCATAGGCC
>SEQ ID NO 1: Alistipes putredinis; Strain 26
GCTCAGCTTGCCTAGGTCGCTCCTTGCGGTCACGAACTTCAGGCACCCCCGGCTCCCATGGCTTGACGGGCGGTGT
GTACAAGGCCCGGGAACGTATTCACCGCGCCATGGCTGATGCGCGATTACTAGCGAATCCAACTTCATGGAGGCGG
GTTTCAGCCTCCAATCCGAACTGAGATAGGCTTTCGAGATTCGCATCCCATCGCTGGGTAGCTGCCCTCTGTACCT
ACCATTGTAACACGTGTGTAGCCCCGGACGTAAGGGCCGTGCTGATTTGACGTCATCCCCACCTTCCTCTCGGCTT
ACACCGGCAGTCCCGCCAGAGTGCCCAGCTTCACCTGATGGCAACTAACGGTAGGGGTTGCGCTCGTTATGGGACT
TAACCCGACACCTCACGGCACGAGCTGACGACAACCATGCAGCACCTAGTTTCGCGCCCCGAAGGGAAATCCTCTT
TCAAGAATCGTCGCTAACTTTCAAGCCCGGGTAAGGTTCCTCGCGTATCATCGAATTAAACCACATGTTCCTCCGC
TTGTGCGGGCCCCCGTCAATTCCTTTGAGTTTCATTCTTGCGAACGTACTCCCCAGGTGGATAACTTATCGCTTTC
GCTTAGTCACCGACTGTGTATCGCCGACAACGAGTTATCATCGTTTACTGCGTGGACTACCAGGGTATCTAATCCT
GTTTGCTCCCCACGCTTTCGTGCCTCAACGTCAGATATAGTTTGGTAAGCTGCCTTCGCAATCGGTGTTCTGTATG
ATCTCTAAGCATTTCACCGCTACACCATACATTCCGCCTACCGCAACTACTCTCTAGCTCAACAGTATTAGAGGCA
CGTTCAGGGTTGAGCCCCGAAATTTCACCTCTAACTTATCAAACCGCCTACGCACCCTTTAAACCCAATAAATCCG
GATAACGCTTGAATCCTCCGTATTACCGCGGCTGCTGGCACGGAGTTAGCCGATCCTTATTCGTACGATACTTTCA
GACAGATACACGTATCTGCGTTTACCCTCGTACA
>SEQ ID NO 16: Eubacterium halli; Strain 39
TCGGCTCCCTTCGAAGCTCCCTCCATAAAGGTTGGGTCACTGGCTTCGGGCATTTCCAACTCCCATGGTGTGACGG
GCGGTGTGTACAAAACCCGGGAACGTATTCACCGCGACATTCTGATTCGCGATTACTAGCGATTCCAGCTTCGTGT
AGTCGGGTTGCAAACTACAGTCCAAACTGGGACGGCCTTTTTGTGGTTTGCTCCCCCTCGCGGGTTTGCCTCACTC
TGTGACCGCCATTGTAGCACGTGTGTCGCCCAAATCATAAGGGGCATGATGATTTGACGTCGTCCCCACCTTCCTC
CAGGTTATCCCTGGCAGTCTCTCCAAAGTGCCCAGCCTTACCTGCTGGCTACTGAAAATAGGGGTTGCGCTCGTTG
CGGGACTTAACCCAACATCTCACAACACAAGCTGACAACAACCATGCACCACCTGTCTCTTCTGTCCCGAAGGAAA
ACTCCCATTACGGAGTGGTCAAAAGGATGTCAAGACCTGGTAAGGTTCTTCGCGTTGCTTCAAATTAAACCACATG
CTCCACCGCTTGTGCGGGTCCCCGTCAATTCCTTTGAGTTTCATTCTTGCAAACGTACTCCCCAGGTGGAATACTT
ACTGCGTTAGCGGCGGCACCGAAGCCTATACGGCCCCGACACCTAGTATTCATCGTTTACGGCGTGGACTACCAGG
GTATCTAATCCTGTTTGCTCCCCACGCTTTCGTGCCTCAGTGTCAGTAACAGTCCAGCAGGCCGCCTTCGCCACTG
GTGTTCCTCCTAATATCTACGCATTTCACCGCTACACTAGGAATTCCGCCTGCTTCTCCTGTACTCTAGCTAAGCA
GTTTCAAATGCAGCTCCGGGGTTGAGCCCGGGCTTTCACATCTGACTTGCACTGCCACCTACGCACCCTTTACACC
AATAAATCCGGATAACGCTTGCTCCATACGTATTACCGCGGCTGCTGGCACGTATTAGCCCGGAGCTTCTAATCAG
GTACCGGCATTATCTCCCTGCTGATAGA
>SEQ ID NO 17: Faecalibacterium prausnitzii; Strain 40
TGGGCTGGCGGCGCGCTACACATGCAGTCGAACGAGCGAGAGAGAGCTTGCTTTCTCGAGCGAGTGGCGAACGGGT
GAGTAACGCGTGAGGAACCTGCCTCAAAGAGGGGGACAACAGTTGGAAACGACTGCTAATACCGCATAAGCCCACG
ACCCGGCATCGGGTAGAGGGAAAAGGAGCAATCCGCTTTGAGATGGCCTCGCGTCCGATTAGCTAGTTGGTGAGGT
AATGGCCCACCAAGGCGACGATCGGTAGCCGGACTGAGAGGTTGAACGGCCACATTGGGACTGAGACACGGCCCAG
ACTCCTACGGGAGGCAGCAGTGGGGAATATTGCACAATGGGGGAAACCCTGATGCAGCGACGCCGCGTGGAGGAAG
AAGGTCTTCGGATTGTAAACTCCTGTTGTTGAGGAAGATAATGACGGTACTCAACAAGGAAGTGACGGCTAACTAC
GTGCCAGCAGCCGCGGTAAAACGTAGGTCACAAGCGTTGTCCGGAATTACTGGGTGTAAAGGGAGCGCAGGCGGGA
AGACAAGTTGGAAGTGAAATCCATGGGCTCAACCCATGAACTGCTTTCAAAACTGTTTTTCTTGAGTAGTGCAGAG
GTAGGCGGAATTCCCGGTGTAGCGGTGGAATGCGTAGATATCGGGAGGAACACCAGTGGCGAAGGCGGCCTACTGG
GCACCAACTGACGCTGAGGCTCGAAAGTGTGGGTAGCAAACAGGATTAGATACCCTGGTAGTCCACACTGTAAACG
ATGATTACTAGGTGTTGGAGGATTGACCCCTTCAGTGCCGCAGTTAACACAATAAGTAATCCACCTGGGGAGTACG
ACCGCAAGGTTGAAACTCAAAGGAATTGACGGGGGCCCGCACAAGCAGTGGAGTATGTGGTTTAATTCGACGCAAC
GCGAAGAACCTTACCAAGTCTTGACATCCTTGTGACGATGCTAGAAATAGTATTTTTCTTCTGAACACAGAGACAG
GTGGTGCATGGTTGTCGTCAGCTCGTGTCGTGAGATGTTGGGTTAAGTCCCGCAACGAGCGCCAACCCTTATGGTC
AGTTACTACCGCA
>SEQ ID NO 7: Blautia obeum; Strain 37
CCAAAAAGCGCGGCGGCGTGCTTACCATGCAGTCGAACGGGAAACTTTTATTGAAGCTTCGGCAGATTTGGTTGGT
TTCTAGTGGCGGACGGGTGAGTAACGCGTGGGTAACCTGCCTTATACAGGGGGATAACAACCAGAAATGGTTGCTA
ATACCGCATAAGCGCACAGGACCGCATGGTCCGGTGTGAAAAACTCCGGTGGTATAAGATGGACCCGCGTTGGATT
AGCTAGTTGGCAGGGTAACGGCCTACCAAGGCGACGATCCATAGCCGGCCTGAGAGGGTGAACGGCCACATTGGGA
CTGAGACACGGCCCAGACTCCTACGGGAGGCAGCAGTGGGGAATATTGCACAATGGGGGAAACCCTGATGCAGCGA
CGCCGCGTGAAGGAAGAAGTATCTCGGTATGTAAACTTCTATCAGCAGGGAAGATAGTGACGGTACCTGACTAAGA
AGCCCCGGCTAACTACGTGCCAGCAGCCGCGGTAATACGTAGGGGGCAAGCGTTATCCGGATTTACTGGGTGTAAA
GGGAGCGTAGACGGACTGGCAAGTCTGATGTGAAAGGCGGGGGCTCAACCCCTGGACTGCATTGGAAACTGTTAGT
CTTGAGTGCCGGAGAGGTAAGCGGAATTCCTAGTGTAGCGGTGAAATGCGTAGATATTAGGAGGAACACCAGTGGC
GAAGGCGGCTTACTGGACGGTAACTGACGTTGAGGCTCGAAAGCGTGGGGAGCAAACAGGATTAGATACCCTG
>SEQ ID NO 12: Parabacteroides distasonis; Strain 35
TCGGGTTTTTTCCTAGGCCGATCCTTTCGGTTACTGACTTCAGGTACCCCCGGCTCCCATGGCTTGACGGGCGGTG
TGTACAAGGCCCGGGAACGTATTCACCGCGCCATGGCTGATGCGCGATTACTAGCGAATCCAGCTTCACGGAGTCG
GGTTGCAGACTCCGATCCTAACTGAGACGTGGTTTGGGGATTCGCTCCCTGTCGCCAGGTGGCCTCCCTTTGTCCA
CGCCATTGTAACACGTGTGTCGCCCCGGATGTAAGGGCCGTGCTGATTTGACGTCATCCCCGCCTTCCTCGCAGCT
TACGCTGGCAGTCCCACCAGAGTCCTCAGCTTTACCTGTTAGTAACTAGTGGCATGGGTTGCGCTCGTTATGGCAC
TTAAGCCGACACCTCACGGCACGAGCTGACAACAACCATGCACCACCTCGCAAACGGCTATTGCTAAAAAAGGTGT
TTCCACCTCGGTCCTAATGCTTTCAAACCCGGGTAAGGTTCCTCGCGTATCATCGAATTAAACCACATGTTCCTCC
GCTTGTGCTGGCCCCCGTCATTCCTTTGAGTTTCACCGTTGCCGGCGTACTCCCCAGGTGGATCACATAACGCTTT
CCCTGAGCCGCTTACTGTGTATCGTACACACCTAGTGATCATCTTTTACTGCGTGGACTAACAGGGTATCCTAATC
CTGTTTGATCCCCACGCTTTCGTGCATCACGTCAGTCATGGCTTGTGAGCTGCCTTCGCAAACTGGGTTCTGCAAG
A
>SEQ ID NO 11: Clostridium bolteae; Strain 34
GGGCGGGCGGCGTGCTACCATGCAAGTCGAACGAAGCAATTAAATGAAGTTTTCGGATGGAATTTGATTGACTGAG
TGGCGGACGGGTGAGTAACGCGTGGATAACCTGCCTCACACTGGGGGATAACAGTTAGAAATGACTGCTAATACCG
CATAAGCGCACAGTACCGCATGGTACGGTGTGAAAAACTCCGGTGGTGTGAGATGGATCCGCGTCTGATTAGCCAG
TTGGCGGGGTAACGGCCCACCAAAGCGACGATCAGTAGCCGACCTGAGAGGGTGACCGGCCACATTGGGACTGAGA
CACGGCCCAAACTCCTACGGGAGGCAGCAGTGGGGAATATTGCACAATGGGCGAAAGCCTGATGCAGCGACGCCGC
GTGAGTGAAGAAGTATTTCGGTATGTAAAGCTCTATCAGCAGGGAAGAAAATGACGGTACCTGACTAAGAAGCCCC
GGCTAACTACGTGCCAGCAGCCGCGGTAATACGTAGGGGGCAAGCGTTATCCGGATTTACTGGGTGTAAAGGGAGC
GTAGACGGCGAAGCAAGTCTGAAGTGAAAACCCAGGGCTCAACCCTGGGACTGCTTTGGAAACTGTTTTGCTAGAG
TGTCGGAGAGGTAAGTGGAATTCCTAGTGTAGCGGTGAAATGCGTAGATATTAGGAGGAACACCAGTGGCGAAGGC
GGCTTACTGGACGATAACTGACGTTGAGGCTCGAAAGCGTGGGGAGCAAACAGGATTAGATACCCTGGTAGTCCAC
GCCGTAAACGATGAATGCTAGGTGTTGGGGGGCAAAGCCCTTCGGTGCCGTCGCAAACGCAGTAAGCATTCCACCT
GGGGAGTACGTTCGCAAGAATGAAACTCAAGGAATTGACGGGGACCCGCACAAGCGGTGGAGCATGTGGTTTAATT
CGAAGCAACGCGAAGAACTTACCAAGTCTTGACATCCTCTTGACCGGCGTGTAACGGCGCCTTCTCTTCTGGGCAA
GAGAGACAGTGGTGCATGGTTGTCGTCAGCTCGTGTCGTGAGATGTTGGGTTAAGTCCCGCACGAGCGCAACCCTT
TTCCTTATATCACAGGTGAGCTGGGCCCTCTAGGAGACTGCCAGGATACTGAGGAA
>SEQ ID NO 4: Parabacteroides merdae; Strain 29
GTGCTCAGCTTTTACCCTAGGCCGATCCTTGCGGTTACGGACTTCAGGTACCCCCGGCTCCCATGGCTTGACGGGC
GGTGTGTACAAGGCCCGGGAACGTATTCACCGCGCCATGGCTGATGCGCGATTACTAGCGAATCCAGCTTCACGGA
GTCGAGTTGCAGACTCCGATCCGAACTGAGACATGGTTTGGAGATTAGCATCCTGTCGCCAGGTAGCTGCCCTTTG
TCCATGCCATTGTAACACGTGTGTCGCCCCGGATGTAAGGGCCGTGCTGATTTGACGTCATCCCCACCTTCCTCAC
AGCTTACGCTGGCAGTCTCACCAGAGTCCTCAGCTTCACCTGTTAGTAACTAGTGATAAGGGTTGCGCTCGTTATG
GCACTTAAGCCGACACCTCACGGCACGAGCTGACGACAACCATGCAGCACCTCGTAATCTGCTATTGCTAGAAGGA
GTGTTTCCACTCCGGTCAGACTACGTTCAAACCCGGGTAAGGTTCCTCGCGTATCATCGAATTAAACCACATGTTC
CTCCGCTTGTGCGGGCCCCCGTCAATTCCTTTGAGTTTCACCGTTGCCGGCGTACTCCCCAGGTGGATTACTTAAC
GCTTTCGCTGTAGAGCTTACATTGTATCGCAAACTCCTAGTAATCATCGTTTACTGCGTGGACTACCAGGGTATCT
AATCCTGTTTGATCCCCACGCTTTCGTGCTTCAGTGTCAGTTATGGTTTAGTAAGCTGCCTTCGCAATCGGAGTTC
TGCGTGATATCTATGCATTTCACCGCTACACCACGCATTCCGCCTACCTCAAACACACTCAAGTAACCCAGTTTCA
ACGGCAATTTTATGGTTGAGCCACAAACTTTCACCGCTGACTTAAATCACCACCTACGCACCCTTTAACCCAATAA
ATCCGATAACGCTCGCATCCTCCGTATTACCGCGGCTGCTGCCCGGAGTTAGCCGATGCTTATTCATAGGGTACAT
ACAAAAAGGACACGT
>SEQ ID NO 21: Roseburia faecis; Strain 44
GGGGCTGGGCGGCGTGCTTACCATGCAAGTCGAACGAAGCACTCTATTTGATTTTCTTCGGAAATGAAGATTTTGT
GACTGAGTGGCGGACGGGTGAGTAACGCGTGGGTAACCTGCCTCATACAGGGGGATAACAGTTGGAAACGACTGCT
AATACCGCATAAGCGCACAGGATCGCATGATCCGGTGTGAAAAACTCCGGTGGTATGAGATGGACCCGCGTCTGAT
TAGCCAGTTGGCAGGGTAACGGCCTACCAAAGCGACGATCAGTAGCCGACCTGAGAGGGTGACCGGCCACATTGGG
ACTGAGACACGGCCCAAACTCCTACGGGAGGCAGCAGTGGGGAATATTGCACAATGGGGGAAACCCTGATGCAGCG
ACGCCGCGTGAGCGAAGAAGTATTTCGGTATGTAAAGCTCTATCAGCAGGGAAGAAGAATGACGGTACCTGACTAA
GAAGCACCGGCTAAATACGTGCCAGCAGCCGCGGTAATACGTATGGTGCAAGCGTTATCCGGATTTACTGGGTGTA
AAGGGAGCGCAGGCGGTGCGGCAAGTCTGATGTGAAAGCCCGGGGCTCAACCCCGGTACTGCATTGGAAACTGTCG
TACTAGAGTGTCGGAGGGGTAAGTGGAATTCCTAGTGTAGCGGTGAAATGCGTAGATATTAGGAGGAACACCAGTG
GCGAAGGCGGCTTACTGGACGATAACTGACGCTGAGGCTCGAAAGCGTGGGGAGCAAACAGGATTAGATACCCTGG
TAGTCCACGCCGTAAACGATGAATACTAGGTGTCGGGGAGCATTGCTCTTCGGTGCCGCAGCAAACGCAATAAGTA
TTCCACCTGGGGAGTACGTTCGCAAGAATGAAACTCAAAGGAATTGACGGGGACCCGCACAGCGGTGGAGCATGTG
GTTTATTCGAAGCAACGCGAAGAACCTTACCAAGTCTTGACATCCCGATGACAGAGTATGTAATGTACTTTCTCTT
CGAGCATCGGTGACAGTGGGTGCATGGTTGTCGTCACTCGTGTCGTGAGATGTTGGGTTAAGTCCGCAACGAGCGC
AACCCCTGTCCTTAGTAGCAGCGGTG
>SEQ ID NO: 9: Blautia producta/Blautia coccoides; Strain 2
AGAGTTTGATCCTGGCTCAGGATGAACGCTGGCGGCGTGCTTAACACATGCAAGTCGAGCGAAGCATTAAGACAGA
TTTCTTCGGATTGAAGTCTTTGTGACTGAGCGGCGGACGGGTGAGTAACGCGTGGGTAACCTGCCTCATACAGGGG
GATAACAGTTAGAAATGACTGCTAATACCGCATAAGCGCACAGGAcCGCATGGTCTGGTGTGAAAAACTCCGGTGG
TATGAGATGGACCCGCGTCTGATTAGCTAGTTGGAGGGGTAACGGCCCACCAAGGCGACGATCAGTAGCCGGCCTG
AGAGGGTGAACGGCCACATTGGGACTGAGACACGGCCCAGACTCCTACGGGAGGCAGCAGTGGGGAATATTGCACA
ATGGGGGAAACCCTGATGCAGCGACGCCGCGTGAAGGAAGAAGTATCTCGGTATGTAAACTTCTATCAGCAGGGAA
GAAAATGACGGTACCTGACTAAGAAGCCCCGGCTAACTACGTGCCAGCAGCCGCGGTAATACGTAGGGGGCAAGCG
TTATCCGGATTTACTGGGTGTAAAGGGAGCGTAGACGGAAGAGCAAGTCTGATGTGAAAGGCTGGGGCTTAACCCC
AGGACTGCATTGGAAACTGTTGTTCTAGAGTGCCGGAGAGGTAAGCGGAATTCCTAGTGTAGCGGTGAAATGCGTA
GATATTAGGAGGAACACCAGTGGCGAAGGCGGCTTACTGGACGGTAACTGACGTTGAGGCTCGAAAGCGTGGGGAG
CAAACAGGATTAGATACCCTGGTAGTCCACGCCGTAAACGATGAATACTAGGTGTCGGGTGGCAAAGCCATTCGGT
GCCGCAGCAAACGCAATAAGTATTCCACCTGGGGAGTACGTTCGCAAGAATGAAACTCAAAGGAATTGACGGGGAC
CCGCACAAGCGGTGGAGCATGTGGTTTAATTCGAAGCAACGCGAAGAACCTTACCAAGTCTTGACATCCCTCTGAC
CGTCCCGTAAtGGGGgCTTCCCTTCGGGGCAGAGGAGACAGGTGGTGCATGGTTGTCGTCAGCTCGTGTCGTGAGA
TGTTGGGTTAAGTCCCGCAACGAGCGCAACCCTTATCCTTAGTAGCCAGCACATGATGGTGGGCACTCTAGGGAGA
CTGCCGGGGATAACCCGGAGGAAGGCGGGGACGACGTCAAATCATCATGCCCCTTATGATTTGGGCTACACACGTG
CTACAATGGCGTAAACAAAGGGAAGCGAGACAGCGATGTTGAGCGAATCCCAAAAATAACGTCCCAGTTCGGACTG
CAGTCTGCAACTCGACTGCACGAAGCTGGAATCGCTAGTAATCGCGGATCAGAATGCCGCGGTGAATACGTTCCCG
GGTCTTGTACACACCGCCCGTCACACCATGGGAGTCAGTAACGCCCGAAGTCAGTGACCTAACCGAAAGGAAGGAG
CTGCCGAAGGCGGGACCGATAACTGGGGTGAAGTCGTAACAAGGTAGCCGTATCGGAAGGTGCGGCTGGATCACCT
CCTTTCTAAGGAAGAAGAAGTAGAGAAAAGTGTTTCACTGTTGAGTTACCAAGA
SEQ ID NO: 10: Clostridium hathewayi/Hungatella effluvii strain UB-B.2;
Strain 20
ATGAGAGTTCGATCCTGGCTCAGGATGAACGCTGGCGGCGTGCTTAACACATGCAAGTCGAGCGAAGCGGTTTCGA
TGAAGTTTTCGGATGGATTTGAAATCGACTTAGCGGCGGACGGGTGAGTAACGCGTGGGTAACCTGCCTTACACTG
GGGGATAACAGTTAGAAATGACTGCTAATACCGCATAAGCGCACAGGGCCGCATGGTCTGGTGCGAAAAACTCCGG
TGGTGTAAGATGGACCCGCGTCTGATTAGGTAGTTGGTGGGGTAACGGCCCACCAAGCCGACGATCAGTAGCCGAC
CTGAGAGGGTGACCGGCCACATTGGGACTGAGACACGGCCCAAACTCCTACGGGAGGCAGCAGTGGGGAATATTGG
ACAATGGGCGAAAGCCTGATCCAGCGACGCCGCGTGAGTGAAGAAGTATTTCGGTATGTAAAGCTCTATCAGCAGG
GAAGAAAATGACGGTACCTGACTAAGAAGCCCCGGCTAACTACGTGCCAGCAGCCGCGGTAATACGTAGGGGGCAA
GCGTTATCCGGATTTACTGGGTGTAAAGGGAGCGTAGACGGTTAAGCAAGTCTGAAGTGAAAGCCCGGGGCTCAAC
CCCGGTACTGCTTTGGAAACTGTTTGACTTGAGTGCAGGAGAGGTAAGTGGAATTCCTAGTGTAGCGGTGAAATGC
GTAGATATTAGGAGGAACACCAGTGGCGAAGGCGGCTTACTGGACTGTAACTGACGTTGAGGCTCGAAAGCGTGGG
GAGCAAACAGGATTAGATACCCTGGTAGTCCACGCCGTAAACGATGAATACTAGGTGTCGGGGGACAACGTCCTTC
GGTGCCGCCGCTAACGCAATAAGTATTCCACCTGGGGAGTACGTTCGCAAGAATGAAACTCAAAGGAATTGACGGG
GACCCGCACAAGCGGTGGAGCATGTGGTTTAATTCGAAGCAACGCGAAGAACCTTACCAAGTCTTGACATCCCATT
GAAAATCATTTAACCGTGATCCCTCTTCGGAGCAATGGAGACAGGTGGTGCATGGTTGTCGTCAGCTCGTGTCGTG
AGATGTTGGGTTAAGTCCCGCAACGAGCGCAACCCTTATCCTTAGTAGCCAGCACATGATGGTGGGCACTCTGGGG
AGACTGCCAGGGATAACCTGGAGGAAGGTGGGGATGACGTCAAATCATCATGCCCCTTATGATTTGGGCTACACAC
GTGCTACAATGGCGTAAACAAAGGGAAGCAAAGGAGCGATCTGGAGCAAACCCCAAAAATAACGTCTCAGTTCGGA
TTGCAGGCTGCAACTCGCCTGCATGAAGCTGGAATCGCTAGTAATCGCGAATCAGAATGTCGCGGTGAATACGTTC
CCGGGTCTTGTACACACCGCCCGTCACACCATGGGAGTTGGTAACGCCCGAAGTCAGTGACCCAACCGTAAGGAGG
GAGCTGCCGAAGGCGGGACTGATAACTGGGGTGAAGTCGTAACAAGGTAGCCGTATCGGAAGGTGCGGCTGGATCA
CCTCCTTT
SEQ ID NO: 23 Flavonifractor plautii Strain 1
TATTGAGAGTTTGATCCTGGCTCAGGATGAACGCTGGCGGCGTGCTTAACACATGCAAGTCGAACGGGGTGCTCAT
GACGGAGGATTCGTCCAACGGATTGAGTTACCTAGTGGCGGACGGGTGAGTAACGCGTGAGGAACCTGCCTTGGAG
AGGGGAATAACACTCCGAAAGGAGTGCTAATACCGCATGATGCAGTTGGGTCGCATGGCTCTGACTGCCAAAGATT
TATCGCTCTGAGATGGCCTCGCGTCTGATTAGCTAGTAGGCGGGGTAACGGCCCACCTAGGCGACGATCAGTAGCC
GGACTGAGAGGTTGACCGGCCACATTGGGACTGAGACACGGCCCAGACTCCTACGGGAGGCAGCAGTGGGGAATAT
TGGGCAATGGGCGCAAGCCTGACCCAGCAACGCCGCGTGAAGGAAGAAGGCTTTCGGGTTGTAAACTTCTTTTGTC
GGGGACGAAACAAATGACGGTACCCGACGAATAAGCCACGGCTAACTACGTGCCAGCAGCCGCGGTAATACGTAGG
TGGCAAGCGTTATCCGGATTTACTGGGTGTAAAGGGCGTGTAGGCGGGATTGCAAGTCAGATGTGAAAACTGGGGG
CTCAACCTCCAGCCTGCATTTGAAACTGTAGTTCTTGAGTGCTGGAGAGGCAATCGGAATTCCGTGTGTAGCGGTG
AAATGCGTAGATATACGGAGGAACACCAGTGGCGAAGGCGGATTGCTGGACAGTAACTGACGCTGAGGCGCGAAAG
CGTGGGGAGCAAACAGGATTAGATACCCTGGTAGTCCACGCCGTAAACGATGGATACTAGGTGTGGGGGGTCTGAC
CCCCTCCGTGCCGCAGTTAACACAATAAGTATCCCACCTGGGGAGTACGATCGCAAGGTTGAAACTCAAAGGAATT
GACGGGGGCCCGCACAAGCGGTGGAGTATGTGGTTTAATTCGAAGCAACGCGAAGAACCTTACCAGGGCTTGACAT
CCCACTAACGAAGCAGAGATGCATTAGGTGCCCTTCGGGGAAAGTGGAGACAGGTGGTGCATGGTTGTCGTCAGCT
CGTGTCGTGAGATGTTGGGTTAAGTCCCGCAACGAGCGCAACCCTTATTGTTAGTTGCTACGCAAGAGCACTCTAG
CGAGACTGCCGTTGACAAAACGGAGGAAGGTGGGGACGACGTCAAATCATCATGCCCCTTATGTCCTGGGCCACAC
ACGTACTACAATGGTGGTTAACAGAGGGAGGCAATACCGCGAGGTGGAGCAAATCCCTAAAAGCCATCCCAGTTCG
GATTGCAGGCTGAAACCCGCCTGTATGAAGTTGGAATCGCTAGTAATCGCGGATCAGCATGCCGCGGTGAATACGT
TCCCGGGCCTTGTACACACCGCCCGTCACACCATGAGAGTCGGGAACACCCGAAGTCCGTAGCCTAACCGCAAGGA
GGGCGCGGCCGAAGGTGGGTTCGATAATTGGGGTGAAGTCGTAACAAGGTAGCCGTATCGGAAGGTGCGGCTGGAT
CACCTCCTTT
SEQ ID NO: 24 Blautia producta-2 Strain 3
TCAGAGAGTTTGATCCTGGCTCAGGATGAACGCTGGCGGCGTGCTTAACACATGCAAGTCGAGCGAAGCACTAAGA
CAGATTTCTTCGGATTGAAGTCTTTGTGACTGAGCGGCGGACGGGTGAGTAACGCGTGGGTAACCTGCCTCATACA
GGGGGATAACAGTTAGAAATGACTGCTAATACCGCATAAGCGCACAGGACCGCATGGTCTGGTGTGAAAAACTCCG
GTGGTATGAGATGGACCCGCGTCTGATTAGCTAGTTGGAGGGGTAACGGCCCACCAAGGCGACGATCAGTAGCCGG
CCTGAGAGGGTGAACGGCCACATTGGGACTGAGACACGGCCCAGACTCCTACGGGAGGCAGCAGTGGGGAATATTG
CACAATGGGGGAAACCCTGATGCAGCGACGCCGCGTGAAGGAAGAAGTATCTCGGTATGTAAACTTCTATCAGCAG
GGAAGAAAATGACGGTACCTGACTAAGAAGCCCCGGCTAACTACGTGCCAGCAGCCGCGGTAATACGTAGGGGGCA
AGCGTTATCCGGATTTACTGGGTGTAAAGGGAGCGTAGACGGAATAGCAAGTCTGATGTGAAAGGCTGGGGCTTAA
CCCCAGGACTGCATTGGAAACTGTTGTTCTAGAGTGCCGGAGAGGTAAGCGGAATTCCTAGTGTAGCGGTGAAATG
CGTAGATATTAGGAGGAACACCAGTGGCGAAGGCGGCTTACTGGACGGTAACTGACGTTGAGGCTCGAAAGCGTGG
GGAGCAAACAGGATTAGATACCCTGGTAGTCCACGCCGTAAACGATGAATACTAGGTGTCGGGTGGCAAAGCCATT
CGGTGCCGCAGCAAACGCAATAAGTATTCCACCTGGGGAGTACGTTCGCAAGAATGAAACTCAAAGGAATTGACGG
GGACCCGCACAAGCGGTGGAGCATGTGGTTTAATTCGAAGCAACGCGAAGAACCTTACCAAGTCTTGACATCCCTC
TGACCGTCCCGTAACGGGGACTTCCCTTCGGGGCAGAGGAGACAGGTGGTGCATGGTTGTCGTCAGCTCGTGTCGT
GAGATGTTGGGTTAAGTCCCGCAACGAGCGCAACCCTTATCCTTAGTAGCCAGCACATGATGGTGGGCACTCTAGG
GAGACTGCCGGGGATAACCCGGAGGAAGGCGGGGACGACGTCAAATCATCATGCCCCTTATGATTTGGGCTACACA
CGTGCTACAATGGCGTAAACAAAGGGAAGCGAGACAGCGATGTTGAGCGAATCCCAAAAATAACGTCCCAGTTCGG
ACTGCAGTCTGCAACTCGACTGCACGAAGCTGGAATCGCTAGTAATCGCGGATCAGAATGCCGCGGTGAATACGTT
CCCGGGTCTTGTACACACCGCCCGTCACACCATGGGAGTCAGTAACGCCCGAAGTCAGTGACCTAACCGAAAGGAA
GGAGCTGCCGAAGGCGGGACCGATAACTGGGGTGAAGTCGTAACAAGGTAGCCGTATCGGAAGGTGCGGCTGGATC
ACCTCCTTT
SEQ ID NO: 25 Blautia producta-3 Strain 4
TCAGAGAGTTTGATCCTGGCTCAGGATGAACGCTGGCGGCGTGCTTAACACATGCAAGTCGAGCGAAGCACTTAAG
TGGATCTCTTCGGATTGAAACTTATTTGACTGAGCGGCGGACGGGTGAGTAACGCGTGGGTAACCTGCCTCATACA
GGGGGATAACAGTTAGAAATGGCTGCTAATACCGCATAAGCGCACAGGACCGCATGGTCTGGTGTGAAAAACTCCG
GTGGTATGAGATGGACCCGCGTCTGATTAGCTAGTTGGAGGGGTAACGGCCCACCAAGGCGACGATCAGTAGCCGG
CCTGAGAGGGTGAACGGCCACATTGGGACTGAGACACGGCCCAGACTCCTACGGGAGGCAGCAGTGGGGAATATTG
CACAATGGGGGAAACCCTGATGCAGCGACGCCGCGTGAAGGAAGAAGTATCTCGGTATGTAAACTTCTATCAGCAG
GGAAGAAAATGACGGTACCTGACTAAGAAGCCCCGGCTAACTACGTGCCAGCAGCCGCGGTAATACGTAGGGGGCA
AGCGTTATCCGGATTTACTGGGTGTAAAGGGAGCGTAGACGGAAGAGCAAGTCTGATGTGAAAGGCTGGGGCTTAA
CCCCAGGACTGCATTGGAAACTGTTTTTCTAGAGTGCCGGAGAGGTAAGCGGAATTCCTAGTGTAGCGGTGAAATG
CGTAGATATTAGGAGGAACACCAGTGGCGAAGGCGGCTTACTGGACGGTAACTGACGTTGAGGCTCGAAAGCGTGG
GGAGCAAACAGGATTAGATACCCTGGTAGTCCACGCCGTAAACGATGAATACTAGGTGTCGGGTGGCAAAGCCATT
CGGTGCCGCAGCAAACGCAATAAGTATTCCACCTGGGGAGTACGTTCGCAAGAATGAAACTCAAAGGAATTGACGG
GGACCCGCACAAGCGGTGGAGCATGTGGTTTAATTCGAAGCAACGCGAAGAACCTTACCAAGTCTTGACATCCCTC
TGACCGGCCCGTAACGGGGCCTTCCCTTCGGGGCAGAGGAGACAGGTGGTGCATGGTTGTCGTCAGCTCGTGTCGT
GAGATGTTGGGTTAAGTCCCGCAACGAGCGCAACCCCTATCCTTAGTAGCCAGCAGGTGAAGCTGGGCACTCTAGG
GAGACTGCCGGGGATAACCCGGAGGAAGGCGGGGACGACGTCAAATCATCATGCCCCTTATGATTTGGGCTACACA
CGTGCTACAATGGCGTAAACAAAGGGAAGCGAGACAGCGATGTTGAGCAAATCCCAAAAATAACGTCCCAGTTCGG
ACTGCAGTCTGCAACTCGACTGCACGAAGCTGGAATCGCTAGTAATCGCGAATCAGAATGTCGCGGTGAATACGTT
CCCGGGTCTTGTACACACCGCCCGTCACACCATGGGAGTCAGTAACGCCCGAAGTCAGTGACCCAACCTTATAGGA
GGGAGCTGCCGAAGGCGGGACCGATAACTGGGGTGAAGTCGTAACAAGGTAGCCGTATCGGAAGGTGCGGCTGGAT
CACCTCCTTT
SEQ ID NO: 26 Clostridium ramosum Strain 5
Gatgaacgctggcggcgtgcctaatacatgcaagtcgaacgcgagcacttgtgctcgagtggcgaacgggtgagta
atacataagtaacctgccctagacagggggataactattggaaacgatagctaagaccgcataggtacggacactg
catggtgaccgtattaaagtgcctcaaagcactggtagaggatggacttatggcgcattagctggttggcggggta
acggcccaccaaggcgacgatgcgtagccgacctgagagggtgaccggccacactgggactgagacacggcccag
SEQ ID NO: 27 Flavonifractor plautii Strain 6
gatgaacgctggcggcgtgcttaacacatgcaagtcgaacggggtgctcatgacggaggattcgtccaacggattg
agttacccagtggcggacgggtgagtaacgcgtgaggaacctgccttggagaggggaataacactccgaaaggagt
gctaataccgcatgatgcagttgggtcgcatggctctgactgccaaagatttatcgctctgagatggcctcgcgtc
tgattagctagtaggcggggtaacggcccacctaggcgacgatcagtagccggactgagaggttgaccggccacat
tgggactgagacacggccca
SEQ ID NO: 28 Barnesiella/Parabacteroides spp Strain 7
GTGCTCAGCTTTTACCCTAGGCCGATCCTTGCGGTTACGGACTTCAGGTACCCCCGGCTCCCATGGCTTGACGGGC
GGTGTGTACAAGGCCCGGGAACGTATTCACCGCGCCATGGCTGATGCGCGATTACTAGCGAATCCAGCTTCACGGA
GTCGAGTTGCAGACTCCGATCCGAACTGAGACATGGTTTGGAGATTAGCATCCTGTCGCCAGGTAGCTGCCCTTTG
TCCATGCCATTGTAACACGTGTGTCGCCCCGGATGTAAGGGCCGTGCTGATTTGACGTCATCCCCACCTTCCTCAC
AGCTTACGCTGGCAGTCTCACCAGAGTCCTCAGCTTCACCTGTTAGTAACTAGTGATAAGGGTTGCGCTCGTTATG
GCACTTAAGCCGACACCTCACGGCACGAGCTGACGACAACCATGCAGCACCTCGTAATCTGCTATTGCTAGAAGGA
GTGTTTCCACTCCGGTCAGACTACGTTCAAACCCGGGTAAGGTTCCTCGCGTATCATCGAATTAAACCACATGTTC
CTCCGCTTGTGCGGGCCCCCGTCAATTCCTTTGAGTTTCACCGTTGCCGGCGTACTCCCCAGGTGGATTACTTAAC
GCTTTCGCTGTAGAGCTTACATTGTATCGCAAACTCCTAGTAATCATCGTTTACTGCGTGGACTACCAGGGTATCT
AATCCTGTTTGATCCCCACGCTTTCGTGCTTCAGTGTCAGTTATGGTTTAGTAAGCTGCCTTCGCAATCGGAGTTC
TGCGTGATATCTATGCATTTCACCGCTACACCACGCATTCCGCCTACCTCAAACACACTCAAGTAACCCAGTTTCA
ACGGCAATTTTATGGTTGAGCCACAAACTTTCACCGCTGACTTAAATCACCACCTACGCACCCTTTAACCCAATAA
ATCCGATAACGCTCGCATCCTCCGTATTACCGCGGCTGCTGCCCGGAGTTAGCCGATGCTTATTCATAGGGTACAT
ACAAAAAGGACACGT
SEQ ID NO: 29 Clostridium symbiosum Strain 8
ATGAGAGTTTGATCCTGGCTCAGGATGAACGCTGGCGGCGTGCCTAACACATGCAAGTCGAACGAAGCGATTTAA
CGGAAGTTTTCGGATGGAAGTTGAATTGACTGAGTGGCGGACGGGTGAGTAACGCGTGGGTAACCTGCCTTGTAC
TGGGGGACAACAGTTAGAAATGACTGCTAATACCGCATAAGCGCACAGTATCGCATGATACAGTGTGAAAAACTC
CGGTGGTACAAGATGGACCCGCGTCTGATTAGCTAGTTGGTAAGGTAACGGCTTACCAAGGCGACGATCAGTAGC
CGACCTGAGAGGGTGACCGGCCACATTGGGACTGAGACACGGCCCAAACTCCTACGGGAGGCAGCAGTGGGGAAT
ATTGCACAATGGGCGAAAGCCTGATGCAGCGACGCCGCGTGAGTGAAGAAGTATTTCGGTATGTAAAGCTCTATC
AGCAGGGAAGAAAATGACGGTACCTGACTAAGAAGCCCCGGCTAACTACGTGCCAGCAGCCGCGGTAATACGTAG
GGGGCAAGCGTTATCCGGATTTACTGGGTGTAAAGGGAGCGTAGACGGTAAAGCAAGTCTGAAGTGAAAGCCCGC
GGCTCAACTGCGGGACTGCTTTGGAAACTGTTTAACTGGAGTGTCGGAGAGGTAAGTGGAATTCCTAGTGTAGCG
GTGAAATGCGTAGATATTAGGAGGAACACCAGTGGCGAAGGCGACTTACTGGACGATAACTGACGTTGAGGCTCG
AAAGCGTGGGGAGCAAACAGGATTAGATACCCTGGTAGTCCACGCCGTAAACGATGAATACTAGGTGTTGGGGAG
CAAAGCTCTTCGGTGCCGTCGCAAACGCAGTAAGTATTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTACCAGGTC
TTGACATCGATCCGACGGGGGAGTAACGTCCCCTTCCCTTCGGGGCGGAGAAGACAGGTGGTGCATGGTTGTCGT
CAGCTCGTGTCGTGAGATGTTGGGTTAAGTCCCGCAACGAGCGCAACCCTTATTCTAAGTAGCCAGCGGTTCGGC
CGGGAACTCTTGGGAGACTGCCAGGGATAACCTGGAGGAAGGTGGGGATGACGTCAAATCATCATGCCCCTTATG
ATCTGGGCTACACACGTGCTACAATGGCGTAAACAAAGAGAAGCAAGACCGCGAGGTGGAGCAAATCTCAAAAAT
AACGTCTCAGTTCGGACTGCAGGCTGCAACTCGCCTGCACGAAGCTGGAATCGCTAGTAATCGCGAATCAGAATG
TCGCGGTGAATACGTTCCCGGGTCTTGTACACACCGCCCGTCACACCATGGGAGTCAGTAACGCCCGAAGTCAGT
GACCCAACCGCAAGGAGGGAGCTGCCGAAGGCGGGACCGATAACTGGGGTGAAGTCGTAACAAGGTAGCCGTATC
GGAAGGTGCGGCTGGATCACCTCCTTT
SEQ ID NO: 30 Eubacterium fissicatena Strain 23
gatgaacgctggcggcgtgcttaacacatgcaagtcgagcgaagcgctttacttagatttcttcggattgaaagtt
ttgcgactgagcggcggacgggtgagtaacgcgtgggtaacctgcctcatacagggggataacagttagaaatgac
tgctaataccgcataagaccacagtaccgcatggtacagtgggaaaaactccggtggtatgagatggacccgcgtc
tgattagctagttggtaaggtaacggcttaccaaggcgacgatcagtagccgacctgagagggtgaccggccacat
tgggactgagacacggccca
SEQ ID NO: 31 Lachnospiraceae bacterium Strain 25
agagtttgatcctggctcaggataaacgctggcggcgcacataagacatgcaagtcgaacggacttaactcattct
tttagattgagagcggttagtggcggactggtgagtaacacgtaagcaacctgcctatcagaggggaataacagtg
agaaatcattgctaataccgcatatgctcacagtatcacatgatacagtgaggaaaggagcaatccgctgatagat
gggcttgcgcctgattagttagttggtggggtaacggcctaccaagacgacgatcagtagccggactgagaggttg
aacggccacattgggactgagatacggcccagactcctacgggaggcagcagtcgggaatattgcgcaatggagga
aactctgacgcagtgacgccgcgtataggaagaaggttttcggattgtaaactattgtcgttagggaagataaaag
actgtacctaaggaggaagccccggctaactatgtgccagcagccgcggtaatacatagggggcaagcgttatccg
gaattattgggtgtaaagggtgcgtagacggaagaacaagttggttgtgaaatccctcggctcaactgaggaactg
caaccaaaactattctccttgagtgtcggagaggaaagtggaattcctagtgtagcggtgaaatgcgtagatatta
ggaggaacaccagtggcgaaggcgactttctggacgataactgacgttgaggcacgaaagtgtggggagcaaacag
gattagataccctggtagtccacactgtaaacgatggatactaggtgtagggtgtattaagcactctgtgccgccg
ctaacgcattaagtatcccacctggggagtacgaccgcaaggttgaaactcaaaggaattgacgggggcccgcaca
agcagtggagtatgtggtttaattcgaagcaacgcgaagaaccttaccagggcttgacatataccggaatatacta
gagatagtatagtccttcgggactggtatacaggtggtgcatggttgtcgtcagctcgtgtcgtgagatgttgggt
taagtcccgcaacgagcgcaacccctatcgttagttgctagcaggtaatgctgagaactctagcgagactgccggt
gataaatcggaggaaggtggggatgacgtcaaatcatcatgccctttatgtcctgggctacacacgtactacaatg
gccgtaacagagggaagcaatatagtgatatggagcaaaaccctaaaagcggtctcagttcggattgaaggctgaa
attcgccttcatgaagccggaattgctagtaatggcaggtcagcatactgccgtgaatacgttcccgggccttgta
cacaccgcccgtcacaccatgagagttggaaatacccgaagcctgtgagctaactgtaaagaggcagcagtcgaag
gtagagccaatgattggggtgaagtcgtaacaaggtagccgt
SEQ ID NO: 32 Dorea Longicatena Strain 18
AACGAGAGTTTGATCCTGGCTCAGGATGAACGCTGGCGGCGTGCTTAACACATGCAAGTCGAGCGAAGCACTTTG
GAAGATTCTTCGGATGATTTCCTTTGTGACTGAGCGGCGGACGGGTGAGTAACGCGTGGGTAACCTGCCTCATAC
AGGGGGATAACAGTTAGAAATGACTGCTAATACCGCATAAGACCACGGTACCGCATGGTACAGTGGTAAAAACTC
CGGTGGTATGAGATGGACCCGCGTCTGATTAGGTAGTTGGTGGGGTAACGGCCTACCAAGCCGACGATCAGTAGC
CGACCTGAGAGGGTGACCGGCCACATTGGGACTGAGACACGGCCCAGACTCCTACGGGAGGCAGCAGTGGGGAAT
ATTGCACAATGGAGGAAACTCTGATGCAGCGACGCCGCGTGAAGGATGAAGTATTTCGGTATGTAAACTTCTATC
AGCAGGGAAGAAAATGACGGTACCTGACTAAGAAGCCCCGGCTAACTACGTGCCAGCAGCCGCGGTAATACGTAG
GGGGCAAGCGTTATCCGGATTTACTGGGTGTAAAGGGAGCGTAGACGGCACGGCAAGCCAGATGTGAAAGCCCGG
GGCTCAACCCCGGGACTGCATTTGGAACTGCTGAGCTAGAGTGTCGGAGAGGCAAGTGGAATTCCTAGTGTAGCG
GTGAAATGCGTAGATATTAGGAGGAACACCAGTGGCGAAGGCGGCTTGCTGGACGATGACTGACGTTGAGGCTCG
AAAGCGTGGGGAGCAAACAGGATTAGATACCCTGGTAGTCCACGCCGTAAACGATGACTGCTAGGTGTCGGGTGG
CAAAGCCATTCGGTGCCGCAGCTAACGCAATAAGCAGTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTACCTGATC
TTGACATCCCGATGACCGCTTCGTAATGGAAGCTTTTCTTCGGAACATCGGTGACAGGTGGTGCATGGTTGTCGT
CAGCTCGTGTCGTGAGATGTTGGGTTAAGTCCCGCAACGAGCGCAACCCCTATCTTCAGTAGCCAGCAGGTTAAG
CTGGGCACTCTGGAGAGACTGCCAGGGATAACCTGGAGGAAGGTGGGGATGACGTCAAATCATCATGCCCCTTAT
GACCAGGGCTACACACGTGCTACAATGGCGTAAACAAAGAGAAGCGAACTCGCGAGGGTAAGCAAATCTCAAAAA
TAACGTCTCAGTTCGGATTGTAGTCTGCAACTCGACTACATGAAGCTGGAATCGCTAGTAATCGCAGATCAGAAT
GCTGCGGTGAATACGTTCCCGGGTCTTGTACACACCGCCCGTCACACCATGGGAGTCAGTAACGCCCGAAGTCAG
TGACCCAACCGTAAGGAGGGAGCTGCCGAAGGTGGGACCGATAACTGGGGTGAAGTCGTAACAAGGTAGCCGTAT
CGGAAGGTGCGGCTGGATCACCTCCTTT
SEQ ID NO: 33 Blautia producta-4 Strain 10
ATCAGAGAGTTTGATCCTGGCTCAGGATGAACGCTGGCGGCGTGCTTAACACATGCAAGTCGAGCGAAGCACTTA
AGTGGATCTCTTCGGATTGAAACTTATTTGACTGAGCGGCGGACGGGTGAGTAACGCGTGGGTAACCTGCCTCAT
ACAGGGGGATAACAGTTAGAAATGGCTGCTAATACCGCATAAGCGCACAGGACCGCATGGTCTGGTGTGAAAAAC
TCCGGTGGTATGAGATGGACCCGCGTCTGATTAGCTAGTTGGAGGGGTAACGGCCCACCAAGGCGACGATCAGTA
GCCGGCCTGAGAGGGTGAACGGCCACATTGGGACTGAGACACGGCCCAGACTCCTACGGGAGGCAGCAGTGGGGA
ATATTGCACAATGGGGGAAACCCTGATGCAGCGACGCCGCGTGAAGGAAGAAGTATCTCGGTATGTAAACTTCTA
TCAGCAGGGAAGAAAATGACGGTACCTGACTAAGAAGCCCCGGCTAACTACGTGCCAGCAGCCGCGGTAATACGT
AGGGGGCAAGCGTTATCCGGATTTACTGGGTGTAAAGGGAGCGTAGACGGAAGAGCAAGTCTGATGTGAAAGGCT
GGGGCTTAACCCCAGGACTGCATTGGAAACTGTTTTTCTAGAGTGCCGGAGAGGTAAGCGGAATTCCTAGTGTAG
CGGTGAAATGCGTAGATATTAGGAGGAACACCAGTGGCGAAGGCGGCTTACTGGACGGTAACTGACGTTGAGGCT
CGAAAGCGTGGGGAGCAAACAGGATTAGATACCCTGGTAGTCCACGCCGTAAACGATGAATACTAGGTGTCGGGT
GGCAAAGCCATTCGGTGCCGCAGCAAACGCAATAAGTATTCCACCTGGGGAGTACGTTCGCAAGAATGAAACTCA
AAGGAATTGACGGGGACCCGCACAAGCGGTGGAGCATGTGGTTTAATTCGAAGCAACGCGAAGAACCTTACCAAG
TCTTGACATCCCTCTGACCGGCCCGTAACGGGGCCTTCCCTTCGGGGCAGAGGAGACAGGTGGTGCATGGTTGTC
GTCAGCTCGTGTCGTGAGATGTTGGGTTAAGTCCCGCAACGAGCGCAACCCCTATCCTTAGTAGCCAGCAGGTGA
AGCTGGGCACTCTAGGGAGACTGCCGGGGATAACCCGGAGGAAGGCGGGGACGACGTCAAATCATCATGCCCCTT
ATGATTTGGGCTACACACGTGCTACAATGGCGTAAACAAAGGGAAGCGAGACAGCGATGTTGAGCAAATCCCAAA
AATAACGTCCCAGTTCGGACTGCAGTCTGCAACTCGACTGCACGAAGCTGGAATCGCTAGTAATCGCGAATCAGA
ATGTCGCGGTGAATACGTTCCCGGGTCTTGTACACACCGCCCGTCACACCATGGGAGTCAGTAACGCCCGAAGTC
AGTGACCCAACCTTACAGGAGGGAGCTGCCGAAGGCGGGACCGATAACTGGGGTGAAGTCGTAACAAGGTAGCCG
TATCGGAAGGTGCGGCTGGATCACCTCCTTT
SEQ ID NO: 34 E. coli Strain 46
agtttgatcatggctcagattgaacgctggcggcaggcctaacacatgcaagtcgaacggtaacaggaacgagctt
gctgctttgctgacgagtggcggacgggtgagtaatgtctgggaaactgcctgatggagggggataactactggaa
acggtagctaataccgcataacgtcgcaagaccaaagagggggaccttcgggcctcttgccatcggatgtgcccag
atgggattagctagtaggtggggtaaaggctcacctaggcgacgatccctagctggtctgagaggatgaccagcca
cactggaactgagacacggtccagactcctacgggaggcagcagtggggaatattgcacaatgggcgcaagcctga
tgcagccatgccgcgtgtatgaagaaggccttcgggttgtaaagtactttcagcggggaggaagggagtaaagtta
atacctttgctcattgacgttaccgcagaagaannaccggctaactccgtgccagcagccgcggtaatacggaggg
tgcaagcgttaatcggaattactgggcgtaaagngcangcaggcggtttgttaagtcagatgtgaaatccccgggc
tcaacctgggaactgcatctgatactggcaagcttgagtctcgtagaggggggtagaattccaggtgtagcggtga
aatgcgtagagatctggaggaataccggtggcgaaggcggccccctggacgaagactgacgctcaggtgcgaaagc
gtggggagcaaacaggattagataccctggtagtccacgccgtaaacgatgtcgacttggaggttgtgcccttgag
gcgtggcttccgganntaacgcgttaagtcgaccgcctggggagtacggccgcaaggttaaaactcaaatgaattg
acgggggccgcacaagcggtggagcatgtggtttaattcgatgcaacgcgaagaaccttacctggtcttgacatcc
acggaagttttcagagatgagaatgtgccttcgggaaccgtgagacaggtgctgcatggctgtcgtcagctcgtgt
tgtgaaatgttgggttaagtcccgcaacgagcgcaacccttatcctttgttgccagcggtccggccgggaactcaa
aggagactgccagtgataaactggaggaaggtggggatgacgtcaagtcatcatggcccttacgaccagggctaca
cacgtgctacaatggcgcatacaaagagaagcgacctcgcgagagcaagcggacctcataaagtgcgtcgtagtcc
ggattggagtctgcaactcgactccatgaagtcggaatcgctagtaatcgtggatcagaatgccacggtgaatacg
ttcccgggccttgtacacaccgcccgtcacaccatgggagtgggttgcaaaagaagtaggtagcttaaccttcggg
agggcgc
SEQ ID NO: 35 Lactococcus lactis Strain 47
NNAATTTTTGTTGTGCTCATACGTGCAGTTGAGCGCTCGAAGGTTGGTACTTGTACCCTCTGGATGAGCAGCGAAC
GGGTGAGTAACGCGTGGGGAATCTGCCTTTGAGCGGGGGACCACATTTGGAAACGAATGCGAATACCGCATAAAAA
CTTTAAACACAAGTTTTAAGTTTGAAAGATGCAATTGCATCACTCCAAGATGATCCCGCGTTGTATTAGCTAGTTG
GTGAGGGAAAGGCTCCCCACGGCGATCATACATATCCGACCTGAGAGGGTGACCGGCCACATTGGGACTGAGCCAT
GATCAAACTCTGAAAAAGAGGCAGCAGTAGGGAATCTTCGGCAATGGACGAAAGTCTGACCGAGCAACGCCGCGTG
AGTGAAGAAGGTTTTCGGATCGTAAAACTCTGTTGGTAGAGAAGAACGTTGGTGAGAGTGGAAAGCTCATCAAGTG
ACGGTAACTACCCAGAAAGGGACGGCTAACTACGTGCCAGCAGCCGCGGTAATACGTAGGTCCCGAGCGTTGTCCG
GATTTATTGGGCGTAAAGCGAGCGCAGGTGGTTTATTAAGTCTGGTGTAAAAGGCAGTGGCTCAACCATTGTATGC
ATTGGAAACTGGTAGACTTGAGTGCAGGAGAGGAGAGTGGAATTCCATGTGTAGCGGGTGAAATGCGTAGATATAT
GGTAGGAACACCGGGTGGCGAAAGCGGCTCTCTGGCCTGTAACTGACACTGAGGCTCGAAAAGCGTGGGGAGCAAA
AAGGATTAGATACCCTGGTAGTCCACGCCGTA
SEQ ID NO: 36 Lactobacillus ruminis Strain 48
NACTCTGTCACCTTAGGCGGCTGGCTCCAAAAGGTTACCCCACCGACTTTGGGTGTTACAAACTCTCATGGTGTGA
CGGGCGGTGTGTACAAGGCCCGGGAACGTATTCACCGCGACATGCTGATTCGCGATTACTAGCGATTCCGACTTCA
TGCAGGCGAGTTGCAGCCTGCAATCCGAACTGAGAACGGCTTTAAGAGATTAGCTTGCCCTCGCGAGTTAGCGACT
CGTTGTACCGTCCATTGTAGCACGTGTGTAGCCCAGGTCATAAGGGGCATGATGATTTGACGTCATCCCCACCTTC
CTCCGGTTTGTCACCGGCAGTCTCGCCAGAGTGCCCAACTTAATGATGGCAACTGACAATAAGGGTTGCGCTCGTT
GCGGGACTTAACCCAACATCTCACGACACGAGCTGACGACAACCATGCACCACCTGTCATTCTGTCCCCGAAGGGA
ACGTTCCATCTCTGGAATTGTCAGAAGATGTCAAGACCTGGTAAGGTTCTTCGCGTTGCTTCGAATTAAACCACAT
GCTCCACCGCTTGTGCGGGCCCCCGTCAATTCCTTTGAGTTTCAGTCTTGCGACCGTACTCCCCAGGCGGAGTGCT
TAATGCGTTAGCTGCAGCACTGAAGGGCGGAAACCCTCCAACACTTAGCACTCATCGTTTACGGCGTGGACTACCA
GGGTATCTAATCCTGTTTGCTACCCACGCTTTCGAACCTCAGCGTCAGTTACAGACCAGAGAGCCGCTTTCGCCAC
TGGTGTTCTTCCATATATCTACGCATTTCACCGCTACACATGGAGTTCCACTCTCCTCTTCTGCACTCAAGTCTTC
CAGTTTCCAATGCACTACTTCGGTTAAGCCGAAGGCTTTCACATCAGACTTAAAAGACCGCCTGCGTTCCCTTTAC
GCCCATAAATCCGGACACGCTCGCCACCTACGTATTACCGCGGCTGCTGGCACGTAGTTAGCCGTGGCTTTCTGGT
AGA
SEQ ID NO: 37 Lactobacillus ruminis Strain 49
ATTGAGAGTTTGATCCTGGCTCAGGACGAACGCTGGCGGCGTGCCTAATACATGCAAGTCGAACGAAGCTTTCTTT
CACCGAATGCTTGCATTCACCGAAAGAAGCTTAGTGGCGAACGGGTGAGTAACACGTAGGCAACCTGCCCAAAAGA
GGGGGATAACACTTGGAAACAGGTGCTAATACCGCATAACCATGAACACCGCATGATGTTCATGTAAAAGACGGCT
TTTGCTGTCACTTTTGGATGGGCCTGCGGCGTATTAACTTGTTGGTGGGGTAACGGCCTACCAAGGTGATGATACG
TAGCCGAACTGAGAGGTTGATCGGCCACATTGGGACTGAGACACGGCCCAAACTCCTACGGGAGGCAGCAGTAGGG
AATCTTCCACAATGGACGAAAGTCTGATGGAGCAACGCCGCGTGAATGAAGAAGGCCTTCGGGTCGTAAAATTCTG
TTGTCAGAGAAGAACGTGCGTGAGAGTAACTGTTCACGTATTGACGGTATCTGACCAGAAAGCCACGGCTAACTAC
GTGCCAGCAGCCGCGGTAATACGTAGGTGGCGAGCGTTGTCCGGATTTATTGGGCGTAAAGGGAACGCAGGCGGTC
TTTTAAGTCTGATGTGAAAGCCTTCGGCTTAACCGAAGTAGTGCATTGGAAACTGGAAGACTTGAGTGCAGAAGAG
GAGAGTGGAACTCCATGTGTAGCGGTGAAATGCGTAGATATATGGAAGAACACCAGTGGCGAAAGCGGCTCTCTGG
TCTGTAACTGACGCTGAGGTTCGAAAGCGTGGGTAGCAAACAGGATTAGATACCCTGGTAGTCCACGCCGTAAACG
ATGAGTGCTAAGTGTTGGAGGGTTTCCGCCCTTCAGTGCTGCAGCTAACGCATTAAGCACTCCGCCTGGGGAGTAC
GGTCGCAAGACTGAAACTCAAAGGAATTGACGGGGGCCCGCACAAGCGGTGGAGCATGTGGTTTAATTCGAAGCAA
CGCGAAGAACCTTACCAGGTCTTGACATCTTCTGACAATTCCAGAGATGGAACGTTCCCTTCGGGGACAGAATGAC
AGGTGGTGCATGGTTGTCGTCAGCTCGTGTCGTGAGATGTTGGGTTAAGTCCCGCAACGAGCGCAACCCTTATTGT
CAGTTGCCATCATTAAGTTGGGCACTCTGGCGAGACTGCCGGTGACAAACCGGAGGAAGGTGGGGATGACGTCAAA
TCATCATGCCCCTTATGACCTGGGCTACACACGTGCTACAATGGACGGTACAACGAGTCGCTAACTCGCGAGGGCA
AGCTAATCTCTTAAAGCCGTTCTCAGTTCGGATTGCAGGCTGCAACTCGCCTGCATGAAGTCGGAATCGCTAGTAA
TCGCGAATCAGCATGTCGCGGTGAATACGTTCCCGGGCCTTGTACACACCGCCCGTCACACCATGAGAGTTTGTAA
CACCCAAAGTCGGTGGGGTAACCTTTTGGAGCCAGCCGCCTAAGGTGGGACAGATGATTGGGGTGAAGTCGTAACA
AGGTAGCCGTAGGAGAACCTGCGGCTGGATCACCTCCTT
SEQ ID NO: 38 Lactobacillus animalis Strain 50
ATTGAGAGTTTGATCCTGGCTCAGGATGAACGCTGGCGGCGTGCCTAATACATGCAAGTCGAACGAAACTTCTTTA
TCACCGAGTGCTTGCACTCACCGATAAAGAGTTGAGTGGCGAACGGGTGAGTAACACGTGGGCAACCTGCCCAAAA
GAGGGGGATAACACTTGGAAACAGGTGCTAATACCGCATAACCATAGTTACCGCATGGTAACTATGTAAAAGGTGG
CTATGCTACCGCTTTTGGATGGGCCCGCGGCGCATTAGCTAGTTGGTGAGGTAAAGGCTTACCAAGGCAATGATGC
GTAGCCGAACTGAGAGGTTGATCGGCCACATTGGGACTGAGACACGGCCCAAACTCCTACGGGAGGCAGCAGTAGG
GAATCTTCCACAATGGGCGAAAGCCTGATGGAGCAACGCCGCGTGGGTGAAGAAGGTCTTCGGATCGTAAAACCCT
GTTGTTAGAGAAGAAAGTGCGTGAGAGTAACTGTTCACGTTTCGACGGTATCTAACCAGAAAGCCACGGCTAACTA
CGTGCCAGCAGCCGCGGTAATACGTAGGTGGCAAGCGTTATCCGGATTTATTGGGCGTAAAGGGAACGCAGGCGGT
CTTTTAAGTCTGATGTGAAAGCCTTCGGCTTAACCGGAGTAGTGCATTGGAAACTGGGAGACTTGAGTGCAGAAGA
GGAGAGTGGAACTCCATGTGTAGCGGTGAAATGCGTAGATATATGGAAGAACACCAGTGGCGAAAGCGGCTCTCTG
GTCTGTAACTGACGCTGAGGTTCGAAAGCGTGGGTAGCAAACAGGATTAGATACCCTGGTAGTCCACGCCGTAAAC
GATGAATGCTAAGTGTTGGAGGGTTTCCGCCCTTCAGTGCTGCAGCTAACGCAATAAGCATTCCGCCTGGGGAGTA
CGACCGCAAGGTTGAAACTCAAAGGAATTGACGGGGGCCCGCACAAGCGGTGGAGCATGTGGTTTAATTCGAAGCA
ACGCGAAGAACCTTACCAGGTCTTGACATCTTCTGACAATCCTAGAGATAGGACTTTCCCTTCGGGGACAGAATGA
CAGGTGGTGCATGGTTGTCGTCAGCTCGTGTCGTGAGATGTTGGGTTAAGTCCCGCAACGAGCGCAACCCTTATTG
TTAGTTGCCAGCATTAAGTTGGGCACTCTAGCAAGACTGCCGGTGACAAACCGGAGGAAGGTGGGGATGACGTCAA
ATCATCATGCCCCTTATGACCTGGGCTACACACGTGCTACAATGGACGGTACAACGAGTCGCAAGACCGCGAGGTT
TAGCAAATCTCTTAAAGCCGTTCTCAGTTCGGATTGTAGGCTGCAACTCGCCTACATGAAGTCGGAATCGCTAGTA
ATCGCGGATCAGCATGCCGCGGTGAATACGTTCCCGGGCCTTGTACACACCGCCCGTCACACCATGAGAGTTTGTA
ACACCCAAAGCCGGTGGGGTAACCTTTTGGAGCCAGCCGTCTAAGGTGGGACAGATGATTGGGGTGAAGTCGTAAC
AAGGTAGCCGTAGGAGAACCTGCGGCTGGATCACCTCCTTT
SEQ ID NO: 39 Lactobacillus rhamnosus Strain 51
NNCTCTGTTTTGCGTGTGATGCAGTCGACGAGTTCTGATTATTGAAAGGTGCTTGCATCTTGATTTAATTTTGAAC
GAGTGGCGGACGGGTGAGTAACACGTGGGTAACCTGCCCTTAAGTGGGGGATAACATTTGGAAACAGATGCTAATA
CCGCATAAATCCAAGAACCGCATGGTTCTTGGCTGAAAGATGGCGTAAGCTATCGCTTTTGGATGGACCCGCGGCG
TATTAGCTAGTTGGTGAGGTAACGGCTCACCAAGGCAATGATACGTAGCCGAACTGAGAGGTTGATCGGCCACATT
GGGACTGAGACACGGCCCAAACTCCTACGGGAGGCAGCAGTAGGGAATCTTCCACAATGGACGCAAGTCTGATGGA
GCAACGCCGCGTGAGTGAAGAAGGCTTTCGGGTCGTAAAACTCTGTTGTTGGAGAAGAATGGTCGGCAGAGTAACT
GTTGTCGGCGTGACGGTATCCAACCAGAAAGCCACGGCTAACTACGTGCCAGCAGCCGCGGTAATACGTAGGTGGC
AAGCGTTATCCGGATTTATTGGGCGTAAAGCGAGCGCAGGCGGTTTTTTAAGTCTGATGTGAAAGCCCTCGGCTTA
ACCGAGGAAGTGCATCGGAAACTGGGAAACTTGAGTGCAGAAGAGGACAGTGGAACTCCATGTGTAGCGGTGAAAT
GCGTAGATATATGGAAGAACACCAGTGGCGAGGCGGCTGTCTGGTCTGTAACTGACGCTGAGGCTCGAAAGCATGG
GTAGCGAACAGGATTAGATACCCTGGTAGTCCATGCCGTAAACGATGAATGCTAGGTGTTGGAGGGTTTCCGCCCT
TCAGTGCCGCGCTTACGCATTTAGCATTCGCCTGGGGAGTACGACCGCAGGTTGAACCTCAAAGGAATTG
SEQ ID NO: 40 Lactobacillus rhamnosus Strain 52
CCCNNTTGTGTCCTATACTGCAGTCTACAGTCTGAATATTGAAGGTGCTTGCATCTTGATTTAATTTTGAACGAGT
GGCGGACGGGTGAGTAACACGTGGGTAACCTGCCCTTAAGTGGGGGATAACATTTGGAAACAGATGCTAATACCGC
ATAAATCCAAGAACCGCATGGTTCTTGGCTGAAAGATGGCGTAAGCTATCGCTTTTGGATGGACCCGCGGCGTATT
AGCTAGTTGGTGAGGTAACGGCTCACCAAGGCAATGATACGTAGCCGAACTGAGAGGTTGATCGGCCACATTGGGA
CTGAGACACGGCCCAAACTCCTACGGGAGGCAGCAGTAGGGAATCTTCCACAATGGACGCAAGTCTGATGGAGCAA
CGCCGCGTGAGTGAAGAAGGCTTTCGGGTCGTAAAACTCTGTTGTTGGAGAAGAATGGTCGGCAGAGTAACTGTTG
TCGGCGTGACGGTATCCAACCAGAAAGCCACGGCTAACTACGTGCCAGCAGCCGCGGTAATACGTAGGTGGCAAGC
GTTATCCGGATTTATTGGGCGTAAAGCGAGCGCAGGCGGTTTTTTAAGTCTGATGTGAAAGCCCTCGGCTTAACCG
AGGAAGTGCATCGGAAACTGGGAAACTTGAGTGCAGAAGAGGACAGTGGAACTCCATGTGTAGCGGTGAAATGCGT
AGATATATGGAAGAACACCAGTGGCGAAGGCGGCTGTCTGGTCTGTAACTGACGCTGAGGCTCGAAAGCATGGGTA
GCGAACAGGATTAGATACCCTGGTAGTCCATGCCGTAAACGATGAATGCTAGGTGTTGGAGGGTTTCCGCCCTTCA
GTGCCGCAGCTAACGCATTAAGCATTCCGCCTGGGGGAGTACGACCGCAAGGTTGAACTCAAAGGAATTGACGGGG
CCCGCACAAGCGGGGGAGCATGTGGTTTAATTCGAAGCAACGCGAGGACCTTACCAGGTCTTGACATCTTTTGATC
ACCTGAGAGATCAGGTTTCCCCTTCGGGGCAAATGACAGTGGTGCATGGTTGTCGTCAGCTCCGTGTCTGAGATGT
TGGGTAAGTCCGCAACAAGCGCAACCCTTATGACTAGTTGCAGCTTAGTGGGCACTCCTAGTAGACTGCCGGTGAC
AACCGGAGGAAGGGTGGGGATGACTCAATCACTAGCCCTNGGACCTGGGCTACAACNNGTCCTCATG
SEQ ID NO: 41 Lactobacillus rhamnosus Strain 53
GNTCTGGTTTGTTTTGTTGGGGGGTGAAATCTAGTATTGAGGTGCTTGCATCTTGGTTTAATTGTGGAGGAGTGGC
GGACGGGTGAGTAACACGTGGGTAACCTGCCCTTAAGTGGGGGATAACATTTGGAAACAGATGCTAATACCGCATA
AATCCAAGAACCGCATGGTTCTTGGCTGAAAGATGGCGTAAGCTATCGCTTTTGGATGGACCCGCGGCGTATTAGC
TAGTTGGTGAGGTAACGGCTCACCAAGGCAATGATACGTAGCCGAACTGAGAGGTTGATCGGCCACATTGGGACTG
AGACACGGCCCAAACTCCTACGGGAGGCAGCAGTAGGGAATCTTCCACAATGGACGCAAGTCTGATGGAGCAACGC
CGCGTGAGTGAAGAAGGCTTTCGGGTCGTAAAACTCTGTTGTTGGAGAAGAATGGTCGGCAGAGTAACTGTTGTCG
GCGTGACGGTATCCAACCAGAAAGCCACGGCTAACTACGTGCCAGCAGCCGCGGTAATACGTAGGTGGCAAGCGTT
ATCCGGATTTATTGGGCGTAAAGCGAGCGCAGGCGGTTTTTTAAGTCTGATGTGAAAGCCCTCGGCTTAACCGAGG
AAGTGCATCGGAAACTGGGAAACTTGAGTGCAGAAGAGGACAGTGGAACTCCATGTGTAGCGGTGAAATGCGTAGA
TATATGGAAGAACACCAGTGGCGAAGGCGGCTGTCTGGTCTGTAACTGACGCTGAGGCTCGAAAGCATGGGTAGCG
AACAGGATTAGATACCCTGGTAGTCCATGCCGTAAACGATGAATGCTAGGTGTTGGAGGGTTTCCGCCCTTCAGTG
CCGCAGCTAACGCATTAAGCATCCGCCTGGGGAGTACGACCGCAAGGTTGAAACTCAAAGGAATTGACGGGGCCCG
CACAGCGTGGAGCATGTGGTTTAATTCGAAGCAACGCGAGAACCTTACCAGGTCTGACATCTTTGATCACTGAGAG
ATCAGGTTTCCCTTCGGGCAAATGACAGTGGTGCATGGTTGTCGTCAGCTCGTGTCTNGAGATGTTGGGTTAAT
SEQ ID NO: 42 Bacteroides caccae Strain 54
TGGCTCAGGATGAACGCTAGCTACAGGCTTAACACATGCAAGTCGAGGGGCATCAGTTTGGTTTGCTTGCAAACCA
AAGCTGGCGACCGGCGCACGGGTGAGTAACACGTATCCAACCTGCCTCATACTCGGGGATAGCCTTTCGAAAGAAA
GATTAATATCCGATAGCATATATTTCCCGCATGGGTTTTATATTAAAGAAATTCGGTATGAGATGGGGATGCGTTC
CATTAGTTTGTTGGGGGGGTAACGGCCCACCAAGACTACGATGGATAGGGGTTCTGAGAGGAAGGTCCCCCACATT
GGAACTGAGACACGGTCCAAACTCCTACGGGAGGCAGCAGTGAGGAATATTGGTCAATGGACGCGAGTCTGAACCA
GCCAAGTAGCGTGAAGGATGACTGCCCTATGGGTTGTAAACTTCTTTTATATGGGAATAAAGTTGTCCACGTGTGG
ATTTTTGTATGTACCATATGAATAAGGATCGGCTAACTCCGTGCCAGCAGCCGCGGTAATACGGAGGATCCGAGCG
TTATCCGGATTTATTGGGTTTAAAGGGAGCGTAGGCGGATTGTTAAGTCAGTTGTGAAAGTTTGCGGCTCAACCGT
AAAATTGCAGTTGATACTGGCAGTCTTGAGTGCAGTAGAGGTGGGCGGAATTCGTGGTGTAGCGGTGAAATGCTTA
GATATCACGAAGAACTCCGATTGCGAAGGCAGCCACTGGAGTGTAACTGACGCTGATGCTCGAAAGTGTGGGTATC
AAACAGGATTAGATACCCTGGTAGTCCACACAGTAAACGATGAATACTCGCTGTTTGCGATATACAGTAAGCGGCC
AAGCGAAAGCATTAAGTATTCCACCTGGGGAGTACGCCGGCAACGGTGAAACTCAAAGGAATTGACGGGGGCCCGC
ACAAGCGGAGGAACATGTGGTTTAATTCGATGATACGCGAGGAACCTTACCCGGGCTTAAATTGCAAATGAATTAT
GGGGAAACCCATAGGCCGCAAGGCATTTGTGAAGGTGCTGCATGGTTGTCGTCAGCTCGTGCCGTGAGGTGTCGGC
TTAAGTGCCATAACGAGCGCAACCCTTATCTTCAGTTACTAACAGGTCATGCTGAGGACTCTGGAGAGACTGCCGT
CGTAAGATGTGAGGAAGGTGGGGATGACGTCAAATCAGCACGGCCCTTACGTCCGGGGCTACACACGTGTTACAAT
GGGGGGTACAGAAGGCCGCTACCTGGTGACAGGATGCCAATCCCAAAAACCTCTCTCAGTTCGGATCGAAGTCTGC
AACCCGACTTCGTGAAGCTGGATTCGCTAGTAATCGCGCATCAGCCATGGCGCGGTGAATACGTTCCCGGGCCTTG
TACACACCGCCCGTCAAGCCATGAAAGCCGGGGGTACCTGAAGTACGTAACCGCAAGGAGCGTCCTAGGGTAAAAC
TGGTAATTGGGGCTAAGTCGTAACAAGGTA
SEQ ID NO: 43 Bacteroides cellulosilyticus Strain 55
AGAGTTTGATCCTGGCTCAGGATGAACGCTAGCTACAGGCTTAACACATGCAAGTCGAGGGGCAGCATGACCTAGC
AATAGGTTGATGGCGACCGGCGCACGGGTGAGTAACACGTATCCAACCTACCGGTTATTCCGGGATAGCCTTTCGA
AAGAAAGATTAATACCGGATAGTATAACGAGAAGGCATCTTTTTGTTATTAAAGAATTTCGATAACCGATGGGGAT
GCGTTCCATTAGTTTGTTGGCGGGGTAACGGCCCACCAAGACATCGATGGATAGGGGTTCTGAGAGGAAGGTCCCC
CACATTGGAACTGAGACACGGTCCAAACTCCTACGGGAGGCAGCAGTGAGGAATATTGGTCAATGGACGAGAGTCT
GAACCAGCCAAGTAGCGTGAAGGATGACTGCCCTATGGGTTGTAAACTTCTTTTATATGGGAATAAAGTGAGCCAC
GTGTGGCTTTTTGTATGTACCATACGAATAAGGATCGGCTAACTCCGTGCCAGCAGCCGCGGTAATACGGAGGATC
CGAGCGTTATCCGGATTTATTGGGTTTAAAGGGAGCGTAGGCGGACTATTAAGTCAGCTGTGAAAGTTTGCGGCTC
AACCGTAAAATTGCAGTTGATACTGGTCGTCTTGAGTGCAGTAGAGGTAGGCGGAATTCGTGGTGTAGCGGTGAAA
TGCTTAGATATCACGAAGAACTCCGATTGCGAAGGCAGCTTACTGGACTGTAACTGACGCTGATGCTCGAAAGTGT
GGGTATCAAACAGGATTAGATACCCTGGTAGTCCACACAGTAAACGATGAATACTCGCTGTTTGCGATATACGGCA
AGCGGCCAAGCGAAAGCATTAAGTATTCCACCTGGGGAGTACGCCGGCAACGGTGAAACTCAAAGGAATTGACGGG
GGCCCGCACAAGCGGAGGAACATGTGGTTTAATTCGATGATACGCGAGGAACCTTACCCGGGCTTAAATTGCAAAT
GAATATAGTGGAAACATTATAGCCGCAAGGCATTTGTGAAGGTGCTGCATGGTTGTCGTCAGCTCGTGCCGTGAGG
TGTCGGCTTAAGTGCCATAACGAGCGCAACCCTTATCTTTAGTTACTAACAGGTCATGCTGAGGACTCTAGAGAGA
CTGCCGTCGTAAGATGTGAGGAAGGTGGGGATGACGTCAAATCAGCACGGCCCTTACGTCCGGGGCTACACACGTG
TTACAATGGGGGGTACAGAAGGCAGCTACACAGCGATGTGATGCTAATCCCAAAAGCCTCTCTCAGTTCGGATTGG
AGTCTGCAACCCGACTCCATGAAGCTGGATTCGCTAGTAATCGCGCATCAGCCACGGCGCGGTGAATACGTTCCCG
GGCCTTGTACACACCGCCCGTCAAGCCATGAAAGCCGGGGGTACCTGAAGTCCGTAACCGTAAGGAGCGGCCTAGG
GTAAAACTGGTAATTGGGGCTAAGTCGTA
SEQ ID NO: 44 Bacteroides faecis Strain 56
CTCAGGATGAACGCTAGCTACAGGCTTAACACATGCAAGTCGAGGGGCAGCATTTCAGTTTGCTTGCAAACTGGAG
ATGGCGACCGGCGCACGGGTGAGTAACACGTATCCAACCTGCCGATAACTCGGGGATAGCCTTTCGAAAGAAAGAT
TAATACCCGATGGCATAATAGAACCGCATGGTTTTTTTATTAAAGAATTTCGGTTATCGATGGGGATGCGTTCCAT
TAGGCAGTTGGTGAGGTAACGGCTCACCAAACCTTCGATGGATAGGGGTTCTGAGAGGAAGGTCCCCCACATTGGA
ACTGAGACACGGTCCAAACTCCTACGGGAGGCAGCAGTGAGGAATATTGGTCAATGGACGAGAGTCTGAACCAGCC
AAGTAGCGTGAAGGATGACTGCCCTATGGGTTGTAAACTTCTTTTATATGGGAATAAAGTTTTCCACGTGTGGAAT
TTTGTATGTACCATATGAATAAGGATCGGCTAACTCCGTGCCAGCAGCCGCGGTAATACGGAGGATCCGAGCGTTA
TCCGGATTTATTGGGTTTAAAGGGAGCGTAGGTGGACAGTTAAGTCAGTTGTGAAAGTTTGCGGCTCAACCGTAAA
ATTGCAGTTGATACTGGCTGTCTTGAGTACAGTAGAGGTGGGCGGAATTCGTGGTGTAGCGGTGAAATGCTTAGAT
ATCACGAAGAACTCCGATTGCGAAGGCAGCTCACTGGACTGCAACTGACACTGATGCTCGAAAGTGTGGGTATCAA
ACAGGATTAGATACCCTGGTAGTCCACACAGTAAACGATGAATACTCGCTGTTTGCGATATACAGTAAGCGGCCAA
GCGAAAGCATTAAGTATTCCACCTGGGGAGTACGCCGGCAACGGTGAAACTCAAAGGAATTGACGGGGGCCCGCAC
AAGCGGAGGAACATGTGGTTTAATTCGATGATACGCGAGGAACCTTACCCGGGCTTAAATTGCATTTGAATATATT
GGAAACAGTATAGTCGTAAGACAAATGTGAAGGTGCTGCATGGTTGTCGTCAGCTCGTGCCGTGAGGTGTCGGCTT
AAGTGCCATAACGAGCGCAACCCTTATCTTTAGTTACTAACAGGTCATGCTGAGGACTCTAGAGAGACTGCCGTCG
TAAGATGTGAGGAAGGTGGGGATGACGTCAAATCAGCACGGCCCTTACGTCCGGGGCTACACACGTGTTACAATGG
GGGGTACAGAAGGCAGCTACCTGGTGACAGGATGCTAATCCCAAAAGCCTCTCTCAGTTCGGATCGAAGTCTGCAA
CCCGACTTCGTGAAGCTGGATTCGCTAGTAATCGCGCATCAGCCATGGCGCGGTGAATACGTTCCCGGGCCTTGTA
CACACCGCCCGTCAAGCCATGAAAGCCGGGGGTACCTGAAGTACGTAACCGCAAGGAGCGTCCTAGGGTAAAACTG
GTAATTGGGGCTAAGTCGTAACAAGGTA
SEQ ID NO: 45 Bacteroides ovatus Strain 57
CGATATCCGGATTTATTGGAGTTT-
AAGGGAGCGTAGGTGGATTGTTAAGTCAGTTGTGAAAGTTTGCGGCTCAACCGTAAAATTGCAGTTGAAACTGGCA
GTCTTGAGTACAGTAGAGGTGGGCGGAATTCGTGGTGTAGCGGTGAAATGCTTAGATATCACGAAGAACTCCGATT
GCGAAGGCAGCTCACTAGACTGTCACTGACACTGATGCTCGAAAGTGTGGGTATCAAACAGGATTAGATACCCTGG
TAGTCCACACAGTAAACGATGAATACTCGCTGTTTGCGATATACAGTAAGCGGCCAAGCGAAAGCATTAAGTATTC
CACCTGGGGAGTACGCCGGCAACGGTGAAACTCAAAGGAATTGACGGGGGCCCGCACAAGCGGAGGAACATGTGGT
TTAATTCGATGATACGCGAGGAACCTTACCCGGGCTTAAATTGCAACAGAATATATTGGAAACAGTATAGCCGTAA
GGCTGTTGTGAAGGTGCTGCATGGTTGTCGTCAGCTCGTGCCGTGAGGTGTCGGCTTAAGTGCCATAACGAGCGCA
ACCCTTATCTTTAGTTACTAACAGGTKATGCTGAGGACTCTAGAGAGACTGCCGTCGTAAGATGTGAGGAAGGTGG
GGATGACGTCAAATCAGCACGGCCCTTACGTCCGGGGCTACACACGTGTTACAATGGGGGGTACAGAAGGCSGCTA
CCTGGTGACAGGATGCTAATCCCAAAAACCTCTCTCAGTTCGGATCGAAGTCTGCAACCCGACTTCGTGAAGCTGG
ATTCGCTAGTAATCGCGCATCAGCCATGGCGCGGTGAATACGTTCCCGGGCCTTGTACACACCGCCCGTCAAGCCA
TGAAAGCCGGGGGTACCTGAAGTACGTAACCGCAAGGAGCGTCCTAGGGGTAAAACTGGTAATTGGGGC
SEQ ID NO: 46 Bacteroides thetaiotaomicron Strain 58
TTTAAGGGAGCGTAGGTGGACAGTTAAGTCAGTTGTGAAAGTTTGCGGCTCAACCGTAAAATTGCAGTTGATACTG
GCTGTCTTGAGTACAGTAGAGGTGGGCGGAATTCGTGGTGTAGCGGTGAAATGCTTAGATATCACGAAGAACTCCG
ATTGCGAAGGCAGCTCACTGGACTGCAACTGACACTGATGCTCGAAAGTGTGGGTATCAAACAGGATTAGATACCC
TGGTAGTCCACACAGTAAACGATGAATACTCGCTGTTTGCGATATACAGTAAGCGGCCWAGCGAAAGCATTAAGTA
TTCCACCTGGGGAGTACGCCGGCAACGGTGAAACTCAAAGGAATTGACGGGGGCCCGCACAAGCGGAGGAACATGT
GGTTTAATTCGATGATACGCGAGGAACCTTACCCGGGCTTAAATTGCAWWTGAATAWWYTGGAAACAGKWTAGYCG
YAAGRCAWWTGTGAAGGTGCTGCATGGTTGTCGTCAGCTCGTGCCGTGAGGTGTCGGCTTAAGTGCCATAACGAGC
GCAACCCTTATCTTTAGTTACTAACAGGTCATGCTGAGGACTCTAGAGAGACTGCCGTCGTAAGATGTGAGGAAGG
TGGGGATGACGTCAAATCAGCACGGCCCTTACGTCCGGGGCTACACACGTGTTACAATGGGGGGTACAGAAGGCAG
CTACCTGGTGACAGGATGCTAATCCCAAAAGCCTCTCTCAGTTCGGATCGAAGTCTGCAACCCGACTTCGTGAAGC
TGGATTCGCTAGTAATCGCGCATCAGCCATGGCGCGGTGAATACGTTCCCGGGCCTTGTACACACCGCCCGTCAAG
CCATGAAAGCCGGGGGTACCTGAAGTACGTAACCGCA
SEQ ID NO: 47 Bacteroides uniformis Strain 59
CTGGCTCAGGATGAACGCTAGCTACAGGCTTAACACATGCAAGTCGAGGGGCAGCATGAACTTAGCTTGCTAAGTT
TGATGGCGACCGGCGCACGGGTGAGTAACACGTATCCAACCTGCCGATGACTCGGGGATAGCCTTTCGAAAGAAAG
ATTAATACCCGATGGCATAGTTCTTCCGCATGGTAGAACTATTAAAGAATTTCGGTCATCGATGGGGATGCGTTCC
ATTAGGTTGTTGGCGGGGTAACGGCCCACCAAGCCTTCGATGGATAGGGGTTCTGAGAGGAAGGTCCCCCACATTG
GAACTGAGACACGGTCCAAACTCCTACGGGAGGCAGCAGTGAGGAATATTGGTCAATGGACGAGAGTCTGAACCAG
CCAAGTAGCGTGAAGGATGACTGCCCTATGGGTTGTAAACTTCTTTTATACGGGAATAAAGTGAGGCACGTGTGCC
TTTTTGTATGTACCGTATGAATAAGGATCGGCTAACTCCGTGCCAGCAGCCGCGGTAATACGGAGGATCCGAGCGT
TATCCGGATTTATTGGGTTTAAAGGGAGCGTAGGCGGACGCTTAAGTCAGTTGTGAAAGTTTGCGGCTCAACCGTA
AAATTGCAGTTGATACTGGGTGTCTTGAGTACAGTAGAGGCAGGCGGAATTCGTGGTGTAGCGGTGAAATGCTTAG
ATATCACGAAGAACTCCGATTGCGAAGGCAGCTTGCTGGACTGTAACTGACGCTGATGCTCGAAAGTGTGGGTATC
AAACAGGATTAGATACCCTGGTAGTCCACACAGTAAACGATGAATACTCGCTGTTTGCGATATACAGTAAGCGGCC
AAGCGAAAGCGTTAAGTATTCCACCTGGGGAGTACGCCGGCAACGGTGAAACTCAAAGGAATTGACGGGGGCCCGC
ACAAGCGGAGGAACATGTGGTTTAATTCGATGATACGCGAGGAACCTTACCCGGGCTTGAATTGCAACTGAATGAT
GTGGAGACATGTCAGCCGCAAGGCAGTTGTGAAGGTGCTGCATGGTTGTCGTCAGCTCGTGCCGTGAGGTGTCGGC
TTAAGTGCCATAACGAGCGCAACCCTTATCGATAGTTACCATCAGGTTATGCTGGGGACTCTGTCGAGACTGCCGT
CGTAAGATGTGAGGAAGGTGGGGATGACGTCAAATCAGCACGGCCCTTACGTCCGGGGCTACACACGTGTTACAAT
GGGGGGTACAGAAGGCAGCTACACGGCGACGTGATGCTAATCCCTAAAGCCTCTCTCAGTTCGGATTGGAGTCTGC
AACCCGACTCCATGAAGCTGGATTCGCTAGTAATCGCGCATCAGCCACGGCGCGGTGAATACGTTCCCGGGCCTTG
TACACACCGCCCGTCAAGCCATGAAAGCCGGGGGTACCTGAAGTGCGTAACCGCGAGGAGCGCCCTAGGGTAAAAC
TGGTGATTGGGGCTAAGTCGTAACAAGGTA
SEQ ID NO: 48 Bacteroides vulgatus Strain 60
ATGAAGAGTTTGATCCTGGCTCAGGATGAACGCTAGCTACAGGCTTAACACATGCAAGTCGAGGGGCAGCATGGTC
TTAGCTTGCTAAGGCCGATGGCGACCGGCGCACGGGTGAGTAACACGTATCCAACCTGCCGTCTACTCTTGGACAG
CCTTCTGAAAGGAAGATTAATACAAGATGGCATCATGAGTTCACATGTTCACATGATTAAAGGTATTCCGGTAGAC
GATGGGGATGCGTTCCATTAGATAGTAGGCGGGGTAACGGCCCACCTAGTCTTCGATGGATAGGGGTTCTGAGAGG
AAGGTCCCCCACATTGGAACTGAGACACGGTCCAAACTCCTACGGGAGGCAGCAGTGAGGAATATTGGTCAATGGG
CGAGAGCCTGAACCAGCCAAGTAGCGTGAAGGATGACTGCCCTATGGGTTGTAAACTTCTTTTATAAAGGAATAAA
GTCGGGTATGCATACCCGTTTGCATGTACTTTATGAATAAGGATCGGCTAACTCCGTGCCAGCAGCCGCGGTAATA
CGGAGGATCCGAGCGTTATCCGGATTTATTGGGTTTAAAGGGAGCGTAGATGGATGTTTAAGTCAGTTGTGAAAGT
TTGCGGCTCAACCGTAAAATTGCAGTTGATACTGGATATCTTGAGTGCAGTTGAGGCAGGCGGAATTCGTGGTGTA
GCGGTGAAATGCTTAGATATCACGAAGAACTCCGATTGCGAAGGCAGCCTGCTAAGCTGCAACTGACATTGAGGCT
CGAAAGTGTGGGTATCAAACAGGATTAGATACCCTGGTAGTCCACACGGTAAACGATGAATACTCGCTGTTTGCGA
TATACGGCAAGCGGCCAAGCGAAAGCGTTAAGTATTCCACCTGGGGAGTACGCCGGCAACGGTGAAACTCAAAGGA
ATTGACGGGGGCCCGCACAAGCGGAGGAACATGTGGTTTAATTCGATGATACGCGAGGAACCTTACCCGGGCTTAA
ATTGCAGATGAATTACGGTGAAAGCCGTAAGCCGCAAGGCATCTGTGAAGGTGCTGCATGGTTGTCGTCAGCTCGT
GCCGTGAGGTGTCGGCTTAAGTGCCATAACGAGCGCAACCCTTGTTGTCAGTTACTAACAGGTTCCGCTGAGGACT
CTGACAAGACTGCCATCGTAAGATGTGAGGAAGGTGGGGATGACGTCAAATCAGCACGGCCCTTACGTCCGGGGCT
ACACACGTGTTACAATGGGGGGTACAGAGGGCCGCTACCACGCGAGTGGATGCCAATCCCAAAAACCTCTCTCAGT
TCGGACTGGAGTCTGCAACCCGACTCCACGAAGCTGGATTCGCTAGTAATCGCGCATCAGCCACGGCGCGGTGAAT
ACGTTCCCGGGCCTTGTACACACCGCCCGTCAAGCCATGGGAGCCGGGGGTACCTGAAGTGCGTAACCGCGAGGAG
CGCCCTAGGGTAAAACTGGTGACTGGGGCTAAGTCGTAACAAGGTAGCCGTACCGGAAG
SEQ ID NO: 49 Bifidobacterium adolescentis Strain 61
GGGCTCGTAGKCGGTTCGTCGCGTCCGGTGTGAAAGTCCAYCGCTTAACGGTGGATCCGCGCCGGGTACGGGCGGG
CTTGAGTGCGGTAGGGGAGACTGGAATTCCCGGTGTAACGGTGGAATGTGTAGATATCGGGAAGAACACCAATGGC
GAAGGCAGGTCTCTGGGCCGTCACTGACGCTGAGGAGCGAAAGCGTGGGGAGCGAACAGGATTAGATACCCTGGTA
GTCCACGCCGTAAACGGTGGATGCTGGATGTGGGGACCATTCCACGGTCTCCGTGTCGGAGCCAACGCGTTAAGCA
TCCCGCCTGGGGAGTACGGCCGCAAGGCTAAAACTCAAAGAAATTGACGGGGGCCCGCACAAGCGGCGGAGCATGC
GGATTAATTCGATGCAACGCGAAGAACCTTACCTGGGCTTGACATGTTCCCGACAGCCCCAGAGATGGGGCCTCCC
TTCGGGGCGGGTTCACAGGTGGTGCATGGTCGTCGTCAGCTCGTGTCGTGAGATGTTGGGTTAAGTCCCGCAACGA
GCGCAACCCTCGCCCTGTGTTGCCAGCACGTCGTGGTGGGAACTCACGGGGGACCGCCGGGGTCAACTCGGAGGAA
GGTGGGGATGACGTCAGATCATCATGCCCCTTACGTCCAGGGCTTCACGCATGCTACAATGGCCGGTACAACGGGA
TGCGACACT-
GTGAGGTGGAGCGGATCCCTTAAAACCGGTCTCAGTTCGGATTGGAGTCTGCAACCCGACTCCATGAAGGCGGAGT
CGCTAGTAATCGCGGATCAGCAACGCCGCGGTGAATGCGTTCCCGGGCCTTGTACACACCGCCCGTCAAGTCATGA
AAGTGGGTAGCACCCGAAGCCGGTGGCCCATCCTTTTTGGGG
SEQ ID NO: 50 Bifidobacterium longum Strain 62
TGGCTCAGGATGAACGCTGGCGGCGTGCTTAACACATGCAAGTCGAACGGGATCCATCAGGCTTTGCTTGGTGGTG
AGAGTGGCGAACGGGTGAGTAATGCGTGACCGACCTGCCCCATACACCGGAATAGCTCCTGGAAACGGGTGGTAAT
GCCGGATGCTCCAGTTGATCGCATGGTCTTCTGGGAAAGCTTTCGCGGTATGGGATGGGGTCGCGTCCTATCAGCT
TGACGGCGGGGTAACGGCCCACCGTGGCTTCGACGGGTAGCCGGCCTGAGAGGGCGACCGGCCACATTGGGACTGA
GATACGGCCCAGACTCCTACGGGAGGCAGCAGTGGGGAATATTGCACAATGGGCGCAAGCCTGATGCAGCGACGCC
GCGTGAGGGATGGAGGCCTTCGGGTTGTAAACCTCTTTTATCGGGGAGCAAGCGAGAGTGAGTTTACCCGTTGAAT
AAGCACCGGCTAACTACGTGCCAGCAGCCGCGGTAATACGTAGGGTGCAAGCGTTATCCGGAATTATTGGGCGTAA
AGGGCTCGTAGGCGGTTCGTCGCGTCCGGTGTGAAAGTCCATCGCTTAACGGTGGATCCGCGCCGGGTACGGGCGG
GCTTGAGTGCGGTAGGGGAGACTGGAATTCCCGGTGTAACGGTGGAATGTGTAGATATCGGGAAGAACACCAATGG
CGAAGGCAGGTCTCTGGGCCGTTACTGACGCTGAGGAGCGAAAGCGTGGGGAGCGAACAGGATTAGATACCCTGGT
AGTCCACGCCGTAAACGGTGGATGCTGGATGTGGGGCCCGTTCCACGGGTTCCGTGTCGGAGCTAACGCGTTAAGC
ATCCCGCCTGGGGAGTACGGCCGCAAGGCTAAAACTCAAAGAAATTGACGGGGGCCCGCACAAGCGGCGGAGCATG
CGGATTAATTCGATGCAACGCGAAGAACCTTACCTGGGCTTGACATGTTCCCGACGGTCGTAGAGATACGGCTTCC
CTTCGGGGCGGGTTCACAGGTGGTGCATGGTCGTCGTCAGCTCGTGTCGTGAGATGTTGGGTTAAGTCCCGCAACG
AGCGCAACCCTCGCCCCGTGTTGCCAGCGGATTATGCCGGGAACTCACGGGGGACCGCCGGGGTTAACTCGGAGGA
AGGTGGGGATGACGTCAGATCATCATGCCCCTTACGTCCAGGGCTTCACGCATGCTACAATGGCCGGTACAACGGG
ATGCGACGCGGCGACGCGGAGCGGATCCCTGAAAACCGGTCTCAGTTCGGATCGCAGTCTGCAACTCGACTGCGTG
AAGGCGGAGTCGCTAGTAATCGCGAATCAGCAACGTCGCGGTGAATGCGTTCCCGGGCCTTGTACACACCGCCCGT
CAAGTCATGAAAGTGGGCAGCACCCGAAGCCGGTGGCCTAACCCCTTGTGGGATGGAGCCGTCTAAGGTGAGGCTC
GTGATTGGGACTAAGTCGTAACAAGGTAGCCGTACCGGAAGGTGCGGCTGG
SEQ ID NO: 51 Bifidobacterium pseudocatenulatum Strain 63
GGTTCGTCGCGTCCGGTGTGAAAGTCCATCGTTTAACGGTGGATCTGCGCCGGGTACGGGCGGGCTGGAGTGCGGT
AGGGGAGACTGGAATTCCCGGTGTAACGGTGGAATGTGTAGATATCGGGAAGAACACCAATGGCGAAGGCAGGTCT
CTGGGCCGTTACTGACGCTGAGGAGCGAAAGCGTGGGGAGCGAACAGGATTAGATACCCTGGTAGTCCACGCCGTA
AACGGTGGATGCTGGATGTGGGGCCCGTTCCACGGGTTCCGTGWCGGAGCTAACGCGTTAAGCATCCCGCCTGGGG
AGTACGGCCGCAAGGCTAAAACWMAAAKAAATTGACGGGGGCCCGCACAAGCGGCGGAGCATGCGGATTAATTCGA
TGCAACGCGAAGAACCTTACCTGGGCTTGACATGTTCCCGACAGCCGTAGAGATATGGCCTCCCTTCGGGGCGGGT
TCACAGGTGGTGCATGGTCGTCGTCAGCTCGTGTCGTGAGATGTTGGGTTAAGTCCCGCAACGAGCGCAACCCTCG
CCCTGTGTTGCCAGCACGTCATGGTGGGAACTCACGGGGGACCGCCGGGGTCAACTCGGAGGAAGGTGGGGATGAC
GTCAGATCATCATGCCCCTTACGTTCAGGGCTTCACGCATGCTACAATGGCCGGTACAACGGGATGCGACACGGCG
ACGTG
SEQ ID NO: 52 Blautia coccoides Strain 64
TGTGACTGAGCGGCGGACGGGTGAGTAACGCGTGGGTAACCTGCCTCATACAGGGGGATAACAGTTAGAAATGACT
GCTAATACCGCATAAGCGCACAGGACCGCATGGTCTGGTGTGAAAAACTCCGGTGGTATGAGATGGACCCGCGTCT
GATTAGCTAGTTGGAGGGGTAACGGCCCACCAAGGCGACGATCAGTAGCCGGCCTGAGAGGGTGAACGGCCACATT
GGGACTGAGACACGGCCCAGACTCCTACGGGAGGCAGCAGTGGGGAATATTGCACAATGGGGGAAACCCTGATGCA
GCGACGCCGCGTGAAGGAAGAAGTATCTCGGTATGTAAACTTCTATCAGCAGGGAAGAAAATGACGGTACCTGACT
AAGAAGCCCCGGCTAACTACGTGCCAGCAGCCGCGGTAATACGTAGGGGGCAAGCGTTATCCGGATTTACTGGGTG
TAAAGGGAGCGTAGACGGAAGAGCAAGTCTGATGTGAAAGGCTGGGGCTTAACCCCAGGACTGCATTGGAAACTGT
TGTTCTAGAGTGCCGGAGAGGTAAGCGGAATTCCTAGTGTAGCGGTGAAATGCGTAGATATTAGGAGGAACACCAG
TGGCGAAGGCGGCTTACTGGACGGTAACTGACGTTGAGGCTCGAAAGCGTGGGGAGCAAACAGGATTAGATACCCT
GGTAGTCCACGCCGTAAACGATGAATACTAGGTGTCGGGTGGCAAAGCCATTCGGTGCCGCAGCAAACGCAATAAG
TATTCCACCTGGGGAGTACGTTCGCAAGAATGAAACTCAAAGGAATTGACGGGGACCCGCACAAGCGGTGGAGCAT
GTGGTTTAATTCGAAGCAACGCGAAGAACCTTACCAAGTCTTGACATCCCTCTGACCGTCCCGTAACGGGGGCTTC
CCTTCGGGGCAGAGGAGACAGGTGGTGCATGGTTGTCGTCAGCTCGTGTCGTGAGATGTTGGGTTAAGTCCCGCAA
CGAGCGCAACCCTTATCCTTAGTAGCCAGCACATGATGGTGGGCACTCTAGGGAGACTGCCGGGGATAACCCGGAG
GAAGGCGGGGACGACGTCAAATCATCATGCCCCTTATGATTTGGGCTACACACGTGCTACAATGGCGTAAACAAAG
GGAAGCGAGACAGCGATGTTGAGCGAATCCCAAAAATAACGTCCCAGTTCGGACTGCAGTCTGCAACTCGACTGCA
CGAAGCTGGAATCGCTAGTAATCGCGGATCAGAATGCCGCGGTGAATACGTTCCCGGGTCTTGTACACACCGCCCG
TCACACCATGGGAGTCAGTAACGCCCGAAGTCAGTGACCTAACCGAAAGGAAGGAGCTGCCGAAGGCGGGACCGAT
AACTGGGGTGAAGTCGTAACAAGGTA
SEQ ID NO: 53 Clostridium citroniae Strain 65
TCCGGATTTACTGGAGTAGT-
AAGGGAGCGTAGACGGCGAAGCAAGTCTGGAGTGAAAACCCAGGGCTCAACCCTGGGACTGCTTTGGAAACTGTTT
TGCTAGAGTGTCGGAGAGGTAAGTGGAATTCCTAGTGTAGCGGTGAAATGCGTAGATATTAGGAGGAACACCAGTG
GCGAAGGCGGCTTACTGGACGATAACTGACGTTGAGGCTCGAAAGCGTGGGGAGCAAACAGGATTAGATACCCTGG
TAGTCCACGCCGTAAACGATGAATGCTAGGTGTTGGGGGGCAAAGCCCTTCGGTGCCGTCGCAAACGCAATAAGCA
TTCCACCTGGGGAGTACGTTCGCAAGAATGAAACTCAAAGGAATTGACGGGGACCCGCACAAGCGGTGGAGCATGT
GGTTTAATTCGAAGCAACGCGAAGAACCTTACCAAGTCTTGACATCCCCCTGACCGGTCAGTAAAGTGACCTTTCC
TTCGGGACAGGGGAGACAGGTGGTGCATGGTTGTCGTCAGCTCGTGTCGTGAGATGTTGGGTTAAGTCCCGCAACG
AGCGCAACCCTTATCCTTAGTAGCCAGCAGGTAAAGCTGGGCACTCTAGGGAGACTGCCAGGGATAACCTGGAGGA
AGGTGGGGATGACGTCAAATCATCATGCCCCTTATGATTTGGGCTACACACGTGCTACAATGGCGTAAACAAAGGG
AAGCGACCCTGCGAAGGCAAGCAAATCCCAAAAATAACGTCCCAGTTCGGACTGTAGTCTGCAACCCGACTACACG
AAGCTGGAATCGCTAGTAATCGCGAATCAGAATGTCGCGGTGAATACGTTCCCGGGTCTTGTACACACCGCCCGTC
ACACCATGGGAGTCAGCAACGCCCGAAGTCAGTGACCCAACCGAAAGGAGGGAGCTGCCGAA
SEQ ID NO: 54 Clostridium clostridioforme Strain 66
TAATACCGCATAAGCGCACAGTGCCGCATGGCAGTGTGTGAAAAACTCCGGTGGTGTGAGATGGATCCGCGTCTGA
TTAGCCAGTTGGCGGGGTAACGGCCCACCAAAGCGACGATCAGTAGCCGACCTGAGAGGGTGACCGGCCACATTGG
GACTGAGACACGGCCCAAACTCCTACGGGAGGCAGCAGTGGGGAATATTGCACAATGGGCGAAAGCCTGATGCAGC
GACGCCGCGTGAGTGAAGAAGTATTTCGGTATGTAAAGCTCTATCAGCAGGGAAGAAAATGACGGTACCTGACTAA
GAAGCCCCGGCTAACTACGTGCCAGCAGCCGCGGTAATACGTAGGGGGCAAGCGTTATCCGGATTTACTGGGTGTA
AAGGGAGCGTAGACGGCGAAGCAAGTCTGAAGTGAAAACCCGGGGCTCAACCCTGGGACTGCTTTGGAAACTGTTT
TGCTAGAGTGTCGGAGAGGTAAGTGGAATTCCTAGTGTAGCGGTGAAATGCGTAGATATTAGGAGGAACACCAGTG
GCGAAGGCGGCTTACTGGACGATAACTGACGTTGAGGCTCGAAAGCGTGGGGAGCAAACAGGATTAGATACCCTGG
TAGTCCACGCCGTAAACGATGAATGCTAGGTGTTGGGGGGCAAAGCCCTTCGGTGCCGCCGCAAACGCAGTAAGCA
TTCCACCTGGGGAGTACGTTCGCAAGAATGAAACTCAAAGGAATTGACGGGGACCCGCACAAGCGGTGGAGCATGT
GGTTTAATTCGAAGCAACGCGAAGAACCTTACCAAGTCTTGACATCCCCCTGACGGGCCGGTAACGCGGCCTTTCC
TTCGGGACAGGGGAGACAGGTGGTGCATGGTTGTCGTCAGCTCGTGTCGTGAGATGTTGGGTTAAGTCCCGCAACG
AGCGCAACCCTTATCCTTAGTAGCCAGCAGGTAGAGCCGGGCACTCTAGGGAGACTGCCAGGGATAACCTGGAGGA
AGGTGGGGATGACGTCAAATCATCATGCCCCTTATGATTTGGGCTACACACGTGCTACAATGGCGTAAACAAAGGG
AAGCGAGACAGTGATGTGGAGCAAATCCCAAAAATAACGTCCCAGTTCGGACTGTAGTCTGCAACCCGACTACACG
AAGCTGGAATCGCTAGTAATCGCGAATCAGAATGTCGCGGTGAATACGTTCCCGGGTCTTGTACACACCGCCCGTC
ACACCATGGGAGTCAGCAACGCCCGAAGTCAGTGACCCAACCGAAAGGAGGGAGCTGCCGAAGGCGGGGCAGGTAA
CTGGGGTGAAGTCGT
SEQ ID NO: 55 Clostridium innocuum Strain 67
ATGGAGAGTTTGATCCTGGCTCAGGATGAACGCTGGCGGCATGCCTAATACATGCAAGTCGAACGAAGT-
TTCGAGGAAGCTTGCTTCCAAAGAGACTTAGTGGCGAACGGGTGAGTAACACGTAGGTAACCTGCCCATGTGTCCG
GGATAACTGCTGGAAACGGTAGCTAAAACCGGATAGGTATACAGAGCGCATGCTCAGTATATTAAAGCGCCCATCA
AGGCGTGAACATGGATGGACCTGCGGCGCATTAGCTAGTTGGTGAGGTAACGGCCCACCAAGGCGATGATGCGTAG
CCGGCCTGAGAGGGTAAACGGCCACATTGGGACTGAGACACGGCCCAAACTCCTACGGGAGGCAGCAGTAGGGAAT
TTTCGTCAATGGGGGAAACCCTGAACGAGCAATGCCGCGTGAGTGAAGAAGGTCTTCGGATCGTAAAGCTCTGTTG
TAAGTGAAGAACGGCTCATAGAGGAAATGCTATGGGAGTGACGGTAGCTTACCAGAAAGCCACGGCTAACTACGTG
CCAGCAGCCGCGGTAATACGTAGGTGGCAAGCGTTATCCGGAATCATTGGGCGTAAAGGGTGCGTAGGTGGCGTAC
TAAGTCTGTAGTAAAAGGCAATGGCTCAACCATTGTAAGCTATGGAAACTGGTATGCTGGAGTGCAGAAGAGGGCG
ATGGAATTCCATGTGTAGCGGTAAAATGCGTAGATATATGGAGGAACACCAGTGGCGAAGGCGGTCGCCTGGTCTG
TAACTGACACTGAGGCACGAAAGCGTGGGGAGCAAATAGGATTAGATACCCTAGTAGTCCACGCCGTAAACGATGA
GAACTAAGTGTTGGAGGAATTCAGTGCTGCAGTTAACGCAATAAGTTCTCCGCCTGGGGAGTATGCACGCAAGTGT
GAAACTCAAAGGAATTGACGGGGGCCCGCACAAGCGGTGGAGTATGTGGTTTAATTCGAAGCAACGCGAAGAACCT
TACCAGGCCTTGACATGGAAACAAATACCCTAGAGATAGGGGGATAATTATGGATCACACAGGTGGTGCATGGTTG
TCGTCAGCTCGTGTCGTGAGATGTTGGGTTAAGTCCCGCAACGAGCGCAACCCTTGTCGCATGTTACCAGCATCAA
GTTGGGGACTCATGCGAGACTGCCGGTGACAAACCGGAGGAAGGTGGGGATGACGTCAAATCATCATGCCCCTTAT
GGCCTGGGCTACACACGTACTACAATGGCGGCCACAAAGAGCAGCGACACAGTGATGTGAAGCGAATCTCATAAAG
GTCGTCTCAGTTCGGATTGAAGTCTGCAACTCGACTTCATGAAGTCGGAATCGCTAGTAATCGCAGATCAGCATGC
TGCGGTGAATACGTTCTCGGGCCTTGTACACACCGCCCGTCAAACCATGGGAGTCAGTAATACCCGAAGCCGGTGG
CATAACCGTAAGGAGTGAGCCGTCGAAGGTAGGACCGA
SEQ ID NO: 56 Clostridium innocuum Strain 68
ATGGAGAGTTTGATCCTGGCTCAGGATGAACGCTGGCGGCATGCCTAATACATGCAAGTCGAACGAAGT-
TTCGAGGAAGCTTGCTTCCAAAGAGACTTAGTGGCGAACGGGTGAGTAACACGTAGGTAACCTGCCCATGTGTCCG
GGATAACTGCTGGAAACGGTAGCTAAAACCGGATAGGTATACAGAGCGCATGCTCAGTATATTAAAGCGCCCATCA
AGGCGTGAACATGGATGGACCTGCGGCGCATTAGCTAGTTGGTGAGGTAACGGCCCACCAAGGCGATGATGCGTAG
CCGGCCTGAGAGGGTAAACGGCCACATTGGGACTGAGACACGGCCCAAACTCCTACGGGAGGCAGCAGTAGGGAAT
TTTCGTCAATGGGGGAAACCCTGAACGAGCAATGCCGCGTGAGTGAAGAAGGTCTTCGGATCGTAAAGCTCTGTTG
TAAGTGAAGAACGGCTCATAGAGGAAATGCTATGGGAGTGACGGTAGCTTACCAGAAAGCCACGGCTAACTACGTG
CCAGCAGCCGCGGTAATACGTAGGTGGCAAGCGTTATCCGGAATCATTGGGCGTAAAGGGTGCGTAGGTGGCGTAC
TAAGTCTGTAGTAAAAGGCAATGGCTCAACCATTGTAAGCTATGGAAACTGGTATGCTGGAGTGCAGAAGAGGGCG
ATGGAATTCCATGTGTAGCGGTAAAATGCGTAGATATATGGAGGAACACCAGTGGCGAAGGCGGTCGCCTGGTCTG
TAACTGACACTGAGGCACGAAAGCGTGGGGAGCAAATAGGATTAGATACCCTAGTAGTCCACGCCGTAAACGATGA
GAACTAAGTGTTGGAGGAATTCAGTGCTGCAGTTAACGCAATAAGTTCTCCGCCTGGGGAGTATGCACGCAAGTGT
GAAACTCAAAGGAATTGACGGGGGCCCGCACAAGCGGTGGAGTATGTGGTTTAATTCGAAGCAACGCGAAGAACCT
TACCAGGCCTTGACATGGAAACAAATACCCTAGAGATAGGGGGATAATTATGGATCACACAGGTGGTGCATGGTTG
TCGTCAGCTCGTGTCGTGAGATGTTGGGTTAAGTCCCGCAACGAGCGCAACCCTTGTCGCATGTTACCAGCATCAA
GTTGGGGACTCATGCGAGACTGCCGGTGACAAACCGGAGGAAGGTGGGGATGACGTCAAATCATCATGCCCCTTAT
GGCCTGGGCTACACACGTACTACAATGGCGACCACAAAGAGCAGCGACACAGTGATGTGAAGCGAATCTCATAAAG
GTCGTCTCAGTTCGGATTGAAGTCTGCAACTCGACTTCATGAAGTCGGAATCGCTAGTAATCGCAGATCAGCATGC
TGCGGTGAATACGTTCTCGGGCCTTGTACACACCGCCCGTCAAACCATGGGAGTCAGTAATACCCGAAGCCGGTGG
CATAACCGTAAGGAGTGAGCCGTCGAAGGTAGGACCGA
SEQ ID NO: 57 Clostridium sordellii Strain 69
ACACATGCAAGTCGAGCGAACCCTTCGGGGTGAGCGGCGGACGGGTGAGTAACGCGTGGGTAACCTGCCCTGTACA
CACGGATAACATACCGAAAGGTATGCTAATACGGGATRAYATATGAGAGTCGCATGGCTTTTGTATCAAAGCTCCG
GCGGTACAGGATGGACCCGCGTCTGATTAGCTAGTTGGTAAGGTAACGGCTTACCAAGGCAACGATCAGTAGCCGA
CCTGAGAGGGTGATCGGCCACATTGGAACTGAGACACGGTCCAAACTCMTACGGGAGGCAGCAGTGGGGAATATTG
CACAATGGGCGAAAGCCTGATGCAGCAACGCCGCGTGAGCGATGAAGGCCTTCGGGTCGTAAAGCTCTGTCCTCAA
GGAAGATAATGACGGTACTTGAGGAGGAAGCCCCGGCTAACTACGTGCCAGCAGCCGCGGTAATACGTAGGGGGCT
AGCGTTATCCGGAATTACTGGGCGTAAAGGGTGCGTAGGCGGTCTTTCAAGCCAGAAGTGAAAGGCTACGGCTCAA
CCGTAGTAAGCTTTTGGAACTGTAGGACTTGAGTGCAGGAGAGGAGAGTGGAATTCCTAGTGTAGCGGTGAAATGC
GTAGATATTAGGAGGAACACCAGTAGCGAAGGCGGCTCTCTGGACTGTAACTGACGCTGAGGCACGAAAGCGTGGG
GAGCAAACAGGATTAGATACCCTGGTAGTCCACGCCGTAAACGATGAGTACTAGGTGTCGGGGGTTACCCCCCTCG
GTGCCGCAGCTAACGCATTAAGTACTCCGCCTGGGAAGTACGCTCGCAAGAGTGAAACTCAAAGGAATTGACGGGG
ACCCGCACAAGTAGCGGAGCATGTGGTTTAATTCGAAGCAACGCGAAGAACCTTATCTAARCTTGACATC
SEQ ID NO: 58 Coprococcus comes Strain 70
ACRGGAGGCAGCAGTGGGGAATATTGCACAATGGGGGAAACCCTGATGCAGCGACGCCRCGTGAGCGAAGAAGTAT
TKCGGTATGTAAAGCTCTATCAGCAGGGAAGAAAATGACGGTACCTGACTAAGAAGCACCGGCTAAATACGTGCCA
GCAGCCGCGGTAATACGTATGGTGCAAGCGTTATCCGGATTTACTGGGTGTAAAGGGAGCGTAGACGGCTGTGTAA
GTCTGAAGTGAAAGGCGGGGGCTCCCCCCCGGGGACTGCTTTGGAAACTATGCAGCTAGACTGTCGGACAGGTAAG
TGGAATTCCCAGTGTAGCGGTGAAATGCGTAGATATTGGGAGGAACAGCAKTGGGTAAGGCTSCTTACAGGACRAT
SEQ ID NO: 59 Dorea longicatena Strain 71
TAACGCGTGGGTAACCTGCCTCATACAGGGGGATAACAGTTAGAAATGACTGCTAATACCGCATAAGACCACGGTA
CCGCATGGTACAGTGGTAAAAACTCCGGTGGTATGAGATGGACCCGCGTCTGATTAGGTAGTTGGTGGGGTAACGG
CCTACCAAGCCGACGATCAGTAGCCGACCTGAGAGGGTGACCGGCCACATTGGGACTGAGACACGGCCCAGACTCC
TACGGGAGGCAGCAGTGGGGAATATTGCACAATGGAGGAAACTCTGATGCAGCGACGCCGCGTGAAGGATGAAGTA
TTTCGGTATGTAAACTTCTATCAGCAGGGAAGAAAATGACGGTACCTGACTAAGAAGCCCCGGCTAACTACGTGCC
AGCAGCCGCGGTAATACGTAGGGGGCAAGCGTTATCCGGATTTACTGGGTGTAAAGGGAGCGTAGACGGCACGGCA
AGCCAGATGTG-
AAAGCCCGGGGCTCAACCCCGGGACTGCATTTGGAACTGCTGAGCTAGAGTGTCGGAGAGGCAAGTGGAATTCCTA
GTGTAGCGGTGAAATGCGTAGATATTAGGAGGAACACCAGTGGCGAAGGCGGCTTGCTGGACGATGACTGACGTTG
AGGCTCGAAAGCGTGGGGAGCAAACAGGATTAGATACCCTGGTAGTCCACGCCGTAAACGATGACTGCTAGGTGTC
GGGTGGCAAAGCCATTCGGTGCCGCAGCTAACGCAATAAGCAGTCCACCTGGGGAGTACGTTCGCAAGAATGAAAC
TCAAAGGAATTGACGGGGACCCGCACAAGCGGTGGAGCATGTGGTTTAATTCGAAGCAACGCGAAGAACCTTACCT
GATCTTGACATCCCGATGACCGCTTCGTAATGGAAGCTTTTCTTCGGAACATCGGTGACAGGTGGTGCATGGTTGT
CGTCAGCTCGTGTCGTGAGATGTTGGGTTAAGTCCCGCAACGAGCGCAACCCCTATCTTCAGTAGCCAGCAGGTTA
AGCTGGGCACTCTGGAGAGACTGCCAGGGATAACCTGGAGGAAGGTGGGGATGACGTCAAATCATCATGCCCCTTA
TGACCAGGGCTACACACGTGCTACAATGGCGTAAACAAAGAGAAGCGAACTCGCGAGGGTAAGCAAATCTCAAAAA
TAACGTCTCAGTTCGGATTGTAGTCTGCAACTCGACTACATGAAGCTGGAATCGCTAGTAATCGCAGATCAGAATG
CTGCGGTGAATACGTTCCCGGGTCTTGTACACACCGCCCGTCACACCATGGGAGTCAGTAACGCCCGAAGTCAGTG
ACCCAACCGTAAGG
SEQ ID NO: 60 Erysipelatoclostridium ramosum Strain 72
GAGGGAGCAGGCGGCAGCAAGGGTCTGTGGTGAAAGCCTGAAGTTAAACTTCAGTAAGCCATAGAAACCAGGCAGC
TAGAGTGCAGGAGAGGAKCGTGGAATTCCATGTGTAGCGGTGAAATGCGTAGATATATGGAGGAACACCAGTGGCG
AAGGCGACGATCTGGCCTGCAACTGACGCTCAGTCCCGAAAGCGTGGGGAGCAAATAGGATTAGATACCCTAGTAG
TCCACGCCGTAAACGATGAGTACTRAGTGTTGGATGTCAAAGTTCAGTGCTGCAGTTAACGCAATAAGTACTCCGC
CTGAGTAGTACGTTCGCAAGAATGAAACTCAAAGGAATTGACGGGGGCCCGCACAAGCGGTGGAGCATGTGGTTTA
ATTCGAAGCAACGCGAAGAACCTTACCAGGTCTTGACATACTCATAAAGGCTCCAGAGATGGAGAGATAGCTATAT
GAGATACAGGTGGTGCATGGTTGTCGTCAGCTCGTGTCGTGAGATGTTGGGTTAAGTCCCGCAACGAGCGCAACCC
TTATCGTTAGTTACCATCATTAAGTTGGGGACTCTAGCGAGACTGCCAGTGACAAGCTGGAGGAARGCGGGGATGA
CGTCAAATCATCATGCCCCTTATGACCTGGGCTACACACGTGCTACAATGGATGGTGCAGAGGGAAGCGAAGCCGC
GAGGTGAAGCAAAACCCATAAAACCATTCTCAGTTCGGATTGTAGTCTGCARCTCGACTACATGAAGTTGGAATCG
CTAGTAATCGCGAATCARCATGTCGCGATGAATAMGTTCTCGGGCCTT
SEQ ID NO: 61 Eubacterium rectale Strain 73
AGAGTTTGATCCTGGCTCAGGATGAACGCTGGCGGCGTGCTTAACACATGCAAGTCGAACGAAGCACTTTATTTGA
TTTCCTTCGGGACTGATTATTTTGTGACTGAGTGGCGGACGGGTGAGTAACGCGTGGGTAACCTGCCTTGTACAGG
GGGATAACAGTTGGAAACGGCTGCTAATACCGCATAAGCGCACGGCATCGCATGATGCAGTGTGAAAAACTCCGGT
GGTATAAGATGGACCCGCGTTGGATTAGCTAGTTGGTGAGGTAACGGCCCACCAAGGCGACGATCCATAGCCGACC
TGAGAGGGTGACCGGCCACATTGGGACTGAGACACGGCCCAAACTCCTACGGGAGGCAGCAGTGGGGAATATTGCA
CAATGGGCGAAAGCCTGATGCAGCGACGCCGCGTGAGCGAAGAAGTATTTCGGTATGTAAAGCTCTATCAGCAGGG
AAGATAATGACGGTACCTGACTAAGAAGCACCGGCTAAATACGTGCCAGCAGCCGCGGTAATACGTATGGTGCAAG
CGTTATCCGGATTTACTGGGTGTAAAGGGAGCGCAGGCGGTGCGGCAAGTCTGATGTGAAAGCCCGGGGCTCAACC
CCGGTACTGCATTGGAAACTGTCGTACTAGAGTGTCGGAGGGGTAAGCGGAATTCCTAGTGTAGCGGTGAAATGCG
TAGATATTAGGAGGAACACCAGTGGCGAAGGCGGCTTACTGGACGATAACTGACGCTGAGGCTCGAAAGCGTGGGG
AGCAAACAGGATTAGATACCCTGGTAGTCCACGCCGTAAACGATGAATACTAGGTGTTGGGAAGCATTGCTTCTCG
GTGCCGTCGCAAACGCAGTAAGTATTCCACCTGGGGAGTACGTTCGCAAGAATGAAACTCAAAGGAATTGACGGGG
ACCCGCACAAGCGGTGGAGCATGTGGTTTAATTCGAAGCAACGCGAAGAACCTTACCAAGTCTTGACATCCTTCTG
ACCGGTACTTAACCGTACCTTCTCTTCGGAGCAGGAGTGACAGGTGGTGCATGGTTGTCGTCAGCTCGTGTCGTGA
GATGTTGGGTTAAGTCCCGCAACGAGCGCAACCCTTATCTTTAGTAGCCAGCGGTTCGGCCGGGCACTCTAGAGAG
ACTGCCAGGGATAACCTGGAGGAAGGCGGGGATGACGTCAAATCATCATGCCCCTTATGACTTGGGCTACACACGT
GCTACAATGGCGTAAACAAAGGGAAGCAAAGCTGTGAAGCCGAGCAAATCTCAAAAATAACGTCTCAGTTCGGACT
GTAGTCTGCAACCCGACTACACGAAGCTGGAATCGCTAGTAATCGCAGATCAGAATGCTGCGGTGAATACGTTCCC
GGGTCTTGTACACACCGCCCGTCACACCATGGGAGTTGGGAATGCCCGAAGCCAGTGACCTAACCGAAAGGAAGGA
GCTGTCGAAGGCAGGCTCGATAACTGGGGTGAAGTCGTAACAAGGTAGCCGTATCGGAAGGTGCGGCTGGATCACC
T
SEQ ID NO: 62 Odoribacter splanchnicus Strain 74
AGAGTTTGATCCTGGCTCAGGATGAACGCTAGCGACAGGCTTAACACATGCAAGTCGAGGGGCATCATGAGGTAGC
AATACCTTGATGGCGACCGGCGCACGGGTGAGTAACGCGTATGCAACCTGCCCGATACCGGGGTATAGCCCATGGA
AACGTGGATTAACACCCCATAGTACTTTTATCCTGCATGGGATGTGAGTTAAATGTTTAAGGTATCGGATGGGCAT
GCGTCCTATTAGTTAGTTGGCGGGGTAACAGCCCACCAAGACGATGATAGGTAGGGGTTCTGAGAGGAAGGTCCCC
CACATTGGAACTGAGACACGGTCCAAACTCCTACGGGAGGCAGCAGTGAGGAATATTGGTCAATGGACGAGAGTCT
GAACCAGCCAAGTCGCGTGAGGGAAGACTGCCCTATGGGTTGTAAACCTCTTTTATAAGGGAAGAATAAGTTCTAC
GTGTAGAATGATGCCTGTACCTTATGAATAAGCATCGGCTAACTCCGTGCCAGCAGCCGCGGTAATACGGAGGATG
CGAGCGTTATCCGGATTTATTGGGTTTAAAGGGTGCGTAGGCGGTTTATTAAGTTAGTGGTTAAATATTTGAGCTA
AACTCAATTGTGCCATTAATACTGGTAAACTGGAGTACAGACGAGGTAGGCGGAATAAGTTAAGTAGCGGTGAAAT
GCATAGATATAACTTAGAACTCCGATAGCGAAGGCAGCTTACCAGACTGTAACTGACGCTGATGCACGAGAGCGTG
GGTAGCGAACAGGATTAGATACCCTGGTAGTCCACGCCGTAAACGATGCTCACTGGTTCTGTGCGATATATTGTAC
GGGATTAAGCGAAAGTATTAAGTGAGCCACCTGGGGAGTACGTCGGCAACGATGAAACTCAAAGGAATTGACGGGG
GCCCGCACAAGCGGAGGAACATGTGGTTTAATTCGATGATACGCGAGGAACCTTACCTGGGTTTAAATGGGAAATG
TCGTATTTGGAAACAGATATTCTCTTCGGAGCGTTTTTCAAGGTGCTGCATGGTTGTCGTCAGCTCGTGCCGTGAG
GTGTCGGGTTAAGTCCCATAACGAGCGCAACCCTTACCGTTAGTTGCTAGCATGTAATGATGAGCACTCTAACGGG
ACTGCCACCGTAAGGTGAGAGGAAGGCGGGGATGACGTCAAATCAGCACGGCCCTTACACCCAGGGCTACACACGT
GTTACAATGGCCGGTACAGAGGGCCGCTACCAGGTGACTGGATGCCAATCTCAAAAGCCGGTCGTAGTTCGGATTG
GAGTCTGTAACCCGACTCCATGAAGTTGGATTCGCTAGTAATCGCGCATCAGCCATGGCGCGGTGAATACGTTCCC
GGGCCTTGTACACACCGCCCGTCAAGCCATGGAAGCCGGGGGTGCCTGAAGTCCGTAACCGCGAGGATCGGCCTAG
GGCAAAACTGGTAACTGGGGCTAAGTCGTAACAAGGTA
SEQ ID NO: 63 Parabacteroides distasonis Strain 75
CGAGGGGCAGCRCAGGAGT-TAGCAATAC-
CSGGTGGCGACCGGCGCACGGGTGAGTAACGCGTATGCAACTTRCCTATCAGAGGGGGATAACCCGGCGAAAGTCG
GACTAATACCGCATGAAGCAGGGATYCCGCATGGGRATATTTGCTAAAGATTCATCGCTGATAGATAGGCATGCGT
TCCATTAGGCAGTTGGCGGGGTAACRGCCCACCAAACCGACGATGGATAGGGGTTCTGAGAGGAAGGTCCCCCACA
TTGGTACTGAGACACGGACCAAACTCCTACGGGAGGCAGCAGTGAGGAATATTGGTCAATGGSCGWRAGSCTGAAC
CAGCCAAGTCGCGTGAGGGATGAAGGTTCTATGGATCGTAAACCTCTTTTATAAGGGAATAAAGTGCGGGACGTGT
CCYRTTTTGTATGTACCTTATGAATAAGGATCGGCTAACTCCGTGCCAGCAGCCGCGGTAATACGGAGGATCCGAG
CGTTATCCGGATTTATTGGGTTTAAAGGGTGCGTAGGCGGCCTTTTAAGTCAGCGGTGAAAGTCTGTGGCTCAACC
ATAGAATTGCCGTTGAAACTGGGGGGCTTGAGTATGTTTGAGGCAGGCGGAATGCGTGGTGTAGCGGTGAAATGCA
TAGATATCACGCAGAACCCCGWTTGCGAAGGCAGCCTGCCAAGCCGTAACTGACGCGGATGCACGAAAGCGTGGGG
ATCAAACAGGATTAGATACCCTGGTA
SEQ ID NO: 64 Parabacteroides merdae Strain 76
CATGCAAGTCGAGGGGCAGCATGATTTGTAGCAATACAGATTGATGGCGACCGGCGCACGGGTGAGTAACGCGTAT
GCAACTTACCTATCAGAGGGGGATAGCCCGGCGAAAGTCGGATTAATACCCCATAAAACAGGGGTCCCGCATGGGA
ATATTTGTTAAAGATTCATCGCTGATAGATAGGCATGCGTTCCATTAGGCAGTTGGCGGGGTAACGGCCCACCAAA
CCGACGATGGATAGGGGTTCKGAGAGGAAGGTCCCCCACATTGGTACTGAGACACGGACCAAACTCCTACGGGAGG
CAGCAGTGAGGAATATTGGTCAATGGCCGAGAGGCTGAACCAGCCAAGTCGCGTGAAGGAAGAAGGATCTATGGTT
TGTAAACTTCTTTTATAGGGGAATAAAGTGGAGGACGTGTCCTTTTTTGTATGTACCCTATGAATAAGCATCGGCT
AACTCCGTGMSARCMGCCGCGGGAATACGGAAGATGCAGAGCGTTATCCGGATWTATTGGGGTTA
SEQ ID NO: 65 Bacteroides xylanisolvens Strain 77
CATGCAAGTCGAGGGGCAGCATTTTAGTTTGCTTGCAAACTAAAGATGGCGACCGGCGCACGGGTGAGTAACACGT
ATCCAACCTGCCGATAACTCGGGGATAGCCTTTCGAAAGAAAGATTAATATCCGATAGTATATTAAAACCGCATGG
TTTTACTATTAAAGAATTTCGGTTATCGATGGGGATGCGTTCCATTAGTTTGTTGGCGGGGTAACGGCCCACCAAG
ACTACGATGGATAGGGGTTCTGAGAGGAAGGTCCCCCACATTGGAACTGAGACACGGTCCAAACTCCTACGGGAGG
CAGCAGTGAGGAATATTGGTCAATGGACGAGAGTCTGAACCAGCCAAGTAGCGTGAAGGATGACTGCCCTATGGGT
TGTAAACTTCTTTTATATGGGAATAAAGTATTCCACGTGTGGGATTTTGTATGTACCATATGAATAAGGATCGGCT
AACTCCGTGCCAGCAGCCGCGGTAATACGGAGGATCCGAGCGTTATCCGGATTTATTGGGTTTAAAGGGAGCGTAG
GTGGATTGTTAAGTCAGTTGTGAAAGTTTGCGGCTCAACCGTAAAATTGCAGTTGAAACTGGCAGTCTTGAGTACA
GTAGAGGTGGGCGGAATTCGTGGTGTAGCGGTGAAATGCTTAGATATCACGAAGAACTCCGATTGCGAAGGCAGCT
CACTAGACTGCAACTGACACTGATGCTCGAAAGTGTGGGTATCAAACAGGATTAGATACCCTGGTAGTCCACACAG
TAAACGATGAATACTCGCTGTTTGCGATATACAGTAAGCGGCCAAGCGAAAGCATTAAGTATTCCACCTGGGGAGT
ACGCCGGCAACGGTGAAACTCAAAGGAATTGACGGGGGCCCGCACAAGCGGAGGAACATGTGGTTTAATTCGATGA
TACGCGAGGAACCTTACCCGGGCTTAAATTGCATTTGAATAATCTGGAAACAGGTTAGCCGCAAGGCAAATGTGAA
GGTGCTGCATGGTTGTCGTCAGCTCGTGCCGTGAGGTGTCGGCTTAAGTGCCATAACGAGCGCAACCCTTATCTTT
AGTTACTAACAGGTTATGCTGAGGACTCTAGAGAGACTGCCGTCGTAAGATGTGAGGAAGGTGGGGATGACGTCAA
ATCAGCACGGCCCTTACGTCCGGGGCTACACACGTGTTACAATGGGGGGTACAGAAGGCAGCTACCTGGCGACAGG
ATGCTAATCCCAAAAACCTCTCTCAGTTCGGATCGAAGTCTGCAACCCGACTTCGTGAAGCTGGATTCGCTAGTAA
TCGCGCATCAGCCATGGCGCGGTGAATACGTTCCCGGGCCTTGTACACACCGCCCGTCAAGCCATGAAAGCCGGGG
SEQ ID NO: 66 Blautia obeum Strain 78
GGCGTGCTTAACACATGCAAGTCGAACGGGAAACCTTTTATTGAAGCTTCGGCAGATTTAG-
CTGGTTTCTAGTGGCGGACGGGTGAGTAACGCGTGGGTAACCTGCCTTATACAGGGGGATAACAACCAGAAATGGT
TGCTAATACCGCATAAGCGCACAGGACCGCATGGTCCGGTGTGAAAAACTCCGGTGGTATAAGATGGACCCGCGTT
GGATTAGCTAGTTGGCAGGGTAACGGCCTACCAAGGCGACGATCCATAGCCGGCCTGAGAGGGTGAACGGCCACAT
TGGGACTGAGACACGGCCCAGACTCCTACGGGAGGCAGCAGTGGGGAATATTGCACAATGGGGGAAACCCTGATGC
AGCGACGCCGCGTGAAGGAAGAAGTATCTCGGTATGTAAACTTCTATCAGCAGGGAAGATAGTGACGGTACCTGAC
TAAGAAGCCCCGGCTAACTACGTGCCAGCAGCCGCGGTAATACGTAGGGGGCAAGCGTTATCCGGATTTACTGGGT
GTAAAGGGAGCGTAGACGGACTGGCAAGTCTGATGTGAAAGGCGGGGGCTCAACCCCTGGACTGCATTGGAAACTG
TTAGTCTTGAGTGCCGGAGAGGTAAGCGGAATTCCTAGTGTAGCGGTGAAATGCGTAGATATTAGGAGGAACACCA
GTGGCGAAGGCGGCTTACTGGACGGTAACTGACGTTGAGGCTCGAAAGCGTGGGGAGCAAACAGGATTAGATACCC
TGGTAGTCCACGCCGTAAACGATGAATACTAGGTGTTGGGGAGCAAAGCTCTTCGGTGCCGCCGCAAACGCATTAA
GTATTCCACCTGGGGAGTACGTTCGCAAGAATGAAACTCAAAGGAATTGACGGGGACCCGCACAAGCGGTGGAGCA
TGTGGTTTAATTCGAAGCAACGCGAAGAACCTTACCAAGTCTTGACATCCCTCTGACCGTTCCTTAACCGGAACTT
TCCTTCGGGACAGGGGAGACAGGTGGTGCATGGTTGTCGTCAGCTCGTGTCGTGAGATGTTGGGTTAAGTCCCGCA
ACGAGCGCAACCCCTATCCCCAGTAGCCAGCAGTCCGGCTGGGCACTCTGAGGAGACTGCCAGGGATAACCTGGAG
GAAGGCGGGGATGACGTCAAATCATCATGCCCCTTATGATTTGGGCTACACACGTGCTACAATGGCGTAAACAAAG
GGAAGCAAGCCTGCGAAGGTAAGCAAATCCCAAAAATAACGTCCCAGTTCGGACTGCAGTCTGCAACTCGACTGCA
CGAAGCTGGAATCGCTAGTAATCGCGGATCAGAATGCCGCGGTGAATACGTTCCCGGGTCTTGTACACACCGCCCG
TCACACCATGGGAGTCAGTAACGCCCGAAGTCAGTGACCTAACTGC
SEQ ID NO: 67 Alistipes putredinis Strain 79
AGAGTTTGATCCTGGCTCAGGATGAACGCTAGCGGCAGGCTTAACACATGCAAGTCGAGGGGCAGCATAATGGATA
GCAATATCTATGGTGGCGACCGGCGCACGGGTGCGTAACGCGTATGCAACCTACCTTTAACAGGGGGATAACACTG
AGAAATTGGTACTAATACCCCATAATATCATAGAAGGCATCTTTTATGGTTGAAAATTCCGATGGTTAGAGATGGG
CATGCGTTGTATTAGCTAGTTGGTGGGGTAACGGCTCACCAAGGCGACGATACATAGGGGGACTGAGAGGTTAACC
CCCCACACTGGTACTGAGACACGGACCAGACTCCTACGGGAGGCAGCAGTGAGGAATATTGGTCAATGGACGCAAG
TCTGAACCAGCCATGCCGCGTGCAGGATGACGGCTCTATGAGTTGTAAACTGCTTTTGTACGAGGGTAAACGCAGA
TACGTGTATCTGTCTGAAAGTATCGTACGAATAAGGATCGGCTAACTCCGTGCCAGCAGCCGCGGTAATACGGAGG
ATTCAAGCGTTATCCGGATTTATTGGGTTTAAAGGGTGCGTAGGCGGTTTGATAAGTTAGAGGTGAAATTTCGGGG
CTCAACCCTGAACGTGCCTCTAATACTGTTGAGCTAGAGAGTAGTTGCGGTAGGCGGAATGTATGGTGTAGCGGTG
AAATGCTTAGAGATCATACAGAACACCGATTGCGAAGGCAGCTTACCAAACTATATCTGACGTTGAGGCACGAAAG
CGTGGGGAGCAAACAGGATTAGATACCCTGGTAGTCCACGCAGTAAACGATGATAACTCGTTGTCGGCGATACACA
GTCGGTGACTAAGCGAAAGCGATAAGTTATCCACCTGGGGAGTACGTTCGCAAGAATGAAACTCAAAGGAATTGAC
GGGGGCCCGCACAAGCGGAGGAACATGTGGTTTAATTCGATGATACGCGAGGAACCTTACCCGGGCTTGAAAGTTA
GCGACGATTCTTGAAAGAGGATTTCCCTTCGGGGCGCGAAACTAGGTGCTGCATGGTTGTCGTCAGCTCGTGCCGT
GAGGTGTCGGGTTAAGTCCCATAACGAGCGCAACCCCTACCGTTAGTTGCCATCAGGTGAAGCTGGGCACTCTGGC
GGGACTGCCGGTGTAAGCCGAGAGGAAGGTGGGGATGACGTCAAATCAGCACGGCCCTTACGTCCGGGGCTACACA
CGTGTTACAATGGTAGGTACAGAGGGCAGCTACCCAGCGATGGGATGCGAATCTCGAAAGCCTATCTCAGTTCGGA
TTGGAGGCTGAAACCCGCCTCCATGAAGTTGGATTCGCTAGTAATCGCGCATCAGCCATGGCGCGGTGAATACGTT
CCCGGGCCTTGTACACACCGCCCGTCAAGCCATGGGAGCCGGGGGTGCCTGAAGTTCGTGACCGCAAGGAGCGACC
TAGGGCAAAACTGGTGACTGGGGCTAAGTCGTAACAAGGTA
SEQ ID NO: 68 Collinsella aerofaciens Strain 80
AGAGTTCGATCCTGGCTCAGGATGAACGCTGGCGGCGCGCCTAACACATGCAAGTCGAACGGCACCTATCTTCGGA
TAGAAGCGAGTGGCGAACGGCTGAGTAACACGTGGAGAACCTGCCCCCTCCCCCGGGATAGCCGCCCGAAAGGACG
GGTAATACCGGATACCCCGGGGTGCCGCATGGCACCCCGGCTAAAGCCCCGACGGGAGGGGATGGCTCCGCGGCCC
ATCAGGTAGACGGCGGGGTGACGGCCCACCGTGCCGACAACGGGTAGCCGGGTTGAGAGACCGACCGGCCAGATTG
GGACTGAGACACGGCCCAGACTCCTACGGGAGGCAGCAGTGGGGAATCTTGCGCAATGGGGGGAACCCTGACGCAG
CGACGCCGCGTGCGGGACGGAGGCCTTCGGGTCGTAAACCGCTTTCAGCAGGGAAGAGTCAAGACTGTACCTGCAG
AAGAAGCCCCGGCTAACTACGTGCCAGCAGCCGCGGTAATACGTAGGGGGCGAGCGTTATCCGGATTCATTGGGCG
TAAAGCGCGCGTAGGCGGCCCGGCAGGCCGGGGGTCGAAGCGGGGGGCTCAACCCCCCGAAGCCCCCGGAACCTCC
GCGGCTTGGGTCCGGTAGGGGAGGGTGGAACACCCGGTGTAGCGGTGGAATGCGCAGATATCGGGTGGAACACCGG
TGGCGAAGGCGGCCCTCTGGGCCGAGACCGACGCTGAGGCGCGAAAGCTGGGGGAGCGAACAGGATTAGATACCCT
GGTAGTCCCAGCCGTAAACGATGGACGCTAGGTGTGGGGGGACGATCCCCCCGTGCCGCAGCCAACGCATTAAGCG
TCCCGCCTGGGGAGTACGGCCGCAAGGCTAAAACTCAAAGGAATTGACGGGGGCCCGCACAAGCAGCGGAGCATGT
GGCTTAATTCGAAGCAACGCGAAGAACCTTACCAGGGCTTGACATATGGGTGAAGCGGGGGAGACCCCGTGGCCGA
GAGGAGCCCATACAGGTGGTGCATGGCTGTCGTCAGCTCGTGTCGTGAGATGTTGGGTTAAGTCCCGCAACGAGCG
CAACCCCCGCCGCGTGTTGCCATCGGGTGATGCCGGGAACCCACGCGGGACCGCCGCCGTCAAGGCGGAGGAGGGC
GGGGACGACGTCAAGTCATCATGCCCCTTATGCCCTGGGCTGCACACGTGCTACAATGGCCGGTACAGAGGGATGC
CACCCCGCGAGGGGGAGCGGATCCCGGAAAGCCGGCCCCAGTTCGGATTGGGGGCTGCAACCCGCCCCCATGAAGT
CGGAGTTGCTAGTAATCGCGGATCAGCATGCCGCGGTGAATGCGTTCCCGGGCCTTGTACACACCGCCCGTCACAC
CACCCGAGTCGTCTGCACCCGAAGTCGCCGGCCCAACCGAGAGGGGGGAGGCGCCGAAGGTGTGGAGGGTGAGGGG
GGTGAAGTCGTAACAAGGTA
SEQ ID NO: 69 Bacteroides faecis Strain 81
CTCAGGATGAACGCTAGCTACAGGCTTAACACATGCAAGTCGAGGGGCAGCATTCCAGTTTGCTTGCAAACTGGAG
ATGGCGACCGGCGCACGGGTGAGTAACACGTATCCAACCTGCCGATAACTCGGGGATAGCCTTTCGAAAGAAAGAT
TAATACCCGATGGCATAATAGAACCGCATGGTTTGATTATTAAAGAATTTCGGTTATCGATGGGGATGCGTTCCAT
TAGGCAGTTGGTGGGGTAACGGCCCACCAAACCTTCGATGGATAGGGGTTCTGAGAGGAAGGTCCCCCACATTGGA
ACTGAGACACGGTCCAAACTCCTACGGGAGGCAGCAGTGAGGAATATTGGTCAATGGACGAGAGTCTGAACCAGCC
AAGTAGCGTGAAGGATGACTGCCCTATGGGTTGTAAACTTCTTTTATATGGGAATAAAGTGGTCCACGTGTGGATT
TTTGTATGTACCATATGAATAAGGATCGGCTAACTCCGTGCCAGCAGCCGCGGTAATACGGAGGATCCGAGCGTTA
TCCGGATTTATTGGGTTTAAAGGGAGCGTAGGTGGACAGTTAAGTCAGTTGTGAAAGTTTGCGGCTCAACCGTAAA
ATTGCAGTTGATACTGGCTGTCTTGAGTACAGTAGAGGCGGGCGGAATTCGTGGTGTAGCGGTGAAATGCTTAGAT
ATCACGAAGAACTCCGATTGCGAAGGCAGCTCACTGGACTGCAACTGACACTGATGCTCGAAAGTGTGGGTATCAA
ACAGGATTAGATACCCTGGTAGTCCACACAGTAAACGATGAATACTCGCTGTTTGCGATATACAGTAAGCGGCCAA
GCGAAAGCATTAAGTATTCCACCTGGGGAGTACGCCGGCAACGGTGAAACTCAAAGGAATTGACGGGGGCCCGCAC
AAGCGGAGGAACATGTGGTTTAATTCGATGATACGCGAGGAACCTTACCCGGGCTTAAATTGCATTTGAATATATT
GGAAACAGTATAGTCGTAAGACAAATGTGAAGGTGCTGCATGGTTGTCGTCAGCTCGTGCCGTGAGGTGTCGGCTT
AAGTGCCATAACGAGCGCAACCCTTATCTTTAGTTACTAACAGGTCATGCTGAGGACTCTGGAGAGACTGCCGTCG
TAAGATGTGAGGAAGGTGGGGATGACGTCAAATCAGCACGGCCCTTACGTCCGGGGCTACACACGTGTTACAATGG
GGGGTACAGAAGGCCGCTACCTGGTGACAGGATGCTAATCCCAAAAGCCTCTCTCAGTTCGGATCGAAGTCTGCAA
CCCGACTTCGTGAAGCTGGATTCGCTAGTAATCGCGCATCAGCCATGGCGCGGTGAATACGTTCCCGGGCCTTGTA
CACACCGCCCGTCAAGCCATGAAAGCCGGGGGTACCTGAAGTACGTAACCGCAAGGAGCGTCCTAGGGTAAAACTG
GTAATTGGGGCTAAGTCGTAACAAGGTA
SEQ ID NO: 70 Alistipes shahii Strain 82
ACATAGGGGGWSTGWKAGGTTWRCCSCCCACATTSRTACTGAGMCA-
TGAWCMAACTCTMTACGGGARGSAGSAGTGAGGAATATTGGTCRRTGGACGCAAGTCTGAACCAGCCATGCCGSGT
GCRGGAAGACGGCTCKATGAGTKGKAAACTGCTTTTGTACRARRGTAAACGCTCTTACGTGTAAGAGCCTGAAAGT
ATSGTACRAATGAGGATCGGCTAACTCCGTGCCAGCAGCCGCGGTAATACGGAGGATCCAAGCGTTATCCGGATTT
ATTGGGTTTAAAGGGTGCGTAGGCGGGTTGATAAAGTTAGRGG
SEQ ID NO: 71 Anaerostipes caccae Strain 83
GCTT-ACACATG-
CAAGTCGAACGAAGCATTTARGATTGAAGTTTTCGGATGGATTTCCTATATGACTGAGTGGCGGACGGGTGAGTAA
CGCGTGGGGAACCTGCCCTATACAGGGGGATAACAGCTGGAAACGGCTGCTAATACCGCATAAGCGCACAGAATCG
CATGATTCAGTGTGAAAAGCCCTGGCAGTATAGGATGGTCCCGCGTCTGATTAGCTGGTTGGTGAGGTAACGGCTC
ACCAAGGCGACGATCAGTAGCCGGCTTGAGAGAGTGAACGGCCACATTGGGACTGAGACACGGCCCAAACTCCTAC
GGGAGGCAGCAGTGGGGAATATTGCACAATGGGGG-
AAACCCTGATGCAGCGACGCCGCGTGAGTGAAGAAGTATTTCGGTATGTAAAGCTCTATCAGCAGGGAAGAAAACA
GACGGTACCTGACTAAGAAGCCCCGGCTAACTACGTGCCAGCAGCCGCGGTAATACGTAGGGGGCAAGCGTTATCC
GGAATTACTGGGTGTAAAGGGTGCGTAGGTGGCATGGTAAGTCAGAAGTGAAAGCCCGGGGCTTAACCCCGGGACT
GCTTTTGAAACTGTCATGCTGGAGTGCAGGAGAGGTAAGCGGAATTCCTAGTGTAGCGGTGAAATGCGTAGATATT
AGGAGGAACACCAGTGGCGAAGGCGGCTTACTGGACTGTCACTGACACTGATGCACGAAAGCGTGGGGAGCAAACA
GGATTAGATACCCTGGTAGTCCACGCCGTAAACGATGAATACTAGGTGTCGGGGCCGTAGAGGCTTCGGTGCCGCA
GCAAA
SEQ ID NO: 72 Phascolarctobacterium faecium Strain 84
CGGAGAATTTTCATTTCGGTAGAATTCTTAGTGGCGAACGGGTGAGTAACGCGTAGGCAACCTGCCCTTTAGACGG
GGACAACATTCCGAAAGGAGTGCTAATACCGGATGTGATCATCGTGCCGCATGGCAGGATGAAGAAAGATGGCCTC
TACAAGTAAGCTATCGCTAAAGGATGGGCCTGCGTCTGATTAGCTAGTTGGTAGTGTAACGGACTACCAAGGCGAT
GATCAGTAGCCGGTCTGAGAGGATGAACGGCCACATTGGGACTGAGACACGGCCCAAACTCCTACGGGAGGCAGCA
GTGGGGAATCTTCCGCAATGGACGAAAGTCTGACAGAGCAACGCCGCGTGAGTGATGAAGGATTTCGGTCTGTAAA
GCTCTGTTGTTTATGACGAACGTGCAGTGTGTGAACAATGCATTGCAATGACGGTAGTAAACGAGGAAGCCACGGC
TAACTACGTGCCAGCAGCCGCGGTAATACGTAGGTGGCGAGCGTTGTCCGGAATTATTGGGCGTAAAGAGCATGTA
GGCGGCTTAATAAGTCGAGCGTGAAAAATGCGGGGCTCAACCCCGTATGGCGCTGGAAACTGTTAGGCTTGAGTGC
AGGAGAGGAAAGGGGAATTCCCAGTGTAGCGGTGAAATGCGTAGATATTGGGAGGAACACCAGTGGCGAAGGCGCC
TTTCTGGACTGTGTTTGACGCTGAGATGCGAAAGCCAGGGTAGC
SEQ ID NO: 73 Agathobaculum butyriciproducens Strain 85
TAGTGGCGGACGGGTGAGTAACGCGTGAGCAATCTGCCTTTAAGAGGGGGATAACAGTCGGAAACGGCTGCTAATA
CCGCATAAAGCATTGAATTCGCATGTTTTCGATGCCAAAGGAGCAATCCGCTTTTAGATGAGCTCGCGTCTGATTA
GCTAGTTGGCGGGGTAACGGCCCACCAAGGCGACGATCAGTAGCCGGACTGAGAGGTTGAACGGCCACATTGGGAC
TGAGACACGGCCCAGACTCCTACGGGAGGCAGCAGTGGGGAATATTGCGCAATGGGGGRAACCCTGACGCAGCAAC
GCCGCGTGATTGAAGAAGGCCTTCGGGTTGTAAAGATCTTTAATCAGGGACGAA--
AMATGACGGTACCTGAAGAATAAGCTCCGGCTAACTACGTGCCAGCAGCCGCGGTAATACGTAGGGAGCAAGCGTT
ATCCGGATTTACTGGGTGTAAAGGGCGCGCAGGCGGGCCGGCAAGTTGGAAGTGAAATCCGGGGGCTTAACCCCCG
AACTGCTTTCAAAACTGCTGGTCTTGAGTGATGGAGAGGCAGGCGGAATTCCGTGTGTAGCGGTGAAATGCGTAGA
TATACGGAGGAACACCAGTGGCGAAGGCGGCCTGCTGGACATTAACTGACGCTGAGGCGCGAAAGCGTGGGGAGCA
AACAGGATTAGATACCCTGGTAGTCCACGCCGTAAACGATGGATA
SEQ ID NO: 74 Bacteroides fragilis Strain 86
ATGAAGAGTTTGATCCTGGCTCAGGATGAACGCTAGCTACAGGCTTAACACATGCAAGTCGAGGGGCATCAGGAAG
AAAGCTTGCTTTCTTTGCTGGCGACCGGCGCACGGGTGAGTAACACGTATCCAACCTGCCCTTTACTCGGGGATAG
CCTTTCGAAAGAAAGATTAATACCCGATGGCATAATGATTCCGCATGGTTTCATTATTAAAGGATTCCGGTAAAGG
ATGGGGATGCGTTCCATTAGGTTGTTGGTGAGGTAACGGCTCACCAAGCCTTCGATGGATAGGGGTTCTGAGAGGA
AGGTCCCCCACATTGGAACTGAGACACGGTCCAAACTCCTACGGGAGGCAGCAGTGAGGAATATTGGTCAATGGGC
GCTAGCCTGAACCAGCCAAGTAGCGTGAAGGATGAAGGCTCTATGGGTCGTAAACTTCTTTTATATAAGAATAAAG
TGCAGTATGTATACTGTTTTGTATGTATTATATGAATAAGGATCGGCTAACTCCGTGCCAGCAGCCGCGGTAATAC
GGAGGATCCGAGCGTTATCCGGATTTATTGGGTTTAAAGGGAGCGTAGGTGGACTGGTAAGTCAGTTGTGAAAGTT
TGCGGCTCAACCGTAAAATTGCAGTTGATACTGTCAGTCTTGAGTACAGTAGAGGTGGGCGGAATTCGTGGTGTAG
CGGTGAAATGCTTAGATATCACGAAGAACTCCGATTGCGAAGGCAGCTCACTGGACTGCAACTGACACTGATGCTC
GAAAGTGTGGGTATCAAACAGGATTAGATACCCTGGTAGTCCACACAGTAAACGATGAATACTCGCTGTTTGCGAT
ATACAGTAAGCGGCCAAGCGAAAGCATTAAGTATTCCACCTGGGGAGTACGCCGGCAACGGTGAAACTCAAAGGAA
TTGACGGGGGCCCGCACAAGCGGAGGAACATGTGGTTTAATTCGATGATACGCGAGGAACCTTACCCGGGCTTAAA
TTGCAGTGGAATGATGTGGAAACATGTCAGTGAGCAATCACCGCTGTGAAGGTGCTGCATGGTTGTCGTCAGCTCG
TGCCGTGAGGTGTCGGCTTAAGTGCCATAACGAGCGCAACCCTTATCTTTAGTTACTAACAGGTTATGCTGAGGAC
TCTAGAGAGACTGCCGTCGTAAGATGTGAGGAAGGTGGGGATGACGTCAAATCAGCACGGCCCTTACGTCCGGGGC
TACACACGTGTTACAATGGGGGGTACAGAAGGCAGCTAGCGGGTGACCGTATGCTAATCCCAAAAGCCTCTCTCAG
TTCGGATCGAAGTCTGCAACCCGACTTCGTGAAGCTGGATTCGCTAGTAATCGCGCATCAGCCACGGCGCGGTGAA
TACGTTCCCGGGCCTTGTACACACCGCCCGTCAAGCCATGGGAGCCGGGGGTACCTGAAGTACGTAACCGCAAGGA
TCGTCCTAGGGTAAAACTGGTGACTGGGGCTAAGTCGTAACAAGGTAGCCGTACCGGAAGGTGCGGCTGGAACACC
TCCTT
SEQ ID NO: 75 Fusobacterium mortiferum Strain 87
TGGCTCAGGATGAACGCTGACAGAATGCTTAACACATGCAAGTCTACTTGATCCTTCGGGTGATGGTGGCGGACGG
GTGAGTAACGCGTAAAGAACTTGCCCTGCAGTCTGGGACAACATTTGGAAACGAATGCTAATACCGGATATTATGT-
ATTTCTCGCATGAGTTTTACATGAAAGCTATATGCGCTGCAGGAGAGCTTTGCGTCCTATTAGCTAGTTGGTGAGG
TAACGGCTCACCAAGGCCATGATAGGTAGCCGGCCTGAGAGGGTGAACGGCCACAAGGGGACTGAGACACGGCCCT
TACTCCTACGGGAGGCAGCAGTGGGGAATATTGGACAATGGACCAAAAGTCTGATCCAGCAATTCTGTGTGCACGA
TGAAGTTTTTCGGAATGTAAAGTGCTTTCAGTTGGGACGAAGTAAGTGACGGTACCAACAGAAGAAGCGACGGCTA
AATACGTGCCAGCAGCCGCGGTAATACGTATGTCGCAAGCGTTATCCGGATTTATTGGGCGTAAAGCGCGTCTAGG
CGGTTTGGTAAGTCTGATGTGAAAATGCGGGGCTCAACTCCGTATTGCGTTGGAAACTGCTAAACTAGAGTACTGG
AGAGGTGGGCGGAACTACAAGTGTAGAGGTGAAATTCGTAGATATTTGTAGGAATGCCGATGGGGAAGCCAGCCCA
CTGGACAGATACTGACGCTAAAGCGCGAAAGCGTGGGTAGCAAACAGGATTAGATACCCTGGTAGTCCACGCCGTA
AACGATGATTACTAGGTGTTGGGGGTCGAACCTCAGCGCCCAAGCTAACGCGATAAGTAATCCGCCTGGGGAGTAC
GTACGCAAGTATGAAACTCAAAGGAATTGACGGGGACCCGCACAAGCGGTGGAGCATGTGGTTTAATTCGACGCAA
CGCGAGGAACCTTACCAGCGTTTGACATCCTAAGAAATTAGCAGAGATGCTTTTGTGCCCCTTCGGGGGAACTTAG
TGACAGGTGGTGCATGGCTGTCGTCAGCTCGTGT
SEQ ID NO: 76 Paraclostridium benzoelyticum Strain 88
AGAGTTTGATCCTGGCTCAGGATGAACGCTGGCGGCGTGCCTAACACATGCAAGTCGAGCGATCTCTTCGGAGAGA
GCGGCGGACGGGTGAGTAACGCGTGGGTAACCTGCCCTGTACACACGGATAACATACCGAAAGGTATACTAATACG
GGATAACATACGAAAGTCGCATGGCTTTTGTATCAAAGCTCCGGCGGTACAGGATGGACCCGCGTCTGATTAGCTA
GTTGGTAAGGTAATGGCTTACCAAGGCAACGATCAGTAGCCGACCTGAGAGGGTGATCGGCCACACTGGAACTGAG
ACACGGTCCAGACTCCTACGGGAGGCAGCAGTGGGGAATATTGCACAATGGGCGAAAGCCTGATGCAGCAACGCCG
CGTGAGCGATGAAGGCCTTCGGGTCGTAAAGCTCTGTCCTCAAGGAAGATAATGACGGTACTTGAGGAGGAAGCCC
CGGCTAACTACGTGCCAGCAGCCGCGGTAATACGTAGGGGGCTAGCGTTATCCGGAATTACTGGGCGTAAAGGGTG
CGTAGGTGGTTTTTTAAGTCAGAAGTGAAAGGCTACGGCTCAACCGTAGTAAGCTTTTGAAACTAGAGAACTTGAG
TGCAGGAGAGGAGAGTAGAATTCCTAGTGTAGCGGTGAAATGCGTAGATATTAGGAGGAATACCAGTAGCGAAGGC
GGCTCTCTGGACTGTAACTGACACTGAGGCACGAAAGCGTGGGGAGCAAACAGGATTAGATACCCTGGTAGTCCAC
GCCGTAAACGATGAGTACTAGGTGTCGGGGGTTACCCCCCTCGGTGCCGCAGCTAACGCATTAAGTACTCCGCCTG
GGAAGTACGCTCGCAAGAGTGAAACTCAAAGGAATTGACGGGGACCCGCACAAGTAGCGGAGCATGTGGTTTAATT
CGAAGCAACGCGAAGAACCTTACCTAAGCTTGACATCCCACTGACCTCTCCCTAATCGGAGATTTCCCTTCGGGGA
CAGTGGTGACAGGTGGTGCATGGTTGTCGTCAGCTCGTGTCGTGAGATGTTGGGTTAAGTCCCGCAACGAGCGCAA
CCCTTGCCTTTAGTTGCCAGCATTAAGTTGGGCACTCTAGAGGGACTGCCGAGGATAACTCGGAGGAAGGTGGGGA
TGACGTCAAATCATCATGCCCCTTATGCTTAGGGCTACACACGTGCTACAATGGGTGGTACAGAGGGTTGCCAAGC
CGCGAGGTGGAGCTAATCCCTTAAAGCCATTCTCAGTTCGGATTGTAGGCTGAAACTCGCCTACATGAAGCTGGAG
TTACTAGTAATCGCAGATCAGAATGCTGCGGTGAATGCGTTCCCGGGTCTTGTACACACCGCCCGTCACACCATGG
AAGTTGGGGGCGCCCGAAGCCGGTTAGCTAACCTTTTAGGAAGCGGCCGTCGAAGGTGAAACCAATGACTGGGGTG
AAGTCGTAACAAGGTAGCCGTATCGGAAGGTGCGGCTGGATCACCT
SEQ ID NO: 77 Escherichia fergusonii Strain 89
TTGAAGAGTTTGATCATGGCTCAGATTGAACGCTGGCGGCAGGCCTAACACATGCAAGTCGAACGGTAACAGGAAG
CAGCTTGCTGCTTTGCTGACGAGTGGCGGACGGGTGAGTAATGTCTGGGAAACTGCCTGATGGAGGGGGATAACTA
CTGGAAACGGTAGCTAATACCGCATAACGTCGCAAGACCAAAGAGGGGGACCTTCGGGCCTCTTGCCATCGGATGT
GCCCAGATGGGATTAGCTAGTAGGTGGGGTAACGGCTCACCTAGGCGACGATCCCTAGCTGGTCTGAGAGGATGAC
CAGCCACACTGGAACTGAGACACGGTCCAGACTCCTACGGGAGGCAGCAGTGGGGAATATTGCACAATGGGCGCAA
GCCTGATGCAGCCATGCCGCGTGTATGAAGAAGGCCTTCGGGTTGTAAAGTACTTTCAGCGGGGAGGAAGGGAGTA
AAGTTAATACCTTTGCTCATTGACGTTACCCGCAGAAGAAGCACCGGCTAACTCCGTGCCAGCAGCCGCGGTAATA
CGGAGGGTGCAAGCGTTAATCGGAATTACTGGGCGTAAAGCGCACGCAGGCGGTTTGTTAAGTCAGATGTGAAATC
CCCGGGCTCAACCTGGGAACTGCATCTGATACTGGCAAGCTTGAGTCTCGTAGAGGGGGGTAGAATTCCAGGTGTA
GCGGTGAAATGCGTAGAGATCTGGAGGAATACCGGTGGCGAAGGCGGCCCCCTGGACGAAGACTGACGCTCAGGTG
CGAAAGCGTGGGGAGCAAACAGGATTAGATACCCTGGTAGTCCACGCCGTAAACGATGTCGACTTGGAGGTTGTGC
CCTTGAGGCGTGGCTTCCGGAGCTAACGCGTTAAGTCGACCGCCTGGGGAGTACGGCCGCAAGGTTAAAACTCAAA
TGAATTGACGGGGGCCCGCACAAGCGGTGGAGCATGTGGTTTAATTCGATGCAACGCGAAGAACCTTACCTGGTCT
TGACATCCACGGAAGTTTTCAGAGATGAGAATGTGCCTTCGGGAACCGTGAGACAGGTGCTGCATGGCTGTCGTCA
GCTCGTGTTGTGAAATGTTGGGTTAAGTCCCGCAACGAGCGCAACCCTTATCCTTTGTTGCCAGCGGTCCGGCCGG
GAACTCAAAGGAGACTGCCAGTGATAAACTGGAGGAAGGTGGGGATGACGTCAAGTCATCATGGCCCTTACGACCA
GGGCTACACACGTGCTACAATGGCGCATACAAAGAGAAGCGACCTCGCGAGAGCAAGCGGACCTCATAAAGTGCGT
CGTAGTCCGGATTGGAGTCTGCAACTCGACTCCATGAAGTCGGAATCGCTAGTAATCGTGGATCAGAATGCCACGG
TGAATACGTTCCCGGGCCTTGTACACACCGCCCGTCACACCATGGGAGTGGGTTGCAAAAGAAGTAGGTAGCTTAA
CCTTCGGGAGGGCGCTTACCACTTTGTGATTCATGACTGGGGTGAAGTCGTAACAAGGTAACCGTAGGGGAACCTG
CGGTTGGATCACCTCCTT

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Mouse Model of Vancomycin-Resistant Enterococcus faecium and Carbapenem-Resistant Klebsiella pneumoniae

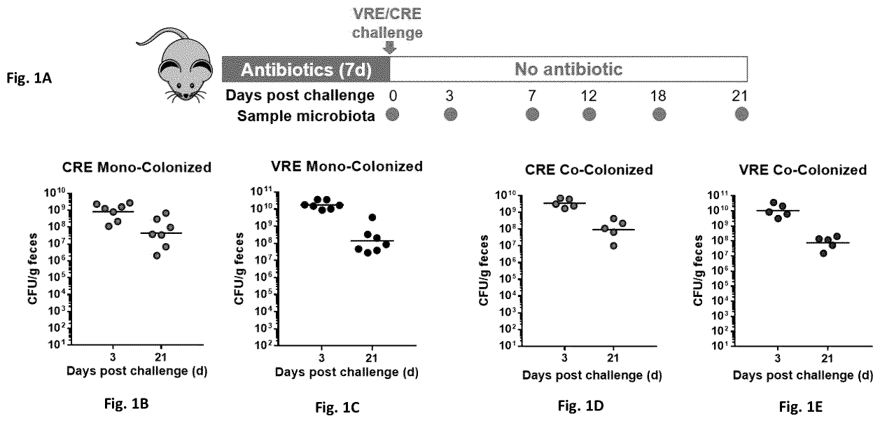

Mouse models of vancomycin-resistant Enterococcus faecium (VRE) and carbapenem-resistant Klebsiella pneumoniae (CRE) colonization were established using the methodology used by Ubeda et al. (JCI 2010 120 (12): 4332) and Caballero et al. (PLoS Pathog. (2015) 11(9): 1005). As shown in FIG. 1A, C57BL/6 mice were treated with broad-spectrum antibiotics (e.g., ampicillin) for seven days prior to the pathogen challenge. The antibiotic treatment created a niche for VRE/CRE to colonize. On day 0, the ampicillin treatment was terminated. Mice were single-housed and challenged with 10⁴-10⁵ CFU of VRE or CRE (mono-colonized) or both VRE and CRE (co-colonized). Fecal samples (pellets) were collected from each mouse at various time points following the challenge to quantify CFUs as a measure of pathogen colonization. The fecal samples were plated on selective media. Mice challenged with VRE, CRE, or both remained densely colonized for 21 days in the absence of continued antibiotic treatment (FIGS. 1B-IE). No spontaneous clearance was observed. CRE and VRE colonized the intestine to the same level following mono- or co-challenge (FIGS. 1B-1E).

### Example 2: Carbapenem-Resistant Klebsiella pneumoniae

As described in Example 1, mouse models of CRE colonization were established following the methodology used by Ubeda et al. (JCI 2010 120 (12): 4332) and Caballero et al. (PLoS Pathog. (2015) 11(9). As shown in FIG. 2A, mice were treated with broad-spectrum antibiotics (ampicillin) for seven days prior to the CRE challenge. On day −7, the ampicillin treatment was terminated. Mice were single-housed and challenged with 10⁴-10⁵ CFU of CRE. Three days after the pathogen challenge (on day 0), mice were randomized into groups, and the first of three treatment doses of fecal microbiota treatment (FMT) or stool fraction library (SFL) was administered. The three-day period between CRE challenge and FMT/SFL administration served as a washout period to clear residual antibiotics and establish CRE colonization in the intestine. Fecal samples were collected at various time points post-treatment to quantify CRE levels (plating on selective media) and to determine strain engraftment by metagenomic sequencing.

First, the model for CRE colonization and clearance was validated. As shown in FIG. 2B, administration of FMT from a donor reduced the CRE burden from densely-colonized mice.

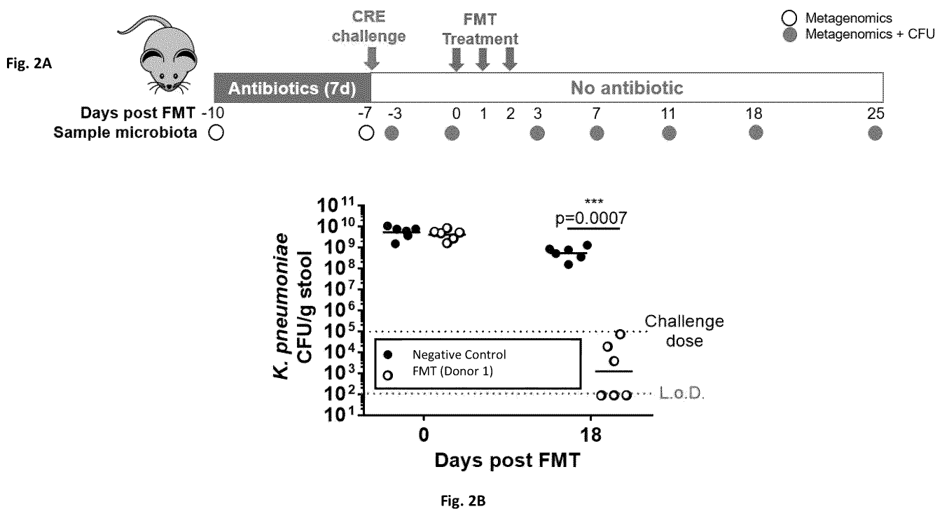

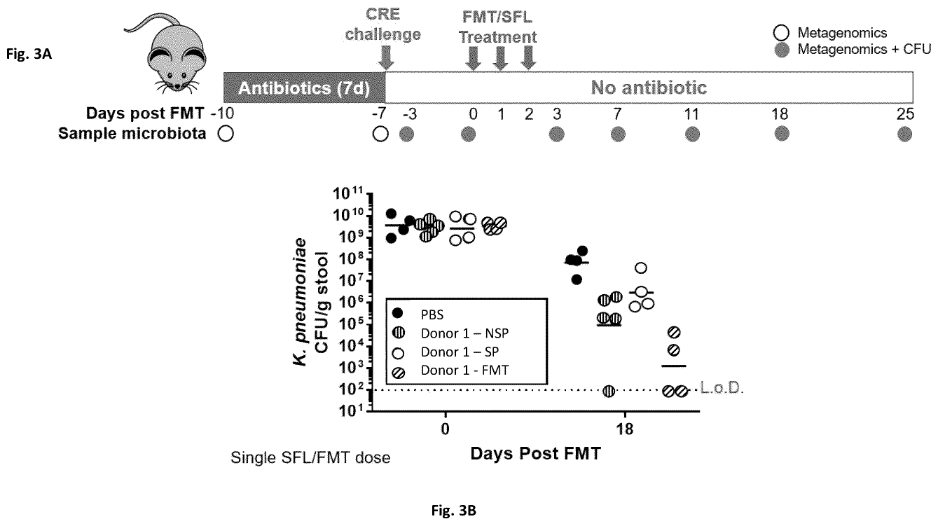

Stool fractions were generated to identify fractions having potent pathogen-antagonistic activity. The stool fraction library, SFL, was used to reduce the time to defined and optimized live bacterial product, LBP, by creating donor-derived bacterial fractions that are less complex, culturable, and stable. To generate stool fractions enriched in non-spore forming (NSP) bacterial including members of the Bacteroidetes, Firmicutes (non-spore and spore-formers), Proteobacteria, and Actinobacteria phyla, a 10% fecal slurry was diluted and plated onto rich solid media. After a three day incubation under anaerobic conditions, multiple plates from the same dilution containing well-separated colonies with distinct morphology were scraped and pooled. Aliquots were frozen at −80° C. until ready for use. Likewise, to generate stool fractions enriched in spore-forming (SP) bacteria, a 10% fecal slurry was treated with 100% ethanol for 60 minutes. Following ethanol treatment, the stool suspension was diluted and plated as described above. Stool fractions and FMT were tested for their ability to reduce the CRE burden (decolonize) in mice colonized with CRE as shown in FIG. 3A.

As shown in FIG. 3B, the stool fractions were able to reduce the CRE burden.

### Example 2A: Composition of Stool Fractions

The stool fractions from two example donors were sequenced to identify the bacterial species present in the fractions. Genomic DNA (gDNA) was extracted from each frozen stool fraction library (SFL) using a modified protocol based on the MasterPure DNA extraction kit (Lucigen). In brief, SFL cells were pelleted and resuspended in lysis buffer containing lysozyme. gDNA was then extracted and purified from the SFL lysate following the manufacturers protocol. The SFL gDNA was then sequenced on the Illumina platform. Sequencing libraries were prepared using the Nextera XT Library Prep kit and sequenced on the Illumina NextSeq instrument. Raw metagenomic FASTQ files for each SFL sample were filtered to remove adapters and low quality sequences then reads were assigned to the nearest taxonomic relative using the One Codex database and software (Minot S S, BioRxiv. 2015 Sep. 25). After every sequence read has been assigned, the One Codex software was used to estimate the relative abundance of the microbial species in each SFL. The One Codex software takes the number of sequences assigned to each species and the relative coverage of those sequences to the reference genome as inputs and applies a non-negative least squares algorithm to calculate the relative abundance of each species. These resulting relative abundance estimates are more accurate than other methods when evaluated against a broad set of known composition communities (McIntyre ABR Genome Biol. 2017 Sep. 21; 18(1):182; blog.onecodex.com/2017/06/30/tool-off/).

The species estimated to be greater than 0.1% abundant in each SFL are shown in Tables 8-11. The taxonomic ID can be matched with a particular species, e.g., on the NCBI taxonomy database. The number in the abundance column in the Tables is in fraction (i.e., 0.1=10%)

The abundance of the indicated bacterial species in the stool fractions of the donors is presented in Tables 8-11. The bolded entries had an abundance of greater than 0.001 (greater than 0.1%).

TABLE 8
Donor 1 Spore fraction

| | | Abundance |
|---|---|---|
| | | as fraction |
| Species | Tax ID | (i.e., 0.1 = 10%) |
| Ruminococcus sp. CAG:60 | 1262964 | 0.114254371 |
| Ruminococcus sp. 5_1_39BFAA | 457412 | 0.084642189 |
| Lachnospiraceae bacterium 5_1_63FAA | 658089 | 0.081860554 |
| Ruminococcus sp. CAG:9 | 1262967 | 0.067730475 |
| Blautia wexlerae | 418240 | 0.058207328 |
| Intestinibacter bartlettii | 261299 | 0.049859469 |
| Clostridium sp. 1_1_41A1FAA | 457397 | 0.036767771 |
| Flavonifractor plautii | 292800 | 0.035268356 |
| Intestinimomas butyriciproducens | 1297617 | 0.032495136 |
| Clostridium sp. ATCC BAA-442 | 649724 | 0.03102409 |
| Clostridiales bacterium VE202-13 | 1530202013 | 0.028246039 |
| Clostridium perfringens | 1502 | 0.027743555 |
| Anaerostipes hadrus | 649756 | 0.027352897 |
| Blautia obeum | 40520 | 0.02699018 |
| Blautia sp. KLE 1732 | 1226324 | 0.025347561 |
| Blautia sp. GD8 | 1737424 | 0.024798118 |
| Firmicutes bacterium CAG:56 | 1263031 | 0.023695258 |
| Ruminococcus sp. SR1/5 | 657323 | 0.019906629 |
| [Ruminococcus] torques | 33039 | 0.01812662 |
| Ruminococcus faecis | 592978 | 0.016073097 |
| Clostridium sp. JCC | 1414720 | 0.014201016 |
| Clostridium bartlettii CAG:1329 | 1263063 | 0.014122199 |
| Anaerotruncus colihominis | 169435 | 0.01240539 |
| Faecalibacterium prausnitzii | 853 | 0.012192349 |
| Ruminococcus sp. CAG:55 | 1262960 | 0.010879193 |
| Lachnospiraceae bacterium 7_1_58FAA | 658087 | 0.010548055 |
| [Clostridium] symbiosum | 1512 | 0.010286688 |
| Firmicutes bacterium CAG:270 | 1263014 | 0.009651414 |
| Agathobacter rectalis | 39491 | 0.008499925 |
| Clostridia bacterium UC5.1-2F7 | 1697792 | 0.008295436 |
| Roseburia faecis | 301302 | 0.006803593 |
| Clostridium sp. GD3 | 1650661 | 0.005870841 |
| Firmicutes bacterium CAG:41 | 1263021 | 0.005576561 |
| Coprococcus sp. ART55/1 | 751585 | 0.005199271 |
| Firmicutes bacterium CAG:145 | 1263005 | 0.003379299 |
| Firmicutes bacterium CAG:212 | 1263009 | 0.003028622 |
| Butyrate-producing bacterium SS3/4 | 245014 | 0.00289582 |
| Turicibacter sp. H121 | 1712675 | 0.002635766 |
| Coprococcus catus | 116085 | 0.00249742 |
| [Clostridium] sordellii | 1505 | 0.002380229 |
| Dorea longicatena | 88431 | 0.002363233 |
| Turicibacter sanguinis | 154288 | 0.001913638 |
| Clostridia bacterium UC5.1-1D1 | 1697794 | 0.001771593 |
| Coprobacillus sp. CAG:235 | 1262854 | 0.001381104 |
| [Eubacterium] eligens | 39485 | 0.001321584 |
| Bifidobacterium longum | 216816 | 0.001284171 |
| Clostridium sp. CAG:7 | 1262832 | 0.001101715 |
| Roseburia sp. CAG:18 | 1262941 | 0.001092103 |
| Blautia sp. CAG:37 | 1262757 | 0.000352532 |
| Eubacterium sp. CAG:202 | 1262884 | 0.000334486 |
| Eubacterium eligens CAG:72 | 1263077 | 0.000263831 |
| Collinsella sp. CAG:166 | 1262850 | 0.000173492 |
| Collinsella sp. 4_8_47FAA | 742722 | 0.000171706 |
| Collinsella aerofaciens | 74426 | 0.000167991 |

TABLE 9
Donor 1 Non-spore fraction

| | | Abundance |
|---|---|---|
| Species | Tax ID | (i.e., 0.1 = 10%) |
| Bacteroides fragilis | 817 | 0.240030795 |
| Bacteroides sp. 1_1_6 | 469586 | 0.109915096 |
| Bacteroides ovatus | 28116 | 0.057356484 |
| Bacteroides vulgatus | 821 | 0.057027174 |
| Parabacteroides merdae CAG:48 | 1263094 | 0.04539845 |
| Bacteroides sp. D20 | 585543 | 0.040816026 |
| Streptococcus salivarius CAG:79 | 1263109 | 0.037047843 |
| Bacteroides uniformis | 820 | 0.036665374 |
| Bifidobacterium longum | 216816 | 0.034175394 |
| Bacteroides sp. 3_1_19 | 469592 | 0.031930786 |
| Parabacteroides sp. D26 | 658662 | 0.030863579 |
| Streptococcus salivarius | 1304 | 0.028772724 |
| Parabacteroides merdae | 46503 | 0.020672573 |
| Parabacteroides distasonis | 823 | 0.019996753 |
| Bifidobacterium bifidum | 1681 | 0.01694393 |
| Bacteroides eggerthii | 28111 | 0.016877293 |
| Parabacteroides johnsonii | 387661 | 0.01683543 |
| Streptococcus sp. SR4 | 1161417 | 0.015911045 |
| Bacteroides sp. UNK.MGS-14 | 1638780 | 0.013589416 |
| Bifidobacterium adolescentis | 1680 | 0.013578653 |
| Bacteroides eggerthii CAG:109 | 1263043 | 0.010184478 |
| Collinsella sp. CAG:166 | 1262850 | 0.0086852 |
| Collinsella sp. 4_8_47FAA | 742722 | 0.008097428 |
| Bilophila wadsworthia | 35833 | 0.007388944 |
| Collinsella aerofaciens | 74426 | 0.007276615 |
| Coprococcus comes CAG:19 | 1263070 | 0.006906287 |
| Odoribacter splanchnicus | 28118 | 0.006038242 |
| Carnobacterium sp. N15.MGS-207 | 1637504 | 0.005593854 |
| Bacteroides salyersiae | 291644 | 0.005332525 |
| Parabacteroides goldsteinii | 328812 | 0.004365227 |
| Phascolarctobacterium sp. CAG:207 | 1262914 | 0.00354678 |
| [Ruminococcus] torques | 33039 | 0.003338547 |
| Coprococcus comes | 410072 | 0.003244754 |
| Odoribacter sp. UNK.MGS-12 | 1638778 | 0.002983057 |
| Ruminococcus sp. CAG:55 | 1262960 | 0.00259416 |
| Bacteroides sp. CAG:189 | 1262737 | 0.001677158 |
| Bacteroides sp. 1_1_30 | 457387 | 0.001649594 |
| Ruminococcus sp. CAG:90 | 1262968 | 0.001606256 |
| Ruminococcus faecis | 592978 | 0.00158367 |
| Ruminococcus sp. SR1/5 | 657323 | 0.00113882 |
| Blautia sp. GD8 | 1737424 | 0.00111962 |
| Bilophila sp. 4_1_30 | 693988 | 0.00111359 |
| Blautia obeum | 40520 | 0.00092913 |
| Blautia wexlerae | 418240 | 0.00083038 |
| Ruminococcus sp. CAG:17 | 1262951 | 0.000670423 |
| Blautia sp. KLE 1732 | 1226324 | 0.000633229 |
| Dorea longicatena | 88431 | 0.00057183 |
| Ruminococcus sp. CAG:60 | 1262964 | 0.000463894 |
| Ruminococcus sp. 5_1_39BFAA | 457412 | 0.000382035 |
| [Eubacterium] hallii | 39488 | 0.000215007 |
| Clostridium sp. JCC | 1414720 | 9.92671E−05 |

TABLE 10
Donor 2 Spore fraction

| | | Abundance |
|---|---|---|
| Species | Tax ID | (i.e., 0.1 = 10%) |
| Ruminococcus sp. N15.MGS-57 | 1637508 | 0.246389057 |
| Ruminococcus bicirculans | 1160721 | 0.228445766 |
| Ruminococcus sp. CAG:57 | 1262962 | 0.208089003 |
| Firmicutes bacterium CAG:41 | 1263021 | 0.066550215 |
| Ruminococcus sp. CAG:9 | 1262967 | 0.040836711 |
| Ruminococcus sp. 5139BFAA | 457412 | 0.027095679 |
| Blautia wexlerae | 418240 | 0.01887964 |
| Ruminococcus sp. CAG:90 | 1262968 | 0.017475529 |
| Roseburia sp. CAG:197 | 1262943 | 0.015525608 |
| Intestinibacter bartlettii | 261299 | 0.01380223 |
| Turicibacter sp. HGF1 | 910310 | 0.011074177 |
| Staphylococcus aureus | 1280 | 0.009583571 |
| Turicibacter sanguinis | 154288 | 0.009314866 |
| Lachnospiraceae bacterium 5_1_63FAA | 658089 | 0.007625159 |
| Anaerostipes hadrus | 649756 | 0.006875226 |
| Ruminococcus sp. SR1/5 | 657323 | 0.006627311 |
| Clostridium sp. 1_1_41A1FAA | 457397 | 0.006165961 |
| Ruminococcus sp. CAG:17 | 1262951 | 0.005129445 |
| Eubacterium hallii CAG:12 | 1263078 | 0.004980707 |
| Eubacterium eligens CAG:72 | 1263077 | 0.004801422 |
| Eubacterium sp. CAG:202 | 1262884 | 0.004712347 |
| Clostridium sp. JCC | 1414720 | 0.004664514 |
| Clostridium bartlettii CAG:1329 | 1263063 | 0.002897256 |
| [Ruminococcus] torques | 33039 | 0.002887768 |
| [Clostridium] sordellii | 1505 | 0.002760016 |
| [Eubacterium] hallii | 39488 | 0.002577268 |
| Coprococcus sp. ART55/1 | 751585 | 0.002478733 |
| Ruminococcus callidus | 40519 | 0.002243859 |
| Blautia sp. GD8 | 1737424 | 0.002118038 |
| Coprococcus sp. CAG:131 | 1262862 | 0.002021396 |
| Clostridium celatum | 36834 | 0.001931035 |
| Ruminococcus sp. JC304 | 1095771 | 0.001888972 |
| Blautia sp. KLE 1732 | 1226324 | 0.001503377 |
| Roseburia faecis | 301302 | 0.001314441 |
| [Eubacterium] eligens | 39485 | 0.001179972 |
| Blautia obeum | 40520 | 0.000867666 |
| Firmicutes bacterium CAG:212 | 1263009 | 0.000684508 |
| Dorea longicatena | 88431 | 0.000498219 |
| Faecalibacterium prausnitzii | 853 | 0.000370047 |
| Turicibacter sp. H121 | 1712675 | 0.000332562 |
| Bacteroides vulgatus | 821 | 0.000312006 |
| Bifidobacterium longum | 216816 | 0.000302065 |
| Collinsella aerofaciens | 74426 | 7.31707E−05 |
| Collinsella sp. 4_8_47FAA | 742722 | 7.29356E−05 |

TABLE 11
Donor 2 Non-spore fraction

| | | Abundance |
|---|---|---|
| Species | Tax ID | (i.e.,0 .1 = 10%) |
| Bifidobacterium longum | 216816 | 0.222453009 |
| Bifidobacterium adolescentis | 1680 | 0.204814337 |
| Bifidobacterium stercoris | 592977 | 0.19792986 |
| Collinsella sp. 4 8 47FAA | 742722 | 0.041064039 |
| Collinsella sp. CAG:166 | 1262850 | 0.040920779 |
| Collinsella aerofaciens | 74426 | 0.038585301 |
| Faecalibacterium prausnitzii | 853 | 0.027117182 |
| Collinsella sp. CAG:289 | 1262851 | 0.021343728 |
| Collinsella sp. MS5 | 1499681 | 0.017218285 |
| Bacteroides sp. 2_1_16 | 469587 | 0.016754777 |
| Butyrate-producing bacterium SSC/2 | 245018 | 0.014826459 |
| Bacteroides uniformis | 820 | 0.01088125 |
| Bacteroides vulgatus | 821 | 0.009174624 |
| Roseburia sp. CAG:18 | 1262941 | 0.009066265 |
| Parabacteroides merdae CAG:48 | 1263094 | 0.007515988 |
| Ruminococcus sp. CAG:55 | 1262960 | 0.007419941 |
| Blautia sp. CAG:37 | 1262757 | 0.006862496 |
| Bacteroides faecis | 674529 | 0.006664491 |
| Dorea formicigenerans CAG:28 | 1263073 | 0.006638101 |
| Coprococcus comes CAG:19 | 1263070 | 0.006034677 |
| Roseburia faecis | 301302 | 0.005997385 |
| Coprococcus comes | 410072 | 0.005897654 |
| [Ruminococcus] torques | 33039 | 0.004537152 |
| Ruminococcus sp. 5_1_39BFAA | 457412 | 0.004245095 |
| Staphylococcus aureus | 1280 | 0.004122705 |
| Coprococcus sp. ART55/1 | 751585 | 0.003809693 |
| Ruminococcus sp. CAG:9 | 1262967 | 0.003691574 |
| Parabacteroides merdae | 46503 | 0.003687796 |
| Ruminococcus faecis | 592978 | 0.003599944 |
| Dorea longicatena | 88431 | 0.003502778 |
| Blautia wexlerae | 418240 | 0.003439925 |
| Coprococcus sp. CAG:131 | 1262862 | 0.003276343 |
| Bacteroides sp. D20 | 585543 | 0.003271357 |
| Lachnospiraceae bacterium CAG:25 | 1262984 | 0.003054647 |
| Dorea formicigenerans | 39486 | 0.002973301 |
| Bacteroides fragilis | 817 | 0.002092517 |
| Parabacteroides distasonis | 823 | 0.002063842 |
| Ruminococcus sp. CAG:17 | 1262951 | 0.001718271 |
| Sutterella sp. CAG:397 | 1262976 | 0.001636647 |
| Ruminococcus sp. JC304 | 1095771 | 0.001289925 |
| Bacteroides massiliensis | 204516 | 0.001287539 |
| Blautia obeum | 40520 | 0.0012123 |
| Eubacterium hallii CAG:12 | 1263078 | 0.001142996 |
| [Eubacterium] hallii | 39488 | 0.000968696 |
| Odoribacter splanchnicus | 28118 | 0.000792204 |
| Bacteroides eggerthii | 28111 | 0.000777778 |
| Anaerostipes hadrus | 649756 | 0.000741052 |
| Bacteroides ovatus | 28116 | 0.000498587 |
| Agathobacter rectalis | 39491 | 0.000470219 |
| Butyrate-producing bacterium SS3/4 | 245014 | 0.000469715 |
| Firmicutes bacterium CAG:270 | 1263014 | 0.000404282 |
| Parabacteroides sp. D26 | 658662 | 0.000322602 |
| Streptococcus salivarius | 1304 | 0.000287024 |
| Eubacterium sp. CAG:202 | 1262884 | 0.000275057 |
| [Clostridium] sordellii | 1505 | 8.86088E−05 |
| Eubacterium eligens CAG:72 | 1263077 | 6.0261E−05 |
| Ruminococcus sp. CAG:60 | 1262964 | 2.47064E−05 |

### Example 2B: Non-Spore Forming Stool Fractions are as Effective as FMT in CRE Clearance

As described herein, stool samples from healthy donors have been found to promote clearance of multidrug-resistant Klebsiella pneumoniae colonization. Non-spore forming (NSP) stool fractions and FMT from healthy donors were tested for their ability to reduce the CRE burden (decolonize) in mice colonized with CRE as shown in FIG. 23A. As shown in FIG. 23B, the NSP fractions were as effective as FMT at reducing the CRE burden. Mice that received the NSP fractions exhibited comparable CRE decolonization rates as mice that received FMT.

### Example 2C: Live Bacterial Products (LBPs) Directed Against CRE

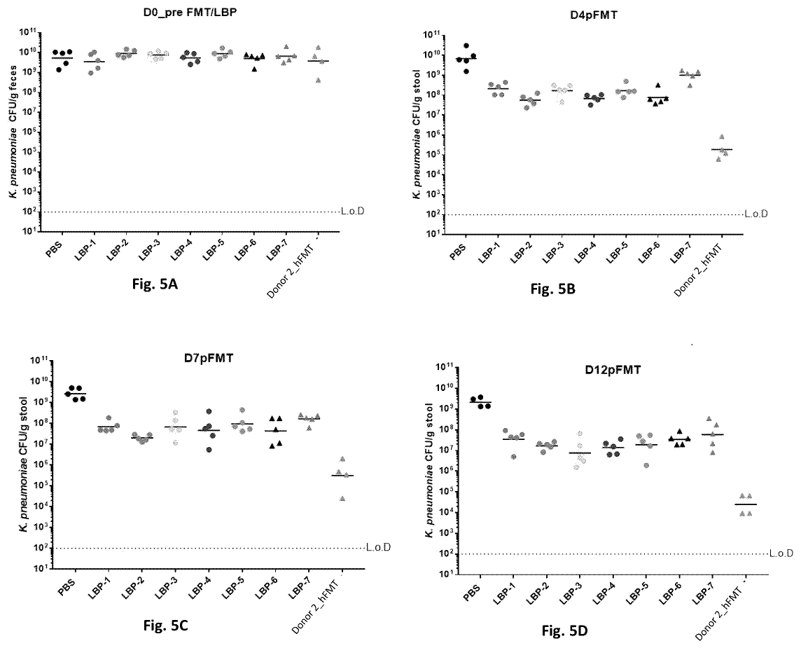

Bacterial strains were assembled into various live bacterial products (LBPs) shown in FIG. 4. The LBPs were tested in the CRE colonization mouse model. As shown in FIGS. 5A-5D, the LBPs and FMT reduced the CRE burden as compared to PBS control.

### Example 2D: Clearance of CRE

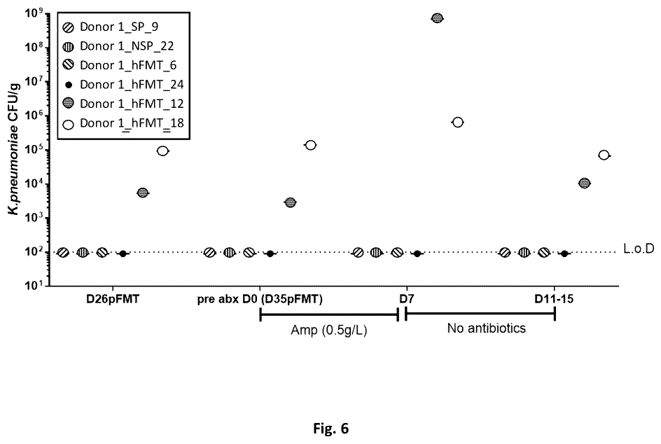

To determine whether CRE clearance by FMT and SFL was true clearance of CRE or an inability to detect low CRE levels, mice that cleared CRE in the experiments described previously received a second round of ampicillin on day 35 post-FMT/SFL treatment. Ampicillin treatment was terminated after 7 days. Mice that had detectable CRE levels (10⁴-10⁵ CFU range) were also ampicillin-treated and used as controls. Fecal samples were collected from each mouse pre-antibiotic administration, on day 7 of ampicillin treatment, and at 2 weeks post-ampicillin treatment to assess the re-expansion, or lack thereof, of CRE. As shown in FIG. 6, CRE levels did not increase following antibiotic treatment of decolonized mice, indicating that FMT/SFL-mediated decolonization of CRE is sufficient to eradicate CRE from the intestines.

### Example 2E: Identification of CRE-Antagonistic Strains

In vitro assays were performed to identify CRE pathogen-antagonistic strains. Soft agar overlay assays are commonly used in classical antibiotic drug discovery programs, and a zone of inhibition indicates suppression of the target strain. The assay complements other in vitro screening systems (e.g., growth competition assay). The top agar layer is embedded with the target strain, which is poured over plate containing a sterile disc spotted with a test strain. After an incubation period, a zone of inhibition surrounding the disc can be measured, and the level of activity of different test strains can be examined.

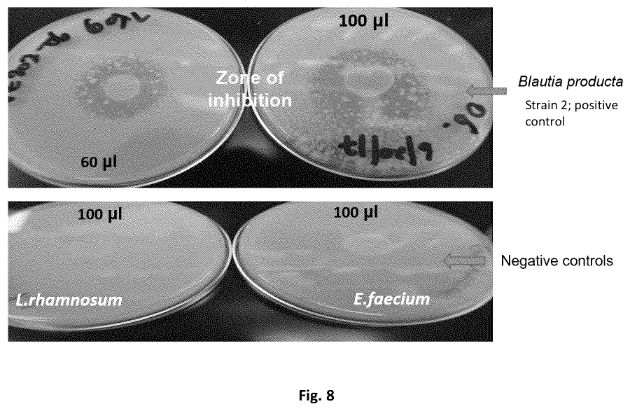

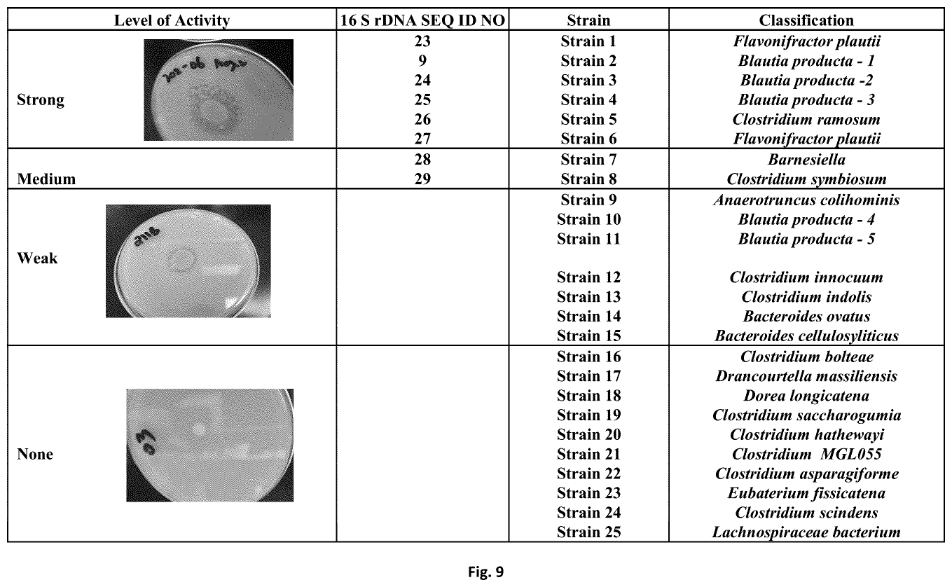

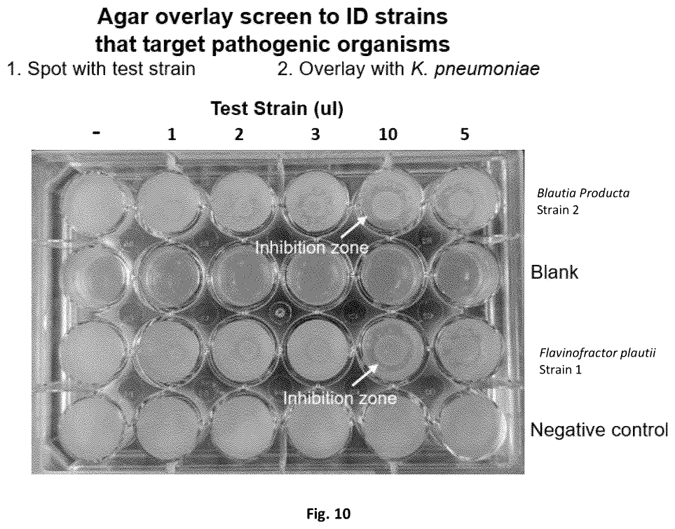

In the example soft agar overlays of FIG. 8, Blautia producta inhibits K. pneumoniae 2814 (CRE) in the assay, shown by the zone of inhibition. The inhibitory activity is both dose-dependent and bacterium-specific. Neither L. rhamnosum nor E. faecium resulted in a zone of inhibition in the assay. FIG. 9 presents a table showing bacterial strains that had CRE suppressing activity in the soft agar overlay assays.

The soft agar overlay assay was performed using a medium throughput screening method, shown in FIG. 10. Using 24-well plates, 72 bacterial strains can be screened per day per pathogen to identify strains that have antagonistic activity against pathogenic organisms.

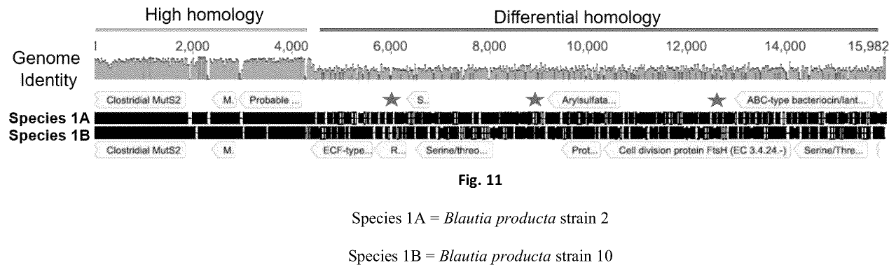

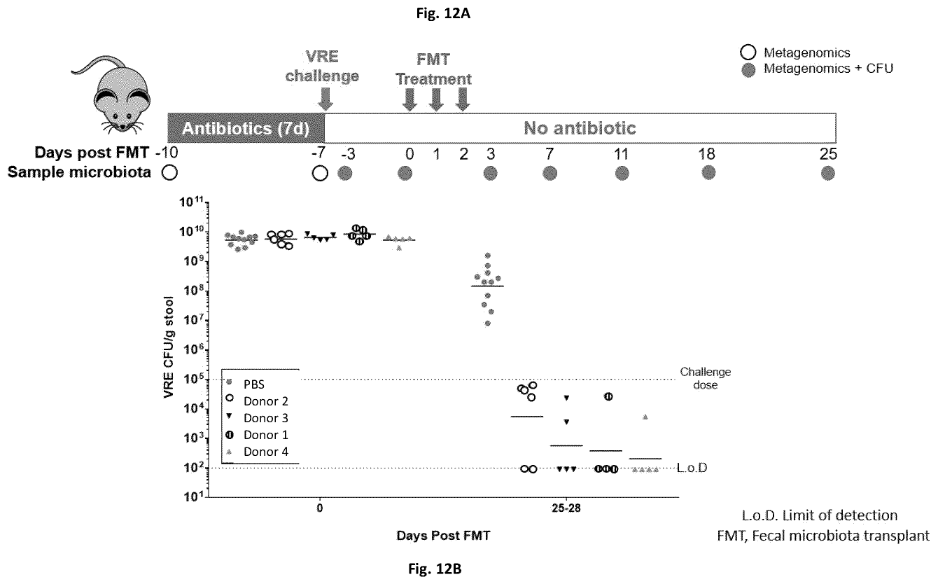

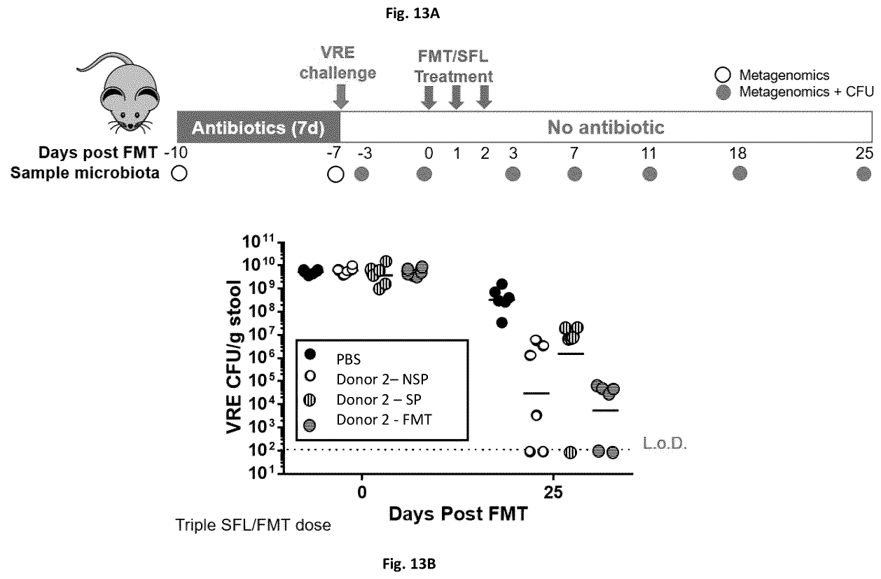

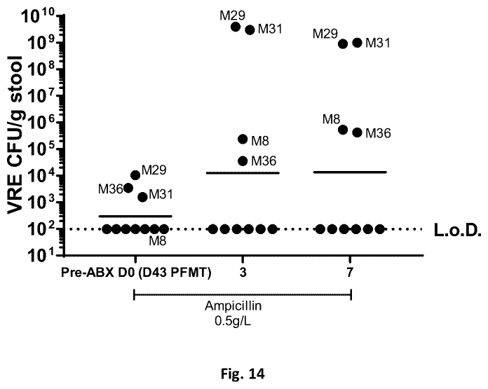

Finally, genomic information from the bacterial strains was examined to determine reveal potential mechanisms of action for the pathogen-antagonistic activity. For example, two bacterial strains of the same species (Blautia producta) were identified having differential pathogen-antagonistic activity. In particular, Strain 2 had pathogen-antagonistic activity and Strain 10 did not. A genome alignment revealed the presence of bacteriocin-associated genes (indicated by stars in FIG. 11) in Strain 2 that were absent in Strain 10. The analysis was performed using anti-SMASH (antibiotics and secondary metabolite analysis shell; Weber et al., 2015).

### Example 3: Colonization Resistance is Maintained Following Clearance of CRE

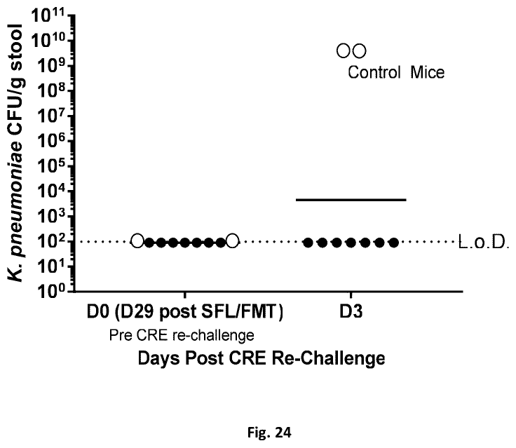

Mice that had previously cleared CRE colonization in the FMT/SFL experiments described in Example 2B (FIGS. 23A and 23B) were assessed to determine if the mice could be re-colonized with subsequent CRE challenge. A subset of mice that had cleared CRE in FIG. 23B at various time points post-FMT/SFL administration were re-challenged with 10⁵ CRE CFU on day 29 post-FMT/SFL treatment. To ensure viability of the CRE inoculum and its ability to expand in the intestine, a cohort of untreated mice were administered ampicillin for 7 days and challenged with CRE. Fecal samples were collected three days post-challenge to assess CRE expansion. As shown in FIG. 24, the mice that had previously cleared CRE were resistant to re-colonization with CRE.

### Example 4: Vancomycin-Resistant Enterococcus faecium

As described in Example 1, mouse models of vancomycin-resistant Enterococcus faecium (VRE) colonization were established. As shown in FIG. 12A, mice were treated with broad-spectrum antibiotics (ampicillin) for seven days prior to the VRE challenge. On day −7, the ampicillin treatment was terminated. Mice were single-housed and challenged with 10⁴-10⁵ CFU of VRE. Seven days after the pathogen challenge (on day 0), mice were randomized into groups, and the first of three treatment doses of fecal microbiota treatment (FMT) or stool fraction library (SFL) was administered on the third day after pathogen challenge. The three day period between VRE challenge and FMT/SPL administration served as a washout period to clear residual antibiotics and establish VRE colonization in the intestine. Fecal samples were collected at various time points post-treatment to quantify VRE levels (plating on selective media) and to determine strain engraftment by metagenomic sequencing. As shown in FIG. 12B, administration of FMT from donors reduced the VRE burden from densely-colonized mice.

Stool fractions were generated from donors with potent pathogen-antagonistic activity, as described in Example 2. The stool fractions were tested for their ability to reduce the VRE burden (decolonize) in mice colonized with VRE as shown in FIG. 13A. As shown in FIG. 13B, the stool fractions were able to reduce the VRE burden.

To determine whether VRE clearance by FMT and SFL was true clearance of VRE or an inability to detect low VRE levels, mice that cleared VRE in the experiments described above received a second round of ampicillin on day 43 post-FMT/SPL treatment. Ampicillin treatment was terminated after 7 days. Mice that had detectable CRE levels (10³-10⁴ CFU range) were also ampicillin-treated and used as controls. Fecal samples were collected from each mouse pre-antibiotic administration and on days 3 and 7 post-ampicillin treatment to assess the re-expansion, or lack thereof, of VRE. As shown in FIG. 14, VRE levels did not increase following antibiotic treatment of the majority of decolonized mice, indicating that FMT/SFL-mediated decolonization of VRE was sufficient to eradicate VRE from the intestines. In particular, 90% of the decolonized mice remained VRE-free despite 7 days of ampicillin treatment, whereas VRE expanded significantly in mice with detectable VRE levels.

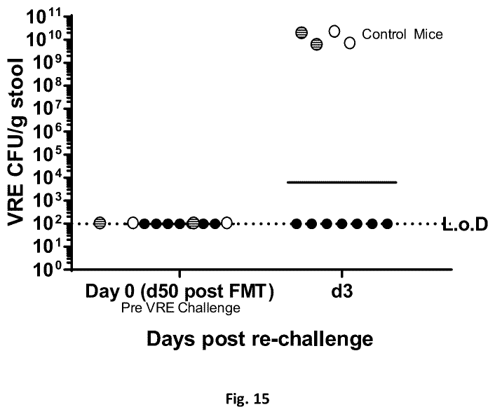

Mice that had cleared VRE colonization in the FMT experiment (FIG. 12) were assessed to determine if the mice could be re-colonized with subsequent VRE challenges. A subset of mice that had cleared VRE in FIG. 12 at various time points post-FMT administration were re-challenged with 10⁴ VRE CFU on day 0, corresponding to day 50 post-FMT. To ensure viability of the VRE inoculum and its ability to expand in the intestine, a cohort of untreated mice were administered ampicillin for 7 days to ensure viability of the VRE inoculum. Fecal samples were collected three days post-challenge to assess VRE expansion. As shown in FIG. 15, the mice that had previously cleared VRE were resistant to re-colonization with VRE.

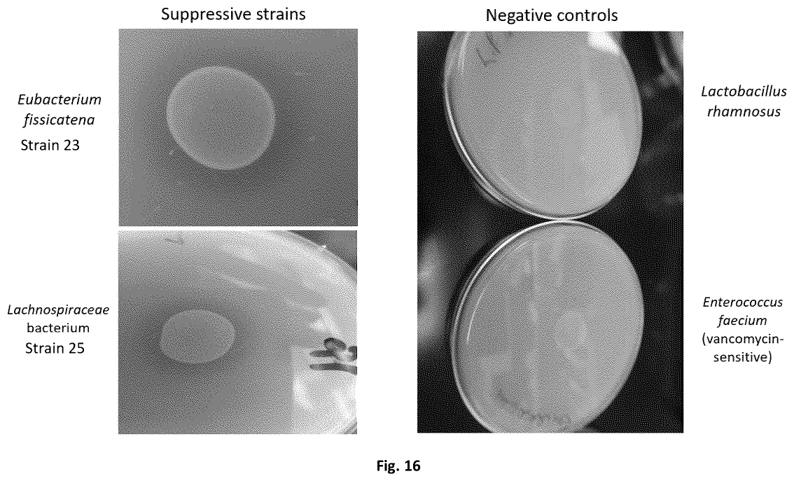

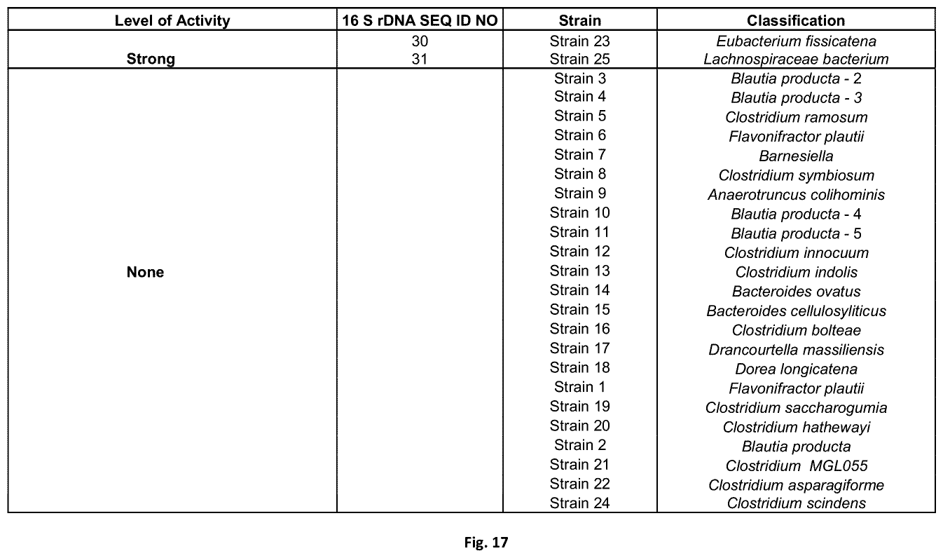

In vitro soft agar assays were performed, as described in Example 2, to identify pathogen-antagonistic bacterial strains having activity against VRE. Example soft agar overlays are presented in FIG. 16 in which Eubacterium fissicatena and Lachnospiraceae bacterium had zones of inhibition whereas Lactobacillus rhamnosus and Enterococcus faecium did not. Results from the soft agar overlay assays are presented in FIG. 17.

### Example 5: Broth Based Competition Assays

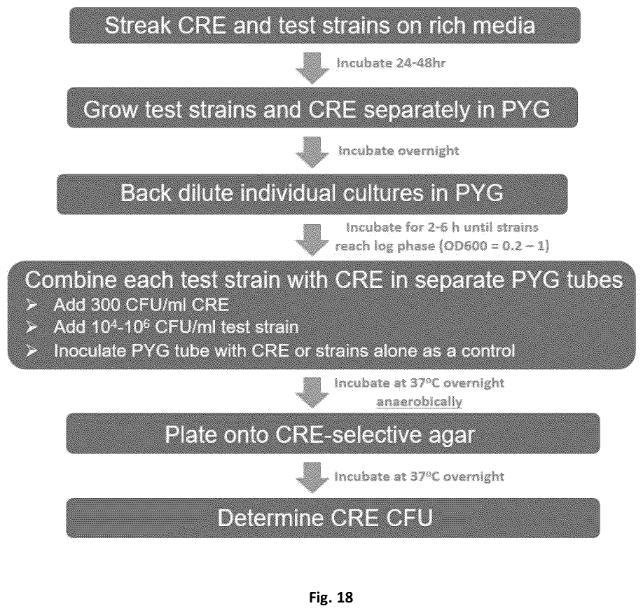

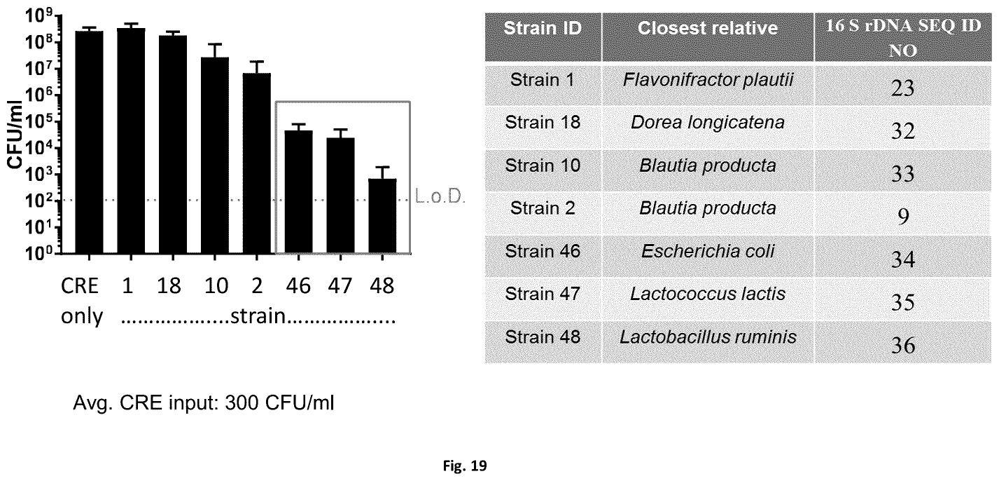

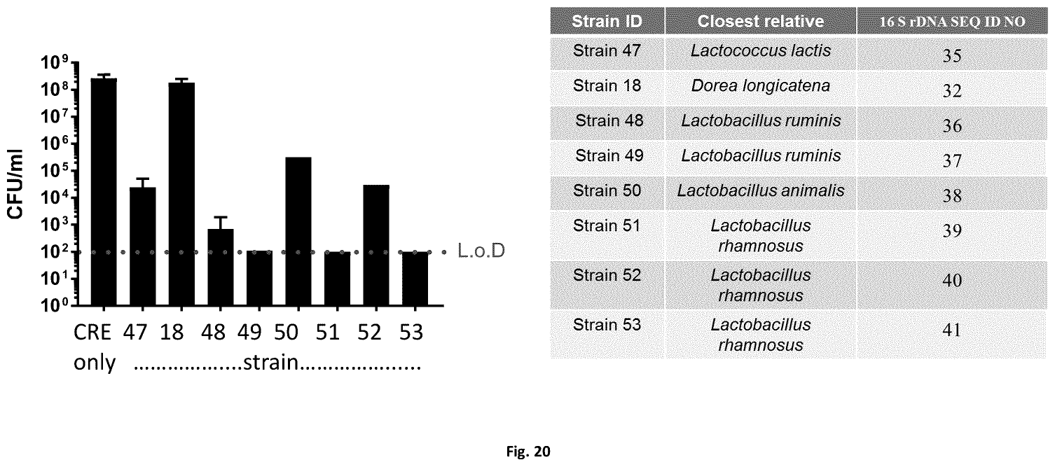

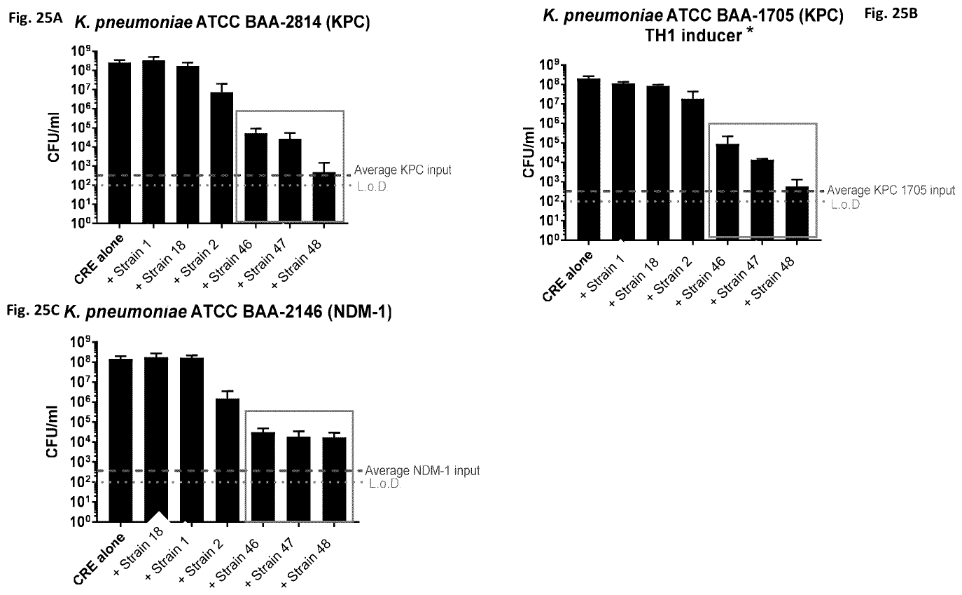

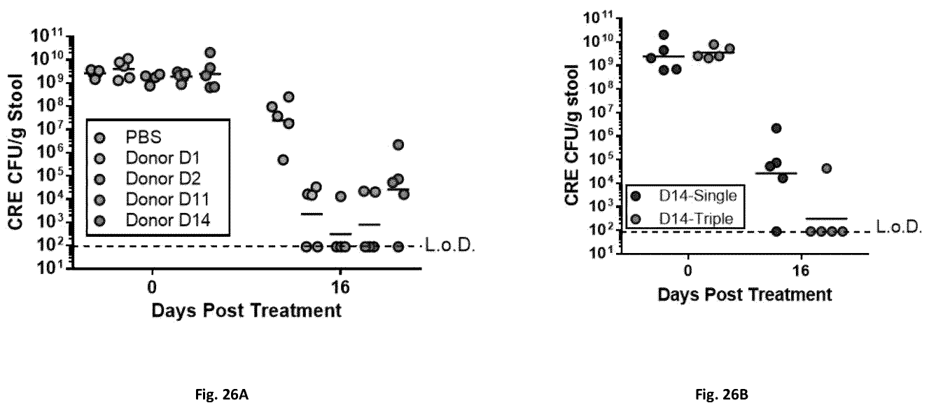

A broth-based competition assay was developed to allow for the determination of MDRO (e.g., CRE and VRE) suppressing ability of bacterial strains of interest. Individual bacterial strains to be tested were grown as a co-culture from early stationary phase liquid cultures with a defined CRE or VRE inoculum under anaerobic conditions. The culture was plated onto CRE or VRE selective media and the number of Colony Forming Units (CFUs) was determined. FIG. 18 shows a schematic of the workflow of this broth-based competition assay with CRE as the MDRO. The results are shown in FIGS. 19, 20, and 25A. FIGS. 19 and 25A shows that Lactobacillus ruminus provided the highest level of suppression of CRE. FIG. 20 shows that some strains of Lactobacillus rhamnosus provided a similar level of suppression as Lactobacillus ruminus. All strains evaluated were donor-derived strains except for E. coli (ATCC-25922) and Lactococcus lactis (ATCC-11454).

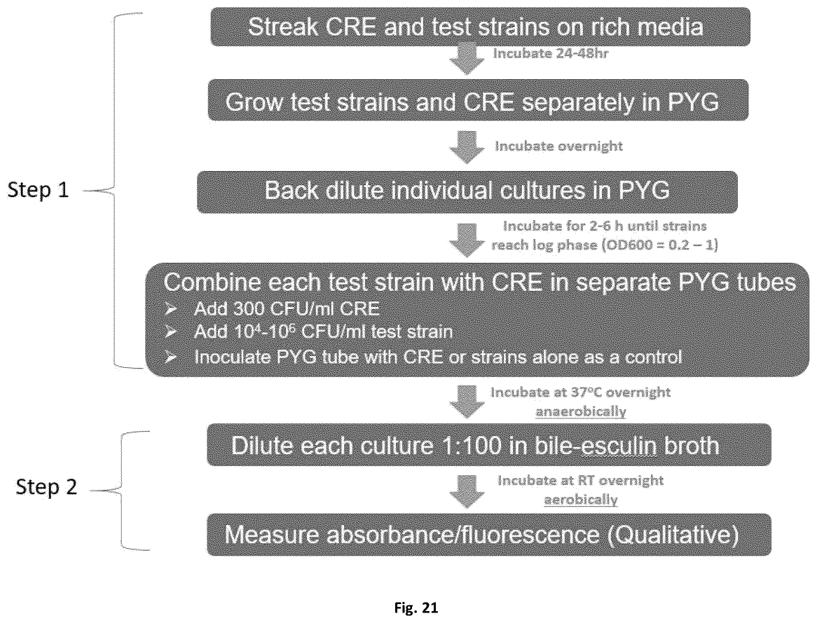

The bacterial strains were also assessed for their ability to suppress different CRE strains, including K. pneumoniae ATCC BAA-2814 (KPC), K. pneumoniae ATCC BAA-1705 (KPC), and K. pneumoniae ATCC BAA-2146 (NDM-1). As shown in FIGS. 25A-25C, several of the bacterial strains had broad-spectrum activity in inhibiting CRE strains. The broth-based competition assay was developed further to allow for suppression level readout through color observation or relative fluorescence units (RFUs). FIG. 21 shows a schematic of the workflow of the broth-based competition assay with visual readout.

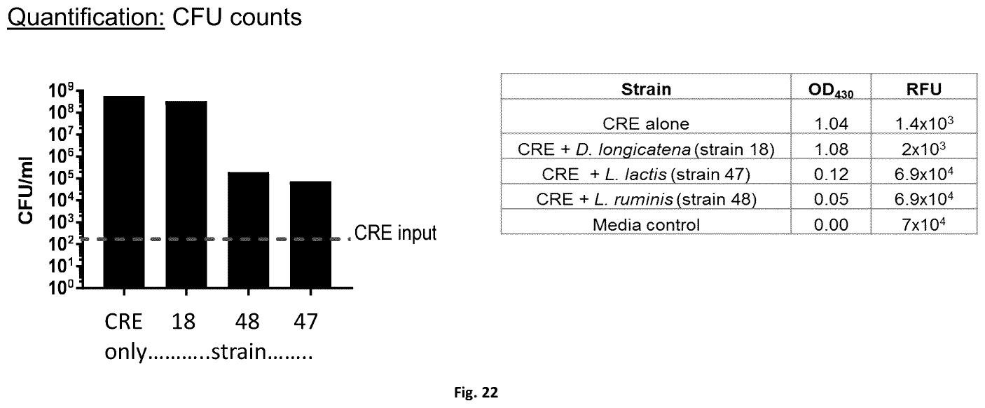

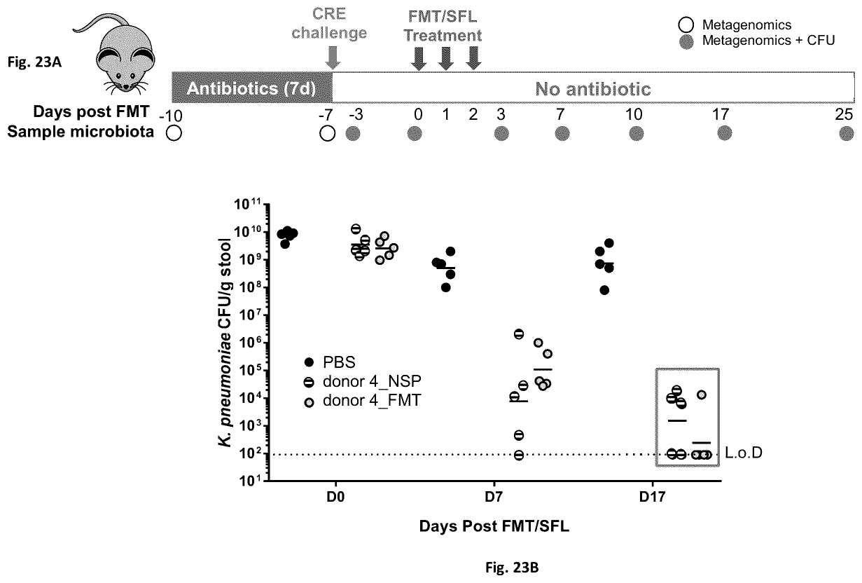

Individual bacterial strains to be tested were grown as a co-culture from early stationary phase liquid cultures with a defined CRE or VRE inoculum under anaerobic conditions. After growth overnight, a small aliquot of each co-culture was transferred into chromogenic, selective media (bio-esculin broth) that changes color from clear to dark as a result of CRE or VRE expansion. Eight hours after transfer into the bio-esculin broth, any change in chromogenicity, which is indicative of CRE/VRE growth, was assessed visually and measured by fluorescence intensity as relative fluorescence units (RFU). The results of a representative experiment are shown in FIG. 22. Pathogens cultured alone or in the presence of inactive strains expanded to 10⁸-10⁹ CFU which corresponded to 10³ RFU. Pathogens grown in the presence of highly suppressive strains resulted in ≥3 log reduction in pathogen expansion and corresponded to 10⁵ RFU, which was similar to the RFU control of the media alone. The assay with visual readout therefore has a sufficient dynamic range to evaluate the suppressive activity of bacterial strains of interest. Similar to the outcome of the CFU broth assay, Lactobacillus rhamnosus and Lactobacillus ruminus provided the strongest level of suppression.

The assay therefore has a sufficient dynamic range to evaluate the suppressive activity of bacterial strains of interest.

### Example 6: FMT Efficacy Against VRE and CRE—Single Dose Vs. Triple Dose

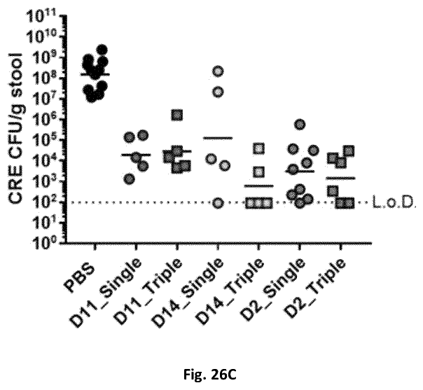

To investigate the efficacy of FMT treatment against vancomycin resistant Enterococci (VRE) and carbapenem resistant Enterobacteriaceae (CRE), C57BL/6 mice were treated with 0.5 g of ampicillin in the drinking water for 7 days and challenged with 10⁵ colony-forming units (CFU) of either VRE (ATCC 700221) or CR-KP (ATCC BAA-2814) on day 7, at which point antibiotic treatment was discontinued. Three days following challenge, subsets of mice were administered a single dose or three consecutive FMT doses from each of four well-characterized donors. CR-KP and VRE colonization levels were measured in fecal samples collected from each mouse longitudinally following the first FMT dose by plating on selective media. While control mice (PBS-treated) remained densely colonized for the duration of the experiment, mice treated with fecal material from all four donors exhibited reduced levels of CR-KP (see FIGS. 26A and 26C) and VRE that were comparable to, or higher than, mouse derived FMT. Notably, a single FMT dose was as efficacious as a triple dose at clearing CR-KP (FIGS. 26A and 26C) and VRE for 3 of the 4 donors. Donor D14, on the other hand, achieved a 75% clearance rate for both CR-KP and VRE when administered to mice as a triple dose compared to 25% when administered as a single dose (see FIGS. 26B and 26C).

### Example 7: Stool Fraction Efficacy Against VRE and CRE

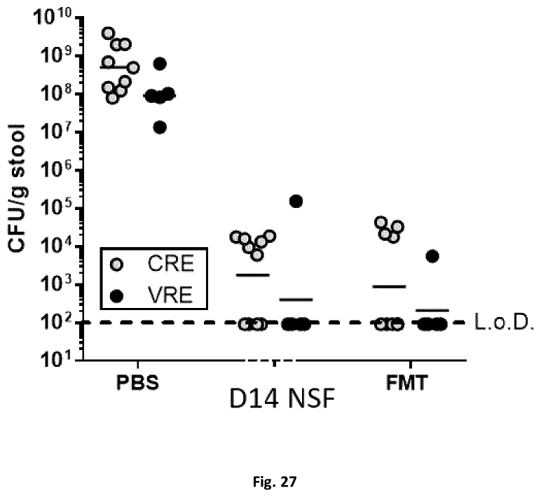

Non-spore forming fractions were generated from donor 3, donor 2, donor 5, donor 1, and donor 4 (“D14”), as described in Example 2. The stool fractions were tested for their ability to reduce the CRE burden in mice colonized with CRE after 17 days of treatment (FIG. 29). The stool fractions were then tested for their ability to reduce the VRE and CRE burden (decolonize) in mice colonized with VRE as shown in FIG. 13A, or CRE, after 25 days of treatment. As shown in FIG. 27, the non-spore forming stool fraction (D14 NSF) was able to reduce the VRE and CRE burdens.

### Example 8: In Vitro Hits for CRE in a Broth-Based Competition Assay

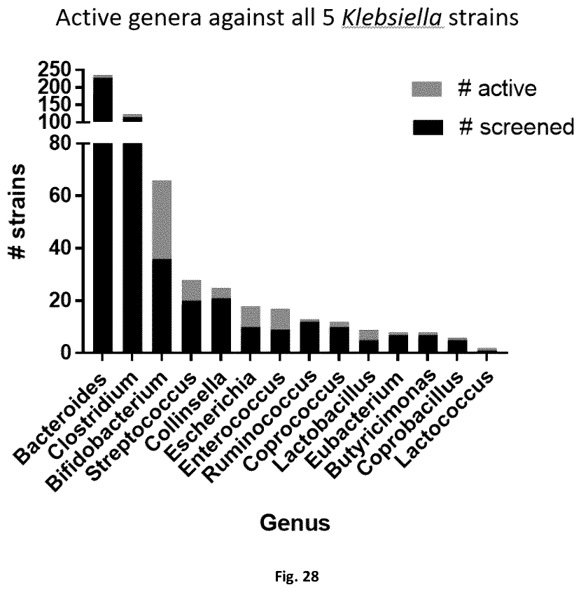

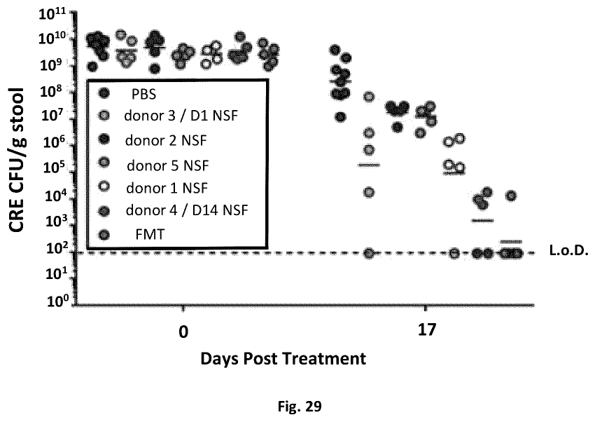

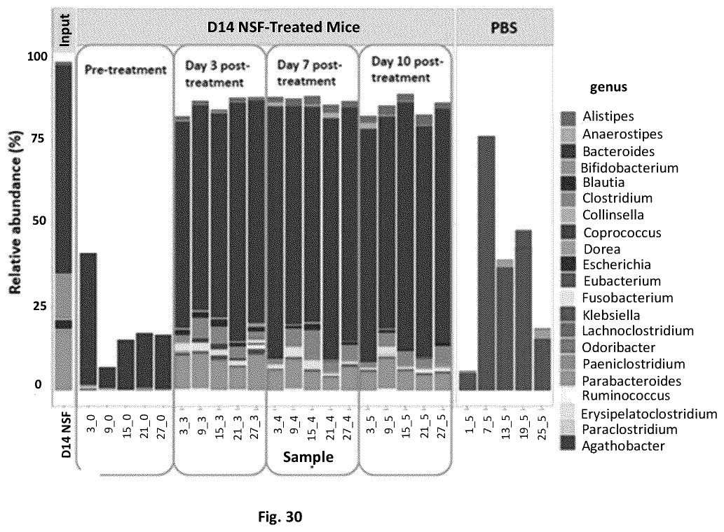

For the broth-based competition assay, individual test strains from early stationary phase liquid cultures first were co-cultured with a defined CR-KP or VRE inoculum anaerobically. The following day, a small aliquot of each co-culture was transferred into chromogenic, selective media that changes color from clear to black as a result of CRE or VRE expansion. Eight hours later, chromogenic changes, indicative of CRE/VRE growth, were assessed visually and measured by fluorescence intensity as relative fluorescence units (RFU). Pathogens cultured alone or in the presence of inactive strains expanded to 10⁸-10⁹ CFU/ml which corresponded to 10³ RFU. Pathogens grown in the presence of highly suppressive strains resulted in ≥3 log reduction in pathogen expansion and corresponded to 10⁵ RFU, which was also the RFU of the media alone. Thus, a RFU scale of 10³ (least active) to 10⁵ (most active) was used to determine the CRE and VRE suppressive activity of test strains. FIG. 28 shows the number of screened strains in the listed bacterial genera and the number of those strains that are active against 5 Klebsiella strains.

### Example 9: Assessing the Relative Abundance and Persistence of Bacterial Strains from Donor 4 (D14) in the Mouse Intestine

The colonization and persistence of bacterial strains from non-spore forming stool fractions from donor 4 (D14 NSF) were investigated in the intestines of mice. C57BL/6 mice were treated with antibiotics for 7 days, challenged with 10⁵ colony-forming units (CFU) of carbapenem-resistant Klebsiella pneumoniae (CR-KP), and administered D14 NSF. The fecal samples were collected at various time points (“output”) and sequenced by whole-genome sequencing, and the bacterial strains were taxonomically classified. D14 NSF (“input”) was also sequenced.

Mice prior to administration of D14 NSF (but after antibiotic treatment and CR-KP colonization) were densely colonized with CR-KP (data not shown), however following D14 NSF administration, the microbiota resembled that of the D14 NSF inoculum (input) and was drastically different from the mouse endogenous microbiota (pre-treatment). FIG. 30. Mice that were administered D14 NSF had undetectable levels of CR-KP on days 7 and 10 following treatment, which correlated with the 4 log reduction or clearance of CP-KP as determined by CFU levels (FIG. 29). The relative abundance of CR-KP ranged from 10-80% in control mice treated with PBS only at day 10 post treatment.

Analysis of the microbiota composition of these mice allowed identification of D14 NSF strains that stably colonized the intestine following complete clearance or significant reduction (≥4 log compared to PBS controls) of CRE fecal burden. As shown in Table 12, 36 bacterial strains from D14 NSF were found to be present at days 3, 7, and 10 post D4 NSF administration.

TABLE 12
Bacterial strains of compositions of 36 bacterial strains
and 23 bacterial strains from non-spore forming fraction
from donor 4 (D14) and the prevalence/abundance of each
in a larger samples size of healthy donors:

| | D14 | % | SEQ | |
|---|---|---|---|---|
| | 23 | Identity | ID | |
| D14 36 strains | strains | by 16S | NO: | Strain |
| Bacteroides caccae | X | 99.7 | 42 | 54 |
| Bacteroides intestinalis/ | X | 99.2 | 43 | 55 |
| Bacteroides cellulosyticus | | | | |
| Bacteroides_faecis | X | 99.3 | 44 | 56 |
| Bacteroides_ovatus | X | 99.1 | 45 | 57 |
| Bacteroides_thetaiotaomicron | X | 98.4 | 46 | 58 |
| Bacteroides uniformis | X | 99.9 | 47 | 59 |
| Bacteroides vulgatus | X | 99.7 | 48 | 60 |
| Bifidobacterium_adolescentis | | 99.324 | 49 | 61 |
| Bifidobacterium_longum | | 99.6 | 50 | 62 |
| Bifidobacterium_pseudocatenulatum | | 99.1 | 51 | 63 |
| Clostridiales bacterium VE202-06/ | X | 100 | 52 | 64 |
| Blautia coccoides/ | | | | |
| Blautia producta | | | | |
| Clostridium_citroniae | X | 99.7 | 53 | 65 |
| Clostridium sp. C105KSO14/ | X | 99.77 | 54 | 66 |
| Clostridium clostridioforme | | | | |
| Clostridiales bacterium VE202-212/ | X | 98.6 | 55 | 67 |
| Clostridium innocuum/ | | | | |
| Eubacterium contortum | | | | |
| Erysipelotrichaceae bacterium 6_1_45/ | X | 98.7 | 56 | 68 |
| Clostridium innocuum | | | | |
| Paeniclostridium sordellii/ | | 99.0 | 57 | 69 |
| Clostridium sordelli | | | | |
| Coprococcus comes | X | 95.3 | 58 | 70 |
| Dorea longicatena | X | 99.7 | 59 | 71 |
| Erysipelatoclostridium_ramosum | X | 98.9 | 60 | 72 |
| Eubacterium_rectale | X | 100 | 61 | 73 |
| Odoribacter sp. UNK.MGS-12/ | | 99.6 | 62 | 74 |
| Odoribacter splanchnicus | | | | |
| Bacteroides sp. 1_1_14/ | | 96.9 | 63 | 75 |
| Parabacteroides merdae/ | | | | |
| Parabacteroides distasonis | | | | |
| Bacteroides sp. UNK.MGS-14/ | | 98.1 | 64 | 76 |
| Parabacteroides merdae | | | | |
| Bacteroides xylanisolvens | X | 99.708 | 65 | 77 |
| Blautia obeum | X | 98.8 | 66 | 78 |
| Alistipes putredinis | X | 100.0 | 67 | 79 |
| Collinsella aerofaciens | | 99.5 | 68 | 80 |
| Eubacterium hallii/ | X | 99.932 | 69 | 81 |
| Bacteroides faecis | | | | |
| Alistipes shahii | X | 89.0 | 70 | 82 |
| Anaerostipes caccae | | 99.4 | 71 | 83 |
| Phascolarctobacterium faecis/ | | 99.3 | 72 | 84 |
| Phascolarctobacterium faecium | | | | |
| Agathobaculum/ | | 98.0 | 73 | 85 |
| Agathobaculum butyriciproducens | | | | |
| Bacteroides sp. 2_1_56FAA | | 99.9 | 74 | 86 |
| (Bacteroides fragilis) | | | | |
| Fusobacterium mortiferum | X | 99.022 | 75 | 87 |
| Paraclostridium bifermentans/ | | 100.0 | 76 | 88 |
| Paraclostridium benzoelyticum | | | | |
| Escherichia sp. 3_2_53FAA/ | X | 99.87 | 77 | 89 |
| Escherichia_fergusonii | | | | |

### Example 10: In Vivo Testing of a Composition of 23 and 36 Bacterial Strains from D14 NSF

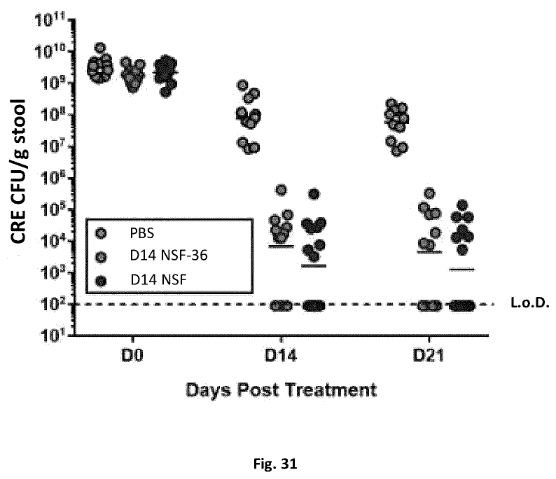

The composition of 36 bacterial strains from D14 NSF (D14 NSF-36) shown in Table 12 was tested to determine whether it could efficiently promote decolonization of carbapenem-resistant Klebsiella pneumoniae (CR-KP, CRE) from the mouse intestine. The composition was established by isolating individual donor strains and combining the individual donor strains into a composition consisting of the 36 bacterial strains. Each of the strains was cultured and combined into a composition prior to administration to antibiotic-treated mice that had been challenged with 10⁵ colony-forming units (CFU) of CR-KP. As shown in FIG. 31, all mice were colonized with CR-KP prior to administration of the composition or D14 NSF (D0). 100% of mice treated with the composition of 36 strains from D14 NSF exhibited, on average, a ≥4 log reduction in CRE CFU levels at days 14 and 21 post treatment, with clearance observed in 40% of mice. The results with the composition of 36 strains were comparable to the decolonization/clearance efficacy observed following administration of D14 NSF.

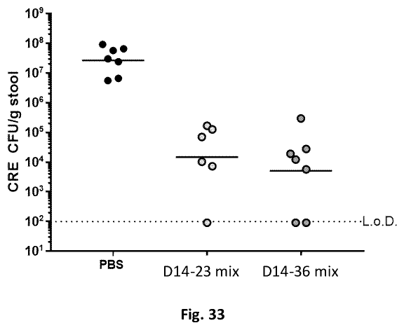

A subset of 23 strains of the 36 strains was also evaluated, using the same experimental setup and conditions. The results are shown in FIG. 33 which compares the results of the 23 strains versus the 36 strains. The 23 strains are indicated in Table 12

### Example 11: In Vitro Activity of a Composition of 36 Bacterial Strains from D4 NSF

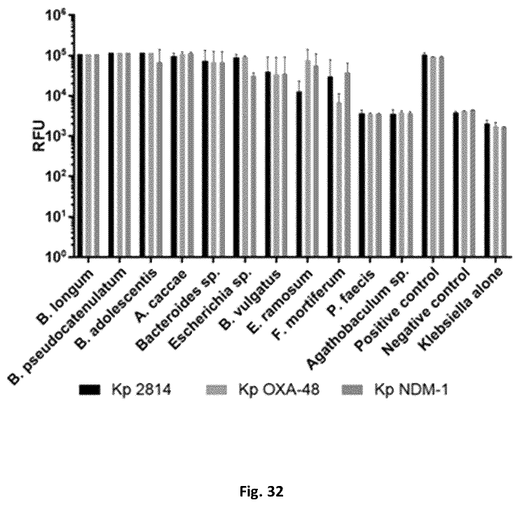

The in vitro activity of a subset of the bacterial strains of the 36 bacterial strain composition (D14 NSF-36, Table 12) against Klebsiella pneumoniae strains was examined as in Example 8. FIG. 32 shows that at least 9 of the 36 strains had direct activity against 4 different Klebsiella pneumoniae strains (Kp 2814, Kp OXA-48, Kp NDM-1, and Kp NDM-2 (data not shown)).

### Example 12: In Vivo Testing of a Composition of 36 and Non-Spore Forming Fraction from D14

Compositions of 36 bacterial strains from D14 (donor 4; “D14-36 mix”) or from the non-spore foming fraction from D14 (donor 4; “D14-NSF”) were tested for the ability to promote decolonization of Klebsiella pneumoniae 2H7 (KP 2H7) from the mouse intestine. The compositions were prepared by combining individual bacterial strains into a composition consisting of the 36 bacterial strains (see, Table 12) or a composition consisting of the non-spore forming frations. The compositions (or PBS control) were administered to antibiotic-treated mice that had been challenged with 10⁵ colony-forming units (CFU) of KP 2H7, and the bacterial burden of KP 2H7 was quantified.

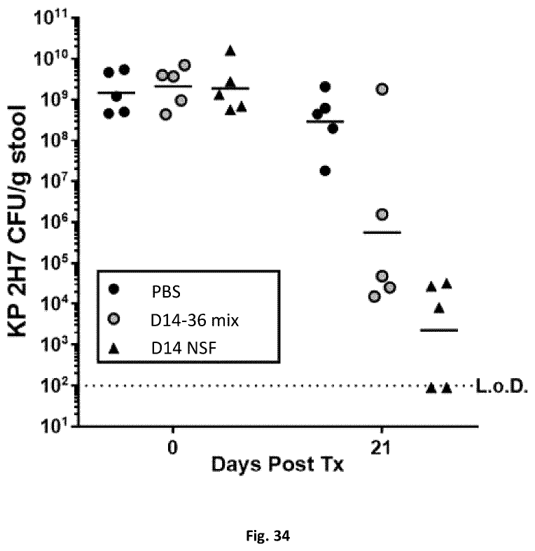

As shown in FIG. 34, mice treated with the D14-36 mix composition exhibited, on average, a ≥3 log reduction in KP 2H7 CFU levels at day 21 post treatment, and mice treated with the non-spore forming fraction exhibited, on average, a ≥5 log reduction in KP 2H7 CFU levels at day 21 post treatment.

1-109. (canceled)

110. A method for suppressing colonization by a pathogenic organism in a subject, the method comprising administering to the subject a therapeutically effective amount of a composition comprising one or more of:
(i) a bacterial strain comprising a 16S rDNA sequence having at least 97% sequence identity to the nucleotide sequence of SEQ ID NO: 48;
(ii) a bacterial strain comprising a 16S rDNA sequence having at least 97% sequence identity to the nucleotide sequence of SEQ ID NO: 49;
(iii) a bacterial strain comprising a 16S rDNA sequence having at least 97% sequence identity to the nucleotide sequence of SEQ ID NO: 50;
(iv) a bacterial strain comprising a 16S rDNA sequence having at least 97% sequence identity to the nucleotide sequence of SEQ ID NO: 51;
(v) a bacterial strain comprising a 16S rDNA sequence having at least 97% sequence identity to the nucleotide sequence of SEQ ID NO: 60;
(vi) a bacterial strain comprising a 16S rDNA sequence having at least 97% sequence identity to the nucleotide sequence of SEQ ID NO: 64;
(vii) a bacterial strain comprising a 16S rDNA sequence having at least 97% sequence identity to the nucleotide sequence of SEQ ID NO: 71;
(viii) a bacterial strain comprising a 16S rDNA sequence having at least 97% sequence identity to the nucleotide sequence of SEQ ID NO: 75; and
(ix) a bacterial strain comprising a 16S rDNA sequence having at least 97% sequence identity to the nucleotide sequence of SEQ ID NO: 77.

111. The method of claim 110, wherein:
the bacterial strain of (i) comprises a 16S rDNA sequence having at least 99% sequence identity to the nucleotide sequence of SEQ ID NO: 48;
the bacterial strain of (ii) comprises a 16S rDNA sequence having at least 99% sequence identity to the nucleotide sequence of SEQ ID NO: 49;
the bacterial strain of (iii) comprises a 16S rDNA sequence having at least 99% sequence identity to the nucleotide sequence of SEQ ID NO: 50;
the bacterial strain of (iv) comprises a 16S rDNA sequence having at least 99% sequence identity to the nucleotide sequence of SEQ ID NO: 51;
the bacterial strain of (v) comprises a 16S rDNA sequence having at least 99% sequence identity to the nucleotide sequence of SEQ ID NO: 60;
the bacterial strain of (vi) comprises a 16S rDNA sequence having at least 99% sequence identity to the nucleotide sequence of SEQ ID NO: 64;
the bacterial strain of (vii) comprises a 16S rDNA sequence having at least 99% sequence identity to the nucleotide sequence of SEQ ID NO: 71;
the bacterial strain of (viii) comprises a 16S rDNA sequence having at least 99% sequence identity to the nucleotide sequence of SEQ ID NO: 75; and
the bacterial strain of (ix) comprises a 16S rDNA sequence having at least 99% sequence identity to the nucleotide sequence of SEQ ID NO: 77.

112. The method of claim 111, wherein the composition further comprises one or more bacterial strains comprising 16S rDNA sequences having at least 99% sequence identity to any one of SEQ ID NOs: 42-46, 52-59, 61-63, 65-70, 72-74, and 76.

113. The method of claim 111, wherein the composition comprises:
the bacterial strain comprising a 16S rDNA sequence having at least 99% sequence identity to SEQ ID NO: 48;
the bacterial strain comprising a 16S rDNA sequence having at least 99% sequence identity to SEQ ID NO: 75; and
the bacterial strain comprising a 16S rDNA sequence having at least 99% sequence identity to SEQ ID NO: 77.

114. The method of claim 113, wherein the composition further comprises:
a bacterial strain comprising a 16S rDNA sequence having at least 99% sequence identity to the nucleotide sequence of SEQ ID NO: 52;
a bacterial strain comprising a 16S rDNA sequence having at least 99% sequence identity to the nucleotide sequence of SEQ ID NO: 55;
a bacterial strain comprising a 16S rDNA sequence having at least 99% sequence identity to the nucleotide sequence of SEQ ID NO: 62;
a bacterial strain comprising a 16S rDNA sequence having at least 99% sequence identity to the nucleotide sequence of SEQ ID NO: 67; and
a bacterial strain comprising a 16S rDNA sequence having at least 99% sequence identity to the nucleotide sequence of SEQ ID NO: 72.

115. The method of claim 114, wherein the composition further comprises:
a bacterial strain comprising a 16S rDNA sequence having at least 99% sequence identity to SEQ ID NO: 23; and
a bacterial strain comprising a 16S rDNA sequence having at least 99% sequence identity to SEQ ID NO: 26.

116. The method of claim 110, wherein the pathogenic organism is a pathobiont.

117. The method of claim 116, wherein the pathobiont induces a Th1 response in a subject.

118. The method of claim 110, wherein the pathogenic organism belongs to the family Enterobacteriace.

119. The method of claim 110, wherein the pathogenic organism belongs to the genus Enterococcus.

120. The method of claim 110, wherein the pathogenic organism belongs to the genus Shigella.

121. The method of claim 110, wherein the pathogenic organism belongs to the species Klebsiella pneumoniae.

122. The method of claim 110, wherein the pathogenic organism belongs to the species Escherichia coli.

123. The method of claim 110, wherein the administration suppresses replication and/or survival of the pathogenic organism.

124. The method of claim 110, wherein the composition is a pharmaceutical composition comprising a pharmaceutically acceptable excipient.

125. The method of claim 124, wherein the pharmaceutical composition is formulated for delivery to the intestine.

126. The method of claim 124, wherein the pharmaceutical composition comprises a pH-sensitive composition comprising one or more enteric polymers.

127. A composition comprising a purified bacterial mixture, the purified bacterial mixture comprising:
(i) a bacterial strain comprising a 16S rDNA sequence having at least 97% sequence identity to the nucleotide sequence of SEQ ID NO: 48;
(ii) a bacterial strain comprising a 16S rDNA sequence having at least 97% sequence identity to the nucleotide sequence of SEQ ID NO: 49;
(iii) a bacterial strain comprising a 16S rDNA sequence having at least 97% sequence identity to the nucleotide sequence of SEQ ID NO: 50;
(iv) a bacterial strain comprising a 16S rDNA sequence having at least 97% sequence identity to the nucleotide sequence of SEQ ID NO: 51;
(v) a bacterial strain comprising a 16S rDNA sequence having at least 97% sequence identity to the nucleotide sequence of SEQ ID NO: 60;
(vi) a bacterial strain comprising a 16S rDNA sequence having at least 97% sequence identity to the nucleotide sequence of SEQ ID NO: 64;
(vii) a bacterial strain comprising a 16S rDNA sequence having at least 97% sequence identity to the nucleotide sequence of SEQ ID NO: 71;
(viii) a bacterial strain comprising a 16S rDNA sequence having at least 97% sequence identity to the nucleotide sequence of SEQ ID NO: 75; and/or
(ix) a bacterial strain comprising a 16S rDNA sequence having at least 97% sequence identity to the nucleotide sequence of SEQ ID NO: 77.

128. A method of treating a disease or disorder associated with bacterial colonization in a subject, the method comprising administering to the subject a therapeutically effective amount of the composition of claim 127.

129. The method of claim 128, wherein the disease or disorder is:
(a) associated with a Th1 immune response;
(b) associated with use of a proton pump inhibitor;
(c) an autoimmune disease or an inflammatory bowel disease; and/or
(d) non-alcoholic steatohepatitis (NASH), primary sclerosing cholangitis (PSC), non-alcoholic fatty liver disease (NAFLD), gastroesophageal reflux disease (GERD), or alcoholism.

130. A method of suppressing colonization of an intestine of a subject with oral microbiome bacteria, the method comprising administering to the subject a therapeutically effective amount of the composition of claim 127.


SEQUENCE LISTING (77 sequences)

SEQ ID NO: 1 - DNA, 1022 bp, Alistipes putredinis
  source 1..1022: mol_type = genomic DNA
gctcagcttgcctaggtcgctccttgcggtcacgaacttcaggcacccccggctcccatggcttgacgggcggtgtgtacaaggcccgggaacgtattcaccgcgccatggctgatgcgcgattactagcgaatccaacttcatggaggcgggtttcagcctccaatccgaactgagataggctttcgagattcgcatcccatcgctgggtagctgccctctgtacctaccattgtaacacgtgtgtagccccggacgtaagggccgtgctgatttgacgtcatccccaccttcctctcggcttacaccggcagtcccgccagagtgcccagcttcacctgatggcaactaacggtaggggttgcgctcgttatgggacttaacccgacacctcacggcacgagctgacgacaaccatgcagcacctagtttcgcgccccgaagggaaatcctctttcaagaatcgtcgctaactttcaagcccgggtaaggttcctcgcgtatcatcgaattaaaccacatgttcctccgcttgtgcgggcccccgtcaattcctttgagtttcattcttgcgaacgtactccccaggtggataacttatcgctttcgcttagtcaccgactgtgtatcgccgacaacgagttatcatcgtttactgcgtggactaccagggtatctaatcctgtttgctccccacgctttcgtgcctcaacgtcagatatagtttggtaagctgccttcgcaatcggtgttctgtatgatctctaagcatttcaccgctacaccatacattccgcctaccgcaactactctctagctcaacagtattagaggcacgttcagggttgagccccgaaatttcacctctaacttatcaaaccgcctacgcaccctttaaacccaataaatccggataacgcttgaatcctccgtattaccgcggctgctggcacggagttagccgatccttattcgtacgatactttcagacagatacacgtatctgcgtttaccctcgtaca

SEQ ID NO: 2 - DNA, 1084 bp, Bacteroides uniformis
  source 1..1084: mol_type = genomic DNA
gagcgctaggcttacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgcgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcgggaggaacatgtggtttaattcgatgatacgcgaggaaccttacccgggcttgaattgcaactgaatgatgtggagacatgtcagccgcaagcagttgtgaaggtgctgcatggttgtcgtcagctcgtgccgtgaggtgtcggcttaagtgccataacgagcgcaaccctatcgtagtaccat

SEQ ID NO: 3 - DNA, 769 bp, Bacteroides vulgatus
  source 1..769: mol_type = genomic DNA
acaggttttttccctaagggcgctcctcgcggttacgcacttcaggtacccccggctcccatggcttgacgggcggtgtgtacaaggcccgggaacgtattcaccgcgccgtggctgatgcgcgattactagcgaatccagcttcgtggagtcgggttgcagactccagtccgaactgagagaggtttttgggattggcatccactcgcgtggtagcggccctctgtaccccccattgtaacacgtgtgtagccccggacgtaagggccgtgctgatttgacgtcatccccaccttcctcacatcttacgatggcagtcttgtcagagtcctcagcggaacctgttagtaactgacaacaagggttgcgctcgttatggcacttaagccgacacctcacggcacgagctgacgacaaccatgcagcaccttcacagatgccttgcggcttacggctttcaccgtaattcatctgcaatttaagcccgggtaaggttcctcgcgtatcatcgaattaaaccacatgttcctccgcttgtgcgggcccccgtcaattcctttgagtttcaccgttgccggcgtactccccaggtggaatacttaacgctttcgcttggccgcttgctgtaatgcacaaacagcgagtattcatcgtttaccgtgtggactaccagggtatctaaatcctgtttgatacccacactttcgagcctcaatgtcagttgcagcttagcaggctgcctttattatcggagttcttcgtgatatct

SEQ ID NO: 4 - DNA, 1003 bp, Parabacteroides merdae
  source 1..1003: mol_type = genomic DNA
gtgctcagcttttaccctaggccgatccttgcggttacggacttcaggtacccccggctcccatggcttgacgggcggtgtgtacaaggcccgggaacgtattcaccgcgccatggctgatgcgcgattactagcgaatccagcttcacggagtcgagttgcagactccgatccgaactgagacatggtttggagattagcatcctgtcgccaggtagctgccctttgtccatgccattgtaacacgtgtgtcgccccggatgtaagggccgtgctgatttgacgtcatccccaccttcctcacagcttacgctggcagtctcaccagagtcctcagcttcacctgttagtaactagtgataagggttgcgctcgttatggcacttaagccgacacctcacggcacgagctgacgacaaccatgcagcacctcgtaatctgctattgctagaaggagtgtttccactccggtcagactacgttcaaacccgggtaaggttcctcgcgtatcatcgaattaaaccacatgttcctccgcttgtgcgggcccccgtcaattcctttgagtttcaccgttgccggcgtactccccaggtggattacttaacgctttcgctgtagagcttacattgtatcgcaaactcctagtaatcatcgtttactgcgtggactaccagggtatctaatcctgtttgatccccacgctttcgtgcttcagtgtcagttatggtttagtaagctgccttcgcaatcggagttctgcgtgatatctatgcatttcaccgctacaccacgcattccgcctacctcaaacacactcaagtaacccagtttcaacggcaattttatggttgagccacaaactttcaccgctgacttaaatcaccacctacgcaccctttaacccaataaatccgataacgctcgcatcctccgtattaccgcggctgctgcccggagttagccgatgcttattcatagggtacatacaaaaaggacacgt

SEQ ID NO: 5 - DNA, 895 bp, Bifidobacterium longum
  source 1..895: mol_type = genomic DNA
gggacggatctcccttagacggctccatcccacaaggggttaggccaccggcttcgggtgctgcccactttcatgacttgacgggcggtgtgtacaaggcccgggaacgcattcaccgcgacgttgctgattcgcgattactagcgactccgccttcacgcagtcgagttgcagactgcgatccgaactgagaccggttttcagggatccgctccgcgtcgccgcgtcgcatcccgttgtaccggccattgtagcatgcgtgaagccctggacgtaaggggcatgatgatctgacgtcatccccaccttcctccgagttaaccccggcggtcccccgtgagttcccggcataatccgctggcaacacggggcgagggttgcgctcgttgcgggacttaacccaacatctcacgacacgagctgacgacgaccatgcaccacctgtgaacccgccccgaagggaagccgtatctctacgaccgtcgggaacatgtcaagcccaggtaaggttcttcgcgttgcatcgaattaatccgcatgctccgccgcatgtgcgggcccccgtcaatttctttgagttttagccttgcggccgtactccccaggcgggatgcttaacgcgttagctccgacacggaacccgtggaacgggccccacatccagcatccaccgtttacggcgtggactaccagggtatctaatcctgttcgctccccacgctttcgctcctcagcgtcagtaacggcccagagacctgccttcgccattggtgttcttcccgatatctacacattccaccgttacaccgggaattccagtctcccctaccgcactcaacccgccgtaccggcgcggatccccggtaagcgatggactttcacaccggacgcgagg

SEQ ID NO: 6 - DNA, 1035 bp, Bifidobacterium adolescentis
  source 1..1035: mol_type = genomic DNA
gaattcgagtctcaccttagacggctccccccaaaaggttgggccaccggcttcgggtgctacccactttcatgacttgacgggcggtgtgtacaaggcccgggaacgcattcaccgcggcgttgctgatccgcgattactagcgactccgccttcatggagtcgggttgcagactccaatccgaactgagaccggttttaagggatccgctccacctcgcggtgtcgcatcccgttgtaccggccattgtagcatgcgtgaagccctggacgtaaggggcatgatgatctgacgtcatccccaccttcctccgagttgaccccggcggtcccccgtgagttcccaccacgacgtgctggcaacacagggcgagggttgcgctcgttgcgggacttaacccaacatctcacgacacgagctgacgacgaccatgcaccacctgtgaacccgccccgaagggaggccccatctctggggctgtcgggaacatgtcaagcccaggtaaggttcttcgcgttgcatcgaattaatccgcatgctccgccgcttgtgcgggcccccgtcaatttctttgagttttagccttgcggccgtactccccaggcgggatgcttaacgcgttggctccgacacggagaccgtggaatggtccccacatccagcatccaccgtttacggcgtggactaccagggtatctaatcctgttcgctccccacgctttcgctcctcagcgtcagtgacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataattccggatacgcttgcaccctacgtattaccgcggctgctggcacgtagttagccggtgcttattcgaaaggtacactcactccggagggcttgcttccagtcaaaa

SEQ ID NO: 7 - DNA, 757 bp, unidentified
  source 1..757: mol_type = genomic DNA; note = Blautia obeum
ccaaaaagcgcggcggcgtgcttaccatgcagtcgaacgggaaacttttattgaagcttcggcagatttggttggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctg

SEQ ID NO: 8 - DNA, 1113 bp, Blautia wexlerae
  misc_difference 2: note = n is a, c, g, or t
  misc_difference 5: note = n is a, c, g, or t
  source 1..1113: mol_type = genomic DNA
gngantggcggcgtgcttaccatgcagtcgaacgggaaatacttcattgaaacttcggtggatttaatttatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggggagcatagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccggatccttaatcgggatctttccttcgggacaggcgagacaggggtgcatggttgtcgtcagctcgtgtcgtgagatgttgggtaagtccgcacgagcgcaaccctatcctcagtagcagcatttaagtgggcactctgggggagactgcc

SEQ ID NO: 9 - DNA, 1574 bp, Blautia producta
  source 1..1574: mol_type = genomic DNA
agagtttgatcctggctcaggatgaacgctggcggcgtgcttaacacatgcaagtcgagcgaagcattaagacagatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaatgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggtagccgtatcggaaggtgcggctggatcacctcctttctaaggaagaagaagtagagaaaagtgtttcactgttgagttaccaaga

SEQ ID NO: 10 - DNA, 1528 bp, Clostridium hathewayi
  source 1..1528: mol_type = genomic DNA; note = Hungatella effluvii strain
atgagagttcgatcctggctcaggatgaacgctggcggcgtgcttaacacatgcaagtcgagcgaagcggtttcgatgaagttttcggatggatttgaaatcgacttagcggcggacgggtgagtaacgcgtgggtaacctgccttacactgggggataacagttagaaatgactgctaataccgcataagcgcacagggccgcatggtctggtgcgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaacgtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaatcatttaaccgtgatccctcttcggagcaatggagacaggtggtgcatggttgtcgtcagctcgtgtcgtgagatgttgggttaagtcccgcaacgagcgcaacccttatccttagtagccagcacatgatggtgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagctgccgaaggcgggactgataactggggtgaagtcgtaacaaggtagccgtatcggaaggtgcggctggatcacctccttt

SEQ ID NO: 11 - DNA, 1120 bp, Clostridium bolteae
  source 1..1120: mol_type = genomic DNA
gggcgggcggcgtgctaccatgcaagtcgaacgaagcaattaaatgaagttttcggatggaatttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaggaattgacggggacccgcacaagcggtggagcatgtggtttaattcgaagcaacgcgaagaacttaccaagtcttgacatcctcttgaccggcgtgtaacggcgccttctcttctgggcaagagagacagtggtgcatggttgtcgtcagctcgtgtcgtgagatgttgggttaagtcccgcacgagcgcaacccttttccttatatcacaggtgagctgggccctctaggagactgccaggatactgaggaa

SEQ ID NO: 12 - DNA, 761 bp, Parabacteroides distasonis
  source 1..761: mol_type = genomic DNA
tcgggttttttcctaggccgatcctttcggttactgacttcaggtacccccggctcccatggcttgacgggcggtgtgtacaaggcccgggaacgtattcaccgcgccatggctgatgcgcgattactagcgaatccagcttcacggagtcgggttgcagactccgatcctaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagctttacctgttagtaactagtggcatgggttgcgctcgttatggcacttaagccgacacctcacggcacgagctgacaacaaccatgcaccacctcgcaaacggctattgctaaaaaaggtgtttccacctcggtcctaatgctttcaaacccgggtaaggttcctcgcgtatcatcgaattaaaccacatgttcctccgcttgtgctggcccccgtcattcctttgagtttcaccgttgccggcgtactccccaggtggatcacataacgctttccctgagccgcttactgtgtatcgtacacacctagtgatcatcttttactgcgtggactaacagggtatcctaatcctgtttgatccccacgctttcgtgcatcacgtcagtcatggcttgtgagctgccttcgcaaactgggttctgcaaga

SEQ ID NO: 13 - DNA, 1022 bp, Collinsella aerofaciens
  source 1..1022: mol_type = genomic DNA
ccgtcaaccttcggcgcctcccccctcgcggttgggccggcgacttcgggtgcagacgactcgggtggtgtgacgggcggtgtgtacaaggcccgggaacgcattcaccgcggcatgctgatccgcgattactagcaactccgacttcatgggggcgggttgcagcccccaatccgaactggggccggctttccgggatccgctccccctcgcggggtggcatccctctgtaccggccattgtagcacgtgtgcagcccagggcataaggggcatgatgacttgacgtcgtccccgccctcctccgccttgacggcggcggtcccgcgtgggttcccggcatcacccgatggcaacacgcggcgggggttgcgctcgttgcgggacttaacccaacatctcacgacacgagctgacgacagccatgcaccacctgtatgggctcctctcggccacggggtctcccccgcttcacccatatgtcaagccctggtaaggttcttcgcgttgcttcgaattaagccacatgctccgctgcttgtgcgggcccccgtcaattcctttgagttttagccttgcggccgtactccccaggcgggacgcttaatgcgttggctgcggcacggggggatcgtccccccacacctagcgtccatcgtttacggctgggactaccagggtatctaatcctgttcgctcccccagctttcgcgcctcagcgtcggtctcggcccagagggccgccttcgccaccggtgttccacccgatatctgcgcattccaccgctacaccgggtgttccaccctcccctaccggacccaagccgcggaggttccgggggcttcggggggttgagccccccgcttcgaccccggcctgccgggccgcctacgcgcgctttacgcccaatgaatccggataacgctcgccccctacgtattacgcggctgctggcacgtagttagccggggcttcttctgcaggtacagtcttgactcttccctgctgaaagcggtttacga

SEQ ID NO: 14 - DNA, 918 bp, Coprococcus comes
  source 1..918: mol_type = genomic DNA
tggctgcggcgtgcttaccatgcaagtcgaacgaagcacttatctttgattcttcggatgaagaggtttgtgactgagtggcggacgggtgagtaacgcgtgggtaacctgcctcatacagggggataacagttagaaatgactgctaataccgcataagaccacggagccgcatggctcagtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacagggattagataccctggtagtccacgccgtaaacgatgactactaagtgtcggggagcaaaagctcttcggtgccgcagcaaacgcaataagtagtccacctgggggagtacgtcgcaagaatgaaactcaaaggaattgaccggggacccgcacaacggtggagcatgtggtttaattc

SEQ ID NO: 15 - DNA, 1009 bp, Dorea longicatena
  source 1..1009: mol_type = genomic DNA
cggatcggtcaccttcggcagctccctccttacggttgggtcactgacttcgggcgttactgactcccatggtgtgacgggcggtgtgtacaagacccgggaacgtattcaccgcagcattctgatctgcgattactagcgattccagcttcatgtagtcgagttgcagactacaatccgaactgagacgttatttttgagatttgcttaccctcgcgagttcgcttctctttgtttacgccattgtagcacgtgtgtagccctggtcataaggggcatgatgatttgacgtcatccccaccttcctccaggttatccctggcagtctctccagagtgcccagcttaacctgctggctactgaagataggggttgcgctcgttgcgggacttaacccaacatctcacgacacgagctgacgacaaccatgcaccacctgtcaccgatgttccgaagaaaagcttccattacgaagcggtcatcgggatgtcaagatcaggtaaggttcttcgcgttgcttcgaattaaaccacatgctccaccgcttgtgcgggtccccgtcaattcctttgagtttcattcttgcgaacgtactccccaggtggactgcttattgcgttagctgcggcaccgaatggctttgccacccgacacctagcagtcatcgtttacggcgtggactaccagggtatctaatcctgtttgctccccacgctttcgagcctcaacgtcagtcatcgtccagcaagccgccttcgccactggtgttcctcctaatatctacgcatttcaccgctacactaggaattccacttgcctctccgacactctagctcagcagttccaaatgcagtcccggggttgagccccgggctttcacatctggcttgccgtgccgtctacgctccctttacacccagtaaatcccggataacgcttgccccctacgtattaccggcggctgctggcacgtagttagcccggggcttcttagtcaaggtaccgtcat

SEQ ID NO: 16 - DNA, 1016 bp, unidentified
  source 1..1016: mol_type = genomic DNA; note = Eubacterium halli
tcggctcccttcgaagctccctccataaaggttgggtcactggcttcgggcatttccaactcccatggtgtgacgggcggtgtgtacaaaacccgggaacgtattcaccgcgacattctgattcgcgattactagcgattccagcttcgtgtagtcgggttgcaaactacagtccaaactgggacggcctttttgtggtttgctccccctcgcgggtttgcctcactctgtgaccgccattgtagcacgtgtgtcgcccaaatcataaggggcatgatgatttgacgtcgtccccaccttcctccaggttatccctggcagtctctccaaagtgcccagccttacctgctggctactgaaaataggggttgcgctcgttgcgggacttaacccaacatctcacaacacaagctgacaacaaccatgcaccacctgtctcttctgtcccgaaggaaaactcccattacggagtggtcaaaaggatgtcaagacctggtaaggttcttcgcgttgcttcaaattaaaccacatgctccaccgcttgtgcgggtccccgtcaattcctttgagtttcattcttgcaaacgtactccccaggtggaatacttactgcgttagcggcggcaccgaagcctatacggccccgacacctagtattcatcgtttacggcgtggactaccagggtatctaatcctgtttgctccccacgctttcgtgcctcagtgtcagtaacagtccagcaggccgccttcgccactggtgttcctcctaatatctacgcatttcaccgctacactaggaattccgcctgcttctcctgtactctagctaagcagtttcaaatgcagctccggggttgagcccgggctttcacatctgacttgcactgccacctacgcaccctttacaccaataaatccggataacgcttgctccatacgtattaccgcggctgctggcacgtattagcccggagcttctaatcaggtaccggcattatctccctgctgataga

SEQ ID NO: 17 - DNA, 1077 bp, Faecalibacterium prausnitzii
  source 1..1077: mol_type = genomic DNA
tgggctggcggcgcgctacacatgcagtcgaacgagcgagagagagcttgctttctcgagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaatggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatccttgtgacgatgctagaaatagtatttttcttctgaacacagagacaggtggtgcatggttgtcgtcagctcgtgtcgtgagatgttgggttaagtcccgcaacgagcgccaacccttatggtcagttactaccgca

SEQ ID NO: 18 - DNA, 1019 bp, Parabacteroides merdae
  source 1..1019: mol_type = genomic DNA
tagatttacctaggccgatccttgcggttacggacttcaggtacccccggctcccatggcttgacgggcggtgtgtacaaggcccgggaacgtattcaccgcgccatggctgatgcgcgattactagcgaatccagcttcacggagtcgagttgcagactccgatccgaactgagacatggtttggagattagcatcctgtcaccaggtagctgccctttgtccatgccattgtaacacgtgtgtcgccccggatgtaagggccgtgctgatttgacgtcatccccaccttcctcacagcttacgctggcagtctcaccagagtcctcagcttcacctgttagtaactagtgataagggttgcgctcgttatggcacttaagccgacacctcacggcacgagctgacgacaaccatgcagcacctcgtaatctgctattgctagaaagagtgtttccactccggtcagactacgttcaaacccgggtaaggttcctcgcgtatcatcgaattaaaccacatgttcctccgcttgtgcgggcccccgtcaattcctttgagtttcaccgttgccggcgtactccccaggtggattacttaacgctttcgctgtagagcttacattgtatcgcaaactcctagtaatcatcgtttactgcgtggactaccagggtatctaatcctgtttgatccccacgctttcgtgcttcagtgtcagttatggtttagtaagctgccttcgcaatcggagttctgcgtgatatctatgcatttcaccgctacaccacgcattccgcctacctcaaacacactcaagtaacccagtttcaacggcaattttatggttgagccacaaactttcaccgctgacttaaatcaccacctacgcaccctttaaacccaataaatccggataacgctcgcatcctccgtattaccgcggctgctggcacggagttagccgatgcttattcatagggtacatacaaaaagacacgtcctccactttattccccttta

SEQ ID NO: 19 - DNA, 1036 bp, Parabacteroides distasonis
  source 1..1036: mol_type = genomic DNA
tcagcatgacctaggccgatcctcgcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcttccctttgtccacgccattgtaacacgtgtgtcgccccggatgtaagggccgtgctgatttgacgtcatccccgccttcctcgcagcttacgctggcagtcccaccagagtcctcagcatcacctgttagtaactagtggcaagggttgcgctcgttatggcacttaagccgacacctcacggcacgagctgacgacaaccatgcagcacctcgcaaacggctattgctagaagaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacactgtatcgcaaacagctagtgatcatcgtttactgcgtggactaccagggtatctaatcctgtttgatccccacgctttcgtgcatcagcgtcagtcatggcttggcaggctgccttcgcaatcggggttctgcgtgatatctaagcatttcaccgctacaccacgcattccgcctgcctcaaacatactcaagcctcccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacaggaaacgtccacaactttattcccttataaagaggtttacgat

SEQ ID NO: 20 - DNA, 1031 bp, Prevotella copri
  source 1..1031: mol_type = genomic DNA
cttagctttcgcctaggccgctccttacggtcacggactttaggcgcccccggctttcatggcttgacgggcggtgtgtacaaggcccgggaacgtattcaccgcgccatggctgatgcgcgattactagcgaatccagcttcgtggggtcgggttgcagaccccagtccgaactgagacaggctttaaggatttgatcctttttgcaagggaccgtctctctgtacctgccattgtaacacgtgtgtagccccggacgtaagggccgtgctgatttgacgtcatccccaccttcctcacaccttacggtggcagtgtccccagagtgcccagcttaacctgatggcaactaaggagaggggttgcgctcgttatggcacttaagccgacacctcacggcacgagctgacgacaaccatgcagcaccttcacagaggccccgaagggcgtcattgtctccaaatccttcctctgcaattcaagcccgggtaaggttcctcgcgtatcatcgaattaaaccacatgttcctccgcttgtgcgggcccccgtcaattcctttgagtttcaccgttgccggcgtactccccaggtgggatgcttaatgctttcgcttggccgctgacctattcagaccaacagcgggcatccatcgtttaccgtgcggactaccagggtatctaatcctgttcgatacccgcactttcgagcttcagcgtcagttgcgctccagtgagctgccttcgcaatcggagttcttcgtgatatctaagcatttcaccgctacaccacgaattccgcccactttgtgcgtactcaaggaaaccagttcgcgctgcagtgcaacgttgagcgtctaatttcacaacacgcttaatctccggctacgctccctttaaccaaaaaaaccagataacgccggacctccgtattaccgcggctgctggccggaattagccggccctatcataaggtacatgcaaaaagctaccaaactcactttttccctttacaagagttacaaccataggcc

SEQ ID NO: 21 - DNA, 1090 bp, Roseburia faecis
  source 1..1090: mol_type = genomic DNA
ggggctgggcggcgtgcttaccatgcaagtcgaacgaagcactctatttgattttcttcggaaatgaagattttgtgactgagtggcggacgggtgagtaacgcgtgggtaacctgcctcatacagggggataacagttggaaacgactgctaataccgcataagcgcacaggatcgcatgatccggtgtgaaaaactccggtggtatgagatggacccgcgtctgattagccagttggcagggtaacggcctaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacagcggtggagcatgtggtttattcgaagcaacgcgaagaaccttaccaagtcttgacatcccgatgacagagtatgtaatgtactttctcttcgagcatcggtgacagtgggtgcatggttgtcgtcactcgtgtcgtgagatgttgggttaagtccgcaacgagcgcaacccctgtccttagtagcagcggtg

SEQ ID NO: 22 - DNA, 1056 bp, Ruminococcus faecis
  misc_difference 4: note = n is a, c, g, or t
  source 1..1056: mol_type = genomic DNA
gcgntcggtcaccttcggcagctccctccttacggttgggtcactgacttcgggcgttactgactcccatggtgtgacgggcggtgtgtacaagacccgggaacgtattcaccgcgacattctgattcgcgattactagcgattccagcttcatgtagtcgagttgcagactacaatccgaactgagacgttatttttgggatttgctcgacctcgcggttctgcctccctttgtttacgccattgtagcacgtgtgtagccctgctcataaggggcatgatgatttgacgtcatccccaccttcctccaggttatccctggcagtctctctagagtgcccggccaaaccgctggctactaaagataggggttgcgctcgttgcgggacttaacccaacatctcacgacacgagctgacgacaaccatgcaccacctgtcatccctgtcccgaaggaaaggcaacattacttgccggtcagggagatgtcaagagcaggtaaggttcttcgcgttgcttcgaattaaaccacatgctccaccgcttgtgcgggtccccgtcaattcctttgagtttcattcttgcgaacgtactccccaggtggactacttattgcgtttgctgcggcaccgaacagctttgctgcccgacacctagtagtcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggtactctagattgacagtttccaatgcagtcccggggttgagccccgggttttcacatcagacttgccactccgtctacgctccctttacacccagtaaatccggataacgcttgcaccatacgtattaccgcggctgctggcacgtatttagcctgtgcttctagtcaggtaccgtcattttcttccctgctgatagagctttacataccgaatacttcatcccctcccgcg

SEQ ID NO: 23 - DNA, 1530 bp, Flavonifractor plautii
  source 1..1530: mol_type = genomic DNA
tattgagagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

SEQ ID NO: 24 - DNA, 1529 bp, Blautia producta
  source 1..1529: mol_type = genomic DNA
tcagagagtttgatcctggctcaggatgaacgctggcggcgtgcttaacacatgcaagtcgagcgaagcactaagacagatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaatagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacggggacttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggtagccgtatcggaaggtgcggctggatcacctccttt

SEQ ID NO: 25 - DNA, 1530 bp, Blautia producta
  source 1..1530: mol_type = genomic DNA
tcagagagtttgatcctggctcaggatgaacgctggcggcgtgcttaacacatgcaagtcgagcgaagcacttaagtggatctcttcggattgaaacttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccggcccgtaacggggccttcccttcggggcagaggagacaggtggtgcatggttgtcgtcagctcgtgtcgtgagatgttgggttaagtcccgcaacgagcgcaacccctatccttagtagccagcaggtgaagctgggcactctagggagactgccggggataacccggaggaaggcggggacgacgtcaaatcatcatgccccttatgatttgggctacacacgtgctacaatggcgtaaacaaagggaagcgagacagcgatgttgagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttataggagggagctgccgaaggcgggaccgataactggggtgaagtcgtaacaaggtagccgtatcggaaggtgcggctggatcacctccttt

SEQ ID NO: 26 - DNA, 303 bp, Clostridium ramosum
  source 1..303: mol_type = genomic DNA
gatgaacgctggcggcgtgcctaatacatgcaagtcgaacgcgagcacttgtgctcgagtggcgaacgggtgagtaatacataagtaacctgccctagacagggggataactattggaaacgatagctaagaccgcataggtacggacactgcatggtgaccgtattaaagtgcctcaaagcactggtagaggatggacttatggcgcattagctggttggcggggtaacggcccaccaaggcgacgatgcgtagccgacctgagagggtgaccggccacactgggactgagacacggcccag

SEQ ID NO: 27 - DNA, 324 bp, Flavonifractor plautii
  source 1..324: mol_type = genomic DNA
gatgaacgctggcggcgtgcttaacacatgcaagtcgaacggggtgctcatgacggaggattcgtccaacggattgagttacccagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggccca

SEQ ID NO: 28 - DNA, 1003 bp, unidentified; identical to SEQ ID NO: 4
  source 1..1003: mol_type = genomic DNA; note = Barnesiella/Parabacteroides spp

SEQ ID NO: 29 - DNA, 1527 bp, Clostridium symbiosum
  source 1..1527: mol_type = genomic DNA
atgagagtttgatcctggctcaggatgaacgctggcggcgtgcctaacacatgcaagtcgaacgaagcgatttaacggaagttttcggatggaagttgaattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtgaagtcgtaacaaggtagccgtatcggaaggtgcggctggatcacctccttt

SEQ ID NO: 30 - DNA, 324 bp, Eubacterium fissicatena
  source 1..324: mol_type = genomic DNA
gatgaacgctggcggcgtgcttaacacatgcaagtcgagcgaagcgctttacttagatttcttcggattgaaagttttgcgactgagcggcggacgggtgagtaacgcgtgggtaacctgcctcatacagggggataacagttagaaatgactgctaataccgcataagaccacagtaccgcatggtacagtgggaaaaactccggtggtatgagatggacccgcgtctgattagctagttggtaaggtaacggcttaccaaggcgacgatcagtagccgacctgagagggtgaccggccacattgggactgagacacggccca

SEQ ID NO: 31 - DNA, 1486 bp, unidentified
  source 1..1486: mol_type = genomic DNA; note = Lachnospiraceae bacterium
agagtttgatcctggctcaggataaacgctggcggcgcacataagacatgcaagtcgaacggacttaactcattcttttagattgagagcggttagtggcggactggtgagtaacacgtaagcaacctgcctatcagaggggaataacagtgagaaatcattgctaataccgcatatgctcacagtatcacatgatacagtgaggaaaggagcaatccgctgatagatgggcttgcgcctgattagttagttggtggggtaacggcctaccaagacgacgatcagtagccggactgagaggttgaacggccacattgggactgagatacggcccagactcctacgggaggcagcagtcgggaatattgcgcaatggaggaaactctgacgcagtgacgccgcgtataggaagaaggttttcggattgtaaactattgtcgttagggaagataaaagactgtacctaaggaggaagccccggctaactatgtgccagcagccgcggtaatacatagggggcaagcgttatccggaattattgggtgtaaagggtgcgtagacggaagaacaagttggttgtgaaatccctcggctcaactgaggaactgcaaccaaaactattctccttgagtgtcggagaggaaagtggaattcctagtgtagcggtgaaatgcgtagatattaggaggaacaccagtggcgaaggcgactttctggacgataactgacgttgaggcacgaaagtgtggggagcaaacaggattagataccctggtagtccacactgtaaacgatggatactaggtgtagggtgtattaagcactctgtgccgccgctaacgcattaagtatcccacctggggagtacgaccgcaaggttgaaactcaaaggaattgacgggggcccgcacaagcagtggagtatgtggtttaattcgaagcaacgcgaagaaccttaccagggcttgacatataccggaatatactagagatagtatagtccttcgggactggtatacaggtggtgcatggttgtcgtcagctcgtgtcgtgagatgttgggttaagtcccgcaacgagcgcaacccctatcgttagttgctagcaggtaatgctgagaactctagcgagactgccggtgataaatcggaggaaggtggggatgacgtcaaatcatcatgccctttatgtcctgggctacacacgtactacaatggccgtaacagagggaagcaatatagtgatatggagcaaaaccctaaaagcggtctcagttcggattgaaggctgaaattcgccttcatgaagccggaattgctagtaatggcaggtcagcatactgccgtgaatacgttcccgggccttgtacacaccgcccgtcacaccatgagagttggaaatacccgaagcctgtgagctaactgtaaagaggcagcagtcgaaggtagagccaatgattggggtgaagtcgtaacaaggtagccgt

SEQ ID NO: 32 - DNA, 1528 bp, unidentified
  source 1..1528: mol_type = genomic DNA; note = Dorea Longicatena
aacgagagtttgatcctggctcaggatgaacgctggcggcgtgcttaacacatgcaagtcgagcgaagcactttggaagattcttcggatgatttcctttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 33 - DNA, 1531 bp, Blautia producta
  source 1..1531: mol_type = genomic DNA
atcagagagtttgatcctggctcaggatgaacgctggcggcgtgcttaacacatgcaagtcgagcgaagcacttaagtggatctcttcggattgaaacttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccggcccgtaacggggccttcccttcggggcagaggagacaggtggtgcatggttgtcgtcagctcgtgtcgtgagatgttgggttaagtcccgcaacgagcgcaacccctatccttagtagccagcaggtgaagctgggcactctagggagactgccggggataacccggaggaaggcggggacgacgtcaaatcatcatgccccttatgatttgggctacacacgtgctacaatggcgtaaacaaagggaagcgagacagcgatgttgagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttacaggagggagctgccgaaggcgggaccgataactggggtgaagtcgtaacaaggtagccgtatcggaaggtgcggctggatcacctccttt

SEQ ID NO: 34 - DNA, 1451 bp, Escherichia coli
  misc_difference 490..491: note = n is a, c, g, or t
  misc_difference 566: note = n is a, c, g, or t
  misc_difference 570: note = n is a, c, g, or t
  misc_difference 851..852: note = n is a, c, g, or t
  source 1..1451: mol_type = genomic DNA
agtttgatcatggctcagattgaacgctggcggcaggcctaacacatgcaagtcgaacggtaacaggaacgagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttaccgcagaagaannaccggctaactccgtgccagcagccgcggtaatacggagggtgcaagcgttaatcggaattactgggcgtaaagngcangcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccgganntaacgcgttaagtcgaccgcctggggagtacggccgcaaggttaaaactcaaatgaattgacgggggccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgc

SEQ ID NO: 35 - DNA, 792 bp, Lactococcus lactis
  misc_difference 1..2: note = n is a, c, g, or t
  source 1..792: mol_type = genomic DNA
nnaatttttgttgtgctcatacgtgcagttgagcgctcgaaggttggtacttgtaccctctggatgagcagcgaacgggtgagtaacgcgtggggaatctgcctttgagcgggggaccacatttggaaacgaatgcgaataccgcataaaaactttaaacacaagttttaagtttgaaagatgcaattgcatcactccaagatgatcccgcgttgtattagctagttggtgagggaaaggctccccacggcgatcatacatatccgacctgagagggtgaccggccacattgggactgagccatgatcaaactctgaaaaagaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcgggtgaaatgcgtagatatatggtaggaacaccgggtggcgaaagcggctctctggcctgtaactgacactgaggctcgaaaagcgtggggagcaaaaaggattagataccctggtagtccacgccgta

SEQ ID NO: 36 - DNA, 991 bp, Lactobacillus ruminis
  misc_difference 1: note = n is a, c, g, or t
  source 1..991: mol_type = genomic DNA
nactctgtcaccttaggcggctggctccaaaaggttaccccaccgactttgggtgttacaaactctcatggtgtgacgggcggtgtgtacaaggcccgggaacgtattcaccgcgacatgctgattcgcgattactagcgattccgacttcatgcaggcgagttgcagcctgcaatccgaactgagaacggctttaagagattagcttgccctcgcgagttagcgactcgttgtaccgtccattgtagcacgtgtgtagcccaggtcataaggggcatgatgatttgacgtcatccccaccttcctccggtttgtcaccggcagtctcgccagagtgcccaacttaatgatggcaactgacaataagggttgcgctcgttgcgggacttaacccaacatctcacgacacgagctgacgacaaccatgcaccacctgtcattctgtccccgaagggaacgttccatctctggaattgtcagaagatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactgaagggcggaaaccctccaacacttagcactcatcgtttacggcgtggactaccagggtatctaatcctgtttgctacccacgctttcgaacctcagcgtcagttacagaccagagagccgctttcgccactggtgttcttccatatatctacgcatttcaccgctacacatggagttccactctcctcttctgcactcaagtcttccagtttccaatgcactacttcggttaagccgaaggctttcacatcagacttaaaagaccgcctgcgttccctttacgcccataaatccggacacgctcgccacctacgtattaccgcggctgctggcacgtagttagccgtggctttctggtaga

SEQ ID NO: 37 - DNA, 1559 bp, Lactobacillus ruminis
  source 1..1559: mol_type = genomic DNA
attgagagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgttcatgtaaaagacggcttttgctgtcacttttggatgggcctgcggcgtattaacttgttggtggggtaacggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacgtgcgtgagagtaactgttcacgtattgacggtatctgaccagaaagccacggctaactacgtgccagcagccgcggtaatacgtaggtggcgagcgttgtccggatttattgggcgtaaagggaacgcaggcggtcttttaagtctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgcaacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgcgagggcaagctaatctcttaaagccgttctcagttcggattgcaggctgcaactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccttttggagccagccgcctaaggtgggacagatgattggggtgaagtcgtaacaaggtagccgtaggagaacctgcggctggatcacctcctt

SEQ ID NO: 38 - DNA, 1561 bp, Lactobacillus animalis
  source 1..1561: mol_type = genomic DNA
attgagagtttgatcctggctcaggatgaacgctggcggcgtgcctaatacatgcaagtcgaacgaaacttctttatcaccgagtgcttgcactcaccgataaagagttgagtggcgaacgggtgagtaacacgtgggcaacctgcccaaaagagggggataacacttggaaacaggtgctaataccgcataaccatagttaccgcatggtaactatgtaaaaggtggctatgctaccgcttttggatgggcccgcggcgcattagctagttggtgaggtaaaggcttaccaaggcaatgatgcgtagccgaactgagaggttgatcggccacattgggactgagacacggcccaaactcctacgggaggcagcagtagggaatcttccacaatgggcgaaagcctgatggagcaacgccgcgtgggtgaagaaggtcttcggatcgtaaaaccctgttgttagagaagaaagtgcgtgagagtaactgttcacgtttcgacggtatctaaccagaaagccacggctaactacgtgccagcagccgcggtaatacgtaggtggcaagcgttatccggatttattgggcgtaaagggaacgcaggcggtcttttaagtctgatgtgaaagccttcggcttaaccggagtagtgcattggaaactgggagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaagtgttggagggtttccgcccttcagtgctgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcttctgacaatcctagagataggactttcccttcggggacagaatgacaggtggtgcatggttgtcgtcagctcgtgtcgtgagatgttgggttaagtcccgcaacgagcgcaacccttattgttagttgccagcattaagttgggcactctagcaagactgccggtgacaaaccggaggaaggtggggatgacgtcaaatcatcatgccccttatgacctgggctacacacgtgctacaatggacggtacaacgagtcgcaagaccgcgaggtttagcaaatctcttaaagccgttctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagccggtggggtaaccttttggagccagccgtctaaggtgggacagatgattggggtgaagtcgtaacaaggtagccgtaggagaacctgcggctggatcacctccttt

SEQ ID NO: 39 - DNA, 906 bp, Lactobacillus rhamnosus
  misc_difference 1..2: note = n is a, c, g, or t
  source 1..906: mol_type = genomic DNA
nnctctgttttgcgtgtgatgcagtcgacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaggcggctgtctggtctgtaactgacgctgaggctcgaaagcatgggtagcgaacaggattagataccctggtagtccatgccgtaaacgatgaatgctaggtgttggagggtttccgcccttcagtgccgcgcttacgcatttagcattcgcctggggagtacgaccgcaggttgaacctcaaaggaattg

SEQ ID NO: 40 - DNA, 1207 bp, Lactobacillus rhamnosus
  misc_difference 4..5: note = n is a, c, g, or t
  misc_difference 1180: note = n is a, c, g, or t
  misc_difference 1197..1198: note = n is a, c, g, or t
  source 1..1207: mol_type = genomic DNA
cccnnttgtgtcctatactgcagtctacagtctgaatattgaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctgggggagtacgaccgcaaggttgaactcaaaggaattgacggggcccgcacaagcgggggagcatgtggtttaattcgaagcaacgcgaggaccttaccaggtcttgacatcttttgatcacctgagagatcaggtttccccttcggggcaaatgacagtggtgcatggttgtcgtcagctccgtgtctgagatgttgggtaagtccgcaacaagcgcaacccttatgactagttgcagcttagtgggcactcctagtagactgccggtgacaaccggaggaagggtggggatgactcaatcactagccctnggacctgggctacaacnngtcctcatg

SEQ ID NO: 41 - DNA, 1062 bp, Lactobacillus rhamnosus
  misc_difference 2: note = n is a, c, g, or t
  misc_difference 1046: note = n is a, c, g, or t
  source 1..1062: mol_type = genomic DNA
gntctggtttgttttgttggggggtgaaatctagtattgaggtgcttgcatcttggtttaattgtggaggagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcatccgcctggggagtacgaccgcaaggttgaaactcaaaggaattgacggggcccgcacagcgtggagcatgtggtttaattcgaagcaacgcgagaaccttaccaggtctgacatctttgatcactgagagatcaggtttcccttcgggcaaatgacagtggtgcatggttgtcgtcagctcgtgtctngagatgttgggttaat

SEQ ID NO: 42 - DNA, 1474 bp, Bacteroides caccae
  source 1..1474: mol_type = genomic DNA
tggctcaggatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttgggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagttgtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagccactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccataggccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 43 - DNA, 1473 bp, Bacteroides cellulosilyticus
  source 1..1473: mol_type = genomic DNA
agagtttgatcctggctcaggatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatctttttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacggcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatagtggaaacattatagccgcaaggcatttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgtaaggagcggcctagggtaaaactggtaattggggctaagtcgta

SEQ ID NO: 44 - DNA, 1472 bp, Bacteroides faecis
  source 1..1472: mol_type = genomic DNA
ctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggcataatagaaccgcatggtttttttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 45 - DNA, 929 bp, Bacteroides ovatus
  misc_difference 583: note = k is g or t
  misc_difference 704: note = s is g or c
  source 1..929: mol_type = genomic DNA
cgatatccggatttattggagtttaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtkatgctgaggactctagagagactgccgtcgtaagatgtgaggaaggtggggatgacgtcaaatcagcacggcccttacgtccggggctacacacgtgttacaatggggggtacagaaggcsgctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctaggggtaaaactggtaattggggc

SEQ ID NO: 46 - DNA, 873 bp, Bacteroides thetaiotaomicron
  misc_difference 287: note = w is a or t
  misc_difference 429..430: note = w is a or t
  misc_difference 437..438: note = w is a or t
  misc_difference 439: note = y is c or t
  misc_difference 449: note = k is g or t
  misc_difference 450: note = w is a or t
  misc_difference 454: note = y is c or t
  misc_difference 457: note = y is c or t
  misc_difference 461: note = r is a or g
  misc_difference 464..465: note = w is a or t
  source 1..873: mol_type = genomic DNA
tttaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccwagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcawwtgaatawwytggaaacagkwtagycgyaagrcawwtgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgca

SEQ ID NO: 47 - DNA, 1474 bp, Bacteroides uniformis
  source 1..1474: mol_type = genomic DNA
ctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcgaggagcgccctagggtaaaactggtgattggggctaagtcgtaacaaggta

SEQ ID NO: 48 - DNA, 1503 bp, Bacteroides vulgatus
  source 1..1503: mol_type = genomic DNA
atgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagttcacatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaag

SEQ ID NO: 49 - DNA, 887 bp, Bifidobacterium adolescentis
  misc_difference 11: note = k is g or t
  misc_difference 40: note = y is c or t
  source 1..887: mol_type = genomic DNA
gggctcgtagkcggttcgtcgcgtccggtgtgaaagtccaycgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccatcctttttgggg

SEQ ID NO: 50 - DNA, 1495 bp, Bifidobacterium longum
  source 1..1495: mol_type = genomic DNA
tggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctgg

SEQ ID NO: 51 - DNA, 689 bp, Bifidobacterium pseudocatenulatum
  misc_difference 272: note = w is a or t
  misc_difference 327: note = w is a or t
  misc_difference 328: note = m is a or c
  misc_difference 332: note = k is g or t
  source 1..689: mol_type = genomic DNA
ggttcgtcgcgtccggtgtgaaagtccatcgtttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgwcggagctaacgcgttaagcatcccgcctggggagtacggccgcaaggctaaaacwmaaakaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgttcagggcttcacgcatgctacaatggccggtacaacgggatgcgacacggcgacgtg

SEQ ID NO: 52 - DNA, 1394 bp, Blautia coccoides
  source 1..1394: mol_type = genomic DNA
tgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggta

SEQ ID NO: 53 - DNA, 918 bp, Clostridium citroniae
  source 1..918: mol_type = genomic DNA
tccggatttactggagtagtaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaa

SEQ ID NO: 54 - DNA, 1307 bp, Clostridium clostridioforme
  source 1..1307: mol_type = genomic DNA
taataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccggggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcaggtagagccgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgt

SEQ ID NO: 55 - DNA, 1475 bp, Clostridium innocuum
  source 1..1475: mol_type = genomic DNA
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcggccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccga

SEQ ID NO: 56 - DNA, 1475 bp, Clostridium innocuum
  source 1..1475: mol_type = genomic DNA
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccga

SEQ ID NO: 57 - DNA, 906 bp, Clostridium sordellii
  misc_difference 114: note = r is a or g
  misc_difference 116: note = y is c or t
  misc_difference 277: note = m is a or c
  misc_difference 897: note = r is a or g
  source 1..906: mol_type = genomic DNA
acacatgcaagtcgagcgaacccttcggggtgagcggcggacgggtgagtaacgcgtgggtaacctgccctgtacacacggataacataccgaaaggtatgctaatacgggatrayatatgagagtcgcatggcttttgtatcaaagctccggcggtacaggatggacccgcgtctgattagctagttggtaaggtaacggcttaccaaggcaacgatcagtagccgacctgagagggtgatcggccacattggaactgagacacggtccaaactcmtacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggcggtctttcaagccagaagtgaaaggctacggctcaaccgtagtaagcttttggaactgtaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttatctaarcttgacatc

SEQ ID NO: 58 - DNA, 380 bp, Coprococcus comes
  misc_difference 59: note = r is a or g
  misc_difference 78: note = k is g or t
  misc_difference 355: note = k is g or t
  misc_difference 367: note = s is g or c
  misc_difference 378: note = r is a or g
  source 1..380: mol_type = genomic DNA
acrggaggcagcagtggggaatattgcacaatgggggaaaccctgatgcagcgacgccrcgtgagcgaagaagtattkcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaaggcgggggctcccccccggggactgctttggaaactatgcagctagactgtcggacaggtaagtggaattcccagtgtagcggtgaaatgcgtagatattgggaggaacagcaktgggtaaggctscttacaggacrat

SEQ ID NO: 59 - DNA, 1317 bp, Dorea longicatena
  source 1..1317: mol_type = genomic DNA
taacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaagg

SEQ ID NO: 60 - DNA, 808 bp, unidentified
  misc_difference 94: note = k is g or t
  misc_difference 253: note = r is a or g
  misc_difference 598: note = r is a or g
  misc_difference 736: note = r is a or g
  misc_difference 778: note = r is a or g
  misc_difference 795: note = m is a or c
  source 1..808: mol_type = genomic DNA; note = Erysipelatoclostridium ramosum
gagggagcaggcggcagcaagggtctgtggtgaaagcctgaagttaaacttcagtaagccatagaaaccaggcagctagagtgcaggagaggakcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactragtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaargcggggatgacgtcaaatcatcatgccccttatgacctgggctacacacgtgctacaatggatggtgcagagggaagcgaagccgcgaggtgaagcaaaacccataaaaccattctcagttcggattgtagtctgcarctcgactacatgaagttggaatcgctagtaatcgcgaatcarcatgtcgcgatgaatamgttctcgggcctt

SEQ ID NO: 61 - DNA, 1521 bp, Eubacterium rectale
  source 1..1521: mol_type = genomic DNA
agagtttgatcctggctcaggatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtgaagtcgtaacaaggtagccgtatcggaaggtgcggctggatcacct

SEQ ID NO: 62 - DNA, 1482 bp, Odoribacter splanchnicus
  source 1..1482: mol_type = genomic DNA
agagtttgatcctggctcaggatgaacgctagcgacaggcttaacacatgcaagtcgaggggcatcatgaggtagcaataccttgatggcgaccggcgcacgggtgagtaacgcgtatgcaacctgcccgataccggggtatagcccatggaaacgtggattaacaccccatagtacttttatcctgcatgggatgtgagttaaatgtttaaggtatcggatgggcatgcgtcctattagttagttggcggggtaacagcccaccaagacgatgataggtaggggttctgagaggaaggtcccccacattggaactgagacacggtccaaactcctacgggaggcagcagtgaggaatattggtcaatggacgagagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacaggattagataccctggtagtccacgccgtaaacgatgctcactggttctgtgcgatatattgtacgggattaagcgaaagtattaagtgagccacctggggagtacgtcggcaacgatgaaactcaaaggaattgacgggggcccgcacaagcggaggaacatgtggtttaattcgatgatacgcgaggaaccttacctgggtttaaatgggaaatgtcgtatttggaaacagatattctcttcggagcgtttttcaaggtgctgcatggttgtcgtcagctcgtgccgtgaggtgtcgggttaagtcccataacgagcgcaacccttaccgttagttgctagcatgtaatgatgagcactctaacgggactgccaccgtaaggtgagaggaaggcggggatgacgtcaaatcagcacggcccttacacccagggctacacacgtgttacaatggccggtacagagggccgctaccaggtgactggatgccaatctcaaaagccggtcgtagttcggattggagtctgtaacccgactccatgaagttggattcgctagtaatcgcgcatcagccatggcgcggtgaatacgttcccgggccttgtacacaccgcccgtcaagccatggaagccgggggtgcctgaagtccgtaaccgcgaggatcggcctagggcaaaactggtaactggggctaagtcgtaacaaggta

SEQ ID NO: 63 - DNA, 738 bp, Parabacteroides distasonis
  misc_difference 12: note = r is a or g
  misc_difference 30: note = s is g or c
  misc_difference 72: note = r is a or g
  misc_difference 130: note = y is c or t
  misc_difference 140: note = r is a or g
  misc_difference 206..306: note = r is a or g
  misc_difference 319: note = s is g or c
  misc_difference 322: note = w is a or t
  misc_difference 323: note = r is a or g
  misc_difference 411: note = y is c or t
  misc_difference 412: note = r is a or g
  misc_difference 658: note = w is a or t
  source 1..738: mol_type = genomic DNA
cgaggggcagcrcaggagttagcaataccsggtggcgaccggcgcacgggtgagtaacgcgtatgcaacttrcctatcagagggggataacccggcgaaagtcggactaataccgcatgaagcagggatyccgcatgggratatttgctaaagattcatcgctgatagataggcatgcgttccattaggcagttggcggggtaacrgcccaccaaaccgacgatggataggggttctgagaggaaggtcccccacattggtactgagacacggaccaaactcctacgggaggcagcagtgaggaatattggtcaatggscgwragsctgaaccagccaagtcgcgtgagggatgaaggttctatggatcgtaaacctcttttataagggaataaagtgcgggacgtgtccyrttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgwttgcgaaggcagcctgccaagccgtaactgacgcggatgcacgaaagcgtggggatcaaacaggattagataccctggta

SEQ ID NO: 64 - DNA, 521 bp, Parabacteroides merdae
  misc_difference 249: note = k is g or t
  misc_difference 466: note = m is a or c
  misc_difference 466: note = m is a or c
  misc_difference 467: note = s is g or c
  misc_difference 469: note = r is a or g
  misc_difference 510: note = w is a or t
  source 1..521: mol_type = genomic DNA
catgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttckgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgmsarcmgccgcgggaatacggaagatgcagagcgttatccggatwtattggggtta

SEQ ID NO: 65 - DNA, 1368 bp, Bacteroides xylanisolvens
  source 1..1368: mol_type = genomic DNA
catgcaagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggg

SEQ ID NO: 66 - DNA, 1399 bp, unidentified
  source 1..1399: mol_type = genomic DNA; note = Blautia obeum
ggcgtgcttaacacatgcaagtcgaacgggaaaccttttattgaagcttcggcagatttagctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgttccttaaccggaactttccttcgggacaggggagacaggtggtgcatggttgtcgtcagctcgtgtcgtgagatgttgggttaagtcccgcaacgagcgcaacccctatccccagtagccagcagtccggctgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgc

SEQ ID NO: 67 - DNA, 1485 bp, Alistipes putredinis
  source 1..1485: mol_type = genomic DNA
agagtttgatcctggctcaggatgaacgctagcggcaggcttaacacatgcaagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctgaagttcgtgaccgcaaggagcgacctagggcaaaactggtgactggggctaagtcgtaacaaggta

SEQ ID NO: 68 - DNA, 1464 bp, Collinsella aerofaciens
  source 1..1464: mol_type = genomic DNA
agagttcgatcctggctcaggatgaacgctggcggcgcgcctaacacatgcaagtcgaacggcacctatcttcggatagaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgccggcccaaccgagaggggggaggcgccgaaggtgtggagggtgaggggggtgaagtcgtaacaaggta

SEQ ID NO: 69 - DNA, 1472 bp, Bacteroides faecis
  source 1..1472: mol_type = genomic DNA
ctcaggatgaacgctagctacaggcttaacacatgcaagtcgaggggcagcattccagtttgcttgcaaactggagatggcgaccggcgcacgggtgagtaacacgtatccaacctgccgataactcggggatagcctttcgaaagaaagattaatacccgatggcataatagaaccgcatggtttgattattaaagaatttcggttatcgatggggatgcgttccattaggcagttggtggggtaacggcccaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtggtccacgtgtggatttttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggcgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 70 - DNA, 317 bp, Alistipes shahii
  misc_difference 11: note = w is a or t
  misc_difference 12: note = s is g or c
  misc_difference 15: note = w is a or t
  misc_difference 16: note = w is g or t
  misc_difference 22: note = w is g or t
  misc_difference 23: note = r is a or g
  misc_difference 26: note = s is g or c
  misc_difference 27: note = r is a or g
  misc_difference 35: note = s is g or c
  misc_difference 36: note = r is a or g
  misc_difference 44: note = m is a or c
  misc_difference 50: note = w is a or t
  misc_difference 52: note = m is a or c
  misc_difference 59: note = m is a or c
  misc_difference 67: note = r is a or g
  misc_difference 90..91: note = r is a or g
  misc_difference 120: note = s is g or c
  misc_difference 125: note = r is a or g
  misc_difference 138: note = k is g or t
  misc_difference 145: note = k is g or t
  misc_difference 147: note = k is g or t
  misc_difference 163: note = r is a or g
  misc_difference 165..166: note = r is a or g
  misc_difference 201: note = s is g or c
  misc_difference 206: note = r is a or g
  misc_difference 315: note = r is a or g
  source 1..317: mol_type = genomic DNA
acatagggggwstgwkaggttwrccscccacattsrtactgagmcatgawcmaactctmtacgggargsagsagtgaggaatattggtcrrtggacgcaagtctgaaccagccatgccgsgtgcrggaagacggctckatgagtkgkaaactgcttttgtacrarrgtaaacgctcttacgtgtaagagcctgaaagtatsgtacraatgaggatcggctaactccgtgccagcagccgcggtaatacggaggatccaagcgttatccggatttattgggtttaaagggtgcgtaggcgggttgataaagttagrgg

SEQ ID NO: 71 - DNA, 811 bp, Anaerostipes caccae
  misc_difference 32: note = r is a or g
  source 1..811: mol_type = genomic DNA
gcttacacatgcaagtcgaacgaagcatttargattgaagttttcggatggatttcctatatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaa

SEQ ID NO: 72 - DNA, 728 bp, Phascolarctobacterium faecium
  source 1..728: mol_type = genomic DNA
cggagaattttcatttcggtagaattcttagtggcgaacgggtgagtaacgcgtaggcaacctgccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacagagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtttgacgctgagatgcgaaagccagggtagc

SEQ ID NO: 73 - DNA, 707 bp, unidentified
  misc_difference 287: note = r is a or g
  misc_difference 360: note = m is a or c
  source 1..707: mol_type = genomic DNA; note = Agathobaculum butyriciproducens
tagtggcggacgggtgagtaacgcgtgagcaatctgcctttaagagggggataacagtcggaaacggctgctaataccgcataaagcattgaattcgcatgttttcgatgccaaaggagcaatccgcttttagatgagctcgcgtctgattagctagttggcggggtaacggcccaccaaggcgacgatcagtagccggactgagaggttgaacggccacattgggactgagacacggcccagactcctacgggaggcagcagtggggaatattgcgcaatgggggraaccctgacgcagcaacgccgcgtgattgaagaaggccttcgggttgtaaagatctttaatcagggacgaaamatgacggtacctgaagaataagctccggctaactacgtgccagcagccgcggtaatacgtagggagcaagcgttatccggatttactgggtgtaaagggcgcgcaggcgggccggcaagttggaagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggata

SEQ ID NO: 74 - DNA, 1525 bp, Bacteroides fragilis
  source 1..1525: mol_type = genomic DNA
atgaagagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatggcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaagcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggctaagtcgtaacaaggtagccgtaccggaaggtgcggctggaacacctcctt

SEQ ID NO: 75 - DNA, 1022 bp, Fusobacterium mortiferum
  source 1..1022: mol_type = genomic DNA
tggctcaggatgaacgctgacagaatgcttaacacatgcaagtctacttgatccttcgggtgatggtggcggacgggtgagtaacgcgtaaagaacttgccctgcagtctgggacaacatttggaaacgaatgctaataccggatattatgtatttctcgcatgagttttacatgaaagctatatgcgctgcaggagagctttgcgtcctattagctagttggtgaggtaacggctcaccaaggccatgataggtagccggcctgagagggtgaacggccacaaggggactgagacacggcccttactcctacgggaggcagcagtggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgctaaactagagtactggagaggtgggcggaactacaagtgtagaggtgaaattcgtagatatttgtaggaatgccgatggggaagccagcccactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctaagaaattagcagagatgcttttgtgccccttcgggggaacttagtgacaggtggtgcatggctgtcgtcagctcgtgt

SEQ ID NO: 76 - DNA, 1490 bp, unidentified
  source 1..1490: mol_type = genomic DNA; note = Paraclostridium benzoelyticum
agagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatacgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaaaccaatgactggggtgaagtcgtaacaaggtagccgtatcggaaggtgcggctggatcacct

SEQ ID NO: 77 - DNA, 1538 bp, Escherichia fergusonii
  source 1..1538: mol_type = genomic DNA
ttgaagagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt